(19) United States
(12) Patent Application Publication
(10) Pub. No.: US 2019/0192697 A1
(43) Pub. Date: Jun. 27, 2019

(54) CHEMICAL PROBES OF LYSYL OXIDASK-LIKE 2 AND USES THEREOF

(71) Applicant: PHARMAKEA, INC., San Diego, CA (US)

(72) Inventors: Martin W. ROWBOTTOM, San Diego, CA (US); John Howard HUTCHINSON, San Diego, CA (US); Jillian Frances EVANS, San Diego, CA (US); Gretchen BAIN, San Diego, CA (US)

(21) Appl. No.: 16/331,076

(22) PCT Filed: Sep. 6, 2017
(86) PCT No.: PCT/US2017/050313

§ 371 Date: Mar. 6, 2019

Related U.S. Application Data
(60) Provisional application No. 62/384,642, filed on Sep. 7, 2016.

(51) Int. Cl. A61K 49/00; A61K 49/08; A61K 49/10; A61K 51/04; G01N 33/50; G01N 33/573

(52) U.S. Cl. CPC A61K 49/0032; A61K 49/0052; A61K 49/085; A61K 49/10; A61K 2123/00; A61K 51/0497; G01N 33/5088; G01N 33/573; A61K 51/0455

(57) ABSTRACT

Described herein are probe compounds that interact with the LOXL2 receptor, methods of making such probe compounds, and methods of using such probe compounds in vitro and in vivo.

### RELATED APPLICATIONS

This application claims the benefit of U.S. Provisional Patent Application No. 62/384,642 entitled “CHEMICAL PROBES OF LYSYL OXIDASE-LIKE 2 AND USES THEREOF” filed on Sep. 7, 2016, which is incorporated by reference in its entirety.

### FIELD OF THE INVENTION

Described herein are probe compounds for lysyl oxidase-like 2 (LOXL2), methods of making such compounds, and methods of using such LOXL2 probe compounds.

### BACKGROUND OF THE INVENTION

Lysyl oxidase like-2 (LOXL2) is an amine oxidase enzyme that catalyzes crosslinking of extracellular matrix proteins. LOXL2 is also involved in intracellular processes such as mediating epithelial-to-mesenchymal transition of cells. LOXL2 signaling is implicated in, for example, in fibrotic diseases and cancer.

### SUMMARY OF THE INVENTION

Probe compounds described herein are useful for the profiling of LOXL2 within a complex cellular environment. In some embodiments, probe compounds described herein are used to evaluate the interactions of LOXL2 inhibitors with the proteome. The proteome is defined as the combination or the assembly of all the proteins expressed by a given organism, biological system, tissue or cell at a given time under given conditions. The methods and probe compounds described herein can be applied to advance the fields of biomarker discovery, in vivo imaging, and small molecule screening and drug target discovery.

Probe compounds described herein comprise three elements: (i) a reactive group or ‘warhead’; (ii) a linker region; and (iii) a tag. The reactive group or ‘warhead’ provides selectivity for LOXL2. In some embodiments, the linker can be designed to control specificity of the probe compound for target tissues or cells. The tag is used for the detection, isolation, or detection and isolation of the probe compound from a complex cellular environment.

In one aspect, provided herein is a probe compound comprising:
(a) a small molecule lysyl oxidase like-2 (LOXL2) inhibitor (LOXL2i);
(b) a tag moiety (Q) for the detection, isolation, or detection and isolation of the small molecule LOXL2i bound to LOXL2; and
(c) an optional linker (L) that separates the LOXL2 inhibitor from the tag moiety.

In some embodiments, the small molecule LOXL2i is selective for LOXL2 versus lysyl oxidase (LOX). In some embodiments, the small molecule LOXL2i binds to the lysine tyrosylquinone (LTQ)-dependent amine oxidase of LOXL2.

In some embodiments, the small molecule LOXL2i is a substituted or unsubstituted heterocyclylmethylamine compound, or a substituted or unsubstituted arylmethylamine compound.

In some embodiments, the small molecule LOXL2i is a substituted or unsubstituted heteroarylmethylamine.

In some embodiments, the small molecule LOXL2i is a substituted or unsubstituted (monocyclic heteroaryl)methylamine.

In some embodiments, the small molecule LOXL2i is a substituted or unsubstituted (6-membered monocyclic heteroaryl)methylamine. In some embodiments, the small molecule LOXL2i is a substituted or unsubstituted pyridinylmethylamine, substituted or unsubstituted pyrimidinylmethylamine, substituted or unsubstituted pyrazinylmethylamine, substituted or unsubstituted pyridazinylmethylamine, or substituted or unsubstituted triazinylmethylamine.

In some embodiments, the small molecule LOXL2i is a substituted or unsubstituted pyridinylmethylamine or a substituted or unsubstituted pyrimidinylmethylamine. In some embodiments, the small molecule LOXL2i is a substituted or unsubstituted pyridinylmethylamine. In some embodiments, the small molecule LOXL2i is a substituted or unsubstituted pyrimidinylmethylamine.

In some embodiments, the small molecule LOXL2i is a substituted or unsubstituted (5-membered monocyclic heteroaryl)methylamine. In some embodiments, the small molecule LOXL2i is a substituted or unsubstituted (5-membered monocyclic heteroaryl)methylamine that is a substituted or unsubstituted imidazolylmethylamine, substituted or unsubstituted pyrazolylmethylamine, substituted or unsubstituted triazolylmethylamine, substituted or unsubstituted furylmethylamine, substituted or unsubstituted thienylmethylamine, substituted or unsubstituted isoxazolylmethylamine, substituted or unsubstituted thiazolylmethylamine, substituted or unsubstituted oxazolylmethylamine, substituted or unsubstituted isothiazolyl methylamine, substituted or unsubstituted pyrrolylmethylamine, substituted or unsubstituted oxadiazolylmethylamine, substituted or unsubstituted thiadiazolyl methylamine, or substituted or unsubstituted furazanylmethylamine.

In some embodiments, the small molecule LOXL2i is a substituted or unsubstituted (bicyclic heteroaryl)methylamine. In some embodiments, the small molecule LOXL2i is a substituted or unsubstituted (bicyclic heteroaryl)methylamine that is a is substituted or unsubstituted indolizinylmethylamine, substituted or unsubstituted indolylmethylamine, substituted or unsubstituted benzofuranylmethylamine, substituted or unsubstituted benzothiophenylmethylamine, substituted or unsubstituted indazolylmethylamine, benzimidazolylmethylamine, substituted or unsubstituted purinylmethylamine, substituted or unsubstituted quinolizinylmethylamine, substituted or unsubstituted quinolinylmethylamine, substituted or unsubstituted isoquinolinylmethylamine, substituted or unsubstituted cinnolinylmethylamine, substituted or unsubstituted phthalazinylmethylamine, substituted or unsubstituted quinazolinylmethylamine, substituted or unsubstituted quinoxalinylmethylamine, substituted or unsubstituted 1,8-naphthyridinylmethylamine, or substituted or unsubstituted pteridinylmethylamine.

In some embodiments, the small molecule LOXL2i is a substituted or unsubstituted bicyclic heterocyclylmethylamine that is a substituted or unsubstituted (bicyclic heterocyclyl)methylamine. In some embodiments, the bicyclic heterocyclyl is substituted or unsubstituted quinolinonylmethylamine, substituted or unsubstituted isoquinolinonylmethylamine, substituted or unsubstituted chromonylmethylamine, or substituted or unsubstituted coumarinylmethylamine.

In some embodiments, the small molecule LOXL2i is a substituted or unsubstituted phenylmethylamine or a substituted or unsubstituted naphthylmethylamine.

In some embodiments, the tag moiety (Q) for the detection, isolation, or detection and isolation of the small molecule LOXL2i bound to LOXL2 is selected from the group consisting of: a solid support, a reporter group, a tag used for affinity purification, a tag used for sorting or immobilizing the compound of Formula (I) on a solid support, a hapten, a fluorescent moiety, radioactive moiety, magnetic resonance imaging (Mill) moiety, colorometric moiety, luminescent moiety, bioluminescent moiety, chemiluminescent moiety, oligonucleotide or combination thereof.

In one aspect, the probe compound described herein is a compound that has the following structure of Formula (I):

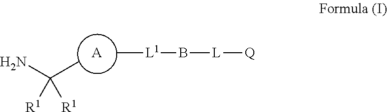

wherein,
Q is a tag moiety for the detection, isolation, or detection and isolation of the compound of Formula (I) in a biological sample;
or Q is absent provided that the compound of Formula (I) comprises a radioactive or an isotopic variant of any atom in the compound of Formula (I);
L is absent or a linker;
each R¹ is independently H, D, or F;
ring A is an unsubstituted or substituted aryl, or an unsubstituted or substituted heterocycle, wherein if ring A is substituted then ring A is substituted with 1, 2, or 3 R^{a} groups;
L¹ is X¹—Y¹—, —Y¹—X¹—, or Y¹;
   X¹ is —O—, —S—, —S(═O)—, —S(═O)₂—, —C(═O)—, —C(═O)O—, —C(═O)NR²—, —NR²C(═O)—, or —NR₂—;
      R² is H, C₁-C₆alkyl, C₁-C₆fluoroalkyl, or C₁-C₆deuteroalkyl;
   Y¹ is absent, or C₁-C₆alkylene;
B is absent or an unsubstituted or substituted monocyclic carbocycle, unsubstituted or substituted bicyclic carbocycle, unsubstituted or substituted monocyclic heterocycle, or unsubstituted or substituted bicyclic heterocycle, wherein if B is substituted then B is substituted with one or more R^{b};
each R^{a}, and R^{b} is independently selected from the group consisting of H, D, halogen, CN, —OR⁵, —SR⁵, —S(═O)R⁴, —S(═O)₂R⁴, —S(═O)₂N(R⁵)₂, NR⁵S(═O)₂R⁴, C(═O)R⁴, OC(═O)R⁴, CO₂R⁵, OCO₂R⁴, N(R⁴)₂, OC(═O)N(R⁵)₂, —NHC(═O)R⁴, —NHC(═O)OR⁴, C₁-C₆alkyl, C₁-C₆fluoroalkyl, C₁-C₆deuteroalkyl, C₁-C₆heteroalkyl, substituted or unsubstituted C₃-C₁₀cycloalkyl, substituted or unsubstituted C₂-C₁₀heterocycloalkyl, substituted or unsubstituted aryl, or substituted or unsubstituted heteroaryl;
each R⁴ is independently selected from C₁-C₆alkyl, C₁-C₆fluoroalkyl, C₁-C₆deuteroalkyl, C₁-C₆heteroalkyl, substituted or unsubstituted C₃-C₁₀cycloalkyl, substituted or unsubstituted C₂-C₁₀heterocycloalkyl, substituted or unsubstituted aryl, and substituted or unsubstituted heteroaryl;
each R⁵ is independently selected from H, C₁-C₆alkyl, C₁-C₆fluoroalkyl, C₁-C₆deuteroalkyl, C₁-C₆heteroalkyl, substituted or unsubstituted C₃-C₁₀cycloalkyl, substituted or unsubstituted C₂-C₁₀heterocycloalkyl, substituted or unsubstituted aryl, substituted or unsubstituted heteroaryl, —C₁-C₄alkylene-(substituted or unsubstituted C₃-C₈cycloalkyl), substituted or unsubstituted C₂-C₈heterocycloalkyl, —C₁-C₄alkylene-(substituted or unsubstituted C₂-C₈heterocycloalkyl), substituted or unsubstituted aryl, —C₁-C₄alkylene-(substituted or unsubstituted aryl), substituted or unsubstituted heteroaryl, and —C₁-C₄alkylene-(substituted or unsubstituted heteroaryl);
or two R⁵ on the same N atom are taken together with the N atom to which they are attached to a substituted or unsubstituted N-containing heterocycle.

In some embodiments, L is absent or a linker with the formula -L²-C-L³-;
L² is X²—Y²—, —Y²—X²—, or Y²;
   X² is —O—, —S—, —S(═O)—, —S(═O)₂—, —S(═O)₂NR³—, —C(═O)—, —C(═O)O—, —C(═O)NR³—, —NR³C(═O)—, —NR³S(═O)₂—, or —NR³—;
      R³ is H, C₁-C₆fluoroalkyl, or C₁-C₆deuteroalkyl;
   Y² is absent, or C₁-C₆alkylene;
C is absent, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₈cycloalkyl, —C₁-C₄alkylene-(substituted or unsubstituted C₃-C₈cycloalkyl), substituted or unsubstituted C₂-C₈heterocycloalkyl, —C₁-C₄alkylene-(substituted or unsubstituted C₂-C₈heterocycloalkyl), substituted or unsubstituted aryl, —C₁-C₄alkylene-(substituted or unsubstituted aryl), substituted or unsubstituted heteroaryl, or —C₁-C₄alkylene-(substituted or unsubstituted heteroaryl); wherein if C is substituted then C is substituted with one or more R^{c};
or when C and R³ are attached to the same N-atom then C and R³ are taken together with the N atom to which they are attached to form ring D, wherein ring D is a substituted or unsubstituted N-containing heterocycle, wherein if ring D is substituted then ring D is substituted with 1, 2, or 3 R^{d};
each R^{c}, and R^{d}, is independently selected from the group consisting of D, halogen, CN, —OR⁵, —SR⁵, —S(═O)R⁴, —S(═O)₂R⁴, —S(═O)₂N(R⁵)₂, NR⁵S(═O)₂R⁴, C(═O)R⁴, OC(═O)R⁴, CO₂R⁵, OCO₂R⁴, N(R⁴)₂, OC(═O)N(R⁵)₂, —NHC(═O)R⁴, —NHC(═O)OR⁴, C₁-C₆fluoroalkyl, C₁-C₆deuteroalkyl, C₁-C₆heteroalkyl, substituted or unsubstituted C₃-C₁₀cycloalkyl, substituted or unsubstituted C₂-C₁₀heterocycloalkyl, substituted or unsubstituted aryl, or substituted or unsubstituted heteroaryl;
or two R^{d} groups attached to the same carbon atom are taken together with carbon atom to which they are attached to form either a substituted or unsubstituted carbocycle or substituted or unsubstituted heterocycle;
L³ is absent or -L⁴-L⁵-L⁶-L⁷-;
   L⁴ is absent, —O—, —S—, —S(═O)—, —S(═O)_{2—}, —CH(OH)—, —C(═O)—, —C(═O)NH—, —NHC(═O)—, —C(═O)O—, —OC(═O)—, —CH(═N)—, —CH(═N—NH)—, —CCH₃(═N)—, —CCH₃(═N—NH)—, —OC(═O)NH—, —NHC(═O)NH—, —NHC(═O)O—, —(CH₂)ₚ—, —(OCH₂CH₂)ₚ—, or —(OCH₂CH₂)ₚ—, p is 1, 2, 3, 4, 5, or 6;
   L⁵ is absent, unsubstituted or substituted alkylene, unsubstituted or substituted heteroalkylene, unsubstituted or substituted alkenylene, unsubstituted or substituted alkynylene, unsubstituted or substituted cycloalkylene, unsubstituted or substituted heterocycloalkylene, unsubstituted or substituted arylene, unsubstituted or substituted heteroarylene, —(OCH₂CH₂)ₚ—, or —(OCH₂CH₂)ₚ—, p is 1, 2, 3, 4, 5, 6, 7, 8, 9, 10, 11, or 12;
   L⁶ is absent, —O—, —S—, —S(O)—, —S(O)_{2—}, —NR⁴—, —CH(OH)—, —C(═O)—, —C(═O)NH—, —NHC(═O)—, —C(═O)O—, —OC(═O)—, —OC(═O)NH—, —NHC(═O)NH—, or —NHC(═O)O—;
   L⁷ is absent, unsubstituted or substituted alkylene, unsubstituted or substituted heteroalkylene.

In some embodiments, ring A is an unsubstituted or substituted heterocycle, wherein if ring A is substituted then ring A is substituted with 1, 2, or 3 R^{a} groups.

In some embodiments, ring A is an unsubstituted or substituted monocyclic aromatic heterocycle, wherein if ring A is substituted then ring A is substituted with 1, 2, or 3 R^{a} groups.

In some embodiments, ring A is an unsubstituted or substituted monocyclic aromatic 6-membered heterocycle or an unsubstituted or substituted monocyclic aromatic 5-membered heterocycle, wherein if ring A is substituted then ring A is substituted with 1, 2, or 3 R^{a} groups.

In some embodiments, ring A is an unsubstituted or substituted pyridinyl, an unsubstituted or substituted pyrimidinyl, an unsubstituted or substituted pyrazinyl, an unsubstituted or substituted pyridazinyl, or an unsubstituted or substituted triazinyl, wherein if ring A is substituted then ring A is substituted with 1, 2, or 3 R^{a} groups.

In some embodiments, ring A is an unsubstituted or substituted pyridinyl, or an unsubstituted or substituted pyrimidinyl, wherein if ring A is substituted then ring A is substituted with R^{a}.

In some embodiments, the compound of Formula (I) has the following structure of Formula (II) or Formula (III):

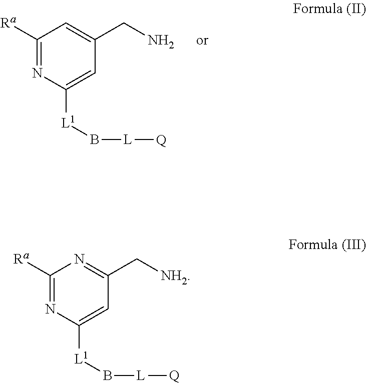

In some embodiments, the compound of Formula (I) has the following structure of Formula (IIa):

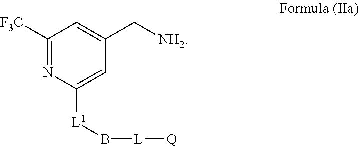

In some embodiments, ring A is an unsubstituted or substituted monocyclic aromatic 5-membered heterocycle that is an unsubstituted or substituted imidazolyl, an unsubstituted or substituted pyrazolyl, an unsubstituted or substituted triazolyl, an unsubstituted or substituted tetrazolyl, an unsubstituted or substituted furyl, an unsubstituted or substituted thienyl, an unsubstituted or substituted isoxazolyl, an unsubstituted or substituted thiazolyl, an unsubstituted or substituted oxazolyl, an unsubstituted or substituted isothiazolyl, an unsubstituted or substituted pyrrolyl, an unsubstituted or substituted oxadiazolyl, an unsubstituted or substituted thiadiazolyl, or an unsubstituted or substituted furazanyl.

In some embodiments, ring A is an unsubstituted or substituted bicyclic heterocycle.

In some embodiments, ring A is an unsubstituted or substituted quinolinone, unsubstituted or substituted isoquinolinone, unsubstituted or substituted chromone, or unsubstituted or substituted coumarin.

In some embodiments, the compound of Formula (I) has the following structure of Formula (IV), Formula (V), Formula (VI), Formula (VII), or Formula (VIII):

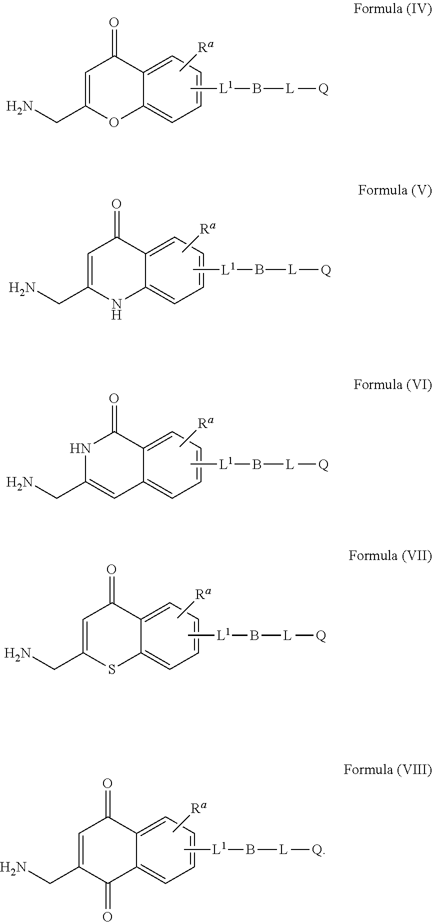

In some embodiments, ring A is an unsubstituted or substituted indolizinyl, unsubstituted or substituted indolyl, unsubstituted or substituted benzofuranyl, unsubstituted or substituted benzothiophenyl, unsubstituted or substituted indazolyl, unsubstituted or substituted benzimidazolyl, unsubstituted or substituted purinyl, unsubstituted or substituted quinolizinyl, unsubstituted or substituted quinolinyl, unsubstituted or substituted isoquinolinyl, unsubstituted or substituted cinnolinyl, unsubstituted or substituted phthalazinyl, unsubstituted or substituted quinazolinyl, unsubstituted or substituted quinoxalinyl, unsubstituted or substituted 1,8-naphthyridinyl, or unsubstituted or substituted pteridinyl.

In some embodiments, the compound of Formula (I) has the following structure of Formula (IX), Formula (X), Formula (XI), or Formula (XII):

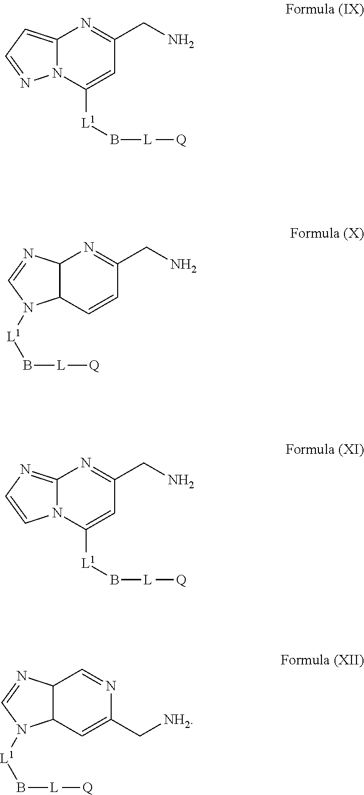

In some embodiments, ring A is an unsubstituted or substituted phenyl, or an unsubstituted or substituted naphthyl.

In some embodiments, the compound of Formula (I) has the structure of Formula (IIb):

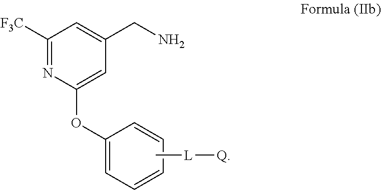

In some embodiments, the compound of Formula (I) has the structure of Formula (IIc):

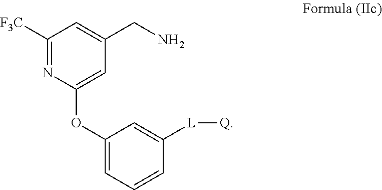

In some embodiments, Q is a tag moiety for the detection, isolation, or detection and isolation of the compound of Formula (I) in a biological sample that is selected from the group consisting of: a solid support, a reporter group, a tag used for affinity purification, a tag used for sorting or immobilizing the compound of Formula (I) on a solid support, a hapten, a fluorescent moiety, radioactive moiety, magnetic resonance imaging (MRI) moiety, colorometric moiety, luminescent moiety, bioluminescent moiety, chemiluminescent moiety, oligonucleotide or combination thereof; or Q is absent provided that the compound of Formula (I) comprises a radioactive or an isotopic variant of any atom in the compound of Formula (I).

In some embodiments, Q is a tag used for affinity purification that is capable of specific binding to a known protein to produce a tightly bound complex.

In some embodiments, Q is a tag that is capable of specific binding to avidin or streptavidin. In some embodiments, Q is biotin or desthiobiotin.

In some embodiments, Q is a hapten selected from biotin, a coumarin dye, a rhodamine dye, a xanthene dye (such as fluorescein), a cyanine dye, a BODIPY dye, a Lucifer yellow dye, digoxigenin, dansyl, or dintrophenyl.

In some embodiments, Q is a tag moiety that is selected from the group consisting of: a fluorescent moiety, radioactive moiety, colorometric moiety, luminescent moiety, chemiluminescent moiety, or combination thereof.

In some embodiments, Q is a tag moiety that is a fluorescent moiety. In some embodiments, Q is a tag moiety that is a fluorescent moiety selected from the group consisting of xanthene dyes, cyanine dyes, squaraine dyes, ring-substituted squaraine dyes, naphthalene dyes, coumarin dyes, oxadiazole dyes, anthracene dyes, oxazine dyes, acridine dyes, arylmethine dyes, BODIPY dyes, and tetrapyrrole dyes. In some embodiments, Q is a fluorescent moiety selected from the group consisting fluorescein dyes, rhodamine dyes, Oregon green dyes, eosin dyes, Texas red dyes, cyanine dyes, indocarbocyanine dyes, oxacarbocyanine dyes, thiacarbocyanine dyes, merocyanine dyes, Seta, SeTau, Square dyes, dansyl dyes, prodan dyes, coumarin dyes, BODIPY dyes, pyridyloxazole dyes, nitrobenzoxadiazole dyes, benzoxadiazole dyes, DRAQ5, DRAQ7, CyTRAK Orange cascade blue, Nile red, Nile blue, cresyl violet, oxazine 170, proflavin dyes, acridine orange dyes, acridine yellow dyes, auramine dyes, crystal violet dyes, malachite green dyes, porphin dyes, phthalocyanine dyes, and bilirubin dyes. In some embodiments, Q is xanthene, cyanine, squaraine, naphthalene, coumarin, oxadiazole, anthracene, pyrene, oxazine, acridine, arylmethine, tetrapyrrole, dansyl, BODIPY. In some embodiments, Q is cyanine, coumarin, or dansyl. In some embodiments, Q is xanthene, cyanine 2, cyanine 3, cyanine 3B, cyanine 3.5, cyanine 5, cyanine 5.5, cyanine7, squaraine, naphthalene, coumarin, oxadiazole, anthracene, pyrene, oxazine, acridine, arylmethine, tetrapyrrole, dansyl, BODIPY FL, BODIPY R6G, BODIPY TMR, BODIPY 581/591, BODIPY TR, BODIPY 630/650, or BODIPY 650/665.

In some embodiments, Q is a tag moiety that is a chemiluminescent moiety. In some embodiments, Q is a chemiluminescent moiety that generates light or a colored product upon treatment with peroxide or a peroxidase. In some embodiments, Q is luminol, isoluminol, N-(4-aminobutyl)-N-ethyl isoluminol (ABEI), N-(4-aminobutyl)-N-methyl isoluminol (ABMI), 2,2′-azino-bis(3-ethylbenzothiazoline-6-sulphonic acid) (ABTS), 3,3′,5,5′-Tetramethylbenzidine (TMB), 3,3′-diaminobenzidine (DAB), o-phenylenediamine dihydrochloride (OPD), AmplexRed, AEC, or homovanillic acid.

In some embodiments, Q is a chemiluminescent moiety that generates light or a colored product upon treatment with horseradish peroxidase (HRP). In some embodiments, Q is 3,3′-diaminobenzidine (DAB), 3,3′,5,5′-tetramethylbenzidine (TMB), 2,2′-Azinobis [3-ethylbenzothiazoline-6-sulfonic acid] (ABTS), o-phenylenediamine dihydrochloride (OPD).

In some embodiments, Q is a substrate for a luciferase enzyme. In some embodiments, Q is D-luciferin, or coelenterazine.

In some embodiments, Q is a chemiluminescent moiety that generates light or a colored product upon treatment with alkaline phosphatase (AP). In some embodiments, Q is nitro blue tetrazolium chloride (NBT), 5-bromo-4-chloro-3-indolyl phosphate (BCIP), or p-Nitrophenyl Phosphate (PNPP).

In some embodiments, Q is a chemiluminescent moiety that generates light or a colored product upon treatment with glucose oxidase. In some embodiments, Q is nitro blue tetrazolium chloride (NBT).

In some embodiments, Q is a chemiluminescent moiety that generates light or a colored product upon treatment with β-galactosidase. In some embodiments, Q is 5-bromo-4-chloro-3-indoyl-β-D-galactopyranoside (BCIG or X-Gal).

In some embodiments, Q is absent and the compound of Formula (I) comprises a radioactive or an isotopic variant of any atom in the compound of Formula (I).

In some embodiments, the compound of Formula (I) comprises a radioactive or an isotopic variant of any atom in the compound of Formula (I) and is suitable for use in positron emission tomography (PET) analysis. In some embodiments, Q is absent and the compound of Formula (I) comprises one or more atoms selected from tritium (³H), fluorine-18 (¹⁸F), carbon-11 (¹¹C), carbon-14 (¹⁴C), nitrogen-13 (¹³N), oxygen-15 (¹⁵O), or sulfur-35 (³⁵S). In some embodiments, Q comprises a chelated radioactive isotope.

In some embodiments, Q comprises a chelated radioactive isotope that is suitable for positron emission tomography (PET) analysis. In some embodiments, Q comprises a chelated radioactive isotope, wherein Q is a diethylenetriaminepentaacetic acid (DTPA) chelate, 1,4,7,10-tetraazacyclododecane-1,4,7,10-tetraacetic acid (DOTA) chelate, or 1,4,7-triazacyclononane-1,4,7-trisacetic acid (NOTA) chelate or 1,4,7,10-tetraazacyclododecane-1,4,7,10-tetramethyl-1,4,7,10-tetraacetic acid (DOTMA) chelate or a radioactive isotope. In some embodiments, Q comprises a chelated radioactive isotope that is copper-64 (⁶⁴Cu), gallium-68 (⁶⁸Ga), or technetium-99m (^{99m}Tc).

In some embodiments, Q is a magnetic resonance imaging (Mill) moiety. In some embodiments, Q comprises a chelate of an atom that is suitable for magnetic resonance imaging (MRI). In some embodiments, Q comprises a chelate of an atom that is suitable for magnetic resonance imaging (MRI) that is a diethylenetriaminepentaacetic acid (DTPA) chelate, 1,4,7,10-tetraazacyclododecane-1,4,7,10-tetraacetic acid (DOTA) chelate, 1,4,7-triazacyclononane-1,4,7-trisacetic acid (NOTA) chelate, or 1,4,7,10-tetraazacyclododecane-1,4,7,10-tetramethyl-1,4,7,10-tetraacetic acid (DOTMA) chelate. In some embodiments, Q comprises a chelate of copper, gallium, thulium, europium, gadolinium, or manganese. In some embodiments, Q comprises a chelate of gadolinium that is selected from gadoterate, gadodiamide, gadobenate, gadopentetate, gadoteridol, gadoversetamide, gadoxetate, gadobutrol, or gadofosveset.

In some embodiments, Q is a solid support. In some embodiments, Q is a solid support that is a nanoparticle, bead, or resin. In some embodiments, Q is a nanoparticle or bead comprising one or more metals selected from iron, cobalt, nickel, gadolium, chromium, manganese or gold. In some embodiments, Q is a nanoparticle or bead that is magnetic or paramagnetic. In some embodiments, the magnetic moiety is a ferrite bead.

Any combination of the groups described above for the various variables is contemplated herein. Throughout the specification, groups and substituents thereof are chosen by one skilled in the field to provide stable moieties and compounds.

In one aspect, when it is suitable and practical to do so, probe compounds are formulated in a pharmaceutical composition comprising a compound described herein, or a pharmaceutically acceptable salt, or solvate thereof, and at least one pharmaceutically acceptable excipient. In some embodiments, the pharmaceutical composition is formulated for administration to a mammal by intravenous administration, subcutaneous administration, oral administration, inhalation, nasal administration, dermal administration, or ophthalmic administration. In some embodiments, the pharmaceutical composition is formulated for administration to a mammal by intravenous administration, subcutaneous administration, or oral administration. In some embodiments, the pharmaceutical composition is formulated for administration to a mammal by oral administration. In some embodiments, the pharmaceutical composition is in the form of a tablet, a pill, a capsule, a liquid, a suspension, a gel, a dispersion, a solution, an emulsion, an ointment, or a lotion. In some embodiments, the pharmaceutical composition is in the form of a tablet, a pill, or a capsule.

In any of the aforementioned aspects are further embodiments in which the probe compound described herein, or a pharmaceutically acceptable salt thereof, is: (a) systemically administered to the mammal; and/or (b) administered orally to the mammal; and/or (c) intravenously administered to the mammal; and/or (d) administered by inhalation; and/or (e) administered by nasal administration; or and/or (f) administered by injection to the mammal; and/or (g) administered topically to the mammal; and/or (h) administered by ophthalmic administration; and/or (i) administered rectally to the mammal; and/or (j) administered non-systemically or locally to the mammal.

In any of the aforementioned aspects are further embodiments comprising single administrations of the effective amount of the compound, including further embodiments in which the compound is administered once a day to the mammal or the compound is administered to the mammal multiple times over the span of one day. In some embodiments, the compound is administered on a continuous dosing schedule. In some embodiments, the compound is administered on a continuous daily dosing schedule.

In any of the embodiments disclosed herein, the mammal is a human.

In some embodiments, compounds provided herein are administered to a human.

In some embodiments, compounds provided herein are orally administered.

Kits are also provided. Kits include a probe compound described herein and a container. In some embodiments, the kit includes instructions or information on the use(s) of the probe compound.

Articles of manufacture, which include packaging material, a probe compound described herein, or a pharmaceutically acceptable salt thereof, within the packaging material, and a label that indicates that the compound or composition, or pharmaceutically acceptable salt, or pharmaceutically acceptable solvate thereof, is used for inhibiting the activity of LOXL2, or for the analysis of LOXL2 interaction with a LOXL2, or for the identifying the presence of LOXL2 in a biological sample, are provided.

Other objects, features and advantages of the compounds, methods and compositions described herein will become apparent from the following detailed description. It should be understood, however, that the detailed description and the specific examples, while indicating specific embodiments, are given by way of illustration only, since various changes and modifications within the spirit and scope of the instant disclosure will become apparent to those skilled in the art from this detailed description.

### BRIEF DESCRIPTION OF THE FIGURES

FIG. 1 depicts a general strategy for evaluating target engagement with probe compounds described herein.

FIG. 2 depicts a general strategy for the use of probe compounds containing a biotin moiety to capture and detect, isolate and quantify free (unbound) LOXL2 enzyme from ex vivo biological samples or in vitro systems.

FIG. 3a depicts a strategy for the use of probe compounds containing a fluorescent moiety to detect, and quantify LOXL2.

FIG. 3b depicts a general strategy for the use of probe compounds containing a radiolabeled moiety, or radioactive isotope, (useful for PET imaging) to detect, and quantify LOXL2.

FIG. 3c depicts a general strategy for the use of probe compounds containing a contrast agent moiety (useful for Mill imaging) to detect, and quantify LOXL2.

FIG. 4 depicts a general strategy for the use of probe compounds coupled to a magnetic bead to isolate, detect, and quantify LOXL2.

FIG. 5 shows the results of the ELISA-based quantification of LOXL2 bound to a streptavidin coated plate after capture of the free/unbound LOXL2 using a biotinylated LOXL2 inhibitor (Compound 1-1). A concentration range of human and mouse LOXL2 was tested. Purified recombinant human MAO-B was used as the negative control.

FIG. 6. Western blot analysis of free/unbound LOXL2 captured using Compound 1-7 (chemically-coupled to FG beads). 10 mL plasma from two different human donors was pre-incubated with vehicle or 1 μM or 10 μM Compound 1-13a for 2 hours at 37° C. prior to incubation with LOXL2 inhibitor-coupled FG beads. Arrow indicates full length LOXL2.

FIG. 7. Western blot analysis of free/unbound LOXL2 captured using Compound 1-7 chemically-coupled FG beads. Blood was drawn and plasma isolated pre-administration and at 2, 24 and 48 hours post oral administration of Compound 1-13a (150 mg) or placebo to healthy volunteers. Six subjects were administered Compound 1-13a (Subjects 2 and 4-8) and two were administered placebo (Subjects 1 and 3). Free (non-Compound 1-13a bound) LOXL2 was captured using 8 mL plasma from each subject and analyzed by Western blotting.

FIG. 8. Erenna®-based fluorescence assay for analysis of free/unbound LOXL2. Various concentrations of LOXL2 were diluted in buffer and then quantitated by incubating with compound 1-2 followed by a fluorescently-labeled detection antibody and streptavidin coated magnetic microparticles (MPs). Shown is the mean Detected Events (DE) for 2-3 replicates of each concentration.

FIG. 9. Erenna®-based fluorescence assay for analysis of free/unbound LOXL2. Solutions of 500 pg/mL LOXL2 were pre-incubated with various concentrations of Compound 1-13A (or vehicle) at 37° C. for 1 hour and then free/unbound LOXL2 quantitated by incubating with Compound 1-2 followed by a fluorescently-labeled detection antibody and streptavidin coated magnetic microparticle (MPs). Shown is the mean pg/mL LOXL2 calculated from 2-3 replicates.

FIG. 10a shows the overall % injected dose (% ID)/g tissue biodistribution data for all organs after administration of Compound 1-13 to mice. FIG. 10b shows a closer view of the % injected dose (% ID)/g biodistribution data from FIG. 10a for specific organs.

FIG. 11 shows concentrations of plasma LOXL2 measured in healthy male and female subjects and Scleroderma patients (n=10 each) measured using the Erenna®-based assay using compound 1-2 (*p=0.04 upaired t-test of SSc (female) vs. Healthy (female) as described in Example B-5, Method 2.

FIG. 12 shows % Target Engagement of circulating LOXL2 measured in plasma from healthy human volunteers following dosing with Compound 1-13a versus Compound 1-13a concentration in plasma using the procedure described in Example B-5, method 1.

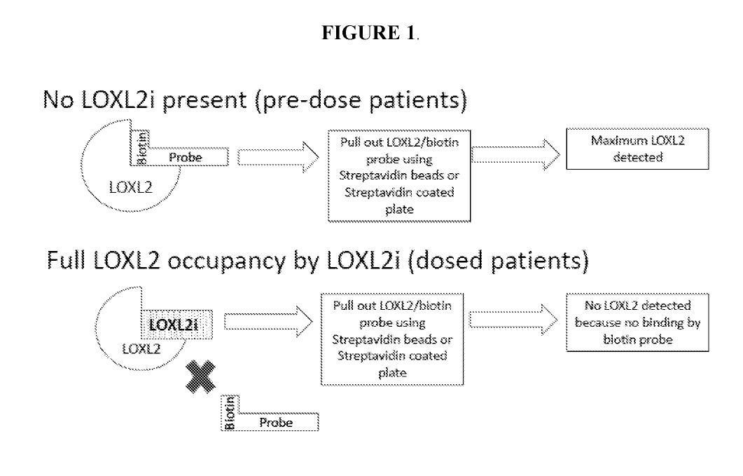

### DETAILED DESCRIPTION OF THE INVENTION

Many treatments for human disease employ small-molecule inhibitors of a specific protein, or proteins, involved in the progression of the disease of interest. It is often useful to be able to quantify the extent of binding of such small-molecule inhibitors to specific proteins in vivo, with the aim of understanding, for instance, the extent of target engagement and its relationship to pharmacodynamics and/or pharmacokinetics. A key aspect of studying and quantifying small-molecule-protein interactions is developing suitable techniques for the detection of such complexes. Many of these techniques involve capturing proteins with a detectable binding partner, such as an appropriate small-molecule inhibitor probe. The small-molecule inhibitor probe includes a moiety that allows for the detection, isolation, or detection and isolation of the probe.

After capturing the protein with the small-molecule probe compounds described herein, the tag moiety is detected directly or undergoes a chemical interaction with a reagent to form a detectable product that may be quantified. In some embodiments, the small molecule probe is a LOXL2 inhibitor (LOXL2i). In some embodiments the captured protein is LOXL2.

Suitable reporter tag moieties include but are not limited to fluorophores, chromophores, drugs, nanoparticles, biopolymers, radiolabeled moieties, antibodies or antibody fragments, affinity moieties, magnetic moieties, albumin binding moieties, contrast agent moieties, or chelating agents. In some embodiments, the reporter tag moiety is a fluorophore, which is detected by a specific wavelength of light. Alternatively, the tag moiety is a biotin moiety, which binds to a binding partner, such as streptavidin (or variants thereof), or streptavidin coated magnetic or non-magnetic beads to form an isolable and detectable molecular complex. Alternatively, in other embodiments, the tag moiety is a magnetic moiety. In some embodiments, the magnetic moiety is a magnetic bead attached through a cleavable linking moiety, allowing isolation and detection of the small-molecule-protein complex. Alternatively, in some embodiments, the tag moiety is a radiolabeled moiety, such as a radioactive isotope. Examples of suitable radioactive isotopes include but are not limited to carbon-11, nitrogen-13, fluorine-18, hydrogen-3 or gallium-68, useful for positron emission tomography (PET) imaging.

Alternatively, in some embodiments, the reporter moiety is a contrast agent moiety, that is suitable for MRI use. Examples of suitable contrast agent moieties, include be are not limited to, thulium, europium, gadolinium, or manganese.

Accordingly, the compounds described herein are LOXL2 inhibitors containing at least one suitable tag moiety that are useful for quantifying unbound lysyl oxidase like-2 (LOXL2) from ex vivo biological samples or in vitro systems. In particular, the compounds of Formula (I) described herein are used to capture and detect free (unbound) LOXL2 enzyme from ex vivo biological samples or in vitro systems. Further, the compounds described herein are useful for the development of target engagement assays. In some instances, the compounds are useful for determining the extent of LOXL2 inhibition in patients after administration of a LOXL2 inhibitor. In some instances, the compounds described herein are useful for assessing the pharmacokinetics of a LOXL2 inhibitor in a mammal and for evaluating the tissue distribution of any one of the compounds disclosed herein in a mammal following administration of the compound.

### Lysyl Oxidase Like-2 (LOXL2)

Lysyl oxidase like-2 (LOXL2) is a member of the lysyl oxidase (LOX) family, which comprises Cu²⁺ and lysine tyrosylquinone (LTQ)-dependent amine oxidases. The family comprises five genes: lox (LOX), loxl1 (lysyl oxidase like-1, LOXL1), loxl2 (LOXL2), loxl3 (lysyl oxidase like-3, LOXL3), and loxl4 (lysyl oxidase like-4, LOXL4). The LOX family is known for catalyzing the oxidative deamination of the ε-amino group of lysines and hydroxylysines in collagen and elastin to promote crosslinking of these molecules. Crosslinking of collagen and elastin is essential for maintaining tensile strength of the extracellular matrix.

LOXL2 has been demonstrated to have intracellular functions aside from its role in remodeling of the extracellular matrix. LOXL2 positively regulates the epithelial-to-mesenchymal transition (EMT) transducer, Snail1, by promoting Snail1 stability and functional activity. LOXL2 contributes positively to the activation of the focal adhesion kinase (FAK) signaling pathway and participates in the organization of focal adhesion complexes. Silencing of loxl2 gene leads to reacquisition of epithelial cell polarity and decreases the migratory and invasive ability of mammary cell lines. The modulation of cell adhesion and cell polarity has been reported to be mediated by intracellular LOXL2. LOXL2 transcriptionally represses E-cadherin as well as tight junction and cell polarity genes by Snail1-dependent and Snail1-independent mechanisms. LOXL2 has been more recently described to be associated with chromatin and reported to be involved in histone H3 deamination, a function that is dependent on the LOXL2 catalytic domain.

In some embodiments, the methods disclosed herein are methods for inhibiting intracellular LOXL2. In some embodiments, the methods disclosed herein are methods for inhibiting extracellular (secreted) LOXL2. In some embodiments, the methods disclosed herein are methods for inhibiting extracellular and intracellular LOXL2.

### Fibrosis

LOXL2 has been shown to be involved in fibrotic processes. Fibrotic processes include an excessive deposition of extracellular matrix components, such as collagen, which alters the physical, biochemical and biomechanical matrix properties leading to defective organ function and organ failure. Tissue fibrosis is also associated with cancer progression by direct promotion of cellular transformation and metastasis. Tumors are typically stiffer than normal tissue and tumor rigidity influences tumor metastasis.

Excessive LOXL2 enzyme activity has been implicated in the increased stiffness of tumors. Elevated LOXL2 is also associated with fibrotic lesions from livers of patients suffering from Wilson disease and primary biliary cirrhosis. Additionally, the administration of a LOXL2-specific monoclonal antibody AB0023 was efficacious in reducing disease in a model of fibrosis. AB0023 was shown to inhibit the production of growth factors and of crosslinked collagenous matrix and TGF-beta signaling.

In some embodiments, probe compounds described herein are used in target validation and disease biology studies involving the administration of LOXL2 inhibitors to mammals with fibrosis. In some embodiments, the probe compounds described herein covalently react with LOXL2.

In some embodiments, probe compounds described herein are used to evaluate the role of LOXL2 inhibitors (LOXL2i) and LOXL2 in the treatment of fibrosis in mammals.

“Fibrosis,” as used herein, refers to the accumulation of extracellular matrix constituents that occurs following trauma, inflammation, tissue repair, immunological reactions, cellular hyperplasia, and neoplasia.

In some embodiments, probe compounds described herein are used to evaluate the reduction of fibrosis in a tissue comprising contacting a fibrotic cell or tissue with a probe compound disclosed herein following administration of a LOXL2i.

In some embodiments, the fibrosis comprises lung fibrosis, liver fibrosis, kidney fibrosis, cardiac fibrosis, peritoneal fibrosis, ocular fibrosis or cutaneous fibrosis. In some embodiments, the fibrosis comprises lung fibrosis. In some embodiments, the fibrosis comprises liver fibrosis. In some embodiments, the fibrosis comprises kidney fibrosis. In some embodiments, the fibrosis comprises cardiac fibrosis. In some embodiments, the fibrosis comprises peritoneal fibrosis. In some embodiments, the fibrosis comprises ocular fibrosis. In some embodiments, the fibrosis comprises cutaneous fibrosis.

In some embodiments, reducing fibrosis, or treatment of a fibrotic condition, includes reducing or inhibiting one or more of: formation or deposition of extracellular matrix proteins; the number of pro-fibrotic cell types (e.g., fibroblast or immune cell numbers); cellular collagen or hydroxyproline content within a fibrotic lesion; expression or activity of a fibrogenic protein; or reducing fibrosis associated with an inflammatory response.

In some embodiments, the fibrotic condition is a fibrotic condition of the lung.

In some embodiments, the fibrotic condition is a fibrotic condition of the liver.

In some embodiments, the fibrotic condition is a fibrotic condition of the heart.

In some embodiments, the fibrotic condition is a fibrotic condition of the kidney.

In some embodiments, the fibrotic condition is a fibrotic condition of the skin.

In some embodiments, the fibrotic condition is a fibrotic condition of the eye.

In some embodiments, the fibrotic condition is a fibrotic condition of the gastrointestinal tract.

In some embodiments, the fibrotic condition is a fibrotic condition of the bone marrow.

In some embodiments, the fibrotic condition is a fibrotic condition of the ear.

In some embodiments, the fibrotic condition is idiopathic. In some embodiments, the fibrotic condition is associated with (e.g., is secondary to) a disease (e.g., an infectious disease, an inflammatory disease, an autoimmune disease, a malignant or cancerous disease, and/or a connective disease); a toxin; an insult (e.g., an environmental hazard (e.g., asbestos, coal dust, polycyclic aromatic hydrocarbons), cigarette smoking, a wound); a medical treatment (e.g., surgical incision, chemotherapy or radiation), or a combination thereof.

In some embodiments, probe compounds described herein are used to evaluate the role of a LOXL2i or LOXL2 in the treatment or prevention of fibrosis in a mammal.

In some embodiments, probe compounds described herein are used to evaluate the role of a LOXL2i or LOXL2 in improving lung function in a mammal. In some embodiments, the mammal has been diagnosed as having lung fibrosis.

In some embodiments, probe compounds described herein are used to evaluate the role of a LOXL2i or LOXL2 in the treatment of idiopathic pulmonary fibrosis in a mammal.

In some embodiments, probe compounds described herein are used to evaluate the role of a LOXL2i or LOXL2 in controlling an abnormal accumulation or activation of cells, fibronectin, collagen or increased fibroblast recruitment in a tissue of a mammal. In some embodiments, the abnormal accumulation or activation of cells, fibronectin, collagen or increased fibroblast recruitment in the tissue results in fibrosis.

In some embodiments, probe compounds described herein are used to evaluate the role of a LOXL2i or LOXL2 in the treatment or prevention of scleroderma in a mammal.

In some embodiments, probe compounds described herein are used to evaluate the role of a LOXL2i or LOXL2 in reducing undesired or abnormal dermal thickening in a mammal. In some embodiments, the dermal thickening is associated with scleroderma.

In some embodiments, probe compounds described herein are used to evaluate the role of a LOXL2i or LOXL2 in controlling an abnormal accumulation or activation of cells, fibronectin, collagen or increased fibroblast recruitment in tissues of a mammal. In some embodiments, the abnormal accumulation or activation of cells, fibronectin, collagen or increased fibroblast recruitment in the dermal tissues results in fibrosis. In some embodiments, probe compounds described herein are used to evaluate the role of a LOXL2i or LOXL2 in the reducing hydroxyproline content in tissues of a mammal with fibrosis. Cancer

LOXL2 has been shown to be involved in signaling related to cancer cell growth, adhesion, motility and invasion. Specifically, LOXL2 induces epithelial-to-mesenchymal transition (EMT) of cells to promote tumor invasion. LOXL2 is also upregulated in hypoxic tumor environments which leads to enhanced invasion of tumor cells. LOXL2 has also been shown to promote angiogenesis in hypoxic tumor environments.

Increased LOXL2 expression is associated with poor prognosis in patients with colon, esophageal tumors, oral squamous cell carcinomas, laryngeal squamous cell carcinomas, and head and neck squamous cell carcinomas. LOXL2 has been proposed to participate in cancers of the breast, colon, gastric, head and neck, lung, and melanoma.

In some embodiments, probe compounds described herein are used to evaluate the role of a LOXL2i or LOXL2 in the treatment of cancer in a mammal.

The term “cancer” as used herein, refers to an abnormal growth of cells that tend to proliferate in an uncontrolled way and, in some cases, to metastasize (spread). Types of cancer include, but are not limited to, solid tumors (such as those of the bladder, bowel, brain, breast, endometrium, heart, kidney, lung, liver, uterus, lymphatic tissue (lymphoma), ovary, pancreas or other endocrine organ (thyroid), prostate, skin (melanoma or basal cell cancer) or hematological tumors (such as the leukemias and lymphomas) at any stage of the disease with or without metastases.

### Uses

In some embodiments, the compounds disclosed herein are used to quantify free (unbound) LOXL2 enzyme from ex vivo biological samples or in vitro systems. In some further embodiments, the compounds described herein are useful for the development of target engagement assays. In some embodiments, the compounds described herein are useful for evaluation of the pharmacodynamics or pharmacokinetics of a LOXL2i.

Provided herein is a method for quantifying LOXL2 expression in a target tissue of a mammal comprising: administering at least one of the probe compounds disclosed herein to the mammal or to cells isolated from the mammal; waiting for a sufficient time for interaction between the probe compound compounds and proteins in the cell lysate or tissue to reach equilibrium; and identifying and quantifying the amount of proteins labelled with the probe compound.

Also provided is a method for assessing the efficacy of a potential LOXL2 inhibitor in a mammal, comprising: administering the potential LOXL2 inhibitor to the mammal; administering the any one of the compounds disclosed herein to the mammal or to cells isolated from the mammal; and measuring the LOXL2 activity of the compound.

Also provided herein is method for assessing the pharmacodynamics of a LOXL2 inhibitor in a mammal, comprising: administering the LOXL2 inhibitor to a plurality of mammals; administering any of the compounds disclosed herein to the plurality of mammals or to cells isolated from a plurality of mammals; and measuring the LOXL2 activity of the compound at different time points following the administration of the LOXL2 inhibitor.

Also provided herein is method for evaluating the tissue distribution of any one of the compounds disclosed herein in a mammal, comprising: administering any one of the compounds disclosed herein to the plurality of mammals; and measuring the activity of the compound at different time points for different types of tissue following the administration of the compound.

As used herein, the term “solid support” means a non-gaseous, non-liquid material having a surface. Thus, a solid support can be a flat surface constructed, for example, of glass, silicon, metal, plastic or a composite; or can be in the form of a bead such as a silica gel, a controlled pore glass, a magnetic or cellulose bead; or can be a pin, including an array of pins suitable for combinatorial synthesis or analysis.

### Compounds

‘Warhead’

Probe compounds described herein include a ‘warhead’ group that permits selective interaction of the probe compound with LOXL2. The warhead is a small molecule lysyl oxidase like-2 (LOXL2) inhibitor (LOXL2i). In some embodiments, warhead is a substituted or unsubstituted heterocyclylmethylamine or a substituted or unsubstituted arylmethylamine.

In some embodiments, the ‘warhead’ group is a substituted or unsubstituted heterocyclylmethylamine. In some embodiments, the ‘warhead’ group is a substituted or unsubstituted heteroarylmethylamine. In some embodiments, the ‘warhead’ group is a substituted or unsubstituted (monocyclic heteroaryl)methylamine. In some embodiments, the monocyclic heteroaryl is a 6-membered monocyclic heteroaryl or a 5-membered monocyclic heteroaryl. In some embodiments, the monocyclic heteroaryl is a 6-membered monocyclic heteroaryl that is pyridinyl, pyrimidinyl, pyrazinyl, pyridazinyl, or triazinyl. In some embodiments, the monocyclic heteroaryl is a 5-membered monocyclic heteroaryl that is imidazolyl, pyrazolyl, triazolyl, tetrazolyl, furyl, thienyl, isoxazolyl, thiazolyl, oxazolyl, isothiazolyl, pyrrolyl, oxadiazolyl, thiadiazolyl, or furazanyl.

In some embodiments, the ‘warhead’ group is a substituted or unsubstituted (bicyclic heteroaryl)methylamine. In some embodiments, the bicyclic heteroaryl is indolizinyl, indolyl, benzofuranyl, benzothiophenyl, indazolyl, benzimidazolyl, purinyl, quinolizinyl, quinolinyl, isoquinolinyl, cinnolinyl, phthalazinyl, quinazolinyl, quinoxalinyl, 1,8-naphthyridinyl, or pteridinyl.

In some embodiments, the ‘warhead’ group is a substituted or unsubstituted (bicyclic heterocyclyl)methylamine. In some embodiments, the bicyclic heterocyclyl is quinolinonyl, isoquinolinonyl, chromonyl, or coumarinyl

In some embodiments, the LOXL2i is a substituted pyridinylmethylamine. In some embodiments, the substituted pyridinylmethylamine is a substituted pyridin-4-ylmethylamine compound. In some embodiments, the substituted pyridinylmethylamine is a compound described in International patent application no. PCT/US2016/020731 titled “Lysysl Oxidase-Like 2 Inhibitors and Uses Thereof” filed on Mar. 3, 2016; which is herein incorporated by reference for such compounds. In some embodiments, the LOXL2i is a compound described in Table 1, Table 2, Table 3, or Table 4, of International patent application no. PCT/US2016/020731.

In some embodiments, the LOXL2i is a substituted pyridinylmethylamine that is:
4-(4-(Aminomethyl)pyridin-2-yl)-N-(2-methoxyethyl)benzamide;
Racemic (4-(4-(aminomethyl)pyridin-2-yl)phenyl)(3-hydroxy-3-(trifluoromethyl)pyrrolidin-1-yl)methanone;
4-(4-(Aminomethyl)pyridin-2-yl)-N-phenylbenzamide;
4-(4-(Aminomethyl)pyridin-2-yl)-N-benzylbenzamide;
3-(4-(Aminomethyl)pyridin-2-yl)-N-(2-methoxyethyl)benzamide;
Racemic-(3-(4-(aminomethyl)pyridin-2-yl)phenyl)(3-hydroxy-3-(trifluoromethyl)pyrrolidin-1-yl)methanone;
3-(4-(Aminomethyl)pyridin-2-yl)-N-phenylbenzamide;
3-(4-(Aminomethyl)pyridin-2-yl)-N-benzylbenzamide;
3-(4-(Aminomethyl)pyridin-2-yl)-N-(5-chloro-2-methylphenyl)benzamide;
3-(4-(Aminomethyl)pyridin-2-yl)-N-(6-chloro-1H-indol-4-yl)benzamide;
4-((4-(Aminomethyl)pyridin-2-yl)oxy)-N-(2-methoxyethyl)benzamide;
Racemic 4-((4-(aminomethyl)pyridin-2-yl)oxy)phenyl)(3-hydroxy-3-(trifluoromethyl)pyrrolidin-1-yl)methanone;
4-((4-(Aminomethyl)pyridin-2-yl)oxy)-N-phenylbenzamide;
4-((4-(Aminomethyl)pyridin-2-yl)oxy)-N-benzylbenzamide;
3-((4-(Aminomethyl)pyridin-2-yl)oxy)-N-(2-methoxyethyl)benzamide;
Racemic-(3-((4-(aminomethyl)pyridin-2-yl)oxy)phenyl)(3-hydroxy-3-(trifluoromethyl)pyrrolidin-1-yl)methanone;
3-((4-(Aminomethyl)pyridin-2-yl)oxy)-N-phenylbenzamide;
3-((4-(Aminomethyl)pyridin-2-yl)oxy)-N-benzylbenzamide;
3-((4-(Aminomethyl)pyridin-2-yl)oxy)-N-(3-methoxyphenyl)benzamide;
3-((4-(Aminomethyl)pyridin-2-yl)oxy)-N-(4-methoxyphenyl)benzamide;
3-((4-(Aminomethyl)pyridin-2-yl)oxy)-N-(3-(trifluoromethyl)phenyl)benzamide;
3-((4-(Aminomethyl)pyridin-2-yl)oxy)-N-(4-(trifluoromethyl)phenyl)benzamide;
3-((4-(Aminomethyl)pyridin-2-yl)oxy)-N-(4-fluorophenyl)benzamide;
3-((4-(Aminomethyl)pyridin-2-yl)oxy)-N-(2,4-difluorophenyl)benzamide;
3-((4-(Aminomethyl)pyridin-2-yl)oxy)-N-(4-bromophenyl)benzamide;
Methyl 4-(3-((4-(aminomethyl)pyridin-2-yl)oxy)benzamido)benzoate;
Ethyl 3-(3-((4-(aminomethyl)pyridin-2-yl)oxy)benzamido)benzoate;
3-(3-((4-(Aminomethyl)pyridin-2-yl)oxy)benzamido)benzoic acid;
4-(3-((4-(Aminomethyl)pyridin-2-yl)oxy)benzamido)benzoic acid;
3-((4-(Aminomethyl)pyridin-2-yl)oxy)-N-(2,4-difluorobenzyl)benzamide;
3-((4-(Aminomethyl)pyridin-2-yl)oxy)-N-(4-(trifluoromethyl)benzyl)benzamide;
3-((4-(Aminomethyl)pyridin-2-yl)oxy)-N-(4-bromobenzyl)benzamide;
Methyl 4-((3-((4-(aminomethyl)pyridin-2-yl)oxy)benzamido)methyl)benzoate;
4-((3-((4-(Aminomethyl)pyridin-2-yl)oxy)benzamido)methyl)benzoic acid;
3-((3-((4-(Aminomethyl)pyridin-2-yl)oxy)benzamido)methyl)benzoic acid;
(R)-3-((4-(Aminomethyl)pyridin-2-yl)oxy)-N-(2-hydroxy-1-phenylethyl)benzamide;
3-((4-(Aminomethyl)pyridin-2-yl)oxy)-N-(3-fluoro-4-(1H-imidazol-1-yl)benzyl)benzamide;
3-((4-(Aminomethyl)pyridin-2-yl)oxy)-N-(4-(4-ethylpiperazin-1-yl)benzyl)benzamide;
3-((4-(Aminomethyl)pyridin-2-yl)oxy)-N-(3-carbamimidoylbenzyl)benzamide;
3-((4-(Aminomethyl)pyridin-2-yl)oxy)-N-phenethylbenzamide;
(3-((4-(Aminomethyl)pyridin-2-yl)oxy)phenyl)(3,4-dihydroisoquinolin-2(1H)-yl)methanone;
3-((4-(Aminomethyl)pyridin-2-yl)oxy)-N-(benzo[b]thiophen-2-ylmethyl)benzamide;
3-((4-(Aminomethyl)pyridin-2-yl)oxy)-N-(pyrazin-2-ylmethyl)benzamide;
3-((4-(Aminomethyl)pyridin-2-yl)oxy)-N-tridecylbenzamide; or
3-(((4-(Aminomethyl)pyridin-2-yl)oxy)methyl)-N-phenylbenzamide.

In some embodiments, the LOXL2i is a substituted pyridinylmethylamine that is:
Racemic-trans-(1-(4-(aminomethyl)pyridin-2-yl)-4-fluoropyrrolidin-3-ol;
Racemic-1-(4-(aminomethyl)pyridin-2-yl)-3-(trifluoromethyl)pyrrolidin-3-ol;
[2,3′-Bipyridin]-4-ylmethanamine;
(2-(4-Fluorophenyl)pyridin-4-yl)methanamine;
3-((4-(Aminomethyl)pyridin-2-yl)oxy)benzoic acid;
4-(4-(Aminomethyl)pyridin-2-yl)benzoic acid;
(2-Phenoxypyridin-4-yl)methanamine;
(2-(3-Phenoxyphenoxy)pyridin-4-yl)methanamine;
4-((4-(Aminomethyl)pyridin-2-yl)oxy)benzoic acid;
(2-((1-Benzyl-1H-indol-4-yl)oxy)pyridin-4-yl)methanamine;
(2-((1-(2,4-Difluorobenzyl)-2-methyl-1H-indol-4-yl)oxy)pyridin-4-yl)methanamine;
(2-((1-(2,4-Difluorobenzyl)-3-methyl-1H-indol-4-yl)oxy)pyridin-4-yl)methanamine;
(2-((1-((6-Methoxypyridin-3-yl)methyl)-2-methyl-1H-indol-4-yl)oxy)pyridin-4-yl)methanamine;
(2-((1-((6-Methoxypyridin-3-yl)methyl)-3-methyl-1H-indol-4-yl)oxy)pyridin-4-yl)methanamine;
(2-((1-((5-Fluoropyridin-2-yl)methyl)-1H-indol-4-yl)oxy)pyridin-4-yl)methanamine;
(2-((1-(Quinolin-2-ylmethyl)-1H-indol-4-yl)oxy)pyridin-4-yl)methanamine;
(2-((1-((2-(Trifluoromethyl)thiazol-5-yl)methyl)-1H-indol-4-yl)oxy)pyridin-4-yl)methanamine;
(2-((1-(4-Fluorophenyl)-1H-indol-4-yl)oxy)pyridin-4-yl)methanamine;
(2-((1-(1-Methyl-1H-pyrazol-4-yl)-1H-indol-4-yl)oxy)pyridin-4-yl)methanamine;
(2-((1-(6-Methoxypyridin-3-yl)-1H-indol-4-yl)oxy)pyridin-4-yl)methanamine;
(2-((1-(6-Fluorobenzo[d]thiazol-2-yl)-1H-indol-4-yl)oxy)pyridin-4-yl)methanamine;
(2-((1-(4-Fluorophenethyl)-1H-indol-4-yl)oxy)pyridin-4-yl)methanamine;
(2-((3-Bromo-1-(4-fluorophenethyl)-1H-indol-4-yl)oxy)pyridin-4-yl)methanamine;
(4-((4-(Aminomethyl)pyridin-2-yl)oxy)-1H-indol-1-yl)(phenyl)methanone;
4-((4-(Aminomethyl)pyridin-2-yl)oxy)-N-phenyl-1H-indole-1-carboxamide;
2-(4-((4-(Aminomethyl)pyridin-2-yl)oxy)-1H-indol-1-yl)-N,N-dimethylacetamide;
2-(4-((4-(Aminomethyl)pyridin-2-yl)oxy)-1H-indol-1-yl)-1-(piperidin-1-yl)ethanone;
(R) or (S)-2-(4-((4-(Aminomethyl)pyridin-2-yl)oxy)-1H-indol-1-yl)-1-(3-hydroxy-3-(trifluoromethyl)piperidin-1-yl)ethan-1-one (Enantiomer 1);
(R) or (S)-2-(4-((4-(Aminomethyl)pyridin-2-yl)oxy)-1H-indol-1-yl)-1-(3-hydroxy-3-(trifluoromethyl)piperidin-1-yl)ethan-1-one (Enantiomer 2);
2-(4-((4-(Aminomethyl)pyridin-2-yl)oxy)-1H-indol-1-yl)-N-methyl-N-phenylacetamide;
2-4-((4-(Aminomethyl)pyridin-2-yl)oxy)-1H-indol-1-yl)-1-(3,4-dihydroquinolin-1(2H)-yl)ethanone;
(2-((1H-Indol-4-yl)oxy)pyridin-4-yl)methanamine;
(2-((1-(6-Fluorobenzo[d]thiazol-2-yl)indolin-4-yl)oxy)pyridin-4-yl)methanamine;
(2-((1-((6-Methoxypyridin-3-yl)methyl)-1H-indol-5-yl)oxy)pyridin-4-yl)methanamine;
(2-((1-(2,4-Difluorobenzyl)-3-methyl-1H-indazol-4-yl)oxy)pyridin-4-yl)methanamine;
5-((4-(Aminomethyl)pyridin-2-yl)oxy)-1-(2,4-difluorobenzyl)-3,4-dihydroquinolin-2(1H)-one;
5-((4-(Aminomethyl)pyridin-2-yl)oxy)-1-((6-methoxypyridin-3-yl)methyl)-3,4-dihydroquinolin-2(1H)-one;
(S)-(3-((4-(Aminomethyl)pyridin-2-yl)oxy)piperidin-1-yl)(phenyl)methanone;
(S)-3-((4-(Aminomethyl)pyridin-2-yl)oxy)-N-phenylpiperidine-1-carboxamide;
Racemic-(4-(aminomethyl)pyridin-2-yl)(3-hydroxy-3-(trifluoromethyl)pyrrolidin-1-yl)methanone;
4-(Aminomethyl)-N-benzylpicolinamide;
N-(4-(Aminomethyl)pyridin-2-yl)-2-fluoro-4-methylbenzamide;
N-(4-(Aminomethyl)pyridin-2-yl)-2-(3,4-dichlorophenyl)acetamide.

In some embodiments, the LOXL2i is a substituted pyridinylmethylamine compound that is:
4-(Aminomethyl)-N,N-dimethyl-6-(3-phenoxyphenoxy)pyridin-2-amine;
4-(Aminomethyl)-N-(2-methoxyethyl)-6-(3-phenoxyphenoxy)pyridin-2-amine; or
(2-(4-Fluorophenyl)-6-(3-phenoxyphenoxy)pyridin-4-yl)methanamine.

In some embodiments, the LOXL2i is a substituted pyridinylmethylamine that is:
2-Fluoro-3-(aminomethyl)pyridine;
3-Fluoro-4-(aminomethyl)pyridine;
2-Fluoro-4-(aminomethyl)pyridine;
2-Trifluoromethyl-4-(aminomethyl)pyridine;
2-Chloro-4-(aminomethyl)pyridine;
2-Benzyloxy-4-(aminomethyl)pyridine;
2-Cyclohexyloxy-4-(aminomethyl)pyridine;
2-(4-Fluorophenoxy)-4-(aminomethyl)pyridine;
2-(2-Trifluoromethylphenoxy)-4-(aminomethyl)pyridine;
2-(1-Pyrrolidino)-4-(aminomethyl)pyridine;
2-(Imidazol-1-yl)-4-(aminomethyl)pyridine;
2-(4-Ethylpiperazin-1-yl)-4-(aminomethyl)pyridine;
4-(Aminomethyl)-N-(2-methoxyethyl)picolinamide;
4-(Aminomethyl)-N-(2,2,2-trifluoroethyl)picolinamide.

In some embodiments, the LOXL2i is a substituted or unsubstituted 6-(trifluoromethyl)pyridin-4-yl)methanamine. In some embodiments, the substituted 6-(trifluoromethyl)pyridin-4-yl)methanamine is a compound described in International patent application no. PCT/US2016/020732 titled “Fluorinated Lysyl Oxidase-Like 2 Inhibitors and Uses Thereof filed on Mar. 3, 2016, which is herein incorporated by reference for such compounds.

In some embodiments, the LOXL-2 inhibitor compound is a compound described in International patent application no. PCT/US2016/020732 titled “Fluorinated Lysyl Oxidase-Like 2 Inhibitors and Uses Thereof filed on Mar. 3, 2016, which is herein incorporated by reference for such compounds.

In some embodiments, the LOXL2i is a compound described in Table 1 of International patent application no. PCT/US2016/020732.

In some embodiments, the LOXL2i is a substituted or unsubstituted 6-(trifluoromethyl)pyridin-4-yl)methanamine that is:
(6-(Trifluoromethyl)-[2,3′-bipyridin]-4-yl)methanamine;
(2-([1,1′-Biphenyl]-3-yloxy)-6-(trifluoromethyl)pyridin-4-yl)methanamine;
(2-(3-Phenoxyphenoxy)-6-(trifluoromethyl)pyridin-4-yl)methanamine;
(2-(3-(Phenoxymethyl)phenoxy)-6-(trifluoromethyl)pyridin-4-yl)methanamine;
3-((4-(Aminomethyl)-6-(trifluoromethyl)pyridin-2-yl)oxy)-N-phenylaniline;
(2-(3-(1H-Pyrazol-4-yl)phenoxy)-6-(trifluoromethyl)pyridin-4-yl)methanamine;
3-((4-(Aminomethyl)-6-(trifluoromethyl)pyridin-2-yl)oxy)-N-phenylbenzamide;
3-((4-(Aminomethyl-d₂)-6-(trifluoromethyl)pyridin-2-yl)oxy)-N-phenylbenzamide;
3-((4-(Aminomethyl)-6-(trifluoromethyl)pyridin-2-yl)oxy)-N-(4-fluorobenzyl)benzamide;
3-((4-(Aminomethyl)-6-(trifluoromethyl)pyridin-2-yl)oxy)-N-(benzo[b]thiophen-2-ylmethyl)benzamide;
(3-((4-(Aminomethyl)-6-(trifluoromethyl)pyridin-2-yl)oxy)phenyl)(3,4-dihydroisoquinolin-2(1H)-yl)methanone;
(3-(1H-Pyrazol-1-yl)azetidin-1-yl)(3-((4-(aminomethyl)-6-(trifluoromethyl)pyridin-2-yl)oxy)phenyl)methanone;
N-((2H-Tetrazol-5-yl)methyl)-3-((4-(aminomethyl)-6-(trifluoromethyl)pyridin-2-yl)oxy)benzamide;
N-(2-(1H-1,2,4-Triazol-1-yl)ethyl)-3-((4-(aminomethyl)-6-(trifluoromethyl)pyridin-2-yl)oxy)benzamide;
N-(2-(1H-Tetrazol-1-yl)ethyl)-3-((4-(aminomethyl)-6-(trifluoromethyl)pyridin-2-yl)oxy)benzamide;
3-((4-(Aminomethyl)-6-(trifluoromethyl)pyridin-2-yl)oxy)-N-(2-hydroxyethyl)benzamide;
(S)-(3-((4-(Aminomethyl)-6-(trifluoromethyl)pyridin-2-yl)oxy)phenyl)(3-hydroxypyrrolidin-1-yl)methanone;
(R)-(3-((4-(Aminomethyl)-6-(trifluoromethyl)pyridin-2-yl)oxy)phenyl)(3-hydroxypyrrolidin-1-yl)methanone;
Racemic-trans-(3-((4-(aminomethyl)-6-(trifluoromethyl)pyridin-2-yl)oxy)phenyl)(3-fluoro-4-hydroxypyrrolidin-1-yl)methanone;
(S,S)-trans-(3-((4-(Aminomethyl)-6-(trifluoromethyl)pyridin-2-yl)oxy)phenyl)(3-fluoro-4-hydroxypyrrolidin-1-yl)methanone;
(R,R)-trans-(3-((4-(Aminomethyl)-6-(trifluoromethyl)pyridin-2-yl)oxy)phenyl)(3-fluoro-4-hydroxypyrrolidin-1-yl)methanone;
(R)-(3-((4-(Aminomethyl)-6-(trifluoromethyl)pyridin-2-yl)oxy)phenyl)(3-aminopyrrolidin-1-yl)methanone;
Racemic-trans-(3-((4-(aminomethyl)-6-(trifluoromethyl)pyridin-2-yl)oxy)phenyl)(-3-(dimethylamino)-4-hydroxypyrrolidin-1-yl)methanone;
(S)-1-(3-((4-(Aminomethyl)-6-(trifluoromethyl)pyridin-2-yl)oxy)benzoyl)pyrrolidine-2-carboxylic acid;
(R)-1-(3-((4-(Aminomethyl)-6-(trifluoromethyl)pyridin-2-yl)oxy)benzoyl)pyrrolidine-2-carboxylic acid;
(R)-(3-((4-(Aminomethyl)-6-(trifluoromethyl)pyridin-2-yl)oxy)phenyl)(3-(hydroxymethyl)pyrrolidin-1-yl)methanone;
8-(3-((4-(Aminomethyl)-6-(trifluoromethyl)pyridin-2-yl)oxy)benzoyl)-1-oxa-3,8-diazaspiro[4.5]decan-2-one; 3-((4-(Aminomethyl)-6-(trifluoromethyl)pyridin-2-yl)oxy)-N-(2-(2-oxooxazolidin-3-yl)ethyl)benzamide;
Racemic-3-((4-(aminomethyl)-6-(trifluoromethyl)pyridin-2-yl)oxy)-N-((5-oxopyrrolidin-2-yl)methyl)benzamide;
3-((4-(Aminomethyl)-6-(trifluoromethyl)pyridin-2-yl)oxy)-N-(2-(methylsulfonyl)ethyl)benzamide;
3-((4-(Aminomethyl)-6-(trifluoromethyl)pyridin-2-yl)oxy)-N-(1-(hydroxymethyl)cyclopropyl)benzamide;
3-((4-(Aminomethyl)-6-(trifluoromethyl)pyridin-2-yl)oxy)-N-(2-hydroxy-2-methylpropyl)benzamide;
(R)-3-((4-(Aminomethyl)-6-(trifluoromethyl)pyridin-2-yl)oxy)-N-(2,3-dihydroxypropyl)benzamide;
3-((4-(Aminomethyl)-6-(trifluoromethyl)pyridin-2-yl)oxy)-N-(2-sulfamoylethyl)benzamide;
3-((4-(Aminomethyl)-6-(trifluoromethyl)pyridin-2-yl)oxy)-N-(2-(dimethylamino)ethyl)benzamide;
Racemic-trans-(3-(((4-(aminomethyl)-6-(trifluoromethyl)pyridin-2-yl)oxy)methyl)phenyl)(3-fluoro-4-hydroxypyrrolidin-1-yl)methanone;
(2-((1-(1-Methyl-1H-pyrazol-4-yl)-1H-indol-4-yl)oxy)-6-(trifluoromethyl)pyridin-4-yl) methanamine;
2-(4-((4-(Aminomethyl)-6-(trifluoromethyl)pyridin-2-yl)oxy)-1H-indol-1-yl)-N-methyl-N-phenylacetamide;
(R)-3-((4-(Aminomethyl)-6-(trifluoromethyl)pyridin-2-yl)oxy)-N-phenylpyrrolidine-1-carboxamide;
(S)-3-((4-(Aminomethyl)-6-(trifluoromethyl)pyridin-2-yl)oxy)-N-phenylpyrrolidine-1-carboxamide;
4-((4-(Aminomethyl)-6-(trifluoromethyl)pyridin-2-yl)oxy)-N-phenylpiperidine-1-carboxamide;
4-(((4-(Aminomethyl)-6-(trifluoromethyl)pyridin-2-yl)oxy)methyl)-N-phenylpiperidine-1-carboxamide;
(R)-3-((4-(Aminomethyl)-6-(trifluoromethyl)pyridin-2-yl)oxy)-N-phenylpiperidine-1-carboxamide;
(S)-3-((4-(Aminomethyl)-6-(trifluoromethyl)pyridin-2-yl)oxy)-N-phenylpiperidine-1-carboxamide;
(S)-3-(((4-(Aminomethyl)-6-(trifluoromethyl)pyridin-2-yl)oxy)methyl)-N-phenylpiperidine-1-carboxamide;
(S)-1-(3-((4-(Aminomethyl)-6-(trifluoromethyl)pyridin-2-yl)oxy)piperidin-1-yl)-2-phenylethanone;
(S)-1-(3-((4-(Aminomethyl)-6-(trifluoromethyl)pyridin-2-yl)oxy)piperidin-1-yl)-2-(3,4-dichlorophenyl)ethanone;
(S)-2-(3-((4-(Aminomethyl)-6-(trifluoromethyl)pyridin-2-yl)oxy)piperidine-1-carbonyl)-4H-chromen-4-one;
(S)-(3-((4-(Aminomethyl)-6-(trifluoromethyl)pyridin-2-yl)oxy)piperidin-1-yl)(pyridin-3-yl)methanone;
(S)-(3-((4-(Aminomethyl)-6-(trifluoromethyl)pyridin-2-yl)oxy)piperidin-1-yl)(pyrimidin-5-yl)methanone;
(S)-(3-((4-(Aminomethyl)-6-(trifluoromethyl)pyridin-2-yl)oxy)piperidin-1-yl)(5-methyl-1,3,4-oxadiazol-2-yl)methanone;
(S)-1-(3-((4-(Aminomethyl)-6-(trifluoromethyl)pyridin-2-yl)oxy)piperidin-1-yl)-2-methylpropan-1-one;
5-((4-(Aminomethyl)-6-(trifluoromethyl)pyridin-2-yl)oxy)-N-(2-(methylsulfonyl)ethyl)nicotinamide;
(R)-(5-((4-(Aminomethyl)-6-(trifluoromethyl)pyridin-2-yl)oxy)pyridin-3-yl)(3-aminopyrrolidin-1-yl)methanone;
Racemic-trans-(5-((4-(aminomethyl)-6-(trifluoromethyl)pyridin-2-yl)oxy)pyridin-3-yl)(-3-fluoro-4-hydroxypyrrolidin-1-yl)methanone;
2-(4-((4-(Aminomethyl)-6-(trifluoromethyl)pyridin-2-yl)oxy)-1H-indol-1-yl)-1-(piperidin-1-yl)ethanone;
tert-Butyl 4-(2-(4-((4-(aminomethyl)-6-(trifluoromethyl)pyridin-2-yl)oxy)-1H-indol-1-yl)acetyl)piperazine-1-carboxylate;
(2-((1H-Indol-4-yl)oxy)-6-(trifluoromethyl)pyridin-4-yl)methanamine;
5-((4-(Aminomethyl)-6-(trifluoromethyl)pyridin-2-yl)oxy)-3,4-dihydroquinolin-2(1H)-one;
5-((4-(Aminomethyl)-6-(trifluoromethyl)pyridin-2-yl)oxy)-1-(2-hydroxyethyl)-3,4-dihydroquinolin-2(1H)-one;
3-((4-(Aminomethyl)-6-(trifluoromethyl)pyridin-2-yl)thio)-N-phenylbenzamide;
3-(((4-(Aminomethyl)-6-(trifluoromethyl)pyridin-2-yl)amino)methyl)-N-phenylbenzamide;
3-(((4-(Aminomethyl)-6-(trifluoromethyl)pyridin-2-yl)amino)methyl)-N-(2-(methylsulfonyl)ethyl)benzamide;
3-((4-(Aminomethyl)-6-(trifluoromethyl)pyridin-2-yl)methyl)-1-benzyl-6-methylpyrimidine-2,4(1H,3H)-dione;
3-((4-(Aminomethyl)-6-(trifluoromethyl)pyridin-2-yl)methyl)-1-(2-hydroxyethyl)-6-methylpyrimidine-2,4(1H,3H)-dione;
4′-(Aminomethyl)-6′-(trifluoromethyl)-2H-[1,2′-bipyridin]-2-one;
1-(3-((4-(Aminomethyl)-6-(trifluoromethyl)pyridin-2-yl)oxy)phenyl)pyridin-2(1H)-one;
3-((4-(Aminomethyl)-6-(trifluoromethyl)pyridin-2-yl)oxy)-N-(2-cyanoethyl)benzamide;
1-(3-((4-(Aminomethyl)-6-(trifluoromethyl)pyridin-2-yl)oxy)benzoyl)azetidine-3-carbonitrile;
3-((4-(Aminomethyl)-6-(trifluoromethyl)pyridin-2-yl)oxy)-N-(oxetan-3-yl)benzamide;
3-((4-(Aminomethyl)-6-(trifluoromethyl)pyridin-2-yl)oxy)-N-((1-hydroxycyclobutyl)methyl)benzamide;
3-((4-(Aminomethyl)-6-(trifluoromethyl)pyridin-2-yl)oxy)-N-(2-hydroxyethyl)-N-methylbenzamide;
(S)-(3-((4-(Aminomethyl)-6-(trifluoromethyl)pyridin-2-yl)oxy)phenyl)(3-(hydroxymethyl)piperidin-1-yl)methanone;
(3-((4-(Aminomethyl)-6-(trifluoromethyl)pyridin-2-yl)oxy)phenyl)(4-(hydroxymethyl)piperidin-1-yl)methanone;
(3-((4-(Aminomethyl)-6-(trifluoromethyl)pyridin-2-yl)oxy)phenyl)(4-hydroxy-4-methylpiperidin-1-yl)methanone;
(3-((4-(Aminomethyl)-6-(trifluoromethyl)pyridin-2-yl)oxy)phenyl)(3-(methoxymethyl)azetidin-1-yl)methanone;
(3-((4-(Aminomethyl)-6-(trifluoromethyl)pyridin-2-yl)oxy)phenyl)(7-oxa-2-azaspiro[3.5]nonan-2-yl)methanone;
3-((4-(Aminomethyl)-6-(trifluoromethyl)pyridin-2-yl)oxy)-N-((3S,4S)-4-hydroxytetrahydro-2H-pyran-3-yl)benzamide;
3-((4-(Aminomethyl)-6-(trifluoromethyl)pyridin-2-yl)oxy)-N-((1R,2S)-2-hydroxycyclopentyl)benzamide;
3-((4-(Aminomethyl)-6-(trifluoromethyl)pyridin-2-yl)oxy)-N-((1S,2R)-2-hydroxycyclopentyl)benzamide;
Racemic-cis-(3-((4-(aminomethyl)-6-(trifluoromethyl)pyridin-2-yl)oxy)phenyl)(3-fluoro-4-hydroxypyrrolidin-1-yl)methanone;
(R)-(3-((4-(Aminomethyl)-6-(trifluoromethyl)pyridin-2-yl)oxy)phenyl)(3-fluoropyrrolidin-1-yl)methanone;
(S)-(3-((4-(Aminomethyl)-6-(trifluoromethyl)pyridin-2-yl)oxy)phenyl)(3-fluoropyrrolidin-1-yl)methanone;
(3R,4R)-1-(3-((4-(Aminomethyl)-6-(trifluoromethyl)pyridin-2-yl)oxy)benzoyl)-4-fluoropyrrolidin-3-ylmethanesulfonate;
(3-((4-(Aminomethyl)-6-(trifluoromethyl)pyridin-2-yl)oxy)phenyl)(2,5-dihydro-1H-pyrrol-1-yl)methanone;
3-((4-(Aminomethyl)-6-(trifluoromethyl)pyridin-2-yl)oxy)-N-(hex-5-yn-1-yl)benzamide;
3-((4-(Aminomethyl)-6-(trifluoromethyl)pyridin-2-yl)oxy)-N-(4-(1-phenyl-1H-1,2,3-triazol-4-yl)butyl)benzamide;
3-((4-(Aminomethyl)-6-(trifluoromethyl)pyridin-2-yl)oxy)-N-hydroxybenzamide;
3-((4-(Aminomethyl)-6-(trifluoromethyl)pyridin-2-yl)oxy)-N-methoxybenzamide;
Methyl (S)-3-(3-((4-(aminomethyl)-6-(trifluoromethyl)pyridin-2-yl)oxy)benzamido)pent-4-ynoate;
(S)-3-(3-((4-(Aminomethyl)-6-(trifluoromethyl)pyridin-2-yl)oxy)benzamido)pent-4-ynoic acid;
Methyl (R)-3-(3-((4-(aminomethyl)-6-(trifluoromethyl)pyridin-2-yl)oxy)benzamido)pent-4-ynoate;
(R)-3-(3-((4-(Aminomethyl)-6-(trifluoromethyl)pyridin-2-yl)oxy)benzamido)pent-4-ynoic acid;
3-((4-(Aminomethyl)-6-(trifluoromethyl)pyridin-2-yl)oxy)benzoic acid;
3-((4-(Aminomethyl)-6-(trifluoromethyl)pyridin-2-yl)oxy)-N-(2-aminophenyl)benzamide;
3-((4-(Aminomethyl)-6-(trifluoromethyl)pyridin-2-yl)oxy)-N-(pyrimidin-5-yl)benzamide;
3-((4-(Aminomethyl)-6-(trifluoromethyl)pyridin-2-yl)oxy)-N-(oxazol-2-yl)benzamide;
3-((4-(Aminomethyl)-6-(trifluoromethyl)pyridin-2-yl)oxy)-N-(1,3,4-oxadiazol-2-yl)benzamide;
Racemic-trans-(3-((4-(aminomethyl)-6-(trifluoromethyl)pyridin-2-yl)oxy)-4-hydroxyphenyl)(3-fluoro-4-hydroxypyrrolidin-1-yl)methanone;
Racemic-trans-(3-((4-(aminomethyl)-6-(trifluoromethyl)pyridin-2-yl)oxy)-5-hydroxyphenyl)(3-fluoro-4-hydroxypyrrolidin-1-yl)methanone;
Racemic-trans-(3-((4-(aminomethyl)-6-(trifluoromethyl)pyridin-2-yl)oxy)-4-(benzyloxy)phenyl)(3-fluoro-4-hydroxypyrrolidin-1-yl)methanone;
Racemic-trans-(3-((4-(aminomethyl)-6-(trifluoromethyl)pyridin-2-yl)oxy)-5-methoxyphenyl)(3-fluoro-4-hydroxypyrrolidin-1-yl)methanone;
2-(4-((4-(Aminomethyl)-6-(trifluoromethyl)pyridin-2-yl)oxy)-1H-indol-1-yl)-1-(piperazin-1-yl)ethan-1-one;
2-(4-((4-(Aminomethyl)-6-(trifluoromethyl)pyridin-2-yl)oxy)-1H-indol-1-yl)-N-carbamimidoylacetamide;
Ethyl 2-(5-((4-(aminomethyl)-6-(trifluoromethyl)pyridin-2-yl)oxy)-2-oxo-3,4-dihydroquinolin-1(2H)-yl)acetate;
2-(5-((4-(Aminomethyl)-6-(trifluoromethyl)pyridin-2-yl)oxy)-2-oxo-3,4-dihydroquinolin-1(2H)-yl)acetic acid;
(S)-(3-(((4-(Aminomethyl)-6-(trifluoromethyl)pyridin-2-yl)amino)methyl)phenyl)(3-hydroxypyrrolidin-1-yl)methanone;
Methyl 3-((4-(aminomethyl)-6-(trifluoromethyl)pyridin-2-yl)carbamoyl)benzoate;
3-((4-(Aminomethyl)-6-(trifluoromethyl)pyridin-2-yl)carbamoyl)benzoic acid;
N¹-(4-(Aminomethyl)-6-(trifluoromethyl)pyridin-2-yl)-N³-phenylisophthalamide;
(S)—N-(4-(Aminomethyl)-6-(trifluoromethyl)pyridin-2-yl)-3-(3-hydroxypyrrolidine-1-carbonyl)benzamide;
N¹-(4-(Aminomethyl)-6-(trifluoromethyl)pyridin-2-yl)-N³-(2-(methylsulfonyl)ethyl)isophthalamide;
3-((4-(Aminomethyl)-6-(trifluoromethyl)pyridin-2-yl)methyl)-6-methyl-1-(pyrimidin-4-ylmethyl)pyrimidine-2,4(1H,3H)-dione;
1-(2-((4-(Aminomethyl)-6-(trifluoromethyl)pyridin-2-yl)oxy)-6-azaspiro[3.4]octan-6-yl)ethanone;
3-((4-(Aminomethyl)-6-(trifluoromethyl)pyridin-2-yl)oxy)-N-(3-ethylphenyl)benzamide;
3-((4-(Aminomethyl)-6-(trifluoromethyl)pyridin-2-yl)oxy)-N-(4-ethylphenyl)benzamide;
3-((4-(Aminomethyl)-6-(trifluoromethyl)pyridin-2-yl)oxy)-N-(3-ethynylphenyl)benzamide;
3-((4-(Aminomethyl)-6-(trifluoromethyl)pyridin-2-yl)oxy)-N-(prop-2-yn-1-yl)benzamide;
3-((4-(Aminomethyl)-6-(trifluoromethyl)pyridin-2-yl)oxy)-N-(4-methyl-2-oxo-2H-chromen-7-yl)benzamide;
(R,S)-cis-(3-((4-(Aminomethyl)-6-(trifluoromethyl)pyridin-2-yl)oxy)phenyl)(3-fluoro-4-hydroxypyrrolidin-1-yl)methanone;
(S,R)-cis-(3-((4-(Aminomethyl)-6-(trifluoromethyl)pyridin-2-yl)oxy)phenyl)(3-fluoro-4-hydroxypyrrolidin-1-yl)methanone; or
4-((4-(Aminomethyl)-6-(trifluoromethyl)pyridin-2-yl)oxy)-N-phenylpicolinamide.

In some embodiments, the LOXL2i is a substituted or unsubstituted pyrimidinylmethylamine. In some embodiments, the substituted or unsubstituted pyrimidinylmethylamine is a substituted or unsubstituted pyrimidin-4-ylmethylamine compound. In some embodiments, the substituted or unsubstituted pyrimidinylmethylamine is a compound described in International Patent Application No. PCT/US2016/039253 titled “Lysyl Oxidase-Like 2 Inhibitors and Uses Thereof” filed on Jun. 24, 2016, which is herein incorporated by reference for such compounds.

In some embodiments, the LOXL2i is a substituted or unsubstituted pyrimidinylmethylamine compound described in Table 1 of International Patent Application No. PCT/US2016/039253.

In some embodiments, the LOXL2i is a substituted or unsubstituted pyrimidinylmethylamine that is:
(6-(4-Fluorophenoxy)pyrimidin-4-yl)methanamine;
(6-(3-Phenoxyphenoxy)pyrimidin-4-yl)methanamine;
3-((6-(Aminomethyl)pyrimidin-4-yl)oxy)-N-phenylbenzamide;
4-(3-((6-(Aminomethyl)pyrimidin-4-yl)oxy)benzamido)benzoic acid;
4-((3-((6-(Aminomethyl)pyrimidin-4-yl)oxy)benzamido)methyl)benzoic acid;
3-((6-(Aminomethyl)pyrimidin-4-yl)oxy)-N-(benzo[b]thiophen-2-ylmethyl)benzamide;
3-(3-((6-(Aminomethyl)pyrimidin-4-yl)oxy)benzamido)propanoic acid;
4-(3-((6-(Aminomethyl)pyrimidin-4-yl)oxy)benzamido)butanoic acid;
3-((6-(Aminomethyl)pyrimidin-4-yl)oxy)-N-(2-hydroxyethyl)benzamide;
(S)-(3-((6-(Aminomethyl)pyrimidin-4-yl)oxy)phenyl)(3-hydroxypyrrolidin-1-yl)methanone;
Racemic-trans-(3-((6-(aminomethyl)pyrimidin-4-yl)oxy)phenyl)(3-fluoro-4-hydroxypyrrolidin-1-yl)methanone;
3-((6-(Aminomethyl)pyrimidin-4-yl)oxy)-N-(2-(methylsulfonyl)ethyl)benzamide;
3-((6-(Aminomethyl)pyrimidin-4-yl)oxy)-N-(2-sulfamoylethyl)benzamide;
2-(5-((6-(Aminomethyl)pyrimidin-4-yl)oxy)-2-oxo-3,4-dihydroquinolin-1(2H)-yl)acetic acid;
(5-(6-(Aminomethyl)pyrimidin-4-yl)oxy)-1-(2-hydroxyethyl)-3,4-dihydroquinolin-2(1H)-one;
5-((6-(Aminomethyl)pyrimidin-4-yl)oxy)-1-((6-methoxypyridin-3-yl)methyl)-3,4-dihydroquinolin-2(1H)-one;
(6-((1H-Indol-6-yl)oxy)pyrimidin-4-yl)methanamine;
(6-((1H-Indol-5-yl)oxy)pyrimidin-4-yl)methanamine;
(6-((1H-Indol-4-yl)oxy)pyrimidin-4-yl)methanamine;
(6-((1-Ethyl-1H-indol-4-yl)oxy)pyrimidin-4-yl)methanamine;
Methyl 2-(4-((6-(aminomethyl)pyrimidin-4-yl)oxy)-1H-indol-1-yl)acetate;
2-(4-((6-(Aminomethyl)pyrimidin-4-yl)oxy)-1H-indol-1-yl)acetic acid;
2-(4-((6-(Aminomethyl)pyrimidin-4-yl)oxy)-1H-indol-1-yl)-1-(piperidin-1-yl)ethan-1-one;
2-(4-((6-(Aminomethyl)pyrimidin-4-yl)oxy)-1H-indol-1-yl)-N-methyl-N-phenylacetamide;
(6-((1-(2-(Methylsulfonyl)ethyl)-1H-indol-4-yl)oxy)pyrimidin-4-yl)methanamine;
(6-((1-Benzyl-1H-indol-4-yl)oxy)pyrimidin-4-yl)methanamine;
Methyl 3-((4-((6-(aminomethyl)pyrimidin-4-yl)oxy)-1H-indol-1-yl)methyl)benzoate;
3-((4-((6-(Aminomethyl)pyrimidin-4-yl)oxy)-1H-indol-1-yl)methyl)benzoic acid;
(6-((1-((6-Methoxypyridin-3-yl)methyl)-1H-indol-5-yl)oxy)pyrimidin-4-yl)methanamine;
(6-((1-((6-Methoxypyridin-3-yl)methyl)-2-methyl-1H-indol-4-yl)oxy)pyrimidin-4-yl)methanamine;
(6-((1-((6-Methoxypyridin-3-yl)methyl)-3-methyl-1H-indol-4-yl)oxy)pyrimidin-4-yl)methanamine;
(6-((1-(1-Methyl-1H-pyrazol-4-yl)-1H-indol-4-yl)oxy)pyrimidin-4-yl)methanamine;
3-(((6-(Aminomethyl)pyrimidin-4-yl)oxy)methyl)-N,N-dimethylbenzamide;
3-(((6-(Aminomethyl)pyrimidin-4-yl)oxy)methyl)-N-(2-hydroxy-2-methylpropyl)benzamide;
Racemic-(3-(((6-(aminomethyl)pyrimidin-4-yl)oxy)methyl)phenyl)(3-hydroxy-3-(trifluoromethyl)piperidin-1-yl)methanone;
Racemic-3-(((6-(aminomethyl)pyrimidin-4-yl)oxy)methyl)-N-((3-methyl-2-oxooxazolidin-5-yl)methyl)benzamide;
3-(((6-(Aminomethyl)pyrimidin-4-yl)oxy)methyl)-N-(2-(methylsulfonyl)ethyl)benzamide;
3-(((6-(Aminomethyl)pyrimidin-4-yl)oxy)methyl)-N-(2-(2-oxooxazolidin-3-yl)ethyl)benzamide;
N-(2-(1H-Pyrazol-1-yl)ethyl)-3-(((6-(aminomethyl)pyrimidin-4-yl)oxy)methyl)benzamide;
3-(((6-(Aminomethyl)pyrimidin-4-yl)oxy)methyl)-N-phenylbenzamide;
3-(((6-(Aminomethyl)pyrimidin-4-yl)oxy)methyl)-N-benzylbenzamide;
3-((6-(Aminomethyl)pyrimidin-4-yl)amino)-N-phenylbenzamide;
3-(((6-(Aminomethyl)pyrimidin-4-yl)amino)methyl)-N-phenylbenzamide;
4-(3-(((6-(Aminomethyl)pyrimidin-4-yl)amino)methyl)benzamido)benzoic acid;
3-(((6-(Aminomethyl)pyrimidin-4-yl)(methyl)amino)methyl)-N-phenylbenzamide;
N-(2-(1H-Tetrazol-1-yl)ethyl)-3-(((6-(aminomethyl)pyrimidin-4-yl)oxy)methyl)benzamide;
5-((6-(Aminomethyl)pyrimidin-4-yl)oxy)-1-(2-(methylsulfonyl)ethyl)-3,4-dihydroquinolin-2(1H)-one;
2-(4-((6-(Aminomethyl)pyrimidin-4-yl)oxy)-1H-indol-1-yl)ethan-1-ol;
(6-((1-(Oxetan-3-yl)-1H-indol-4-yl)oxy)pyrimidin-4-yl)methanamine;
(3-((6-(Aminomethyl)pyrimidin-4-yl)oxy)phenyl)(6-chloroindolin-1-yl)methanone;
(3-((6-(Aminomethyl)pyrimidin-4-yl)oxy)phenyl)(2,3-dihydro-1H-pyrrolo[2,3-b]pyridin-1-yl)methanone;
(3-((6-(Aminomethyl)pyrimidin-4-yl)oxy)phenyl)(2,3-dihydro-1H-pyrrolo[2,3-c]pyridin-1-yl)methanone;
N-((1H-Indol-2-yl)methyl)-3-((6-(aminomethyl)pyrimidin-4-yl)oxy)benzamide;
3-((6-(Aminomethyl)pyrimidin-4-yl)oxy)-N-(pyridin-3-ylmethyl)benzamide;
3-((6-(Aminomethyl)pyrimidin-4-yl)oxy)-N-((2-chloropyridin-4-yl)methyl)benzamide; 3-((6-(Aminomethyl)pyrimidin-4-yl)oxy)-N-benzylbenzamide;
(R)-3-((6-(Aminomethyl)pyrimidin-4-yl)oxy)-N-(2-hydroxy-1-phenylethyl)benzamide;
(3-((6-(Aminomethyl)pyrimidin-4-yl)oxy)phenyl)(4-(pyridin-2-yl)piperazin-1-yl)methanone;
(3-((6-(Aminomethyl)pyrimidin-4-yl)oxy)phenyl)(4,4-dimethylpiperidin-1-yl)methanone;
(6-((1H-Indazol-4-yl)oxy)pyrimidin-4-yl)methanamine;
(1-(6-(Aminomethyl)pyrimidin-4-yl)-1H-indazol-4-yl)methanol;
3-((6-(Aminomethyl)pyrimidin-4-yl)oxy)-N-((4′-fluoro-[1,1′-biphenyl]-4-yl)methyl)benzamide;
3-((6-(Aminomethyl)pyrimidin-4-yl)oxy)-N-(quinolin-2-ylmethyl)benzamide;
3-((6-(Aminomethyl)pyrimidin-4-yl)oxy)-N-((3-chlorobenzo[b]thiophen-2-yl)methyl)benzamide;
3-((6-(Aminomethyl)pyrimidin-4-yl)oxy)-N-((5-fluoro-1H-indol-2-yl)methyl)benzamide;
3-((6-(Aminomethyl)pyrimidin-4-yl)oxy)-N-((1-methyl-1H-indol-2-yl)methyl)benzamide;
N-((1H-Pyrrolo[2,3-b]pyridin-2-yl)methyl)-3-((6-(aminomethyl)pyrimidin-4-yl)oxy)benzamide;
3-((6-(Aminomethyl)pyrimidin-4-yl)oxy)-N-(benzo[d]oxazol-2-ylmethyl)benzamide;
(3-((6-(Aminomethyl)pyrimidin-4-yl)oxy)phenyl)(5,6-dihydro-1,7-naphthyridin-7(8H)-yl)methanone;
(3-((6-(Aminomethyl)pyrimidin-4-yl)oxy)phenyl)(3-methyl-6,7-dihydro-1H-pyrazolo[4,3-c]pyridin-5(4H)-yl)methanone;
(3-((6-(Aminomethyl)pyrimidin-4-yl)oxy)phenyl)(5H-pyrrolo[2,3-b:5,4-c′]dipyridin-7(6H,8H,9H)-yl)methanone;
(3-((6-(Aminomethyl)pyrimidin-4-yl)oxy)phenyl)(4-phenylpiperazin-1-yl)methanone;
(3-((6-(Aminomethyl)pyrimidin-4-yl)oxy)phenyl)(4-(3,5-dichloropyridin-2-yl)piperazin-1-yl)methanone;
(3-((6-(Aminomethyl)pyrimidin-4-yl)oxy)phenyl)(4-hydroxy-4-(trifluoromethyl)piperidin-1-yl)methanone;
(S)-Methyl 3-(3-((6-(aminomethyl)pyrimidin-4-yl)oxy)benzamido)pent-4-ynoate;
(R)-Methyl 3-(3-((6-(aminomethyl)pyrimidin-4-yl)oxy)benzamido)pent-4-ynoate;
3-(((6-(Aminomethyl)pyrimidin-4-yl)oxy)methyl)-N-(3-(trifluoromethyl)phenyl)benzamide;
(R)-3-(((6-(Aminomethyl)pyrimidin-4-yl)oxy)methyl)-N-(2-hydroxy-1-phenylethyl)benzamide;
3-(((6-(Aminomethyl)pyrimidin-4-yl)oxy)methyl)-N-(pyridin-3-ylmethyl)benzamide;
3-(((6-(Aminomethyl)pyrimidin-4-yl)oxy)methyl)-N-((2-chloropyridin-4-yl)methyl)benzamide;
3-(((6-(Aminomethyl)pyrimidin-4-yl)oxy)methyl)-N-(benzo[b]thiophen-2-ylmethyl)benzamide;
3-(((6-(Aminomethyl)pyrimidin-4-yl)oxy)methyl)-N-((5-fluorobenzo[b]thiophen-2-yl)methyl)benzamide;
3-(((6-(Aminomethyl)pyrimidin-4-yl)oxy)methyl)-N-(benzo[d]oxazol-2-ylmethyl)benzamide;
N-((1H-indol-2-yl)methyl)-3-(((6-(aminomethyl)pyrimidin-4-yl)oxy)methyl)benzamide;
3-(((6-(Aminomethyl)pyrimidin-4-yl)oxy)methyl)-N-((5-fluoro-1H-indol-2-yl)methyl)benzamide;
3-(((6-(Aminomethyl)pyrimidin-4-yl)oxy)methyl)-N-((1-methyl-1H-indol-2-yl)methyl)benzamide;
N-((1H-pyrrolo[2,3-b]pyridin-2-yl)methyl)-3-(((6-(aminomethyl)pyrimidin-4-yl)oxy)methyl)benzamide;
(3-(((6-(Aminomethyl)pyrimidin-4-yl)oxy)methyl)phenyl)(5,6-dihydro-1,7-naphthyridin-7(8H)-yl)methanone;
(3-(((6-(Aminomethyl)pyrimidin-4-yl)oxy)methyl)phenyl)(3-methyl-6,7-dihydro-1H-pyrazolo[4,3-c]pyridin-5(4H)-yl)methanone;
(3-(((6-(Aminomethyl)pyrimidin-4-yl)oxy)methyl)phenyl)(5H-pyrrolo[2,3-b:5,4-c′]dipyridin-7(6H,8H,9H)-yl)methanone;
(3-(((6-(Aminomethyl)pyrimidin-4-yl)oxy)methyl)phenyl)(4-phenylpiperazin-1-yl)methanone;
(3-(((6-(Aminomethyl)pyrimidin-4-yl)oxy)methyl)phenyl)(2,3-dihydro-1H-pyrrolo[2,3-b]pyridin-1-yl)methanone;
(3-(((6-(Aminomethyl)pyrimidin-4-yl)oxy)methyl)phenyl)(2,3-dihydro-1H-pyrrolo[2,3-c]pyridin-1-yl)methanone;
(3-(((6-(Aminomethyl)pyrimidin-4-yl)oxy)methyl)phenyl)(4-hydroxy-4-(trifluoromethyl)piperidin-1-yl)methanone;
Racemic-3-(1-((6-(aminomethyl)pyrimidin-4-yl)oxy)ethyl)-N-phenylbenzamide;
3-(((6-(Aminomethyl)pyrimidin-4-yl)amino)methyl)-N-(3-(trifluoromethyl)phenyl)benzamide;
3-(((6-(Aminomethyl)pyrimidin-4-yl)amino)methyl)-N-(benzo[b]thiophen-2-ylmethyl)benzamide;
3-(((6-(Aminomethyl)pyrimidin-4-yl)amino)methyl)-N-((3-chlorobenzo[b]thiophen-2-yl)methyl)benzamide;
(1-(6-(Aminomethyl)pyrimidin-4-yl)-1H-indol-4-yl)methanol;
(3-(((6-(Aminomethyl)pyrimidin-4-yl)amino)methyl)phenyl)(4-phenylpiperazin-1-yl)methanone;
5-((6-(Aminomethyl)pyrimidin-4-yl)oxy)-3,4-dihydroquinolin-2(1H)-one;
(R)-1-(6-(Aminomethyl)pyrimidin-4-yl)-N-phenylpyrrolidine-3-carboxamide;
1-(6-(Aminomethyl)pyrimidin-4-yl)-N-phenylpiperidine-4-carboxamide;
(4-(6-(Aminomethyl)pyrimidin-4-yl)piperazin-1-yl)(phenyl)methanone;
4-(6-(Aminomethyl)pyrimidin-4-yl)-N-phenylpiperazine-1-carboxamide;
1-(4-(6-(Aminomethyl)pyrimidin-4-yl)piperazin-1-yl)-2-phenylethanone; or
1-(6-(Aminomethyl)pyrimidin-4-yl)-5-(benzyloxy)-3,4-dihydroquinolin-2(1H)-one.

In some embodiments, the LOXL2i is a substituted or unsubstituted chromenonylmethylamine compound. In some embodiments, the substituted or unsubstituted chromenonylmethylamine compound is a substituted or unsubstituted chromen-4-onylmethylamine. In some embodiments, the LOXL2i is a compound described in International Patent Application No. PCT/US2016/042826 titled “Lysyl Oxidase-like 2 (LOXL2) Inhibitors and Uses Thereof” filed on Jul. 18, 2016, which is herein incorporated by reference for such compounds.

In some embodiments, the LOXL2i is a compound described in Table 1 of International Patent Application No. PCT/US2016/042826.

In some embodiments, the LOXL2i is:
2-(Aminomethyl)-6-bromo-4H-chromen-4-one;
2-(Aminomethyl)-7-bromo-4H-chromen-4-one;
2-(Aminomethyl)-6-ethynyl-4H-chromen-4-one;
2-(Aminomethyl)-6-(3-hydroxy-3-methylbut-1-yn-1-yl)-4H-chromen-4-one;
2-(Aminomethyl)-6-(3-methylbut-3-en-1-yn-1-yl)-4H-chromen-4-one;
2-(Aminomethyl)-6-phenyl-4H-chromen-4-one;
2-(Aminomethyl)-6-(pyridin-2-yl)-4H-chromen-4-one;
2-(Aminomethyl)-6-(pyridin-3-yl)-4H-chromen-4-one;
2-(Aminomethyl)-6-(quinolin-3-yl)-4H-chromen-4-one;
2-(Aminomethyl)-6-(1H-pyrazol-1-yl)-4H-chromen-4-one;
2-(Aminomethyl)-7-(1H-pyrazol-1-yl)-4H-chromen-4-one;
2-(Aminomethyl)-6-(1-methyl-1H-pyrazol-4-yl)-4H-chromen-4-one;
2-(Aminomethyl)-6-(1-methyl-1H-1,2,3-triazol-4-yl)-4H-chromen-4-one;
2-(Aminomethyl)-6-(1-phenyl-1H-1,2,3-triazol-4-yl)-4H-chromen-4-one;
2-(Aminomethyl)-6-(1-benzyl-1H-1,2,3-triazol-4-yl)-4H-chromen-4-one;
2-(Aminomethyl)-6-(1-(2-hydroxyethyl)-1H-1,2,3-triazol-4-yl)-4H-chromen-4-one;
2-(4-(2-(Aminomethyl)-4-oxo-4H-chromen-6-yl)-1H-1,2,3-triazol-1-yl)-N,N-dimethylacetamide;
2-(Aminomethyl)-N,N-dimethyl-4-oxo-4H-chromene-6-carboxamide;
2-(Aminomethyl)-6-(piperidine-1-carbonyl)-4H-chromen-4-one;
(S)-2-(Aminomethyl)-6-(3-hydroxypyrrolidin-1-yl)-4H-chromen-4-one;
N-(2-(1H-1,2,4-Triazol-1-yl)ethyl)-2-(aminomethyl)-4-oxo-4H-chromene-6-carboxamide;
2-(Aminomethyl)-4-oxo-N-(2-sulfamoylethyl)-4H-chromene-6-carboxamide;
(R)-2-(Aminomethyl)-6-(3-aminopyrrolidine-1-carbonyl)-4H-chromen-4-one;
Methyl (R)-1-(2-(aminomethyl)-4-oxo-4H-chromene-6-carbonyl)pyrrolidine-3-carboxylate;
Racemic-trans-2-(aminomethyl)-6-(3-fluoro-4-hydroxypyrrolidine-1-carbonyl)-4H-chromen-4-one;
2-(Aminomethyl)-N-(2-(methyl sulfonyl)ethyl)-4-oxo-4H-chromene-6-carboxamide;
2-(Aminomethyl)-6-methoxy-4H-chromen-4-one;
2-(Aminomethyl)-7-methoxy-4H-chromen-4-one;
2-(Aminomethyl)-7-(benzyloxy)-4H-chromen-4-one;
2-(Aminomethyl)-6-(benzyloxy)-4H-chromen-4-one;
2-(Aminomethyl)-7-ethynyl-4H-chromen-4-one;
2-((2-(Aminomethyl)-4-oxo-4H-chromen-7-yl)oxy)-N,N-dimethylacetamide;
2-(Aminomethyl)-7-(1-phenyl-1H-1,2,3-triazol-4-yl)-4H-chromen-4-one;
2-((2-(Aminomethyl)-4-oxo-4H-chromen-6-yl)oxy)acetic acid;
2-((2-(Aminomethyl)-4-oxo-4H-chromen-6-yl)oxy)-N,N-dimethylacetamide;
Methyl (S)-1-(2-(aminomethyl)-4-oxo-4H-chromene-6-carbonyl)pyrrolidine-3-carboxylate;
1-(2-(Aminomethyl)-4-oxo-4H-chromene-6-carbonyl)pyrrolidine-3-carboxylic acid;
(R)-1-(2-(Aminomethyl)-4-oxo-4H-chromene-6-carbonyl)pyrrolidine-3-carboxylic acid;
(S)-1-(2-(Aminomethyl)-4-oxo-4H-chromene-6-carbonyl)pyrrolidine-3-carboxylic acid;
2-(Aminomethyl)-7-(1-methyl-1H-1,2,3-triazol-4-yl)-4H-chromen-4-one;
2-(Aminomethyl)-7-(4-phenylpiperazin-1-yl)-4H-chromen-4-one;
2-(Aminomethyl)-7-(4-benzoylpiperazin-1-yl)-4H-chromen-4-one;
2-(Aminomethyl)-7-(3,4-dihydroquinolin-1(2H)-yl)-4H-chromen-4-one;
2-(Aminomethyl)-7-hydroxy-4H-chromen-4-one;
2-(Aminomethyl)-7-isobutoxy-4H-chromen-4-one;
2-(Aminomethyl)-7-(prop-2-yn-1-yloxy)-4H-chromen-4-one;
2-(Aminomethyl)-7-(2-phenoxyethoxy)-4H-chromen-4-one;
2-(Aminomethyl)-7-((1-phenyl-1H-1,2,3-triazol-4-yl)methoxy)-4H-chromen-4-one;
3-(((2-(Aminomethyl)-4-oxo-4H-chromen-7-yl)oxy)methyl)-N-phenylbenzamide;
3-((2-(Aminomethyl)-4-oxo-4H-chromen-7-yl)amino)-N-phenylbenzamide;
2-(Aminomethyl)-8-bromo-4H-chromen-4-one;
2-(Aminomethyl)-8-ethynyl-4H-chromen-4-one;
2-(Aminomethyl)-8-hydroxy-4H-chromen-4-one;
2-(Aminomethyl)-8-(prop-2-yn-1-yloxy)-4H-chromen-4-one;
2-(Aminomethyl)-8-(benzyloxy)-4H-chromen-4-one;
2-(Aminomethyl)-8-phenethoxy-4H-chromen-4-one;
2-(Aminomethyl)-8-(2-phenoxyethoxy)-4H-chromen-4-one;
2-((2-(Aminomethyl)-4-oxo-4H-chromen-8-yl)oxy)-N-phenylacetamide;
3-(((2-(Aminomethyl)-4-oxo-4H-chromen-8-yl)oxy)methyl)-N-phenylbenzamide;
2-(Aminomethyl)-N-(2-hydroxyethyl)-4-oxo-4H-chromene-6-carboxamide;
2-(Aminomethyl)-6-((3S,4S)-3-fluoro-4-hydroxypyrrolidine-1-carbonyl)-4H-chromen-4-one;
2-(Aminomethyl)-6-((3R,4R)-3-fluoro-4-hydroxypyrrolidine-1-carbonyl)-4H-chromen-4-one;
2-(Aminomethyl)-7-((4-(trifluoromethyl)benzyl)oxy)-4H-chromen-4-one;
2-(Aminomethyl)-7-((3-phenylprop-2-yn-1-yl)oxy)-4H-chromen-4-one;
2-(Aminomethyl)-7-((4-methoxybenzyl)oxy)-4H-chromen-4-one;
2-(Aminomethyl)-7-(quinolin-2-ylmethoxy)-4H-chromen-4-one;
2-(Aminomethyl)-7-(benzo[b]thiophen-2-ylmethoxy)-4H-chromen-4-one;
2-(Aminomethyl)-7-(3-(trifluoromethyl)phenoxy)-4H-chromen-4-one;
2-(Aminomethyl)-7-phenoxy-4H-chromen-4-one;
2-(Aminomethyl)-7-(benzyloxy)-6-methoxy-4H-chromen-4-one; or
2-(Aminomethyl)-7-((1-phenyl-1H-pyrazol-4-yl)amino)-4H-chromen-4-one.

In some embodiments, the LOXL2i is a substituted or unsubstituted quinolinonylmethylamine. In some embodiments, the substituted or unsubstituted quinolinonylmethylamine compound is a substituted or unsubstituted quinolin-4-onylmethylamine.

In some embodiments, the LOXL2i is a compound described in International Patent Application No. PCT/US2017/016847 entitled “Quinolinone Lysyl Oxidase-Like 2 Inhibitors and Uses Thereof” filed on February 7, 2017, which is herein incorporated by reference for such compounds.

In some embodiments, the LOXL2i is a compound described in Table 1 of International Patent Application No. PCT/US2017/016847.

In some embodiments, the LOXL2i is:
2-(Aminomethyl)quinolin-4(1H)-one;
2-(Aminomethyl)-1-methylquinolin-4(1H)-one;
2-(Aminomethyl)-6-bromoquinolin-4(1H)-one;
2-(Aminomethyl)-6-ethynylquinolin-4(1H)-one;
2-(Aminomethyl)-6-phenylquinolin-4(1H)-one;
2-(Aminomethyl)-6-(1-phenyl-1H-1,2,3-triazol-4-yl)quinolin-4(1H)-one;
2-(Aminomethyl)-6-(phenylethynyl)quinolin-4(1H)-one;
2-(Aminomethyl)-6-(1H-pyrazol-1-yl)quinolin-4(1H)-one;
2-(Aminomethyl)-6-methoxyquinolin-4(1H)-one;
2-(Aminomethyl)-6-hydroxyquinolin-4(1H)-one;
2-(Aminomethyl)-6-(4-fluorophenyl)quinolin-4(1H)-one;
2-(Aminomethyl)-6-(3-fluorophenyl)quinolin-4(1H)-one;
2-(Aminomethyl)-6-(2-fluorophenyl)quinolin-4(1H)-one;
2-(Aminomethyl)-6-(1H-1,2,3-triazol-1-yl)quinolin-4(1H)-one;
2-(Aminomethyl)-6-(2H-tetrazol-2-yl)quinolin-4(1H)-one;
2-(Aminomethyl)-6-(1H-tetrazol-1-yl)quinolin-4(1H)-one;
2-(Aminomethyl)-6-(2-methyl-2H-tetrazol-5-yl)quinolin-4(1H)-one;
2-(Aminomethyl)-6-((4-fluorophenyl)ethynyl)quinolin-4(1H)-one;
2-(Aminomethyl)-6-((3-fluorophenyl)ethynyl)quinolin-4(1H)-one;
2-(Aminomethyl)-6-((2-fluorophenyl)ethynyl)quinolin-4(1H)-one;
2-(Aminomethyl)-6-phenoxyquinolin-4(1H)-one;
2-(Aminomethyl)-6-(2-fluorophenoxy)quinolin-4(1H)-one;
2-(Aminomethyl)-6-(3-fluorophenoxy)quinolin-4(1H)-one;
2-(Aminomethyl)-6-(4-fluorophenoxy)quinolin-4(1H)-one;
2-(Aminomethyl)-6-(benzyloxy)quinolin-4(1H)-one;
2-(Aminomethyl)-6-((2-fluorobenzyl)oxy)quinolin-4(1H)-one;
2-(Aminomethyl)-6-((3-fluorobenzyl)oxy)quinolin-4(1H)-one;
2-(Aminomethyl)-6-((4-fluorobenzyl)oxy)quinolin-4(1H)-one;
2-(Aminomethyl)-6-(phenylamino)quinolin-4(1H)-one;
2-(Aminomethyl)-6-(benzylamino)quinolin-4(1H)-one;
2-(Aminomethyl)-6-(prop-2-yn-1-yloxy)quinolin-4(1H)-one;
2-(Aminomethyl)-6-((4,4,4-trifluorobut-2-yn-1-yl)oxy)quinolin-4(1H)-one;
2-(Aminomethyl)-6-((3-phenylprop-2-yn-1-yl)oxy)quinolin-4(1H)-one;
2-(Aminomethyl)-6-(prop-2-yn-1-ylamino)quinolin-4(1H)-one;
2-(Aminomethyl)-6-((4,4,4-trifluorobut-2-yn-1-yl)amino)quinolin-4(1H)-one;
2-(Aminomethyl)-6-((3-phenylprop-2-yn-1-yl)amino)quinolin-4(1H)-one;
2-(Aminomethyl)-6-(pyridin-2-yl)quinolin-4(1H)-one;
2-(Aminomethyl)-6-(pyridin-3-yl)quinolin-4(1H)-one;
2-(Aminomethyl)-6-(pyridin-4-yl)quinolin-4(1H)-one;
2-(Aminomethyl)-6-(pyrimidin-5-yl)quinolin-4(1H)-one;
2-(Aminomethyl)-6-(pyrazin-2-yl)quinolin-4(1H)-one;
2-(Aminomethyl)-6-(oxazol-2-yl)quinolin-4(1H)-one;
2-(Aminomethyl)-6-(thiazol-2-yl)quinolin-4(1H)-one;
2′-(Aminomethyl)-[2,6′-biquinolin]-4′(1′H)-one;
2′-(Aminomethyl)-[3,6′-biquinolin]-4′(1′H)-one;
2-(Aminomethyl)-6-(5-methyl-2-oxopyridin-1(2H)-yl)quinolin-4(1H)-one;
2-(Aminomethyl)-6-(1-methyl-1H-pyrazol-4-yl)quinolin-4(1H)-one;
2-(Aminomethyl)-6-(1-phenyl-1H-pyrazol-4-yl)quinolin-4(1H)-one;
2-(Aminomethyl)-6-(4-phenyl-1H-pyrazol-1-yl)quinolin-4(1H)-one;
2-(Aminomethyl)-6-((1-methyl-1H-pyrazol-4-yl)amino)quinolin-4(1H)-one;
2-(Aminomethyl)-6-((1-phenyl-1H-pyrazol-4-yl)amino)quinolin-4(1H)-one;
2-(Aminomethyl)-6-((1-(2-fluorophenyl)-1H-pyrazol-4-yl)amino)quinolin-4(1H)-one;
2-(Aminomethyl)-6-((1-(3-fluorophenyl)-1H-pyrazol-4-yl)amino)quinolin-4(1H)-one;
2-(Aminomethyl)-6-((1-(4-fluorophenyl)-1H-pyrazol-4-yl)amino)quinolin-4(1H)-one;
2′-(Aminomethyl)-3,4-dihydro-2H-[1,6′-biquinolin]-4′(1′H)-one;
2-(Aminomethyl)-6-(4-phenylpiperazin-1-yl)quinolin-4(1H)-one;
2-(Aminomethyl)-6-(pyrrolidine-1-carbonyl)quinolin-4(1H)-one;
2-(Aminomethyl)-6-((3S,4S)-3-fluoro-4-hydroxypyrrolidine-1-carbonyl)quinolin-4(1H)-one;
2-(Aminomethyl)-6-((3R,4R)-3-fluoro-4-hydroxypyrrolidine-1-carbonyl)quinolin-4(1H)-one;
2-(Aminomethyl)-4-oxo-1,4-dihydroquinoline-6-carboxamide;
2-(Aminomethyl)-N-methyl-4-oxo-1,4-dihydroquinoline-6-carboxamide;
2-(Aminomethyl)-4-oxo-1,4-dihydroquinoline-6-carboxylic acid;
2-(Aminomethyl)-6-isopropoxyquinolin-4(1H)-one;
2-(Aminomethyl)-6-isobutoxyquinolin-4(1H)-one;
2-(Aminomethyl)-[6,8′-biquinolin]-4(1H)-one;
2-(Aminomethyl)-6-(5-(benzyloxy)pyridin-3-yl)quinolin-4(1H)-one;
2-(Aminomethyl)-6-(4-chlorophenyl)quinolin-4(1H)-one;
2-(Aminomethyl)-6-(3-chlorophenyl)quinolin-4(1H)-one;
2-(Aminomethyl)-6-(2-chlorophenyl)quinolin-4(1H)-one;
2-(Aminomethyl)-6-(2-chloro-4-fluorophenyl)quinolin-4(1H)-one;
2-(Aminomethyl)-6-(4-chloro-2-fluorophenyl)quinolin-4(1H)-one;
2-(Aminomethyl)-6-(3-fluoro-4-methoxyphenyl)quinolin-4(1H)-one;
2-(Aminomethyl)-6-(2-fluoro-4-methoxyphenyl)quinolin-4(1H)-one;
2-(Aminomethyl)-6-(6-(trifluoromethyl)pyridin-3-yl)quinolin-4(1H)-one;
2-(Aminomethyl)-6-(5-(trifluoromethyl)pyridin-3-yl)quinolin-4(1H)-one;
2-(Aminomethyl)-6-(4-(trifluoromethyl)pyridin-3-yl)quinolin-4(1H)-one;
2-(Aminomethyl)-6-(2-(trifluoromethyl)pyrimidin-5-yl)quinolin-4(1H)-one; or
2-(Aminomethyl)-6-(2-methoxyethoxy)quinolin-4(1H)-one.

The identity of the ‘warhead’ group can alter the specificity of the interaction of the probe compound with LOXL2.

In one aspect, the probe compound described herein is a compound that has the following structure of Formula (I):

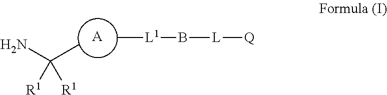

wherein,
Q is a tag moiety for the detection, isolation, or detection and isolation of the compound of Formula (I) in a biological sample;
or Q is absent provided that the compound of Formula (I) comprises a radioactive or an isotopic variant of any atom in the compound of Formula (I);
L is absent or a linker;
each R¹ is independently H, D, or F;
ring A is an unsubstituted or substituted aryl, or an unsubstituted or substituted heterocycle, wherein if ring A is substituted then ring A is substituted with 1, 2, or 3 R^{a} groups;
L¹ is X¹—Y¹—, —Y¹—X¹—, or Y¹;
   X¹ is —O—, —S—, —S(═O)—, —S(═O)₂—, —C(═O)—, —C(═O)O—, —C(═O)NR²—, —NR²C(═O)—, or —NR²—;
      R² is H, C₁-C₆alkyl, C₁-C₆fluoroalkyl, or C₁-C₆deuteroalkyl;
   Y¹ is absent, or C₁-C₆alkylene;
B is absent or an unsubstituted or substituted monocyclic carbocycle, unsubstituted or substituted bicyclic carbocycle, unsubstituted or substituted monocyclic heterocycle, or unsubstituted or substituted bicyclic heterocycle, wherein if B is substituted then B is substituted with one or more R^{b};
each R^{a}, and R^{b} is independently selected from the group consisting of H, D, halogen, CN, —OR⁵, —SR⁵, —S(═O)R⁴, —S(═O)₂R⁴, —S(═O)₂N(R⁵)₂, NR⁵S(═O)₂R⁴, C(═O)R⁴, OC(═O)R⁴, CO₂R⁵, OCO₂R⁴, N(R⁴)₂, OC(═O)N(R⁵)₂, —NHC(═O)R⁴, —NHC(═O)OR⁴, C₁-C₆alkyl, C₁-C₆fluoroalkyl, C₁-C₆deuteroalkyl, C₁-C₆heteroalkyl, substituted or unsubstituted C₃-C₁₀cycloalkyl, substituted or unsubstituted C₂-C₁₀heterocycloalkyl, substituted or unsubstituted aryl, or substituted or unsubstituted heteroaryl;
each R⁴ is independently selected from C₁-C₆alkyl, C₁-C₆fluoroalkyl, C₁-C₆deuteroalkyl, C₁-C₆heteroalkyl, substituted or unsubstituted C₃-C₁₀cycloalkyl, substituted or unsubstituted C₂-C₁₀heterocycloalkyl, substituted or unsubstituted aryl, and substituted or unsubstituted heteroaryl;
each R⁵ is independently selected from H, C₁-C₆alkyl, C₁-C₆fluoroalkyl, C₁-C₆deuteroalkyl, C₁-C₆heteroalkyl, substituted or unsubstituted C₃-C₁₀cycloalkyl, substituted or unsubstituted C₂-C₁₀heterocycloalkyl, substituted or unsubstituted aryl, substituted or unsubstituted heteroaryl, —C₁-C₄alkylene-(substituted or unsubstituted C₃-C₈cycloalkyl), substituted or unsubstituted C₂-C₈heterocycloalkyl, —C₁-C₄alkylene-(substituted or unsubstituted C₂-C₈heterocycloalkyl), substituted or unsubstituted aryl, —C₁-C₄alkylene-(substituted or unsubstituted aryl), substituted or unsubstituted heteroaryl, and —C₁-C₄alkylene-(substituted or unsubstituted heteroaryl); or two R⁵ on the same N atom are taken together with the N atom to which they are attached to a substituted or unsubstituted N-containing heterocycle.

In some embodiments, L is absent or a linker with the formula -L²-C-L³-;
L² is X²—Y²—, —Y²—X²—, or Y²;
   C² is —O—, —S—, —S(═O)—, —S(═O)₂—, —S(═O)₂NR³—, —C(═O)—, —C(═O)O—, —C(═O)NR³—, —NR³C(═O)—, —NR³S(═O)₂—, or —NR³—;
      R³ is H, C₁-C₆alkyl, C₁-C₆fluoroalkyl, or C₁-C₆deuteroalkyl;
   Y² is absent, or C₁-C₆alkylene;
C is absent, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₈cycloalkyl, —C₁-C₄alkylene-(substituted or unsubstituted C₃-C₈cycloalkyl), substituted or unsubstituted C₂-C₈heterocycloalkyl, —C₁-C₄alkylene-(substituted or unsubstituted C₂-C₈heterocycloalkyl), substituted or unsubstituted aryl, —C₁-C₄alkylene-(substituted or unsubstituted aryl), substituted or unsubstituted heteroaryl, or —C₁-C₄alkylene-(substituted or unsubstituted heteroaryl); wherein if C is substituted then C is substituted with one or more R^{c};
or when C and R³ are attached to the same N-atom then C and R³ are taken together with the N atom to which they are attached to form ring D, wherein ring D is a substituted or unsubstituted N-containing heterocycle, wherein if ring D is substituted then ring D is substituted with 1, 2, or 3 R^{d};
each R^{c}, and R^{d}, is independently selected from the group consisting of D, halogen, CN, —OR⁵, —SR⁵, —S(═O)R⁴, —S(═O)₂R⁴, —S(═O)₂N(R⁵)₂, NR⁵S(═O)₂R⁴, C(═O)R⁴, OC(═O)R⁴, CO₂R⁵, OCO₂R⁴, N(R⁴)₂, OC(═O)N(R⁵)₂, —NHC(═O)R⁴, —NHC(═O)OR⁴, C₁-C₆alkyl, C₁-C₆fluoroalkyl, C₁-C₆deuteroalkyl, C₁-C₆heteroalkyl, substituted or unsubstituted C₃-C₁₀cycloalkyl, substituted or unsubstituted C₂-C₁₀heterocycloalkyl, substituted or unsubstituted aryl, or substituted or unsubstituted heteroaryl;
or two R^{d} groups attached to the same carbon atom are taken together with carbon atom to which they are attached to form either a substituted or unsubstituted carbocycle or substituted or unsubstituted heterocycle;
L³ is absent or -L⁴-L⁵-L⁶-L⁷-;
   L⁴ is absent, —O—, —S—, —S(═O)—, —S(═O)_{2—}, —CH(OH)—, —C(═O)—, —C(═O)NH—, —NHC(═O)—, —C(═O)O—, —OC(═O)—, —CH(═N)—, —CH(═N—NH)—, —CCH₃(═N)—, —CCH₃(═N—NH)—, —OC(═O)NH—, —NHC(═O)NH—, —NHC(═O)O—, —(CH₂)ₚ—, —(OCH₂CH₂)ₚ—, or —(OCH₂CH₂)ₚ—, p is 1, 2, 3, 4, 5, or 6;
   L⁵ is absent, unsubstituted or substituted alkylene, unsubstituted or substituted heteroalkylene, unsubstituted or substituted alkenylene, unsubstituted or substituted alkynylene, unsubstituted or substituted cycloalkylene, unsubstituted or substituted heterocycloalkylene, unsubstituted or substituted arylene, unsubstituted or substituted heteroarylene, —(OCH₂CH₂)ₚ—, or —(OCH₂CH₂)ₚ—, p is 1, 2, 3, 4, 5, 6, 7, 8, 9, 10, 11, or 12;
   L⁶ is absent, —O—, —S—, —S(O)—, —S(O)_{2—}, —CH(OH)—, —C(═O)—, —C(═O)NH—, —NHC(═O)—, —C(═O)O—, —OC(═O)—, —OC(═O)NH—, —NHC(═O)NH—, or —NHC(═O)O—;
   L⁷ is absent, unsubstituted or substituted alkylene, unsubstituted or substituted heteroalkylene.

In some embodiments, each R¹ is H.

In some embodiments, each R^{a} is independently selected from the group consisting of H, D, halogen, —CN, —OR⁵, —SR⁵, —S(═O)R⁴, —S(═O)₂R⁴, —S(═O)₂N(R⁵)₂, —NR²S(═O)₂R⁴, —C(═O)R⁴, —OC(═O)R⁴, —CO₂R⁵, —OCO₂R⁵, —N(R⁵)₂, —OC(═O)N(R⁵)₂, —NR²C(═O)R⁴, —NR²C(═O)OR⁵, —CH₃, CH₂CH₃, —CH(CH₃)₂, —C(CH₃)₃, —CH₂F, —CHF₂, —CF₃, C₁-C₆deuteroalkyl, C₁-C₆heteroalkyl, substituted or unsubstituted C₃-C₁₀cycloalkyl, substituted or unsubstituted monocyclic C₂-C₆heterocycloalkyl, substituted or unsubstituted phenyl, or substituted or unsubstituted monocyclic heteroaryl.

In some embodiments, each R^{a} is independently selected from the group consisting of H, D, F, Cl, Br, —CN, —OR⁵, —CO₂R⁵, —N(R⁵)₂, —NR²C(═O)R⁴, —CH₃, —CH₂CH₃, —CH(CH₃)₂, —C(CH₃)₃, —CH₂F, —CHF₂, —CF₃, cyclopropyl, cyclobutyl, cyclopentyl, cyclohexyl, substituted or unsubstituted monocyclic C₂-C₆heterocycloalkyl, substituted or unsubstituted phenyl, or substituted or unsubstituted monocyclic heteroaryl.

In some embodiments, each R^{a} is independently selected from the group consisting of H, D, F, Cl, Br, —CN, —OCH₃, —OCF₃, —CH₃, —CH₂F, —CHF₂, —CF₃, cyclopropyl, cyclobutyl, cyclopentyl, cyclohexyl, substituted or unsubstituted monocyclic C₂-C₆heterocycloalkyl, substituted or unsubstituted phenyl, or substituted or unsubstituted monocyclic heteroaryl.

In some embodiments, each R^{a} is independently selected from the group consisting of H, D, F, Cl, Br, —CN, —OCH₃, —OCF₃, —CH₃, —CH₂F, —CHF₂, —CF₃.

In some embodiments, each R^{a} is independently selected from the group consisting of H, D, or —CF₃.

In some embodiments, ring A is an unsubstituted or substituted heterocycle, wherein if ring A is substituted then ring A is substituted with 1, 2, or 3 R^{a} groups.

In some embodiments, ring A is an unsubstituted or substituted monocyclic aromatic heterocycle, wherein if ring A is substituted then ring A is substituted with 1, 2, or 3 R^{a} groups.

In some embodiments, ring A is an unsubstituted or substituted monocyclic aromatic 6-membered heterocycle or an unsubstituted or substituted monocyclic aromatic 5-membered heterocycle, wherein if ring A is substituted then ring A is substituted with 1, 2, or 3 R^{a} groups.

In some embodiments, ring A is an unsubstituted or substituted pyridinyl, an unsubstituted or substituted pyrimidinyl, an unsubstituted or substituted pyrazinyl, an unsubstituted or substituted pyridazinyl, or an unsubstituted or substituted triazinyl, wherein if ring A is substituted then ring A is substituted with 1, 2, or 3 R^{a} groups.

In some embodiments, ring A is an unsubstituted or substituted pyridinyl, or an unsubstituted or substituted pyrimidinyl, wherein if ring A is substituted then ring A is substituted with R^{a}.

In some embodiments, the compound of Formula (I) has the following structure of Formula (II) or Formula (III):

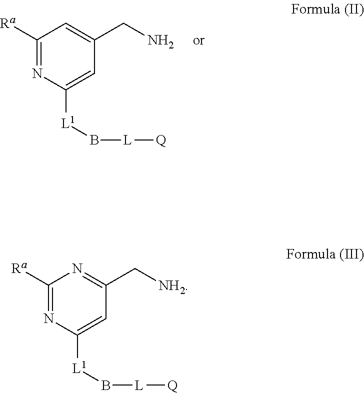

In some embodiments, the compound of Formula (I) has the following structure of Formula (IIa):

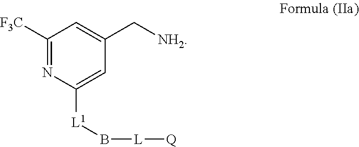

In some embodiments, ring A is an unsubstituted or substituted monocyclic aromatic 5-membered heterocycle that is an unsubstituted or substituted imidazolyl, an unsubstituted or substituted pyrazolyl, an unsubstituted or substituted triazolyl, an unsubstituted or substituted tetrazolyl, an unsubstituted or substituted furyl, an unsubstituted or substituted thienyl, an unsubstituted or substituted isoxazolyl, an unsubstituted or substituted thiazolyl, an unsubstituted or substituted oxazolyl, an unsubstituted or substituted isothiazolyl, an unsubstituted or substituted pyrrolyl, an unsubstituted or substituted oxadiazolyl, an unsubstituted or substituted thiadiazolyl, or an unsubstituted or substituted furazanyl.

In some embodiments, ring A is an unsubstituted or substituted bicyclic heterocycle.

In some embodiments, ring A is an unsubstituted or substituted quinolinone, unsubstituted or substituted isoquinolinone, unsubstituted or substituted chromone, or unsubstituted or substituted coumarin.

In some embodiments, the compound of Formula (I) has the following structure of Formula (IV), Formula (V), Formula (VI), Formula (VII), or Formula (VIII):

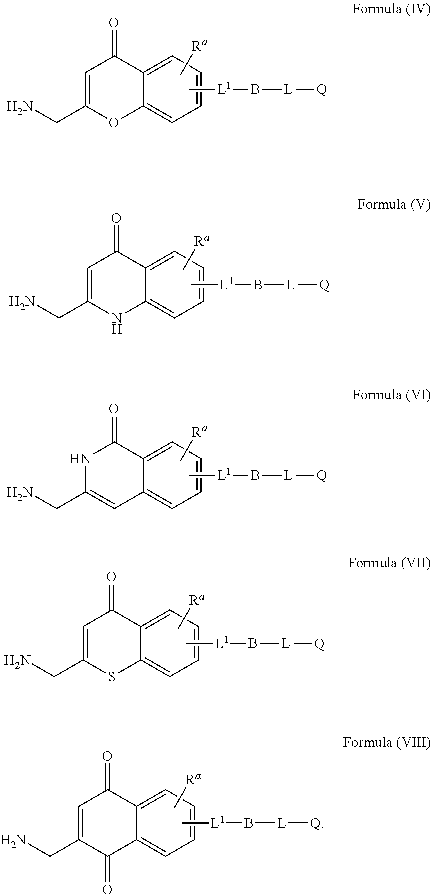

In some embodiments, ring A is an unsubstituted or substituted indolizinyl, unsubstituted or substituted indolyl, unsubstituted or substituted benzofuranyl, unsubstituted or substituted benzothiophenyl, unsubstituted or substituted indazolyl, unsubstituted or substituted benzimidazolyl, unsubstituted or substituted purinyl, unsubstituted or substituted quinolizinyl, unsubstituted or substituted quinolinyl, unsubstituted or substituted isoquinolinyl, unsubstituted or substituted cinnolinyl, unsubstituted or substituted phthalazinyl, unsubstituted or substituted quinazolinyl, unsubstituted or substituted quinoxalinyl, unsubstituted or substituted 1,8-naphthyridinyl, or unsubstituted or substituted pteridinyl.

In some embodiments, the compound of Formula (I) has the following structure of Formula (IX), Formula (X), Formula (XI), or Formula (XII):

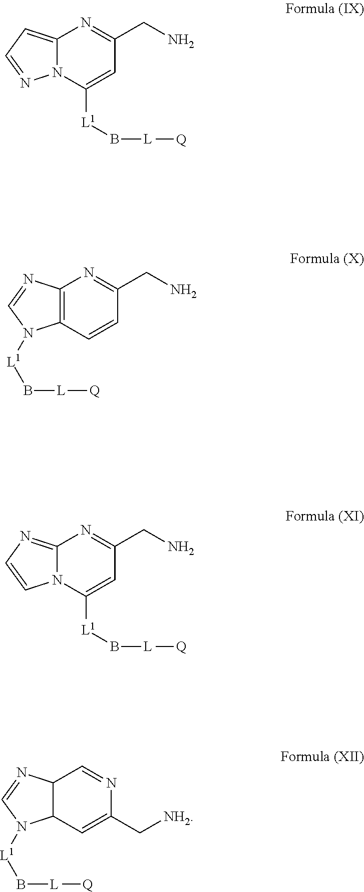

In some embodiments, ring A is an unsubstituted or substituted phenyl, or an unsubstituted or substituted naphthyl.

In some embodiments, L¹ is absent, X¹, or X¹—C₁-C₆alkylene.

In some embodiments, X¹ is —O—.

In some embodiments, L¹ is absent, —O—, or —O—CH₂—, —C(═O)—, —C(═O)NHCH₂—, —NHC(═O)—, —NHC(═O)CH₂—.

In some embodiments, L¹ is —O—, or —O—CH₂—.

In some embodiments, B is monocyclic C₃-C₆carbocycle, bicyclic C₆-C₁₂carbocycle, monocyclic C₁-C₅heterocycle, bicyclic C₅-C₁₀heterocycle.

In some embodiments, B is monocyclic C₃-C₆carbocycle.

In some embodiments, B is phenyl, cyclopropyl, cyclobutyl, cyclopentyl, or cyclohexyl.

In some embodiments, B is phenyl.

In some embodiments, B is

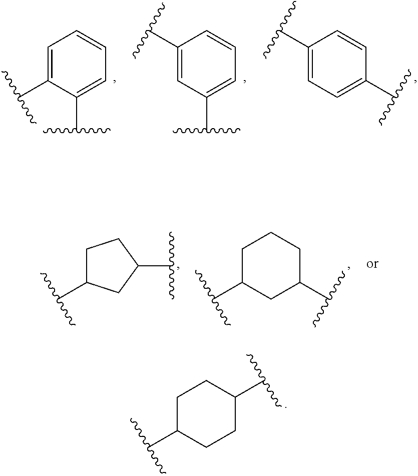

In some embodiments, B is

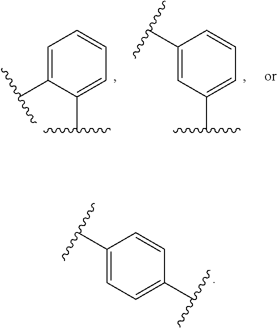

In some embodiments, B is

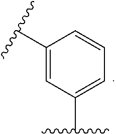 [SMILES: *c1cccc(*)c1]

In some embodiments, B is bicyclic C₉-C₁₀carbocycle.

In some embodiments, B is naphthyl, indanyl, indenyl, or tetrahyodronaphthyl.

In some embodiments, B is a monocyclic heterocycle containing 1-4 N atoms and 0 or 1 O or S atom, monocyclic heterocycle containing 0-4 N atoms and 1 O or S atoms, bicyclic heterocycle containing 1-4 N atoms and 0 or 1 O or S atoms, or bicyclic heterocycle containing 0-4 N atoms and 1 O or S atoms.

In some embodiments, B is pyrrolidinyl, pyrrolidinonyl, tetrahydrofuranyl, tetrahydrofuranonyl, dihydrofuranonyl, dihydrofuranyl, tetrahydrothienyl, oxazolidinonyl, tetrahydropyranyl, dihydropyranyl, tetrahydrothiopyranyl, piperidinyl, morpholinyl, thiomorpholinyl, piperazinyl, aziridinyl, azetidinyl, oxetanyl, thietanyl, homopiperidinyl, oxepanyl, thiepanyl, oxazepinyl, diazepinyl, thiazepinyl, 1,2,3,6-tetrahydropyridinyl, indolinyl, indolinonyl, 1,2,3,4-tetrahydroquinolinyl, 1,2,3,4-tetrahydroisoquinolinyl, 3,4-dihydro-2(1H)-quinolinonyl, furanyl, pyrrolyl, oxazolyl, thiazolyl, imidazolyl, pyrazolyl, triazolyl, tetrazolyl, isoxazolyl, isothiazolyl, oxadiazolyl, thiadiazolyl, pyridinyl, pyrimidinyl, pyrazinyl, pyridazinyl, triazinyl, quinolinyl, isoquinolinyl, quinazolinyl, quinoxalinyl, naphthyridinyl, indolyl, indazolyl, benzoxazolyl, benzisoxazolyl, benzofuranyl, benzothienyl, benzothiazolyl, benzimidazolyl, purinyl, cinnolinyl, phthalazinyl, pteridinyl, pyridopyrimidinyl, pyrazolopyrimidinyl, or azaindolyl.

In some embodiments, B is pyrrolidinyl, pyrrolidinonyl, piperidinyl, piperazinyl, indolinyl, indolinonyl, 1,2,3,4-tetrahydroquinolinyl, 1,2,3,4-tetrahydroisoquinolinyl, 3,4-dihydro-2(1H)-quinolinonyl, pyrrolyl, imidazolyl, pyrazolyl, pyridinyl, pyrimidinyl, pyrazinyl, pyridazinyl, indolyl, indazolyl, or benzimidazolyl.

In some embodiments, B is

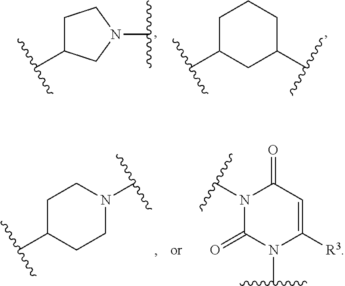

In some embodiments, B is

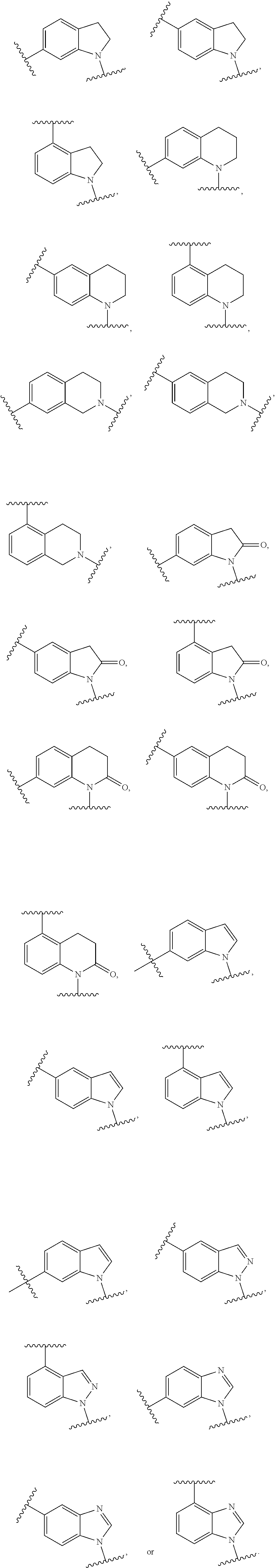

In some embodiments, the compound of Formula (I) has the following structure of Formula (IIb):

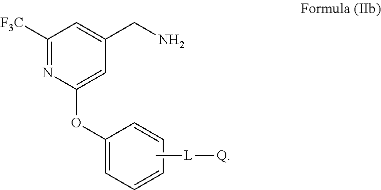

In some embodiments, the compound of Formula (I) has the following structure of Formula (IIc):

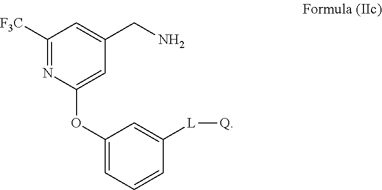

Linker

The linker region or spacer (L) can be viewed as a bridge between the reactive group or “warhead” and the labeling tag (Q). This probe element serves to prevent steric hindrance by the tag that could inhibit the reactivity of the probe compound. In its most basic form, a linker can take the form of an extended alkyl or polyethylene glycol (PEG) spacer. Additionally, the linker can serve as a specificity factor enabling targeting of the probe to specific tissues or organs. In some embodiments, the linker confers added solubility to the compound.

In some embodiments, the warhead and linker L is cleaved from LOXL2 under conditions that denature the protein such as boiling in standard SDS loading buffer.

In some embodiments, linker region (L) is a photocleavable, enzymatically cleavable, acid cleavable, alkaline cleavable, oxidatively cleavable, or reductively cleavable group.

In some embodiments, linker region (L) comprises a chemically, enzymatically or photolytically labile group.

As used herein, a cleavable bond or moiety refers to a bond or moiety that is cleaved or cleavable under the specific conditions, such as chemically, enzymatically or photolytically. For example, such bond is cleavable under conditions of MALDI-MS analysis, such as by a UV or IR laser.

As used herein, a “selectively cleavable” moiety is a moiety that can be selectively cleaved without affecting or altering the composition of the other portions of the compound of interest. For example, a cleavable moiety L of the compounds provided herein is one that can be cleaved by chemical, enzymatic, photolytic, or other means without affecting or altering composition (e.g., the chemical composition) of the conjugated biomolecule, including a protein. “Non-cleavable” moieties are those that cannot be selectively cleaved without affecting or altering the composition of the other portions of the compound of interest.

In some embodiments, when the probe compounds are attached to a solid support, such as a bead, then mass spectrometry can be used for protein identification and characterization. For example, the initial mass spectrum provides the molecular weights of all proteins captured with the probe compounds. The identity of each can then be determined by conventional means (e.g. digestion and analysis or peptide fragments and genome/proteome database searches). Use of the probe compounds allows the researcher to further analyze and characterize the protein, since it is physically isolated from all others (e.g. mass spectrum identification, or x-ray crystallography after removal from beads). To do so, the protein is washed from the solid support (e.g., if using avidin/streptavidin beads, treat the beads with biotin to displace captured proteins) or make use of an incorporated photocleavable linker, or enzymatically or chemically cleavable linker, thereby releasing the captured purified protein from the solid support.

In certain embodiments, the probe compounds for use in the methods provided herein have an L moiety that is not cleavable under conditions used for analysis of biomolecules, including, but not limited to, mass spectrometry, such as matrix assisted laser desorption ionization-time of flight (MALDI-TOF) mass spectrometry. Probe compounds of these embodiments can be used, for example, in methods provided herein for identifying biomolecules in mixtures thereof, for determining biomolecule-biomolecule, including protein-protein, interactions, and for determining biomolecule-small molecule, including protein-drug or protein-drug candidate, interactions. In these embodiments, it is not necessary for the L group to be cleaved for the analysis.

In some embodiments, where the biomolecule and the Q tag moiety function possess low steric hindrance, a spacer L is optional. In certain embodiments, steric hindrance also can enhance selectivity for LOXL2 in conjunction with the ‘warhead’ group. Spacer groups may be hydrophobic or hydrophilic; their length may be varied to achieve efficient interaction with LOXL2 and/or sorting from the biological sample; they may be rigid or flexible.

In certain embodiments, the liker group L is cleaved either prior to or during analysis of the biomolecule, such as a protein. The analysis can include mass spectral analysis, for example MALDI-TOF mass spectral analysis. The cleavable group L is selected so that the group is stable during conjugation to a biomolecule, and sorting, and washing of the conjugated biomolecule; but is susceptible to cleavage under conditions of analysis of the biomolecule, including, but not limited to, mass spectral analysis, for example MALDI-TOF analysis. In certain embodiments, the cleavable group L comprises a disulfide moiety. The disulfide bond can be cleaved under various reducing conditions including, but not limited to, treatment with dithiothreitol and 2-mercaptoethanol.

In another embodiment, L is a photocleavable group, which can be cleaved by a short treatment with UV light of the appropriate wave length either prior to or during mass spectrometry. Photocleavable groups, including those bonds that can be cleaved during MALDI-TOF mass spectrometry by the action of a laser beam, can be used. For example, a trityl ether or an ortho nitro substituted aralkyl, including benzyl, group are susceptible to laser induced bond cleavage during MALDI-TOF mass spectrometry. Other useful photocleavable groups include, but are not limited to, o-nitrobenzyl, phenacyl, and nitrophenylsulfenyl groups. Other photocleavable groups include those disclosed in International Patent Publication no. WO 98/20166.

Other cleavable L groups include acid sensitive groups, where bond cleavage is promoted by formation of a cation upon exposure to mild to strong acids. For these acid-labile groups, cleavage of the group L can be effected either prior to or during analysis, including mass spectrometric analysis, by the acidity of the matrix molecules, or by applying a short treatment of the array with an acid, such as the vapor of trifluoroacetic acid. Exposure of a trityl group to acetic or trifluoroacetic acid produces cleavage of the ether bond either before or during MALDI-TOF mass spectrometry.

In some embodiments, linker L is a non cleavable linker.

In some embodiments, linker L is a cleavable linker. In some embodiments, linker L is a cleavable linker that undergoes cleavage following treatment with a mild reducing agent or hydrazine.

In some embodiments, linker L comprises diazobenzene, levulinoyl ester, disulfide, nitrobenzene sulfonamide, dithiocarbamate, or hydrazone. In some embodiments, linker L comprises diazobenzene, levulinoyl ester, disulfide, or nitrobenzene sulfonamide.

In some embodiments, L is absent or a linker with the formula -L²-C-L³-;
L² is X²—Y²—, —Y²—X²—, or Y²;
   X² is —O—, —S—, —S(═O)—, —S(═O)₂—, —S(═O)₂NR³—, —C(═O)—, —C(═O)O—, —C(═O)NR³—, —NR³C(═O)—, —NR³S(═O)₂—, or —NR—;
      R³ is H, C₁-C₆alkyl, C₁-C₆fluoroalkyl, or C₁-C₆deuteroalkyl;
   Y² is absent, or C₁-C₆alkylene;
C is absent, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₈cycloalkyl, —C₁-C₄alkylene-(substituted or unsubstituted C₃-C₈cycloalkyl), substituted or unsubstituted C₂-C₈heterocycloalkyl, —C₁-C₄alkylene-(substituted or unsubstituted C₂-C₈heterocycloalkyl), substituted or unsubstituted aryl, —C₁-C₄alkylene-(substituted or unsubstituted aryl), substituted or unsubstituted heteroaryl, or —C₁-C₄alkylene-(substituted or unsubstituted heteroaryl); wherein if C is substituted then C is substituted with one or more R^{c};
or when C and R³ are attached to the same N-atom then C and R³ are taken together with the N atom to which they are attached to form ring D, wherein ring D is a substituted or unsubstituted N-containing heterocycle, wherein if ring D is substituted then ring D is substituted with 1, 2, or 3 R^{d};
each R^{c}, and R^{d}, is independently selected from the group consisting of D, halogen, CN, —OR⁵, —SR⁵, —S(═O)R⁴, —S(═O)₂R⁴, —S(═O)₂N(R⁵)₂, NR⁵S(═O)₂R⁴, C(═O)R⁴, OC(═O)R⁴, CO₂R⁵, OCO₂R⁴, N(R⁴)₂, OC(═O)N(R⁵)₂, —NHC(═O)R⁴, —NHC(═O)OR⁴, C₁-C₆alkyl, C₁-C₆fluoroalkyl, C₁-C₆deuteroalkyl, C₁-C₆heteroalkyl, substituted or unsubstituted C₃-C₁₀cycloalkyl, substituted or unsubstituted C₂-C₁₀heterocycloalkyl, substituted or unsubstituted aryl, or substituted or unsubstituted heteroaryl;
or two R^{d} groups attached to the same carbon atom are taken together with carbon atom to which they are attached to form either a substituted or unsubstituted carbocycle or substituted or unsubstituted heterocycle;
L³ is absent or -L⁴-L⁵-L⁶-L⁷-;
   L⁴ is absent, —O—, —S—, —S(═O)—, —S(═O)_{2—}, —NR⁴—, —CH(OH)—, —C(═O)—, —C(═O)NH—, —NHC(═O)—, —C(═O)O—, —OC(═O)—, —CH(═N)—, —CH(═N—NH)—, —CCH₃(═N)—, —CCH₃(═N—NH)—, —OC(═O)NH—, —NHC(═O)NH—, —NHC(═O)O—, —(CH₂)ₚ—, —(OCH₂CH₂)ₚ—, or —(OCH₂CH₂)ₚ—, p is 1, 2, 3, 4, 5, or 6;
   L⁵ is absent, unsubstituted or substituted alkylene, unsubstituted or substituted heteroalkylene, unsubstituted or substituted alkenylene, unsubstituted or substituted alkynylene, unsubstituted or substituted cycloalkylene, unsubstituted or substituted heterocycloalkylene, unsubstituted or substituted arylene, unsubstituted or substituted heteroarylene, —(OCH₂CH₂)ₚ—, or —(OCH₂CH₂)ₚ—, p is 1, 2, 3, 4, 5, 6, 7, 8, 9, 10, 11, or 12;
   L⁶ is absent, —O—, —S—, —S(O)—, —S(O)_{2—}, —NR⁴—, —CH(OH)—, —C(═O)—, —C(═O)NH—, —NHC(═O)—, —C(═O)O—, —OC(═O)—, —OC(═O)NH—, —NHC(═O)NH—, or —NHC(═O)O—;
   L⁷ is absent, unsubstituted or substituted alkylene, unsubstituted or substituted heteroalkylene.

In some embodiments, L is -L²-C-L³-;
L² is —X²—Y²—, —Y²—X²—, or Y²;
X² is —O—, —S(═O)₂NR³—, —C(═O)—, —C(═O)NR³—, —NR³C(═O)—, —NR³S(═O)₂—, or —NR³—;
R³ is H, C₁-C₆alkyl, C₁-C₆fluoroalkyl, or C₁-C₆deuteroalkyl;
Y² is absent, or C₁-C₆alkylene;
C is absent, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₈cycloalkyl, —C₁-C₄alkylene-(substituted or unsubstituted C₃-C₈cycloalkyl), substituted or unsubstituted C₂-C₈heterocycloalkyl, —C₁-C₄alkylene-(substituted or unsubstituted C₂-C₈heterocycloalkyl), substituted or unsubstituted aryl, —C₁-C₄alkylene-(substituted or unsubstituted aryl), substituted or unsubstituted heteroaryl, or —C₁-C₄alkylene-(substituted or unsubstituted heteroaryl); wherein if C is substituted then C is substituted with one or more R^{c};
or when C and R³ are on the same N atom then C and R³ are taken together with the N atom to which they are attached to form ring D, wherein ring D is a substituted or unsubstituted monocyclic N-containing heterocycle, or a substituted or unsubstituted bicyclic N-containing heterocycle, wherein if ring D is substituted then ring D is substituted with 1, 2, or 3 R^{d}.

In some embodiments, L² is —X²—Y²—; X² is —C(═O)NR³—; Y² is absent, or C₁-C₆alkylene.

In some embodiments, C is absent, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₈cycloalkyl, C₁-C₄alkylene-(substituted or unsubstituted C₃-C₈cycloalkyl), substituted or unsubstituted C₂-C₈heterocycloalkyl, —C₁-C₄alkylene-(substituted or unsubstituted C₂-C₈heterocycloalkyl), substituted or unsubstituted aryl, —C₁-C₄alkylene-(substituted or unsubstituted aryl), substituted or unsubstituted heteroaryl, or —C₁-C₄alkylene-(substituted or unsubstituted heteroaryl); wherein if C is substituted then C is substituted with one or more R^{c}; or when C and R³ are on the same N atom then C and R³ are taken together with the N atom to which they are attached to form ring D, wherein ring D is a substituted or unsubstituted aziridinyl, substituted or unsubstituted azetidinyl, substituted or unsubstituted pyrrolidinyl, substituted or unsubstituted pyrrolidinonyl, substituted or unsubstituted piperidinyl, substituted or unsubstituted piperidinonyl, substituted or unsubstituted morpholinyl, substituted or unsubstituted thiomorpholinyl, substituted or unsubstituted piperazinyl, substituted or unsubstituted piperazinonyl, substituted or unsubstituted indolinyl, substituted or unsubstituted indolinonyl, substituted or unsubstituted 1,2,3,4-tetrahydroquinolinyl, substituted or unsubstituted 1,2,3,4-tetrahydroisoquinolinyl, substituted or unsubstituted 3,4-dihydro-2(1H)-quinolinonyl, wherein if ring D is substituted then ring D is substituted with 1, 2, or 3 R^{D}.

In some embodiments, the compound of Formula (I) has the following structure of Formula (IId):

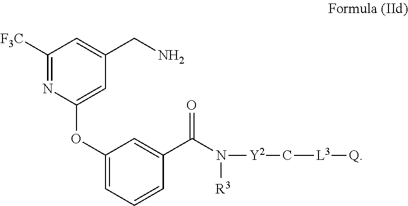

In some embodiments, L³ is absent or a linker that is -L⁴-L⁵-L⁶-L⁷-;
L⁴ is absent, —O—, —S—, —S(═O)—, —S(═O)_{2—}, —NR⁴—, —CH(OH)—, —C(═O)—, —C(═O)NH—, —NHC(═O)—, —C(═O)O—, —OC(═O)—, —CH(═N)—, —CH(═N—NH)—, —CCH₃(═N)—, —CCH₃(═N—NH)—, —OC(═O)NH—, —NHC(═O)NH—, —NHC(═O)O—, —(CH₂)ₚ—, —(OCH₂CH₂)ₚ—, or —(OCH₂CH₂)ₚ—, p is 1, 2, 3, 4, 5, or 6;
L⁵ is absent, unsubstituted or substituted alkylene, unsubstituted or substituted heteroalkylene, unsubstituted or substituted alkenylene, unsubstituted or substituted alkynylene, unsubstituted or substituted cycloalkylene, unsubstituted or substituted heterocycloalkylene, unsubstituted or substituted arylene, unsubstituted or substituted heteroarylene, —(OCH₂CH₂)ₚ—, or —(OCH₂CH₂)ₚ—, p is 1, 2, 3, 4, 5, 6, 7, 8, 9, 10, 11, or 12;
L⁶ is absent, —O—, —S—, —S(O)—, —S(O)_{2—}, —CH(OH)—, —C(═O)—, —C(═O)NH—, —NHC(═O)—, —C(═O)O—, —OC(═O)—, —OC(═O)NH—, —NHC(═O)NH—, or —NHC(═O)O—;
L⁷ is absent, unsubstituted or substituted alkylene, unsubstituted or substituted heteroalkylene.

Tag Moiety (Q)

The tag allows for the identification or purification of modified enzymes. Biotin, fluorescent small molecules, and radioactive isotopes are among tags contemplated for incorporation into the probe compounds as tags, and all three can be used to visualize labeled proteins after SDS-PAGE. Other tags are contemplated. Biotin tags are used for affinity purification of modified enzymes and their subsequent identification through mass spectrometry or for direct visualization using labeled streptavidin molecules. For simpler direct visualization of labeled targets, fluorescent and radiolabeled tags are often used. Fluorescent or radiolabeled tags have an advantage over biotin as they have a higher dynamic range and require less time and handling to generate data. Additionally, multiple fluorescent tags with non-overlapping excitation/emission spectra can be utilized to multiplex sample analysis using gel-based methods.

In some embodiments, Q is a tag moiety for the detection, isolation, or detection and isolation of the compound of Formula (I) in a biological sample; or Q is absent provided that the compound of Formula (I) comprises a radioactive or an isotopic variant of any atom in the compound of Formula (I).

In some embodiments, Q is a tag moiety for the detection, isolation, or detection and isolation of the compound of Formula (I) in a biological sample that is selected from the group consisting of: a solid support, a reporter group, a tag used for affinity purification, a tag used for sorting or immobilizing the compound of Formula (I) on a solid support, a hapten, a fluorescent moiety, radioactive moiety, magnetic resonance imaging (MRI) moiety, colorometric moiety, luminescent moiety, bioluminescent moiety, chemiluminescent moiety, oligonucleotide, antibody, peptide or combination thereof; or Q is absent provided that the compound of Formula (I) comprises a radioactive or an isotopic variant of any atom in the compound of Formula (I).

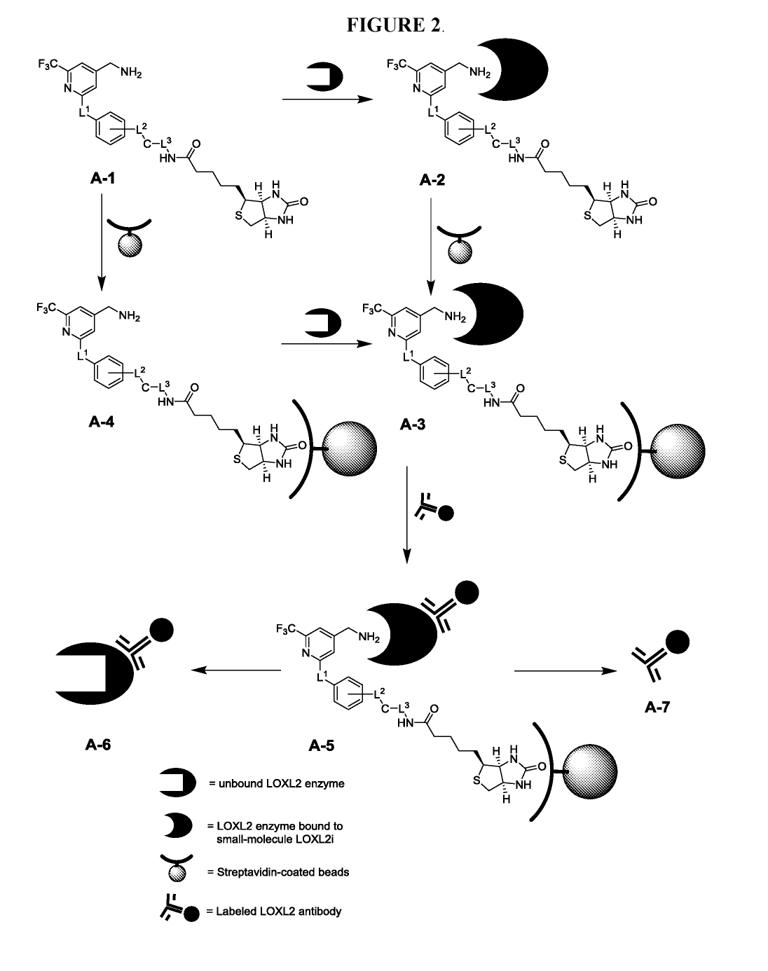

In some embodiments, compounds of Formula (I) described herein are used to capture and detect free (unbound) LOXL2 enzyme from ex vivo biological samples or in vitro systems. In some embodiments, compounds of Formula (I) containing a biotin moiety and having the general structure A-1 are used to isolate, detect, and quantify LOXL2 as shown in FIG. 2.

In some embodiments, treatment of a biological sample or system containing free (unbound) LOXL2, with a biotin-labeled small-molecule LOXL2 inhibitor A-1, provides the small-molecule LOXL2 inhibitor-LOXL2 enzyme complex A-2. In some embodiments, complex A-2 is further captured via the addition of streptavidin-coated beads, to afford complex A-3. In some embodiments, biotin-labeled LOXL2 inhibitor A-1 is treated with streptavidin-coated beads, to afford complex A-4. In some embodiments, treatment of a biological sample or system containing free (unbound) LOXL2, with complex A-4, affords A-3. In some embodiments, treatment of the A-3 containing biological sample, with an appropriately labeled LOXL2 antibody, affords complex A-5. In some embodiments, such labels include fluorescent dyes, fluorescent phycobiliproteins, magnetoresistive nanosensors, or metal-chelating compounds. In some embodiments, bead-containing complex A-5 is isolated from the biological media, and subsequent elution yields the purified labeled LOXL2 protein-antibody complex A-6, which is detected and quantified using appropriate analytical techniques. In some embodiments, bead-containing complex A-5 is isolated from the biological media, and subsequent elution yields the purified antibody A-7, which is detected and quantified using appropriate analytical techniques.

In some embodiments, Q is a tag used for affinity purification that is capable of specific binding to a known protein to produce a tightly bound complex.

In some embodiments, Q is a tag that is capable of specific binding to avidin or streptavidin.

In some embodiments, Q is biotin or desthiobiotin.

In some embodiments, L is

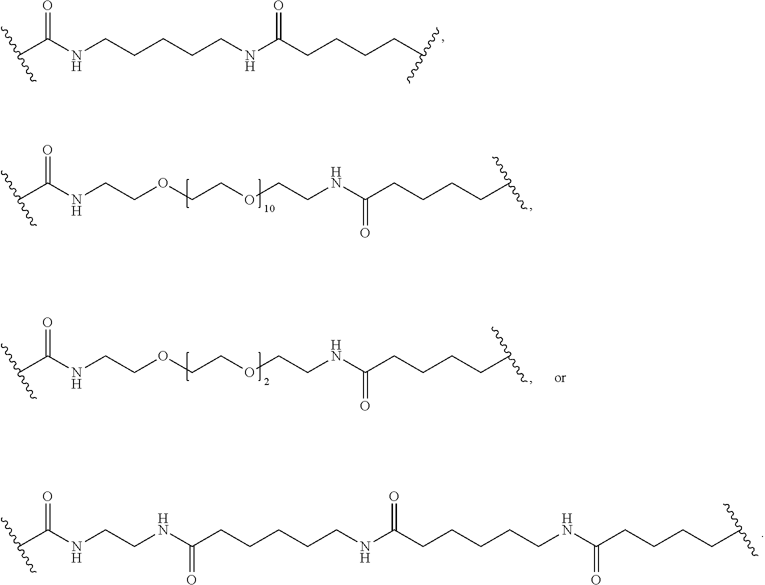

In some embodiments, -L-Q is

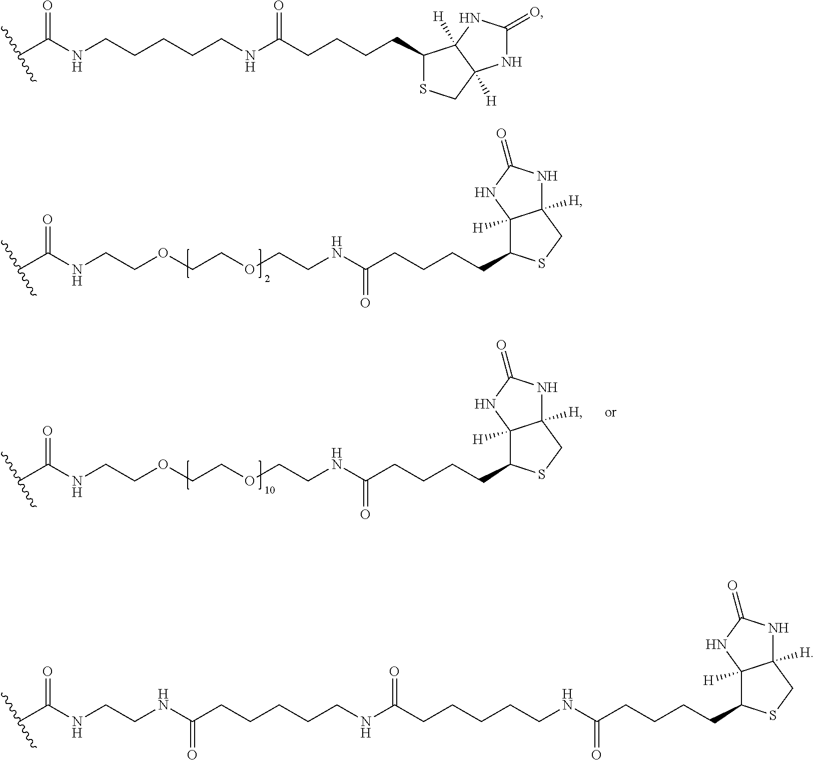

In some embodiments, the affinity moiety binds to avidin or streptavidin. In some embodiments, the affinity moiety is biotin or biotin analog

In some embodiments, Q is a hapten selected from biotin, a coumarin dye, a rhodamine dye, a xanthene dye (such as fluorescein), a cyanine dye, a BODIPY dye, a Lucifer yellow dye, digoxigenin, dansyl, or dintrophenyl.

In some embodiments, when Q is a hapten then fluorescence quenching may be used. Such methods are called fluorescence quenching immunoassays. For example, the fluorescence of fluorescein derivatives decreases (is quenched) when they non-specifically conjugate with proteins, bind to specific (anti-fluorescein) antibodies or when FITC-labelled antigen reacts with a corresponding antibody.

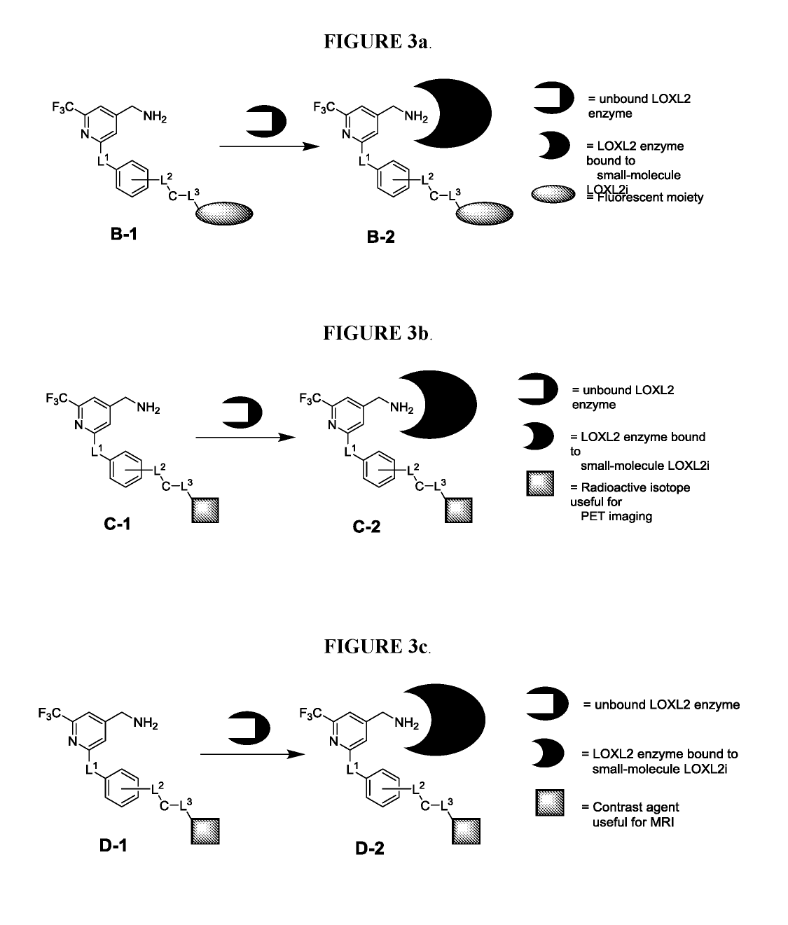

In some embodiments, a compound of Formula (I) containing a fluorescent moiety and having the general structure B-1 is used to detect, and quantify LOXL2 as shown in FIG. 3a.

In some embodiments, treatment of a biological sample or system containing free (unbound) LOXL2, with a small-molecule LOXL2 inhibitor B-1, containing a fluorescent moiety, provides the small-molecule LOXL2 inhibitor-LOXL2 enzyme complex B-2. In some embodiments, complex B-2 is detected and quantified using appropriate fluorescent imaging and analytical techniques.

Examples of suitable fluorophores include but are not limited to xanthenes, such as fluorescein, rhodamine, Oregon green, eosin, and Texas red; cyanines, such as cyanine, Cy2, Cy3, Cy3B, Cy3.5, Cy5, Cy5.5, Cy7, Cy7.5, indocyanine green, and sulfo Cy dyes; squaraines, such as Seta, SeTau, and Square dyes; naphthalenes, such as prodan dyes and dansyl dyes; coumarins, such as hydroxycoumarin, aminocoumarin, and methoxycoumarin; oxadiazoles, such as pyridyloxazole, nitrobenzoxadiazole and benzoxadiazole; anthracenes, such as anthraquinones, DRAQ5, DRAQ7 and CyTRAK Orange; pyrenes, such as cascade blue; oxazines, such as Nile red, Nile blue, cresyl violet, and oxazine 170; acridines, such as proflavin, acridine orange, and acridine yellow; arylmethines, such as auramine, crystal violet, malachite green; tetrapyrroles, such as porphin, phthalocyanine, bilirubin. Other examples also include Alexa Fluor, DyLight Fluor, BODIPY, FluoProbes, SureLight Dyes, HiLyte Fluor, and IRDyes. Moreover, fluorescent proteins, such as green fluorescent protein (GFP), yellow fluorescent protein (YFP), and red fluorescent protein (RFP), are also contemplated for use.

In some embodiments, the fluorophore is a xanthene, cyanine, squaraine, naphthalene, coumarin, oxadiazole, anthracene, pyrene, oxazine, acridine, arylmethine, tetrapyrrole, or dansyl. In some embodiments, the fluorophore is cyanine, coumarin, or dansyl.

In some embodiments, Q is a tag moiety that is selected from the group consisting of: a fluorescent moiety, radioactive moiety, colorometric moiety, luminescent moiety, chemiluminescent moiety, or combination thereof.

In some embodiments, Q is a tag moiety that is a fluorescent moiety.

In some embodiments, Q is a tag moiety that is a fluorescent moiety selected from the group consisting of xanthene dyes, cyanine dyes, squaraine dyes, ring-substituted squaraine dyes, naphthalene dyes, coumarin dyes, oxadiazole dyes, anthracene dyes, oxazine dyes, acridine dyes, arylmethine dyes, BODIPY dyes, and tetrapyrrole dyes.

In some embodiments, Q is a fluorescent moiety selected from fluorescein dyes, rhodamine dyes, Oregon green dyes, eosin dyes, Texas red dyes, cyanine dyes, indocarbocyanine dyes, oxacarbocyanine dyes, thiacarbocyanine dyes, merocyanine dyes, Seta, SeTau, Square dyes, dansyl dyes, prodan dyes, coumarin dyes, BODIPY dyes, pyridyloxazole dyes, nitrobenzoxadiazole dyes, benzoxadiazole dyes, DRAQ5, DRAQ7, CyTRAK Orange cascade blue, Nile red, Nile blue, cresyl violet, oxazine 170, proflavin dyes, acridine orange dyes, acridine yellow dyes, auramine dyes, crystal violet dyes, malachite green dyes, porphin dyes, phthalocyanine dyes, and bilirubin dyes.

In some embodiments, Q is xanthene, cyanine, squaraine, naphthalene, coumarin, oxadiazole, anthracene, pyrene, oxazine, acridine, arylmethine, tetrapyrrole, dansyl, or BODIPY.

In some embodiments, Q is cyanine, coumarin, or dansyl.

In some embodiments, Q is xanthene, cyanine 2, cyanine 3, cyanine 3B, cyanine 3.5, cyanine 5, cyanine 5.5, cyanine7, squaraine, naphthalene, coumarin, oxadiazole, anthracene, pyrene, oxazine, acridine, arylmethine, tetrapyrrole, dansyl, BODIPY FL, BODIPY R6G, BODIPY TMR, BODIPY 581/591, BODIPY TR, BODIPY 630/650, or BODIPY 650/665.

In some embodiments, -L- is —NH—, —C(═O)NH—,

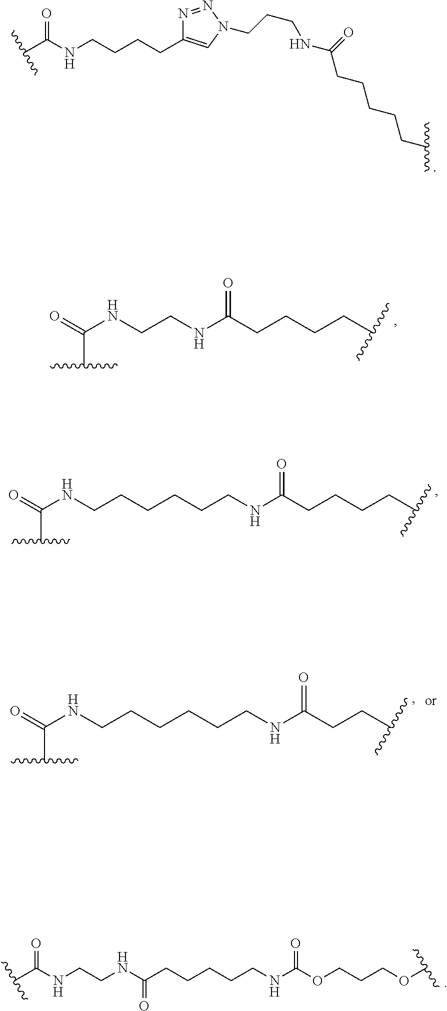 [SMILES: *CCCCCC(=O)NCCCn1cc(CCCCNC(*)=O)nn1 ; *CCCCC(=O)NCCNC(*)=O ; *CCCCC(=O)NCCCCCCNC(*)=O ; *CCC(=O)NCCCCCCNC(*)=O ; *OCCCOC(=O)NCCCCCC(=O)NCCNC(*)=O]

In some embodiments, Q is

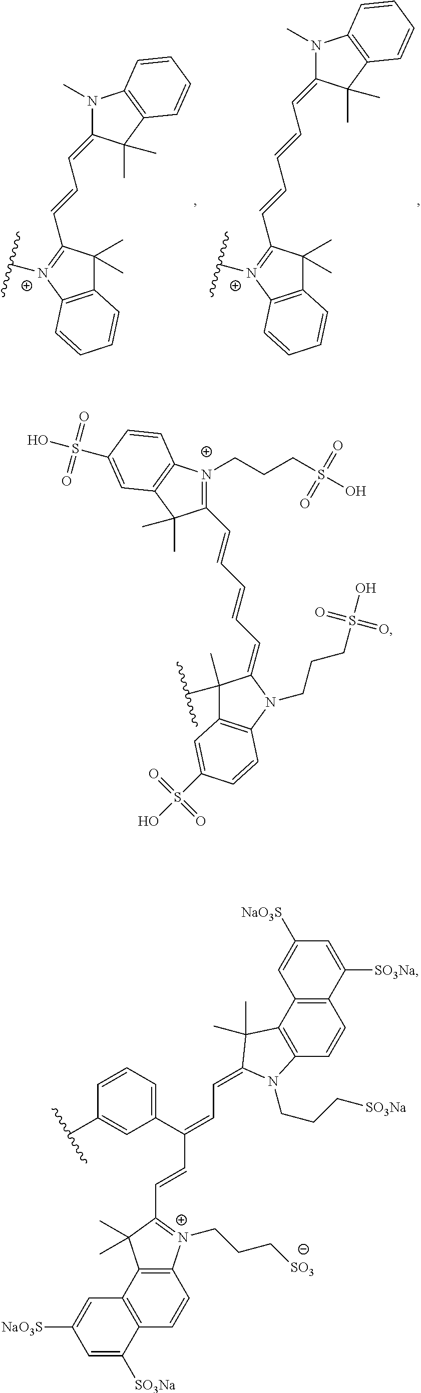

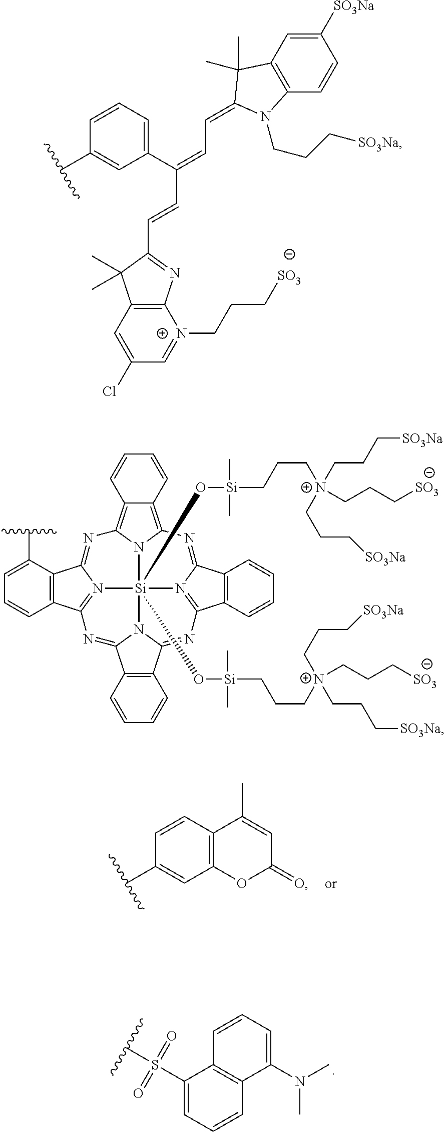

In some embodiments, -L-Q is

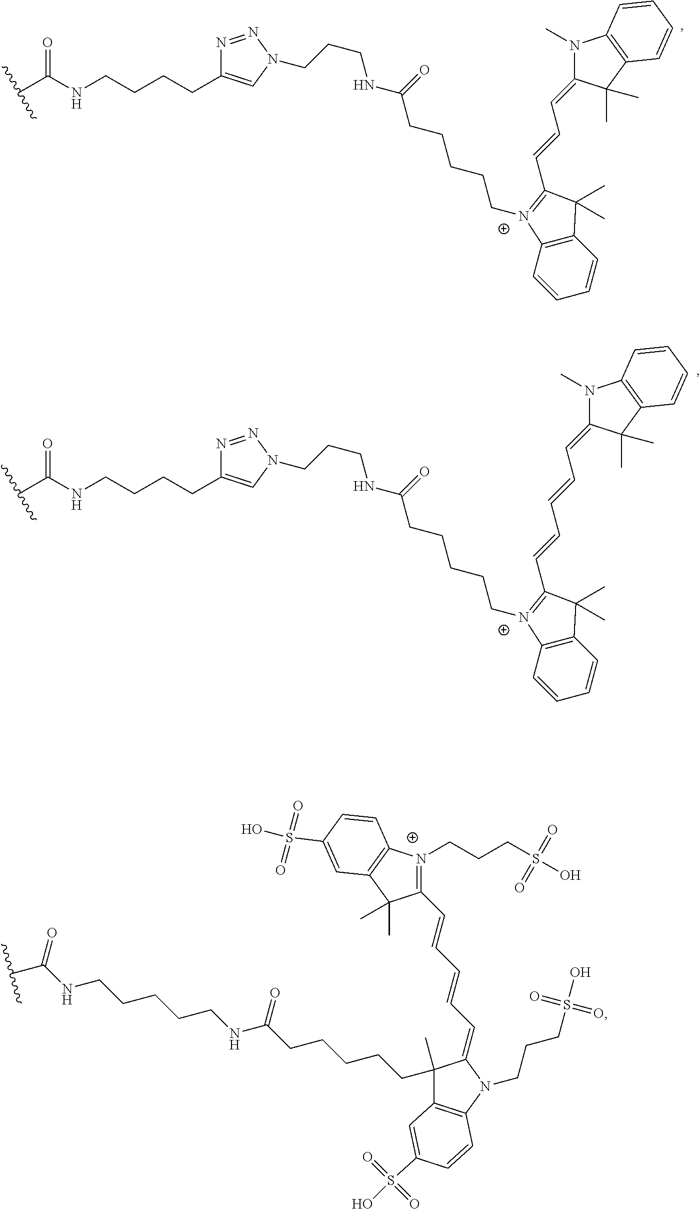

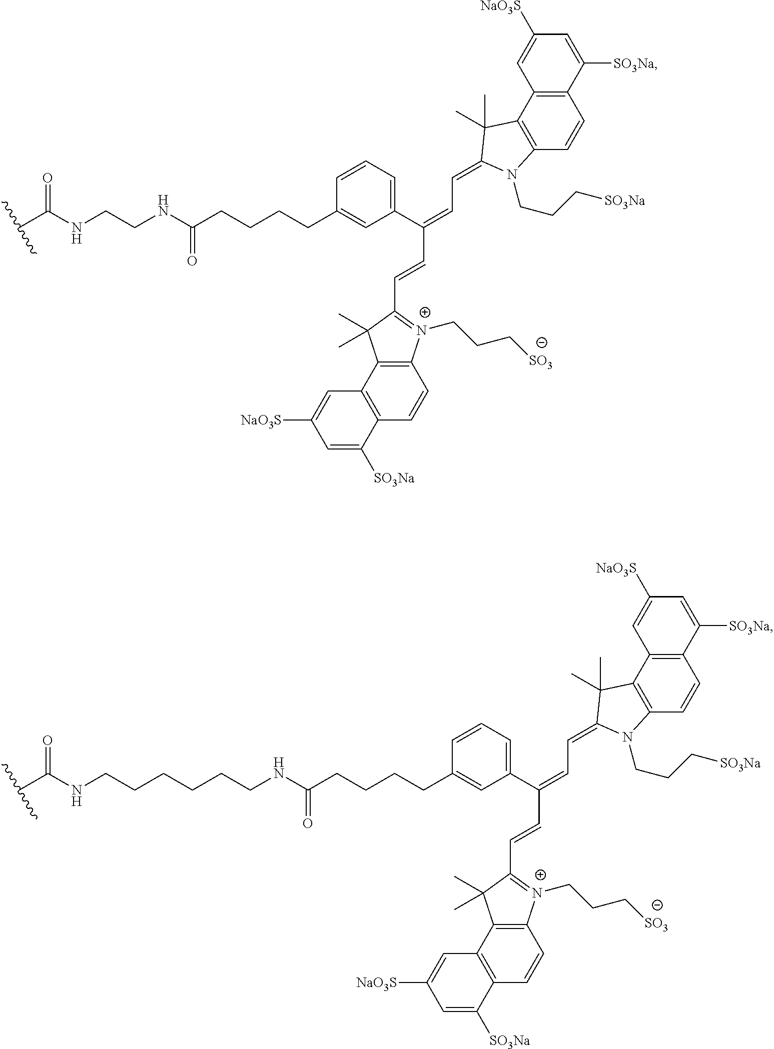

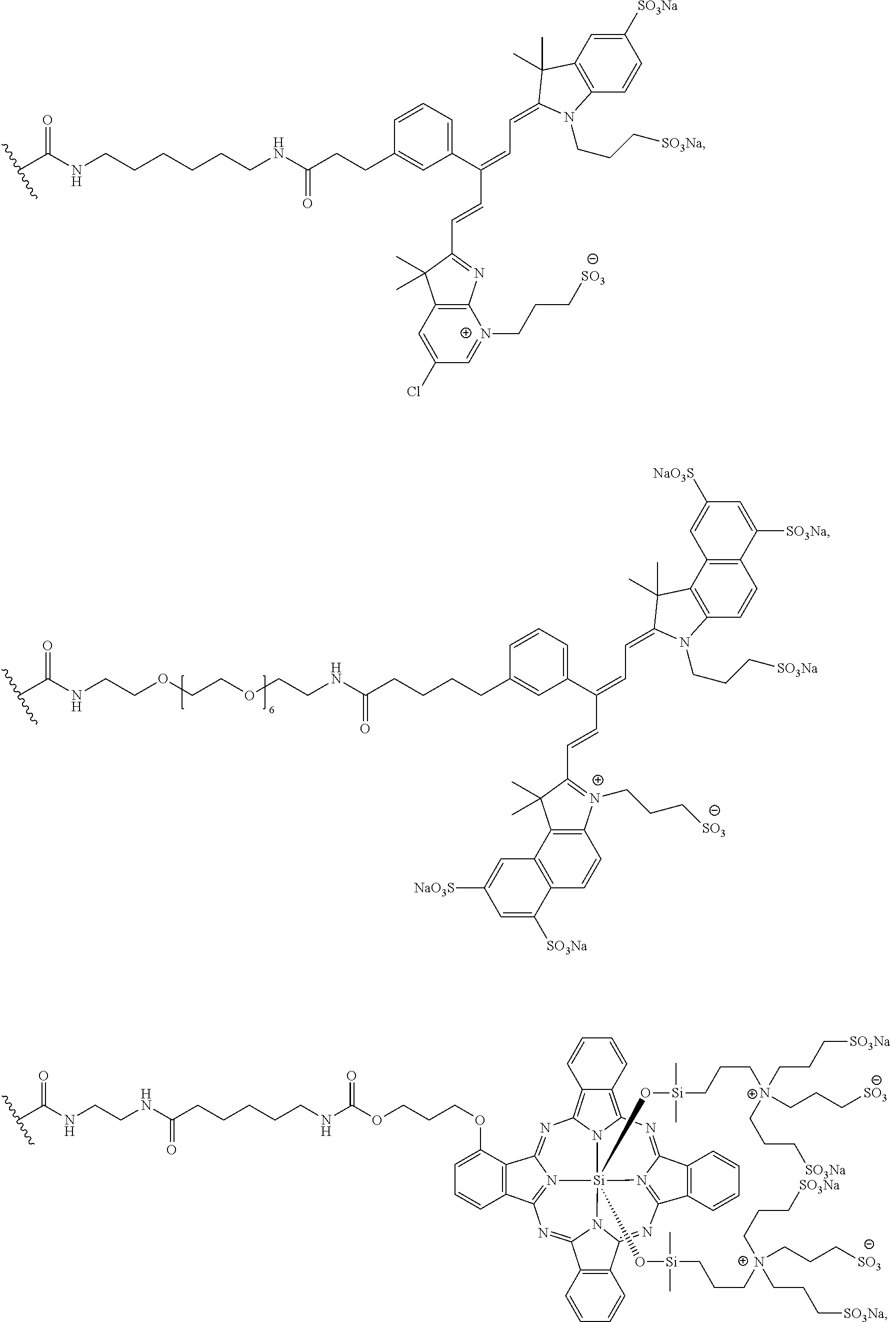

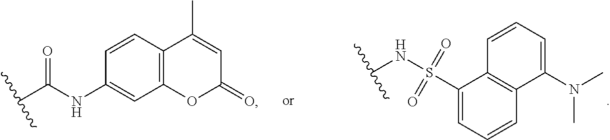 [SMILES: *C(=O)Nc1ccc2c(C)cc(=O)oc2c1 ; *NS(=O)(=O)c1cccc2c(N(C)C)cccc12]

In some embodiments, Q is a tag moiety that is a chemiluminescent moiety.

In some embodiments, Q is a chemiluminescent moiety that generates light or a colored product upon treatment with peroxide or a peroxidase.

In some embodiments, Q is luminol, isoluminol, N-(4-aminobutyl)-N-ethyl isoluminol (ABEI), N-(4-aminobutyl)-N-methyl isoluminol (ABMI), 2,2′-azino-bis(3-ethylbenzothiazoline-6-sulphonic acid) (ABTS), 3,3′,5,5′-Tetramethylbenzidine (TMB), 3,3′-diaminobenzidine (DAB), o-phenylenediamine dihydrochloride (OPD), AmplexRed, AEC, or homovanillic acid.

In some embodiments, Q is a chemiluminescent moiety that generates light or a colored product upon treatment with horseradish peroxidase (HRP).

In some embodiments, Q is 3,3′-diaminobenzidine (DAB), 3,3′,5,5′-tetramethylbenzidine (TMB), 2,2′-azinobis [3-ethylbenzothiazoline-6-sulfonic acid] (ABTS), o-phenylenediamine dihydrochloride (OPD).

In some embodiments, Q is a substrate for a luciferase enzyme.

In some embodiments, Q is D-luciferin, or coelenterazine.

In some embodiments, Q is a chemiluminescent moiety that generates light or a colored product upon treatment with alkaline phosphatase (AP).

In some embodiments, Q is nitro blue tetrazolium chloride (NBT), 5-bromo-4-chloro-3-indolyl phosphate (BCIP), or p-nitrophenyl phosphate (PNPP).

In some embodiments, Q is a chemiluminescent moiety that generates light or a colored product upon treatment with glucose oxidase.

In some embodiments, Q is nitro blue tetrazolium chloride (NBT).

In some embodiments, Q is a chemiluminescent moiety that generates light or a colored product upon treatment with β-galactosidase.

In some embodiments, Q is 5-bromo-4-chloro-3-indoyl-β-D-galactopyranoside (BCIG or X-Gal).

In some embodiments, compounds of Formula (I) containing a radiolabeled moiety, or radioactive isotope, (useful for positron emission tomography (PET) imaging) and having the general structure C-1 are used to detect, and quantify LOXL2 as shown in FIG. 3b.

In some embodiments, treatment of an animal or human that expresses free (unbound) LOXL2 with a small-molecule LOXL2 inhibitor C-1, containing a radiolabeled moiety suitable for use in PET imaging, provides in vivo the small-molecule LOXL2 inhibitor-LOXL2 enzyme complex C-2. In some embodiments the radiolabeled moiety is, for example, carbon-11, nitrogen-13, oxygen-15, fluorine-18, or hydrogen-3. In some embodiments, the radiolabeled moiety is copper-64 or gallium-68. In some embodiments, complex C-2 is detected and quantified using appropriate PET imaging and analytical techniques.

In some embodiments, the radiolabeled moiety is a fluorine radioisotope or a hydrogen radioisotope. In some embodiments, the radiolabeled moiety is ¹⁸F or ³H.

In some embodiments, Q is absent and the compound of Formula (I) comprises a radioactive or an isotopic variant of any atom in the compound of Formula (I).

In some embodiments, the compound of Formula (I) comprises a radioactive or an isotopic variant of any atom in the compound of Formula (I) and is suitable for use in PET analysis.

In some embodiments, Q is absent and the compound of Formula (I) comprises one or more atoms selected from tritium (³H), fluorine-18 (¹⁸F), carbon-11 (¹¹C), carbon-14 (¹⁴C), nitrogen-13 (¹³N), oxygen-15 (¹⁵O), or sulfur-35 (³⁵S).

In some embodiments, -L-Q is

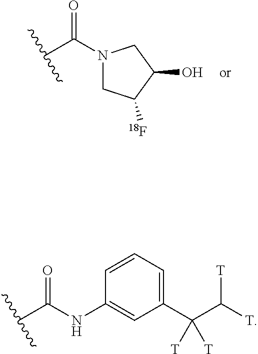

In some embodiments, Q comprises a chelated radioactive isotope.

In some embodiments, Q comprises a chelated radioactive isotope that is suitable for positron emission tomography (PET) analysis.

In some embodiments, Q comprises a chelated radioactive isotope, wherein Q is a diethylenetriaminepentaacetic acid (DTPA) chelate, 1,4,7,10-tetraazacyclododecane-1,4,7,10-tetraacetic acid (DOTA) chelate, or 1,4,7-triazacyclononane-1,4,7-trisacetic acid (NOTA) chelate or 1,4,7,10-tetraazacyclododecane-1,4,7,10-tetramethyl-1,4,7,10-tetraacetic acid (DOTMA) chelate or a radioactive isotope.

In some embodiments, L is

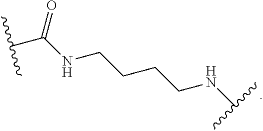 [SMILES: *NCCCCNC(*)=O]

In some embodiments, Q is

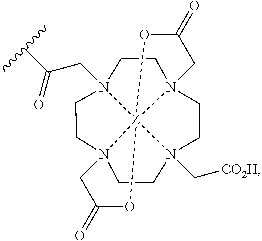

where Z is a radioactive isotope.

In some embodiments, Q comprises a chelated radioactive isotope that is copper-64 (⁶⁴Cu), gallium-68 (⁶⁸Ga), or technetium-99m (^{99m}Tc).

In some embodiments, -L-Q is

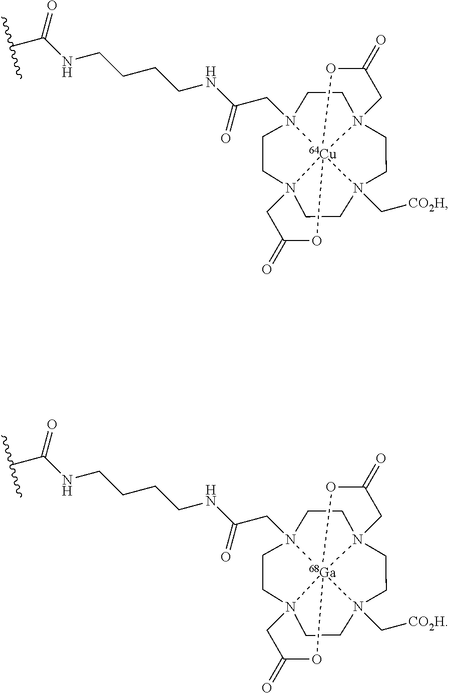

In some embodiments, compounds of Formula (I) containing a contrast agent moiety (useful for MRI imaging) and having the general structure D-1 are used to detect, and quantify LOXL2 as shown FIG. 3c.

In some embodiments, treatment of an animal or human that expresses free (unbound) LOXL2 with a small-molecule LOXL2 inhibitor D-1, containing a contrast agent moiety suitable for use in MRI imaging, provides an in vivo the small-molecule LOXL2 inhibitor-LOXL2 enzyme complex D-2. In some embodiments the contrast agent is, for example, thulium, europium, gadolinium, or manganese. In some embodiments, complex D-2 is detected and quantified using appropriate PET imaging and analytical techniques.

In some embodiments, Q is a magnetic resonance imaging (MRI) moiety.

In some embodiments, Q comprises a chelate of an atom that is suitable for magnetic resonance imaging (MRI).

In some embodiments, Q comprises a chelate of an atom that is suitable for magnetic resonance imaging (MRI) that is a diethylenetriaminepentaacetic acid (DTPA) chelate, 1,4,7,10-tetraazacyclododecane-1,4,7,10-tetraacetic acid (DOTA) chelate, 1,4,7-triazacyclononane-1,4,7-trisacetic acid (NOTA) chelate, or 1,4,7,10-tetraazacyclododecane-1,4,7,10-tetramethyl-1,4,7,10-tetraacetic acid (DOTMA) chelate.

In some embodiments, Q comprises a chelate of copper, gallium, thulium, europium, gadolinium, or manganese.

In some embodiments, Q comprises a chelate of gadolinium that is selected from gadoterate, gadodiamide, gadobenate, gadopentetate, gadoteridol, gadoversetamide, gadoxetate, gadobutrol, or gadofosveset.

In some embodiments, -L-Q is

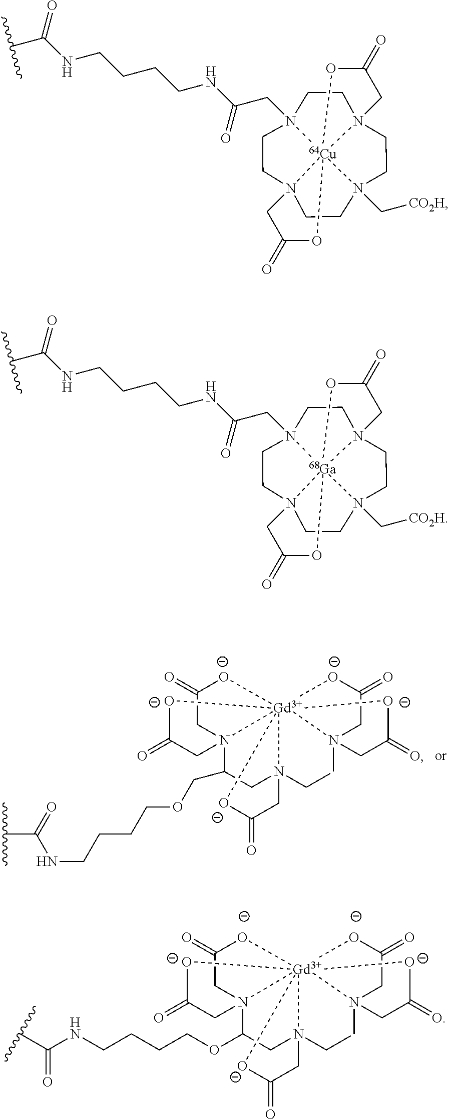

In some embodiments, Q is a solid support.

In some embodiments, Q is a solid support that is a nanoparticle, bead, or resin.

In some embodiments, Q is a nanoparticle or bead comprising one or more metals selected from iron, cobalt, nickel, gadolium, chromium, manganese or gold.

In some embodiments, Q is a nanoparticle or bead that is magnetic or paramagnetic.

In some embodiments, the magnetic moiety is a ferrite magnetic bead.

In some embodiments, -L-Q is

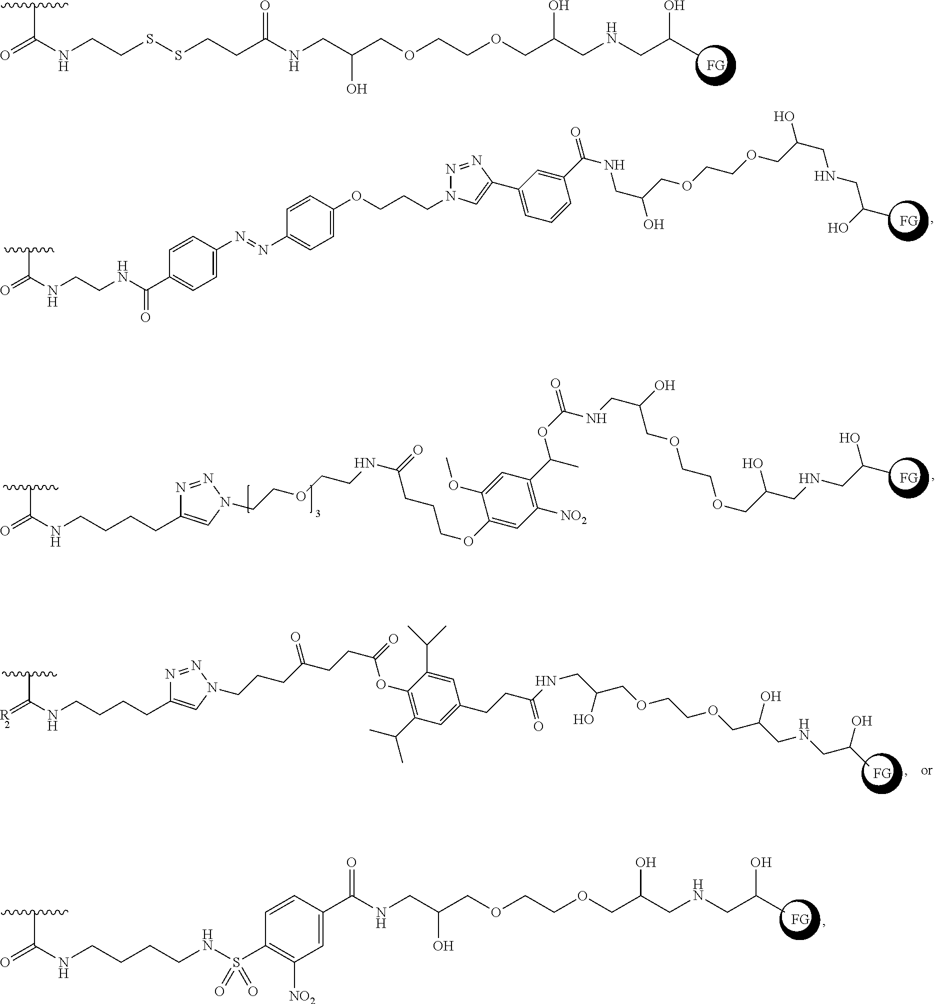

where FG is a ferrite bead.

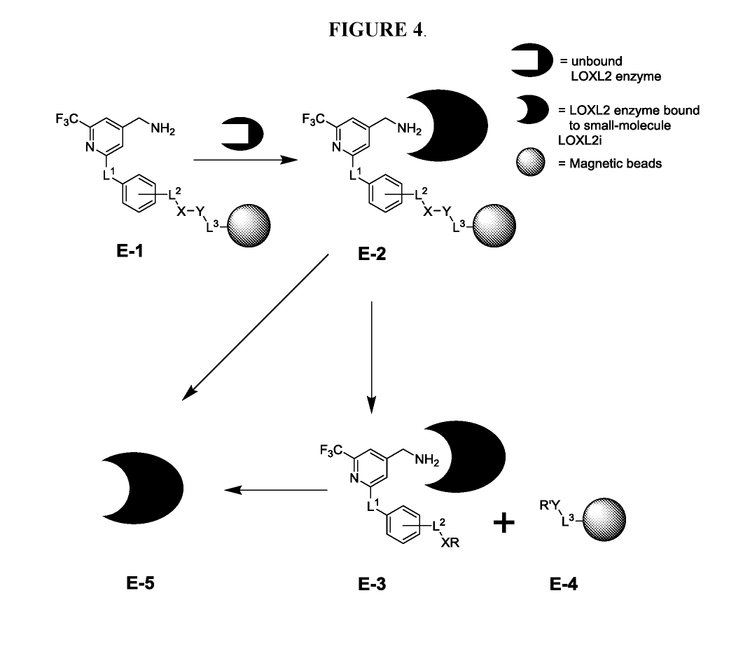

In some embodiments, compounds of Formula (I) containing a magnetic bead and having the general structure E-1 are used to isolate, detect, and quantify LOXL2 as shown in FIG. 4.

In some embodiments, treatment of a biological sample or system containing free (unbound) LOXL2, with a magnetic bead-labeled small-molecule LOXL2 inhibitor E-1, containing a cleavable or non-cleavable linker (X-Y), provides the small-molecule LOXL2 inhibitor-LOXL2 enzyme complex E-2. In some embodiments, the linker X-Y is a chemical or photo cleavable linker (for a review on cleavable linkers see Leriche et al Bioorg. Med. Chem., 2012, 20, p 571-582 and references cited). In some embodiments the linker X-Y is, for example, a disulfide moiety which may be cleaved by biocompatible mild reducing agents such as DTT or TCEP. In some embodiments, the linker X-Y is, for example, a diazobenzene derivative, which is cleaved by biocompatible reducing agents, such as sodium dithionite. In some embodiments, the linker X-Y is, for example, an ester derivative which is cleaved under high pH conditions, or alternatively by treatment with a nucleophile such as hydroxylamine or hydrazine. In some embodiments, the linker X-Y is, for example, an appropriately substituted photo-labile derivative which is cleaved with a specific wavelength of UV light. In some embodiments, bead-containing complex E-2 is isolated from the biological media, and subsequent cleavage of the cleavable linker provides the purified LOXL2 protein-small molecule inhibitor complex E-3 and cleaved magnetic bead-containing moiety E-4. In some embodiments, complex E-3 is directly detected and quantified using appropriate analytical techniques (such as ELISA or Western blotting). In some embodiments, complex E-3 is further eluted to give purified LOXL2 protein E-5, which is detected and quantified using appropriate analytical techniques. In some embodiments, complex E-2 is eluted to yield purified LOXL2 protein E-5, which is detected and quantified using appropriate analytical techniques.

In some embodiments, the magnetic moiety is a ferrite magnetic bead.

In some embodiments, L is a cleavable linker. In some embodiments, L is a cleavable linker that undergoes cleavage under treatment with a mild reducing agent or hydrazine. In some embodiments, L comprises diazobenzene, levulinoyl ester, disulfide, nitrobenzene sulfonamide, dithiocarbamate, or hydrazone. In some embodiments, L comprises diazobenzene, levulinoyl ester, disulfide, or nitrobenzene sulfonamide.

Any combination of the groups described above for the various variables is contemplated herein. Throughout the specification, groups and substituents thereof are chosen by one skilled in the field to provide stable moieties and compounds.

In some embodiments, compounds of Formula (I) include, but are not limited to, those described in Table 1.

TABLE 1

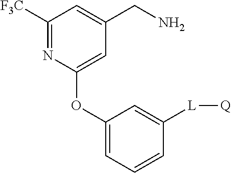

| Compound | |
|---|---|
| Number | -L-Q |
| 1-1 | 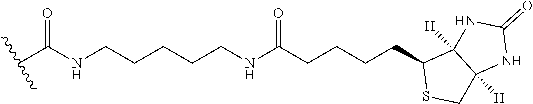 [SMILES: *C(=O)NCCCCCNC(=O)CCCC[C@@H]1SC[C@@H]2NC(=O)N[C@@H]21] |
| 1-2 | 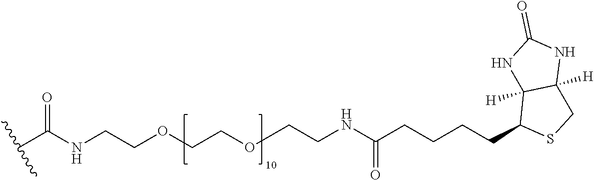 |
| 1-3 | 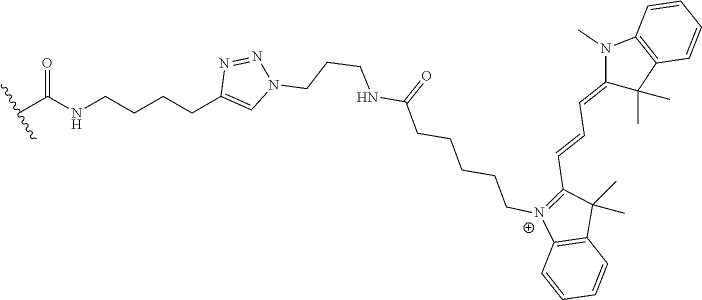 [SMILES: *C(=O)NCCCCc1cn(CCCNC(=O)CCCCC[N+]2=C(/C=C/C=C3/N(C)c4ccccc4C3(C)C)C(C)(C)c3ccccc32)nn1] |
| 1-4 | 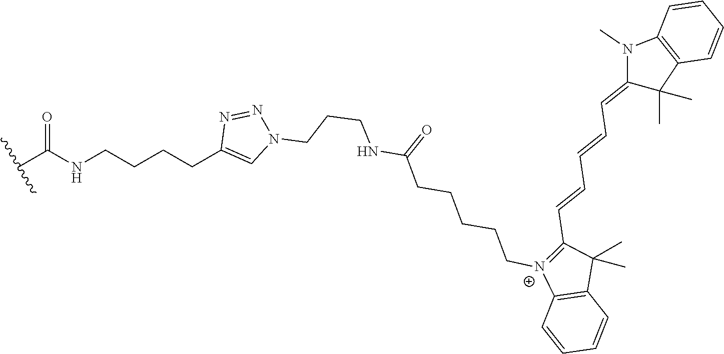 [SMILES: *C(=O)NCCCCc1cn(CCCNC(=O)CCCCC[N+]2=C(/C=C/C=C/C=C3/N(C)c4ccccc4C3(C)C)C(C)(C)c3ccccc32)nn1] |
| 1-5 | 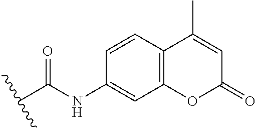 [SMILES: *C(=O)Nc1ccc2c(C)cc(=O)oc2c1] |
| 1-6 | 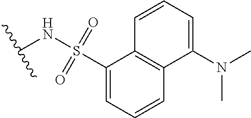 [SMILES: *NS(=O)(=O)c1cccc2c(N(C)C)cccc12] |
| 1-7 | 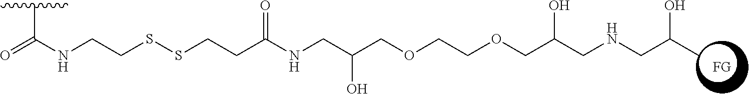 [SMILES: *C(=O)NCCSSCCC(=O)NCC(O)COCCOCC(O)CNCC(C)O] |
| 1-8 | 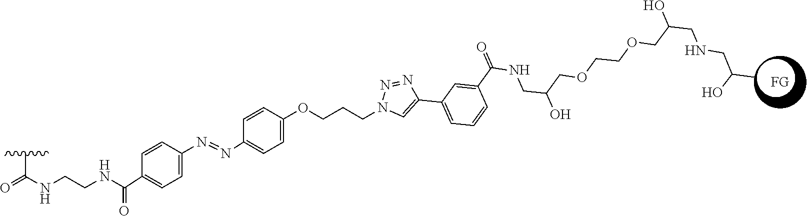 [SMILES: *C(=O)NCCNC(=O)c1ccc(/N=N/c2ccc(OCCCn3cc(-c4cccc(C(=O)NCC(O)COCCOCC(O)CNCC(C)O)c4)nn3)cc2)cc1] |
| 1-9 | 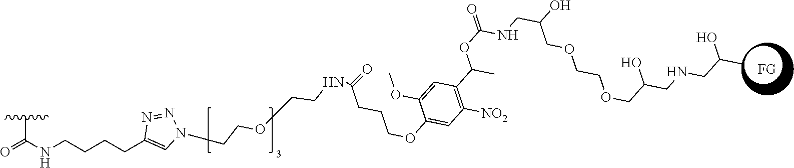 |
| 1-10 | 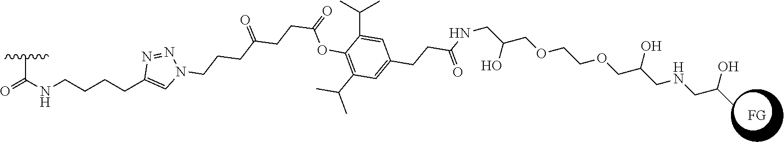 [SMILES: *C(=O)NCCCCc1cn(CCCC(=O)CCC(=O)Oc2c(C(C)C)cc(CCC(=O)NCC(O)COCCOCC(O)CNCC(C)O)cc2C(C)C)nn1] |
| 1-11 | 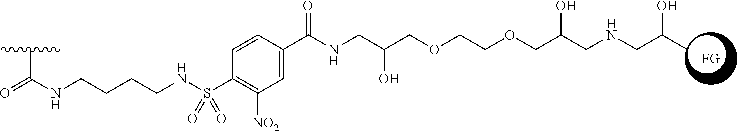 [SMILES: *C(=O)NCCCCNS(=O)(=O)c1ccc(C(=O)NCC(O)COCCOCC(O)CNCC(C)O)cc1[N+](=O)[O-]] |
| 1-12 | 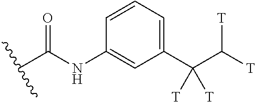 |
| 1-13 | 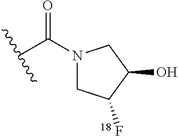 [SMILES: *C(=O)N1C[C@@H](O)[C@H](F)C1] |
| 1-13a | 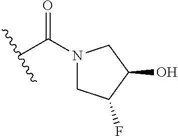 [SMILES: *C(=O)N1C[C@@H](O)[C@H](F)C1] |
| 1-14 | 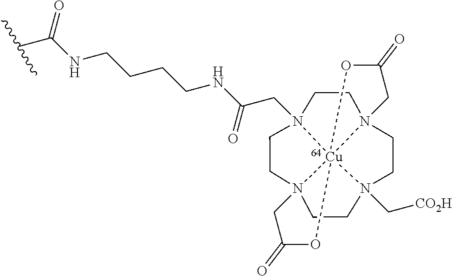 |
| 1-15 | 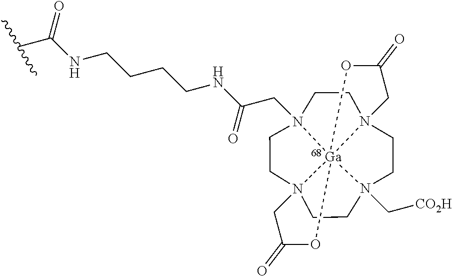 |
| 1-16 | 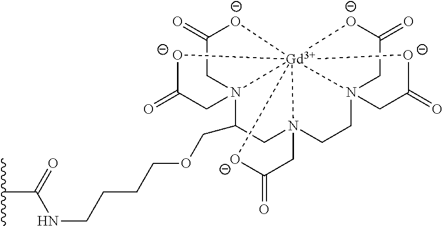 |
| 1-17 | 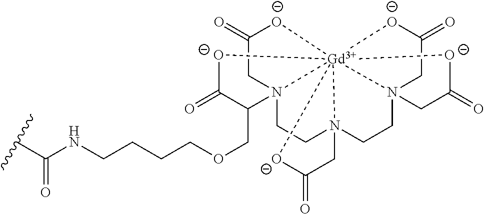 |
| 1-18 | 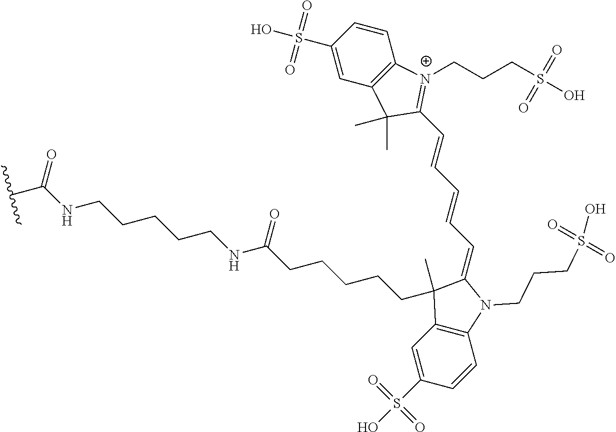 [SMILES: *C(=O)NCCCCCNC(=O)CCCCCC1(C)\C(=C/C=C/C=C/C2=[N+](CCCS(=O)(=O)O)c3ccc(S(=O)(=O)O)cc3C2(C)C)N(CCCS(=O)(=O)O)c2ccc(S(=O)(=O)O)cc21] |
| 1-19 | 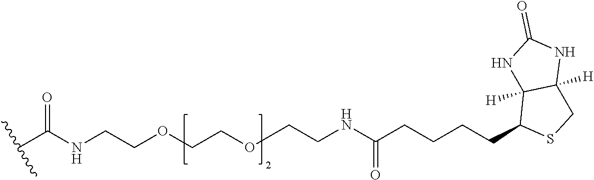 |
| 1-20 | 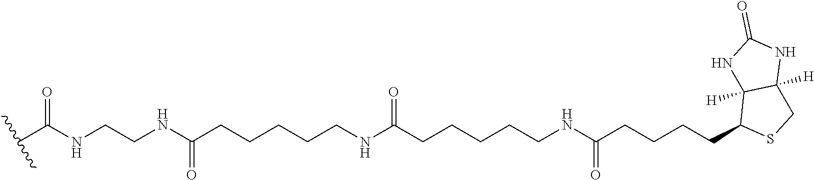 [SMILES: *C(=O)NCCNC(=O)CCCCCNC(=O)CCCCCNC(=O)CCCC[C@@H]1SC[C@@H]2NC(=O)N[C@@H]21] |
| 1-21 | 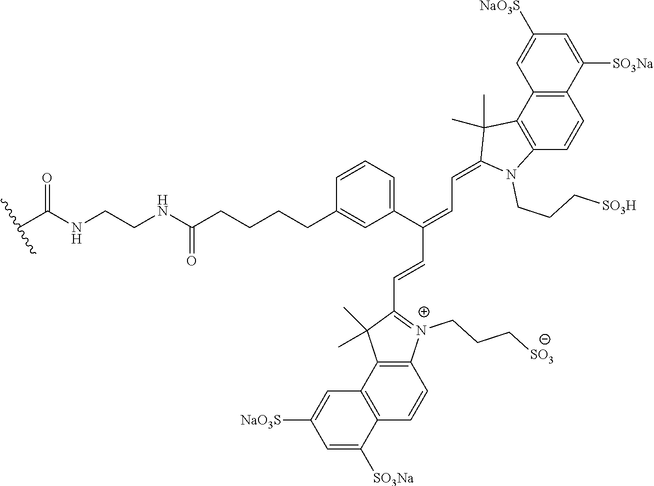 |
| 1-22 | 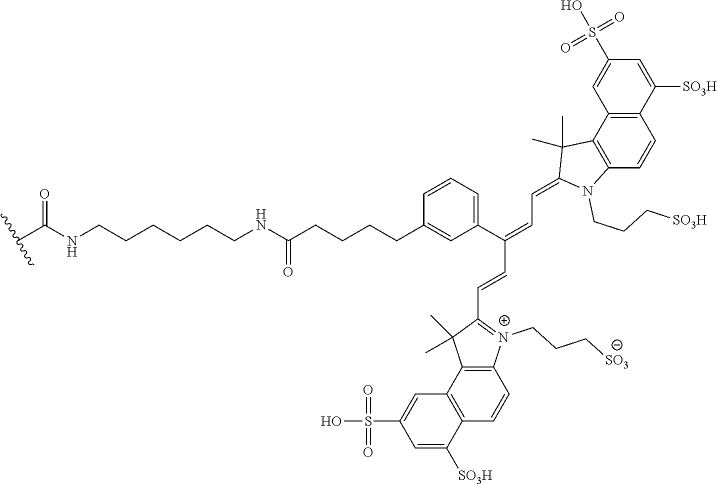 |
| 1-23 | 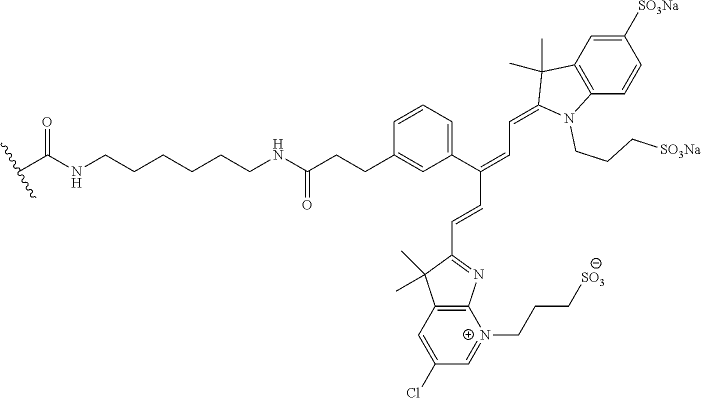 |
| 1-24 | 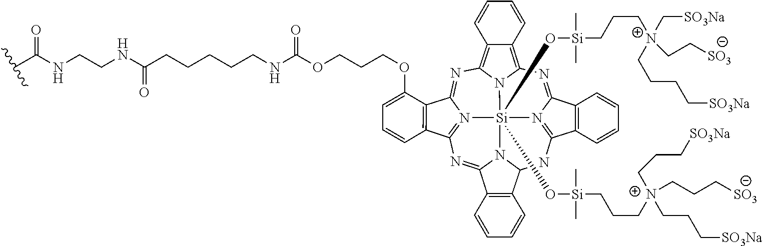 |
| 1-25 | 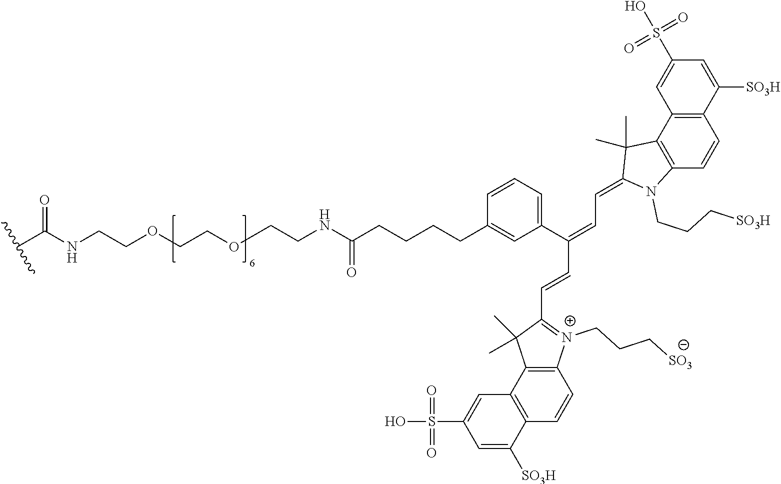 |

In some embodiments, compounds of Formula (I) include, but are not limited to:
3-((4-(Aminomethyl)-6-(trifluoromethyl)pyridin-2-yl)oxy)-N-(5-(5-((3aS,4S,6aR)-2-oxohexahydro-1H-thieno[3,4-d]imidazol-4-yl)pentanamido)pentyl)benzamide (Compound 1-1);
3-((4-(Aminomethyl)-6-(trifluoromethyl)pyridin-2-yl)oxy)-N-(37-oxo-41-((3aS,4S,6aR)-2-oxohexahydro-1H-thieno[3,4-d]imidazol-4-yl)-3,6,9,12,15,18,21,24,27,30,33-undecaoxa-36-azahentetracontyl)benzamide (Compound 1-2);
1-(6-((3-(4-(4-(3-((4-(Aminomethyl)-6-(trifluoromethyl)pyridin-2-yl)oxy)benzamido)butyl)-1H-1,2,3-triazol-1-yl)propyl)amino)-6-oxohexyl)-3,3-dimethyl-2-((1E,3E)-3-(1,3,3-trimethylindolin-2-ylidene)prop-1-en-1-yl)-3H-indol-1-ium (Compound 1-3);
1-(6-((3-(4-(4-(3-((4-(Aminomethyl)-6-(trifluoromethyl)pyridin-2-yl)oxy)benzamido)butyl)-1H-1,2,3-triazol-1-yl)propyl)amino)-6-oxohexyl)-3,3-dimethyl-2-((1E,3E,5E)-5-(1,3,3-trimethylindolin-2-ylidene)penta-1,3-dien-1-yl)-3H-indol-1-ium (Compound 1-4);
3-((4-(Aminomethyl)-6-(trifluoromethyl)pyridin-2-yl)oxy)-N-(4-methyl-2-oxo-2H-chromen-7-yl)benzamide (Compound 1-5);
N-(3-((4-(Aminomethyl)-6-(trifluoromethyl)pyridin-2-yl)oxy)phenyl)-5-(dimethylamino)naphthalene-1-sulfonamide (Compound 1-6);
3-((4-(Aminomethyl)-6-(trifluoromethyl)pyridin-2-yl)oxy)-N-(21-(FG-bead)-10,17,21-trihydroxy-7-oxo-12,15-dioxa-3,4-dithia-8,19-diazahenicosyl)benzamide (Compound 1-7);
(E)-3-((4-(Aminomethyl)-6-(trifluoromethyl)pyridin-2-yl)oxy)-N-(2-(4-((4-(3-(4-(3-((3-(2-(3-((2-(FG-bead)-2-hydroxyethyl)amino)-2-hydroxypropoxy)ethoxy)-2-hydroxypropyl)carbamoyl)phenyl)-1H-1,2,3-triazol-1-yl)propoxy)phenyl)diazenyl)benzamido)ethyl)benzamide (Compound 1-8);
1-(4-(4-((2-(2-(4-(4-(3-((4-(Aminomethyl)-6-(trifluoromethyl)pyridin-2-yl)oxy)benzamido)butyl)-1H-1,2,3-triazol-1-yl)ethoxy)ethyl)amino)-4-oxobutoxy)-5-methoxy-2-nitrophenyl)ethyl (2-hydroxy-3-(2-(2-hydroxy-3-((2-hydroxy-2-(FG-bead)-ethyl)amino)propoxy)ethoxy)propyl)carbamate (Compound 1-9);
4-(1-(FG-bead)-1,5,12-trihydroxy-15-oxo-7,10-dioxa-3,14-diazaheptadecan-17-yl)-2,6-diisopropylphenyl 7-(4-(4-(3-((4-(aminomethyl)-6-(trifluoromethyl)pyridin-2-yl)oxy)benzamido)butyl)-1H-1,2,3-triazol-1-yl)-4-oxoheptanoate (Compound 1-10);
4-(N-(4-(3-((4-(Aminomethyl)-6-(trifluoromethyl)pyridin-2-yl)oxy)benzamido)butyl)sulfamoyl)-N-(3-(2-(3-((2-(FG-bead)-2-hydroxyethyl)amino)-2-hydroxypropoxy)ethoxy)-2-hydroxypropyl)-3-nitrobenzamide (Compound 1-11);
3-((4-(Aminomethyl)-6-(trifluoromethyl)pyridin-2-yl)oxy)-N-(3-(1,1,2,2-tetra-³H-ethyl)phenyl)benzamide (Compound 1-12);
(3-((4-(Aminomethyl)-6-(trifluoromethyl)pyridin-2-yl)oxy)phenyl)((3R,4R)-3-¹⁸fluoro-4-hydroxypyrrolidin-1-yl)methanone (Compound 1-13);
(3-((4-(Aminomethyl)-6-(trifluoromethyl)pyridin-2-yl)oxy)phenyl)((3R,4R)-3-fluoro-4-hydroxypyrrolidin-1-yl)methanone (Compound 1-13a);
2,2′,2″-(10-(2-((4-(3-((4-(Aminomethyl)-6-(trifluoromethyl)pyridin-2-yl)oxy)benzamido)butyl)amino)-2-oxoethyl)-1,4,7,10-tetraazacyclododecane-1,4,7-triyl)triacetic acid-⁶⁴copper complex (Compound 1-14);
2,2′,2″-(10-(2-((4-(3-((4-(Aminomethyl)-6-(trifluoromethyl)pyridin-2-yl)oxy)benzamido)butyl)amino)-2-oxoethyl)-1,4,7,10-tetraazacyclododecane-1,4,7-triyl)triacetic acid-⁶⁸gallium complex (Compound 1-15);
2,2′-((15-(3-((4-(Aminomethyl)-6-(trifluoromethyl)pyridin-2-yl)oxy)phenyl)-1-carboxylato-2,5-bis(carboxylatomethyl)-15-oxo-9-oxa-2,5,14-triazapentadecan-7-yl)azanediyl)diacetate-gadolinium (III) complex (Compound 1-16);
1-(3-((4-(Aminomethyl)-6-(trifluoromethyl)pyridin-2-yl)oxy)phenyl)-9-carboxylato-10,13,16-tris(carboxylatomethyl)-1-oxo-7-oxa-2,10,13,16-tetraazaoctadecan-18-oate-gadolinium (III) complex (Compound 1-17);
2-((1E,3E,5E)-5-(3-(6-((5-(3-((4-(Aminomethyl)-6-(trifluoromethyl)pyridin-2-yl)oxy)benzamido)pentyl)amino)-6-oxohexyl)-3-methyl-5-sulfo-1-(3-sulfopropyl)indolin-2-ylidene)penta-1,3-dien-1-yl)-3,3-dimethyl-5-sulfo-1-(3-sulfopropyl)-3H-indol-1-ium (Compound 1-18);
3-((4-(Aminomethyl)-6-(trifluoromethyl)pyridin-2-yl)oxy)-N-(13-oxo-17-((3aS,4S,6aR)-2-oxohexahydro-1H-thieno[3,4-d]imidazol-4-yl)-3,6,9-trioxa-12-azaheptadecyl)benzamide (Compound 1-19);
3-((4-(Aminomethyl)-6-(trifluoromethyl)pyridin-2-yl)oxy)-N-(2-(6-(6-(5-((3aS,4S,6aR)-2-oxohexahydro-1H-thieno[3,4-d]imidazol-4-yl)pentanamido)hexanamido)hexanamido)ethyl)benzamide (Compound 1-20);
Penta-sodium 2-((1E,3Z,5Z)-3-(3-(5-((2-(3-((4-(aminomethyl)-6-(trifluoromethyl)pyridin-2-yl)oxy)benzamido)ethyl)amino)-5-oxopentyl)phenyl)-5-(1,1-dimethyl-6,8-disulfonato-3-(3-sulfonatopropyl)-1H-benzo[e]indol-2(3H)-ylidene)penta-1,3-dien-1-yl)-1,1-dimethyl-3-(3-sulfonatopropyl)-1H-benzo[e]indol-3-ium-6,8-disulfonate (Compound 1-21);
3-(2-((1E,3Z,5Z)-3-(3-(5-((6-(3-((4-(Aminomethyl)-6-(trifluoromethyl)pyridin-2-yl)oxy)benzamido)hexyl)amino)-5-oxopentyl)phenyl)-5-(1,1-dimethyl-6,8-disulfo-3-(3-sulfopropyl)-1H-benzo[e]indol-2(3H)-ylidene)penta-1,3-dien-1-yl)-1,1-dimethyl-6,8-disulfo-1H-benzo[e]indol-3-ium-3-yl)propane-1-sulfonate (Compound 1-22);
Bis-sodium (Z)-2-((2Z,4E)-3-(3-(3-((6-(3-((4-(aminomethyl)-6-(trifluoromethyl)pyridin-2-yl)oxy)benzamido)hexyl)amino)-3-oxopropyl)phenyl)-5-(5-chloro-3,3-dimethyl-7-(3-sulfonatopropyl)-3H-pyrrolo[2,3-b]pyridin-7-ium-2-yl)penta-2,4-dien-1-ylidene)-3,3-dimethyl-1-(3-sulfonatopropyl)indoline-5-sulfonate (Compound 1-23);
3-(2-((1E,3Z,5Z)-3-(3-(1-(3-((4-(Aminomethyl)-6-(trifluoromethyl)pyridin-2-yl)oxy)phenyl)-1,27-dioxo-5, 8,11,14,17,20,23-heptaoxa-2,26-diazahentriacontan-31-yl)phenyl)-5-(1,1-dimethyl-6,8-disulfo-3-(3-sulfopropyl)-1H-benzo[e]indol-2(3H)-ylidene)penta-1,3-dien-1-yl)-1,1-dimethyl-6,8-disulfo-1H-benzo[e]indol-3-ium-3-yl)propane-1-sulfonate (Compound 1-25).

Any combination of the groups described above for the various variables is contemplated herein. Throughout the specification, groups and substituents thereof are chosen by one skilled in the field to provide stable moieties and compounds.

In one aspect, compounds described herein are in the form of pharmaceutically acceptable salts. As well, active metabolites of these compounds having the same type of activity are included in the scope of the present disclosure. In addition, the compounds described herein can exist in unsolvated as well as solvated forms with pharmaceutically acceptable solvents such as water, ethanol, and the like. The solvated forms of the compounds presented herein are also considered to be disclosed herein.

“Pharmaceutically acceptable,” as used herein, refers a material, such as a carrier or diluent, which does not abrogate the biological activity or properties of the compound, and is relatively nontoxic, i.e., the material is administered to an individual without causing undesirable biological effects or interacting in a deleterious manner with any of the components of the composition in which it is contained.

The term “pharmaceutically acceptable salt” refers to a form of a therapeutically active agent that consists of a cationic form of the therapeutically active agent in combination with a suitable anion, or in alternative embodiments, an anionic form of the therapeutically active agent in combination with a suitable cation. Handbook of Pharmaceutical Salts: Properties, Selection and Use. International Union of Pure and Applied Chemistry, Wiley-VCH 2002. S. M. Berge, L. D. Bighley, D. C. Monkhouse, J. Pharm. Sci. 1977, 66, 1-19. P. H. Stahl and C. G. Wermuth, editors, Handbook of Pharmaceutical Salts: Properties, Selection and Use, Weinheim/Zürich:Wiley-VCH/VHCA, 2002. Pharmaceutical salts typically are more soluble and more rapidly soluble in stomach and intestinal juices than non-ionic species and so are useful in solid dosage forms. Furthermore, because their solubility often is a function of pH, selective dissolution in one or another part of the digestive tract is possible and this capability can be manipulated as one aspect of delayed and sustained release behaviours. Also, because the salt-forming molecule can be in equilibrium with a neutral form, passage through biological membranes can be adjusted.

In some embodiments, pharmaceutically acceptable salts are obtained by reacting a compound described herein with an acid. In some embodiments, the compound described herein (i.e. free base form) is basic and is reacted with an organic acid or an inorganic acid. Inorganic acids include, but are not limited to, hydrochloric acid, hydrobromic acid, sulfuric acid, phosphoric acid, nitric acid, and metaphosphoric acid. Organic acids include, but are not limited to, 1-hydroxy-2-naphthoic acid; 2,2-dichloroacetic acid; 2-hydroxyethanesulfonic acid; 2-oxoglutaric acid; 4-acetamidobenzoic acid; 4-aminosalicylic acid; acetic acid; adipic acid; ascorbic acid (L); aspartic acid (L); benzenesulfonic acid; benzoic acid; camphoric acid (+); camphor-10-sulfonic acid (+); capric acid (decanoic acid); caproic acid (hexanoic acid); caprylic acid (octanoic acid); carbonic acid; cinnamic acid; citric acid; cyclamic acid; dodecylsulfuric acid; ethane-1,2-disulfonic acid; ethanesulfonic acid; formic acid; fumaric acid; galactaric acid; gentisic acid; glucoheptonic acid (D); gluconic acid (D); glucuronic acid (D); glutamic acid; glutaric acid; glycerophosphoric acid; glycolic acid; hippuric acid; isobutyric acid; lactic acid (DL); lactobionic acid; lauric acid; maleic acid; malic acid (−L); malonic acid; mandelic acid (DL); methanesulfonic acid; monomethyl fumarate; naphthalene-1,5-disulfonic acid; naphthalene-2-sulfonic acid; nicotinic acid; oleic acid; oxalic acid; palmitic acid; pamoic acid; phosphoric acid; proprionic acid; pyroglutamic acid (−L); salicylic acid; sebacic acid; stearic acid; succinic acid; sulfuric acid; tartaric acid (+L); thiocyanic acid; toluenesulfonic acid (p); and undecylenic acid.

In some embodiments, a compound described herein is prepared as a chloride salt, sulfate salt, bromide salt, mesylate salt, maleate salt, citrate salt or phosphate salt. In some embodiments, a compound described herein is prepared as a hydrochloride salt.

In some embodiments, pharmaceutically acceptable salts are obtained by reacting a compound described herein with a base. In some embodiments, the compound described herein is acidic and is reacted with a base. In such situations, an acidic proton of the compound described herein is replaced by a metal ion, e.g., lithium, sodium, potassium, magnesium, calcium, or an aluminum ion. In some cases, compounds described herein coordinate with an organic base, such as, but not limited to, ethanolamine, diethanolamine, triethanolamine, tromethamine, meglumine, N-methylglucamine, dicyclohexylamine, tris(hydroxymethyl)methylamine. In other cases, compounds described herein form salts with amino acids such as, but not limited to, arginine, lysine, and the like. Acceptable inorganic bases used to form salts with compounds that include an acidic proton, include, but are not limited to, aluminum hydroxide, calcium hydroxide, potassium hydroxide, sodium carbonate, potassium carbonate, sodium hydroxide, lithium hydroxide, and the like. In some embodiments, the compounds provided herein are prepared as a sodium salt, calcium salt, potassium salt, magnesium salt, meglumine salt, N-methylglucamine salt or ammonium salt. In some embodiments, the compounds provided herein are prepared as a sodium salt.

It should be understood that a reference to a pharmaceutically acceptable salt includes the solvent addition forms. In some embodiments, solvates contain either stoichiometric or non-stoichiometric amounts of a solvent, and are formed during the process of crystallization with pharmaceutically acceptable solvents such as water, ethanol, and the like. Hydrates are formed when the solvent is water, or alcoholates are formed when the solvent is alcohol. Solvates of compounds described herein are conveniently prepared or formed during the processes described herein. In addition, the compounds provided herein optionally exist in unsolvated as well as solvated forms.

The methods and formulations described herein include the use of N-oxides (if appropriate), crystalline forms (also known as polymorphs), or pharmaceutically acceptable salts of compounds described herein, as well as active metabolites of these compounds having the same type of activity.

In some embodiments, sites on the organic radicals (e.g. alkyl groups, aromatic rings) of compounds described herein are susceptible to various metabolic reactions. Incorporation of appropriate substituents on the organic radicals will reduce, minimize or eliminate this metabolic pathway. In specific embodiments, the appropriate substituent to decrease or eliminate the susceptibility of the aromatic ring to metabolic reactions is, by way of example only, a halogen, deuterium, an alkyl group, a haloalkyl group, or a deuteroalkyl group.

In another embodiment, the compounds described herein are labeled isotopically (e.g. with a radioisotope) or by another other means, including, but not limited to, the use of chromophores or fluorescent moieties, bioluminescent labels, or chemiluminescent labels.

Compounds described herein include isotopically-labeled compounds, which are identical to those recited in the various formulae and structures presented herein, but for the fact that one or more atoms are replaced by an atom having an atomic mass or mass number different from the atomic mass or mass number usually found in nature. Examples of isotopes that can be incorporated into the present compounds include isotopes of hydrogen, carbon, nitrogen, oxygen, fluorine and chlorine, such as, for example, ²H, ³H, ¹¹C, ¹³C, ¹⁴C, ¹⁵N, ¹⁸O, ¹⁷O, ³⁵S, ¹⁸F, ³⁶Cl. In one aspect, isotopically-labeled compounds described herein, for example those into which radioactive isotopes such as ³H and ¹⁴C are incorporated, are useful in drug and/or substrate tissue distribution assays. In one aspect, substitution with isotopes such as deuterium affords certain therapeutic advantages resulting from greater metabolic stability, such as, for example, increased in vivo half-life or reduced dosage requirements.

In some embodiments, the compounds described herein possess one or more stereocenters and each stereocenter exists independently in either the R or S configuration. The compounds presented herein include all diastereomeric, enantiomeric, atropisomers, and epimeric forms as well as the appropriate mixtures thereof. The compounds and methods provided herein include all cis, trans, syn, anti, entgegen (E), and zusammen (Z) isomers as well as the appropriate mixtures thereof.

Individual stereoisomers are obtained, if desired, by methods such as, stereoselective synthesis and/or the separation of stereoisomers by chiral chromatographic columns. In certain embodiments, compounds described herein are prepared as their individual stereoisomers by reacting a racemic mixture of the compound with an optically active resolving agent to form a pair of diastereoisomeric compounds/salts, separating the diastereomers and recovering the optically pure enantiomers. In some embodiments, resolution of enantiomers is carried out using covalent diastereomeric derivatives of the compounds described herein. In another embodiment, diastereomers are separated by separation/resolution techniques based upon differences in solubility. In other embodiments, separation of steroisomers is performed by chromatography or by the forming diastereomeric salts and separation by recrystallization, or chromatography, or any combination thereof. Jean Jacques, Andre Collet, Samuel H. Wilen, “Enantiomers, Racemates and Resolutions”, John Wiley And Sons, Inc., 1981. In some embodiments, stereoisomers are obtained by stereoselective synthesis.

### Synthesis of Compounds

Compounds of Formula (I) described herein are synthesized using standard synthetic techniques or using methods known in the art in combination with methods described herein.

Unless otherwise indicated, conventional methods of mass spectroscopy, NMR, HPLC, protein chemistry, biochemistry, recombinant DNA techniques and pharmacology are employed.

Compounds are prepared using standard organic chemistry techniques such as those described in, for example, March's Advanced Organic Chemistry, 6^{th} Edition, John Wiley and Sons, Inc. Standard procedures for the use of protecting groups to temporarily protect functionality such as amines, alcohols, and carboxylic acids are described in, for example, Protecting Groups in Organic Synthesis, 3^{rd} Edition, John Wiley and Sons, Inc. Alternative reaction conditions for the synthetic transformations described herein may be employed such as variation of solvent, reaction temperature, reaction time, as well as different chemical reagents and other reaction conditions. The starting materials are available from commercial sources or are readily prepared.

Pyridines are prepared using well known synthetic routes (see Allais et al Chem. Rev., 2014, 114, p 10829-10868 and references cited) and these are further functionalized to provide 2-substituted pyridines using a variety of methods. In some embodiments, 2-chloropyridines are obtained from direct chlorination of a pyridine using a suitable chlorination reagent. In some embodiments, the chlorination reagent is Cl₂. In some embodiments, 2-chloropyridines are prepared from the treatment of 2-hydroxypyridines with POCl₃. In other embodiments, 2-chloropyridines are prepared by the chlorination of a pyridine-N-oxide with a suitable chlorination reagent. In some embodiments, the chlorination reagent is POCl₃, phosgene or triphosgene. 2-Trifluoromethyl substituted pyridines are prepared by a variety of routes. In some embodiments, 2-iodopyridine is reacted with (trifluoromethyl)copper to afford 2-trifluoromethylpyridine (see Cottet and Schlosser Eur. J. Org. Chem., 2002, 2, p 327-330).

In some embodiments, the amide-linked compounds of Formula (I) having the general structure 1-8 are prepared as shown in Scheme 1.

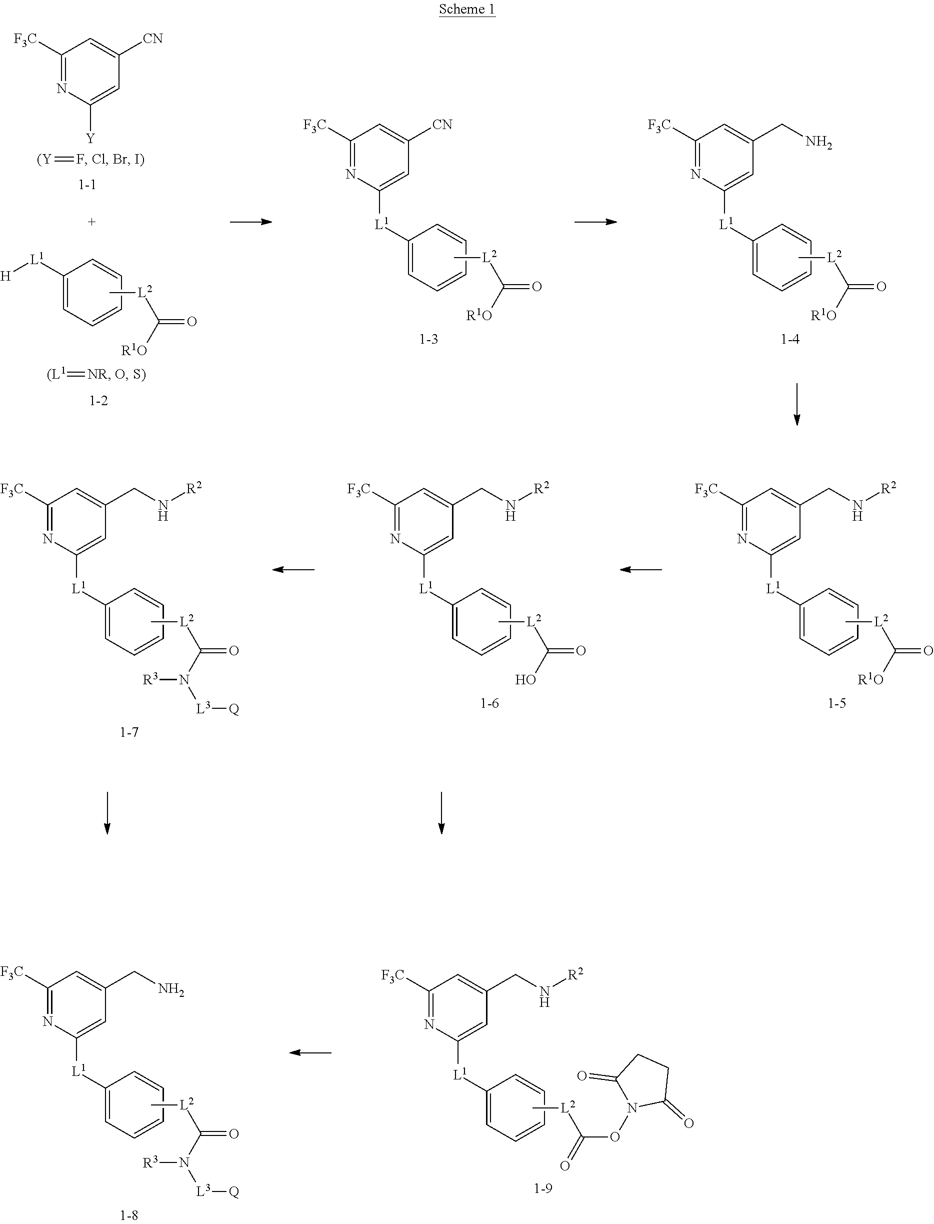

In some embodiments, 4-cyano-2-halo pyridine 1-1 is treated with an appropriately substituted phenyl derivative 1-2 in the presence of a suitable base using a suitable polar solvent to provide 1-3. In some embodiments, the suitable base is K₂CO₃ or alternatively KO^{t}Bu. In some embodiments, the suitable polar solvent is DMF. In some embodiments, 4-cyanopyridine derivatives 1-3 are converted, using suitable reducing agents, to the corresponding methylamino derivatives 1-4. In some embodiments, the suitable reducing agent is NaBH₄/CoCl₂ in an appropriate solvent, such as THF-MeOH, or alternatively hydrogen/palladium on carbon in a suitable solvent, such as EtOAc-MeOH. In some embodiments, the amino moiety of 1-4 is protected with a suitable protecting group to afford 1-5. In some embodiments, treatment of amine-derivative 1-4 with Fmoc-Cl in the presence of a suitable organic base, such as pyridine or DIEA, and in a suitable solvent such as THF or DCM, provides 1-5 (where R²=Fmoc). In other embodiments, treatment of amine-derivative 1-4 with Boc₂O in the presence of a suitable organic base, such as pyridine or DIEA, and in a suitable solvent, such as THF or DCM, provides 1-5 (where R²=Boc). In some embodiments, the ester is hydrolyzed using aqueous LiOH with a suitable organic solvent to afford acid 1-6. In some embodiments, the suitable organic solvent is MeOH or THF. In some embodiments, standard peptide coupling reaction conditions between carboxylic acid 1-6 and an appropriately substituted amine HNR³(L³-Q) yields amide-derivatives 1-7. In some embodiments, the use of standard amine-deprotection conditions provides 1-8. In some embodiments, standard coupling conditions between carboxylic acid 1-6 and N-hydroxysuccinimide affords activated ester 1-9.

In some embodiments, reaction of 1-9 with an appropriately substituted amine HNR³(L³-Q) in a suitable solvent such as DMF, followed by amine-deprotection provides 1-8.

In some embodiments, the amide or sulfonamide-linked compounds of Formula (I) having the general structure 2-8 or 2-10, respectively, are prepared as shown in Scheme 2.

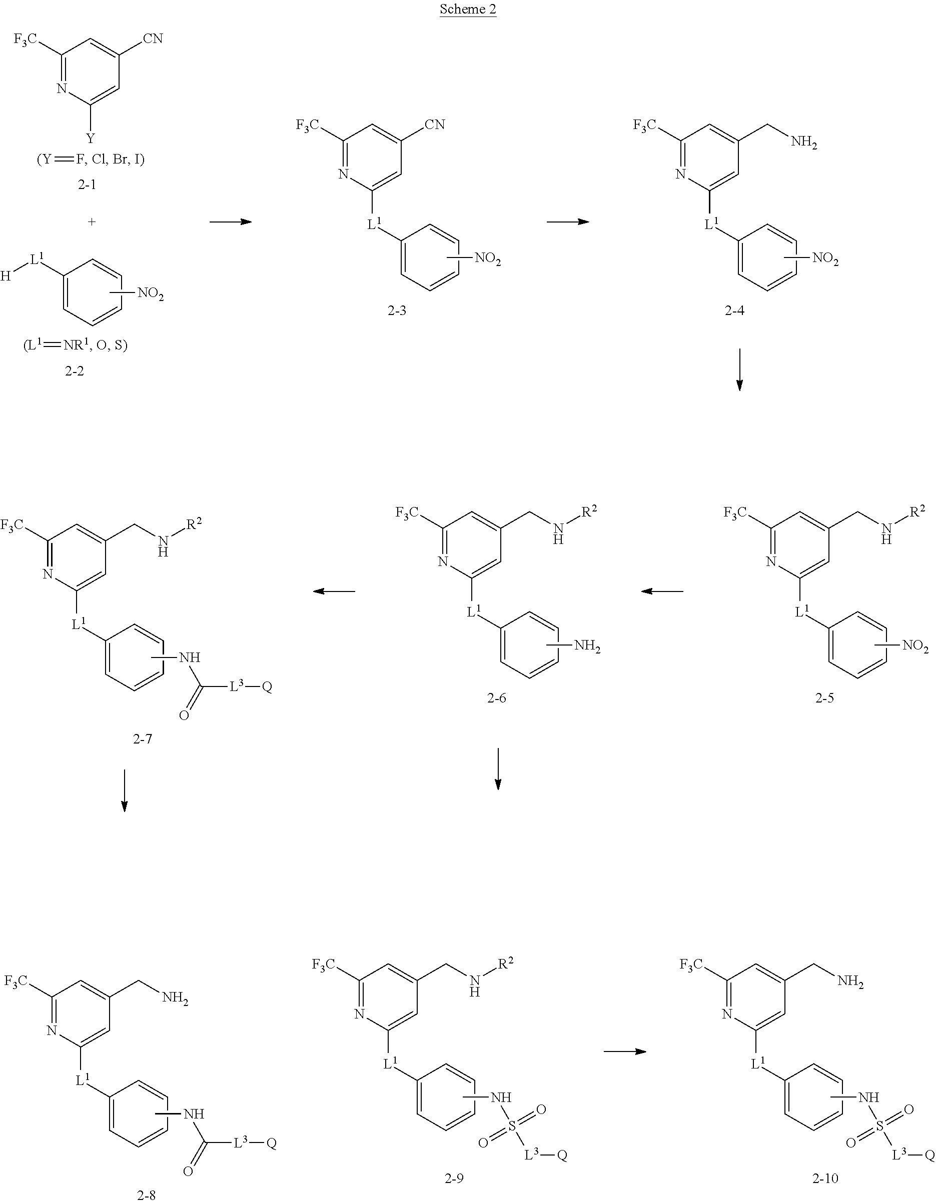

In some embodiments, 4-cyano-2-halo pyridine 2-1 is treated with a substituted nitrophenyl derivative 2-2 in the presence of a suitable base using a suitable polar solvent to provide 2-3. In some embodiments, the suitable base is K₂CO₃ or alternatively KO^{t}Bu. In some embodiments, the suitable polar solvent is DMF. In some embodiments, 4-cyanopyridine derivative 2-3 is converted, using suitable reducing agents, to the corresponding methylamino derivatives 2-4. In some embodiments, the reducing agent is NaBH₄/CoCl₂ in a suitable solvent, such as THF-MeOH, or alternatively BH₃-DMS in a suitable solvent, such as THF. In some embodiments, the amino moiety of 2-4 is protected with a suitable protecting group to afford 2-5. In some embodiments, treatment of amine-derivative 2-4 with Fmoc-Cl in the presence of a suitable organic base, such as pyridine or DIEA, and in a suitable solvent, such as THF or DCM, provides 2-5 (where R²=Fmoc). In other embodiments, treatment of amine-derivative 2-4 with Boc₂O in the presence of a suitable organic base, such as pyridine or DIEA, and in a suitable solvent such as THF or DCM, provides 2-5 (where R²=Boc). In some embodiments, 2-5 is converted to aniline-derivative 2-6, via treatment with a suitable reducing agent. In some embodiments, the suitable reducing agent is Na₂S₂O₆ in THF-H₂O, or alternatively hydrogen/palladium on carbon in a suitable solvent, such as EtOAc-MeOH. In some embodiments, standard coupling reaction conditions between aniline 2-6 and an appropriately substituted carboxylic acid Q-L³-CO₂H or acid chloride Q-L³-COCl, or activated ester such as Q-L³-CO-(N-oxy-succinimide), provides amide-derivatives 2-7. In some embodiments, the use of standard amine-deprotection conditions affords 2-8. In some embodiments, reaction of aniline 2-6 with an appropriately substituted sulfonyl chloride Q-L³-SO₂Cl in the presence of a suitable organic base, such as pyridine or DIEA, and in a suitable solvent, such as DMF or DCM, provides sulfonamide 2-9. In some embodiments, treatment of 2-9 under standard amine-deprotection conditions provides 2-10.

In some embodiments, the triazole-linked compounds of Formula (I) having the general structure 3-5 or 3-10 are prepared as shown in Scheme 3.

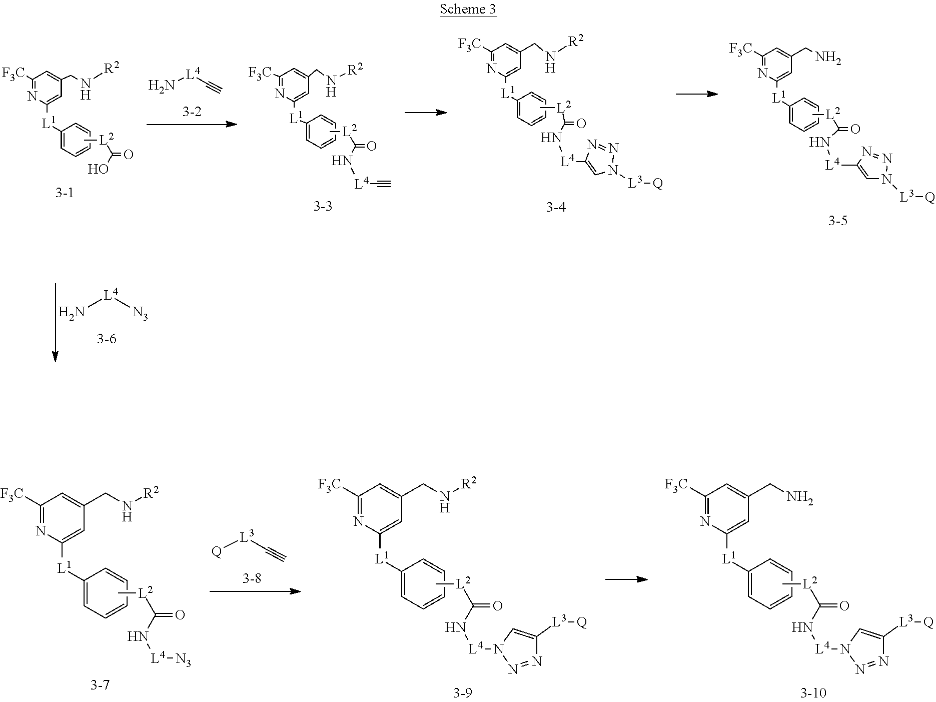

In some embodiments, standard peptide coupling conditions between carboxylic acid 3-1 (prepared as shown in Scheme 1) and an appropriately substituted amine 3-2 yield alkyne-containing amide-derivatives 3-3. In some embodiments, treatment of alkyne derivative 3-3 with an appropriately substituted azide N₃-L³-Q in the presence of a catalyst such as CuSO₄ or CuI, and in the presence of sodium ascorbate and benzoic acid, in a suitable solvent such as ^{t}BuOH—H₂O or DMSO—H₂O, yields triazole derivatives 3-4. In some embodiments, the use of standard amine-deprotection conditions provides 3-5. In some embodiments, standard peptide coupling reaction conditions between carboxylic acid 3-1 and an appropriately substituted amine 3-6 affords azide-containing amide-derivatives 3-7. In some embodiments, treatment of azide derivative 3-7 with an appropriately substituted alkyne 3-8 in the presence of a suitable catalyst such as CuSO₄ or CuI, and in the presence of sodium ascorbate and benzoic acid, in a suitable solvent such as ^{t}BuOH—H₂O or DMSO—H₂O, affords triazole derivatives 3-9. In some embodiments, treatment of 3-9 under standard amine-deprotection conditions affords 3-10.

In some embodiments, the triazole-linked compounds of Formula (I) having the general structure 4-3 are prepared as shown in Scheme 4.

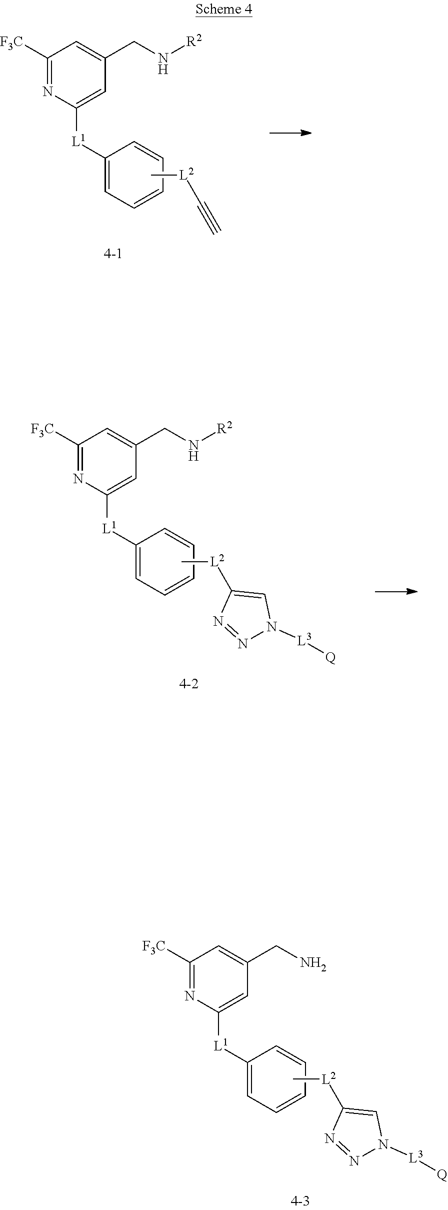

In some embodiments, treatment of alkyne derivative 4-1 (prepared using general procedures outlined in Scheme 1) with an appropriately substituted azide N₃-L³-Q in the presence of a catalyst such as CuSO₄ or CuI, and in the presence of sodium ascorbate and benzoic acid, in a suitable solvent such as ^{t}BuOH—H₂O or DMSO—H₂O, yields triazole derivatives 4-2. In some embodiments, the use of standard amine-deprotection conditions provides 4-3.

In some embodiments, the triazole-linked compounds of Formula (I) having the general structure 5-4 are prepared as shown in Scheme 5.

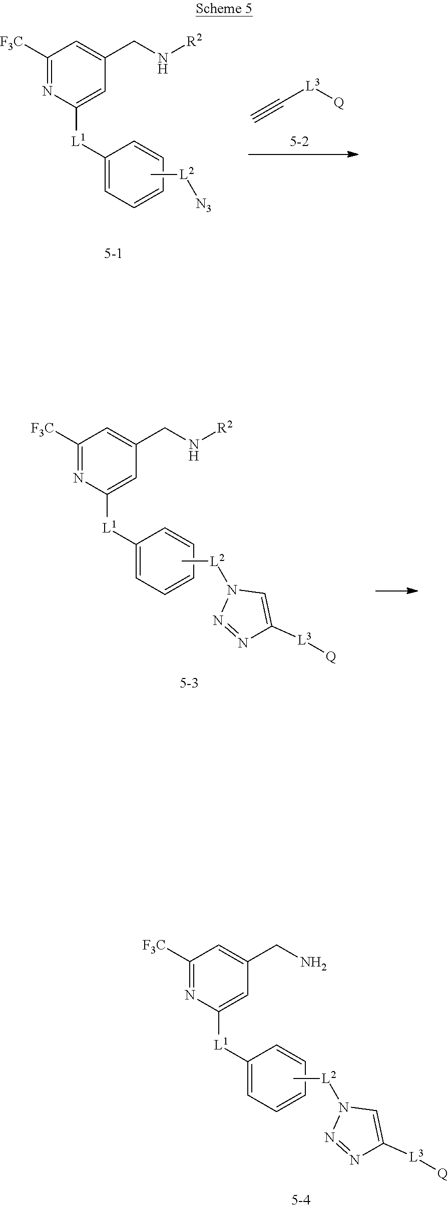

In some embodiments, treatment of azide derivative 5-1 (prepared using general procedures outlined in Scheme 1) with an appropriately substituted alkyne 5-2 in the presence of a suitable catalyst such as CuSO₄ or CuI, and in the presence of sodium ascorbate and benzoic acid, in a suitable solvent such as ^{t}BuOH—H₂O or DMSO—H₂O, yields triazole derivatives 5-3. In some embodiments, the use of standard amine-deprotection conditions provides 5-4.

In some embodiments, the pyridazine-linked compounds of Formula (I) having the general structure 6-4 are prepared as shown in Scheme 6.

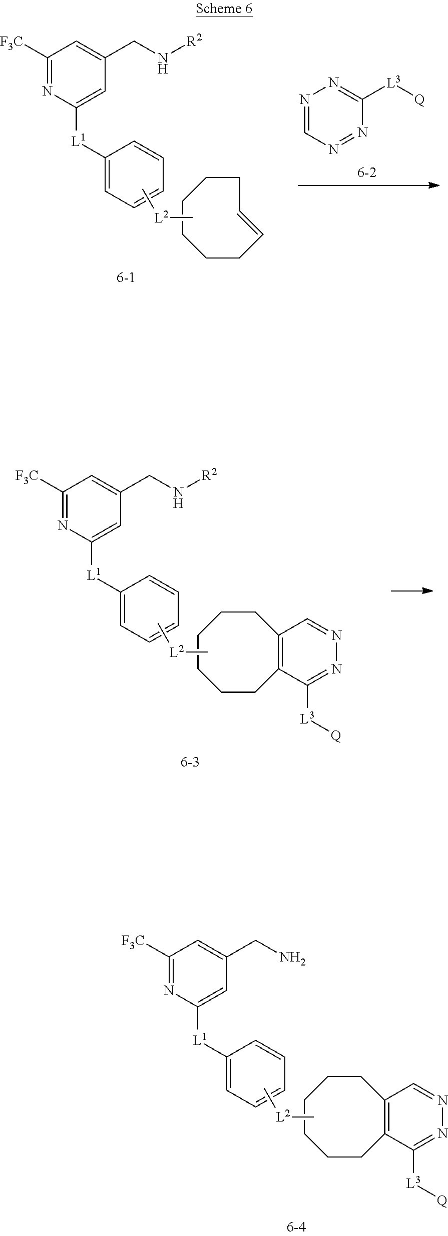

In some embodiments, treatment of a trans-cyclooctene (TCO) derivative 6-1 (prepared using general procedures outlined in Scheme 1) with an appropriately substituted tetrazine derivative 6-2 in a suitable solvent such as H₂O or MeCN—H₂O, yields pyridazine derivatives 6-3 (see Knall and Slugovc, Chem. Soc. Rev., 2013, 42, p 5131-5142 and references cited therein). In some embodiments, the use of standard amine-deprotection conditions provides 6-4.

In some embodiments, the pyridazine-linked compounds of Formula (I) having the general structure 7-4 are prepared as shown in Scheme 7.

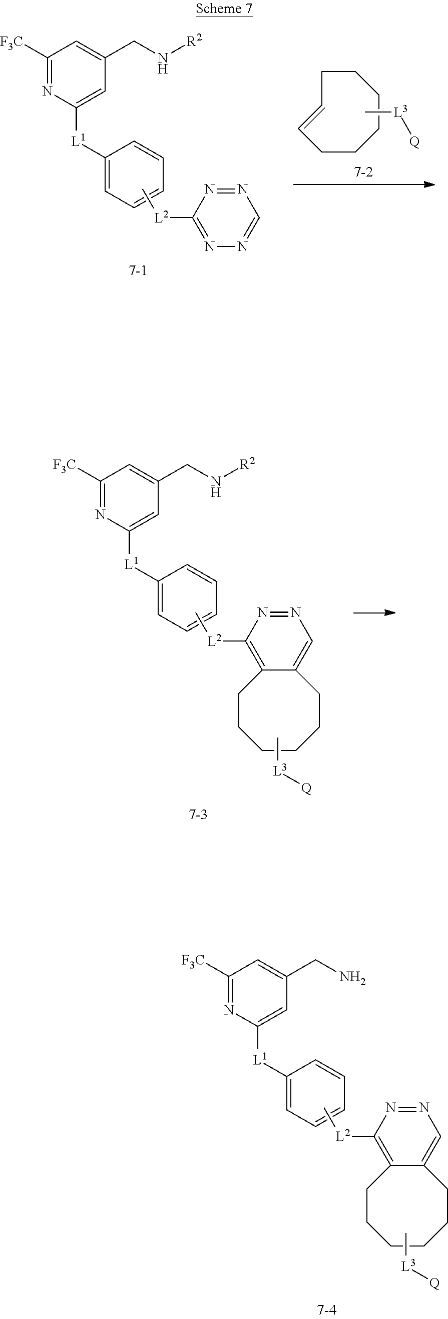

In some embodiments, treatment of a tetrazine derivative 7-1 (prepared using general procedures outlined in Scheme 1) with an appropriately substituted trans-cyclooctene (TCO) derivative 7-2 in a suitable solvent such as H₂O or MeCN—H₂O, yields pyridazine derivatives 7-3. In some embodiments, the use of standard amine-deprotection conditions affords 7-4.

In some embodiments, compounds are prepared as described in the Examples.

### Certain Terminology

Unless otherwise stated, the following terms used in this application have the definitions given below. The use of the term “including” as well as other forms, such as “include”, “includes,” and “included,” is not limiting. The section headings used herein are for organizational purposes only and are not to be construed as limiting the subject matter described.

As used herein, C₁-Cₓ includes C₁-C₂, C₁-C₃ . . . C₁-Cₓ. By way of example only, a group designated as “C₁-C₄” indicates that there are one to four carbon atoms in the moiety, i.e. groups containing 1 carbon atom, 2 carbon atoms, 3 carbon atoms or 4 carbon atoms. Thus, by way of example only, “C₁-C₄ alkyl” indicates that there are one to four carbon atoms in the alkyl group, i.e., the alkyl group is selected from among methyl, ethyl, propyl, iso-propyl, n-butyl, iso-butyl, sec-butyl, and t-butyl.

An “alkyl” group refers to an aliphatic hydrocarbon group. The alkyl group is branched or straight chain. In some embodiments, the “alkyl” group has 1 to 10 carbon atoms, i.e. a C₁-C₁₀alkyl. Whenever it appears herein, a numerical range such as “1 to 10” refers to each integer in the given range; e.g., “1 to 10 carbon atoms” means that the alkyl group consists of 1 carbon atom, 2 carbon atoms, 3 carbon atoms, etc., up to and including 10 carbon atoms, although the present definition also covers the occurrence of the term “alkyl” where no numerical range is designated. In some embodiments, an alkyl is a C₁-C₆alkyl. In one aspect the alkyl is methyl, ethyl, propyl, iso-propyl, n-butyl, iso-butyl, sec-butyl, or t-butyl. Typical alkyl groups include, but are in no way limited to, methyl, ethyl, propyl, isopropyl, butyl, isobutyl, sec-butyl, tertiary butyl, pentyl, neopentyl, or hexyl.

An “alkylene” group refers refers to a divalent alkyl radical. Any of the above mentioned monovalent alkyl groups may be an alkylene by abstraction of a second hydrogen atom from the alkyl. In some embodiments, an alkelene is a C₁-C₆alkylene. In other embodiments, an alkylene is a C₁-C₄alkylene. Typical alkylene groups include, but are not limited to, —CH₂—, —CH(CH₃)—, —C(CH₃)₂—, —CH₂CH₂—, —CH₂CH(CH₃)—, —CH₂C(CH₃)₂—, —CH₂CH₂CH₂—, —CH₂CH₂CH₂CH₂—, and the like.

“Deuteroalkyl” refers to an alkyl group where 1 or more hydrogen atoms of an alkyl are replaced with deuterium.

The term “alkenyl” refers to a type of alkyl group in which at least one carbon-carbon double bond is present. In one embodiment, an alkenyl group has the formula —C(R)═CR₂, wherein R refers to the remaining portions of the alkenyl group, which may be the same or different. In some embodiments, R is H or an alkyl. Non-limiting examples of an alkenyl group include —CH═CH₂, —C(CH₃)═CH₂, —CH═CHCH₃, —C(CH₃)═CHCH₃, and —CH₂CH═CH₂.

The term “alkynyl” refers to a type of alkyl group in which at least one carbon-carbon triple bond is present. In one embodiment, an alkynyl group has the formula —C≡C—R, wherein R refers to the remaining portions of the alkynyl group. In some embodiments, R is H or an alkyl. Non-limiting examples of an alkynyl group include —C≡CH, —C≡CCH₃—C≡CCH₂CH₃, —CH₂C≡CH.

An “alkoxy” group refers to a (alkyl)O— group, where alkyl is as defined herein.

The term “alkylamine” refers to the —N(alkyl)ₓH_{y} group, where x is 0 and y is 2, or where x is 1 and y is 1, or where x is 2 and y is 0.

The term “aromatic” refers to a planar ring having a delocalized 7c-electron system containing 4n+2π electrons, where n is an integer. The term “aromatic” includes both carbocyclic aryl (“aryl”, e.g., phenyl) and heterocyclic aryl (or “heteroaryl” or “heteroaromatic”) groups (e.g., pyridine). The term includes monocyclic or fused-ring polycyclic (i.e., rings which share adjacent pairs of carbon atoms) groups.

The term “carbocyclic” or “carbocycle” refers to a ring or ring system where the atoms forming the backbone of the ring are all carbon atoms. The term thus distinguishes carbocyclic from “heterocyclic” rings or “heterocycles” in which the ring backbone contains at least one atom which is different from carbon. In some embodiments, at least one of the two rings of a bicyclic carbocycle is aromatic. In some embodiments, both rings of a bicyclic carbocycle are aromatic.

As used herein, the term “aryl” refers to an aromatic ring wherein each of the atoms forming the ring is a carbon atom. In one aspect, aryl is phenyl or a naphthyl. In some embodiments, an aryl is a phenyl. In some embodiments, an aryl is a C₆-C₁₀aryl. Depending on the structure, an aryl group is a monoradical or a diradical (i.e., an arylene group).

The term “cycloalkyl” refers to a monocyclic or polycyclic aliphatic, non-aromatic radical, wherein each of the atoms forming the ring (i.e. skeletal atoms) is a carbon atom. In some embodiments, cycloalkyls are spirocyclic or bridged compounds. In some embodiments, cycloalkyls are optionally fused with an aromatic ring, and the point of attachment is at a carbon that is not an aromatic ring carbon atom. Cycloalkyl groups include groups having from 3 to 10 ring atoms. In some embodiments, cycloalkyl groups are selected from among cyclopropyl, cyclobutyl, cyclopentyl, cyclopentenyl, cyclohexyl, cyclohexenyl, cycloheptyl, cyclooctyl, spiro[2.2]pentyl, norbornyl and bicyclo[1.1.1]pentyl. In some embodiments, a cycloalkyl is a C₃-C₆cycloalkyl.

The term “halo” or, alternatively, “halogen” or “halide” means fluoro, chloro, bromo or iodo. In some embodiments, halo is fluoro, chloro, or bromo.

The term “fluoroalkyl” refers to an alkyl in which one or more hydrogen atoms are replaced by a fluorine atom. In one aspect, a fluoralkyl is a C₁-C₆fluoroalkyl.

The term “heteroalkyl” refers to an alkyl group in which one or more skeletal atoms of the alkyl are selected from an atom other than carbon, e.g., oxygen, nitrogen (e.g. —NH—, —N(alkyl)-, sulfur, or combinations thereof. A heteroalkyl is attached to the rest of the molecule at a carbon atom of the heteroalkyl. In one aspect, a heteroalkyl is a C₁-C₆heteroalkyl.

The term “heterocycle” or “heterocyclic” refers to heteroaromatic rings (also known as heteroaryls) and heterocycloalkyl rings (also known as heteroalicyclic groups) containing one to four heteroatoms in the ring(s), where each heteroatom in the ring(s) is selected from O, S and N, wherein each heterocyclic group has from 3 to 10 atoms in its ring system, and with the proviso that any ring does not contain two adjacent 0 or S atoms. Non-aromatic heterocyclic groups (also known as heterocycloalkyls) include rings having 3 to 10 atoms in its ring system and aromatic heterocyclic groups include rings having 5 to 10 atoms in its ring system. The heterocyclic groups include benzo-fused ring systems. Examples of non-aromatic heterocyclic groups are pyrrolidinyl, tetrahydrofuranyl, dihydrofuranyl, tetrahydrothienyl, oxazolidinonyl, tetrahydropyranyl, dihydropyranyl, tetrahydrothiopyranyl, piperidinyl, morpholinyl, thiomorpholinyl, thioxanyl, piperazinyl, aziridinyl, azetidinyl, oxetanyl, thietanyl, homopiperidinyl, oxepanyl, thiepanyl, oxazepinyl, diazepinyl, thiazepinyl, 1,2,3,6-tetrahydropyridinyl, pyrrolin-2-yl, pyrrolin-3-yl, indolinyl, 2H-pyranyl, 4H-pyranyl, dioxanyl, 1,3-dioxolanyl, pyrazolinyl, dithianyl, dithiolanyl, dihydropyranyl, dihydrothienyl, dihydrofuranyl, pyrazolidinyl, imidazolinyl, imidazolidinyl, 3-azabicyclo[3.1.0]hexanyl, 3-azabicyclo[4.1.0]heptanyl, 3H-indolyl, indolin-2-onyl, isoindolin-1-onyl, isoindoline-1,3-dionyl, 3,4-dihydroisoquinolin-1(2H)-onyl, 3,4-dihydroquinolin-2(1H)-onyl, isoindoline-1,3-dithionyl, benzo[d]oxazol-2(3H)-onyl, 1H-benzo[d]imidazol-2(3H)-onyl, benzo[d]thiazol-2(3H)-onyl, and quinolizinyl. Examples of aromatic heterocyclic groups are pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, thiadiazolyl, furazanyl, benzofurazanyl, benzothiophenyl, benzothiazolyl, benzoxazolyl, quinazolinyl, quinoxalinyl, naphthyridinyl, and furopyridinyl. The foregoing groups are either C-attached (or C-linked) or N-attached where such is possible. For instance, a group derived from pyrrole includes both pyrrol-1-yl (N-attached) or pyrrol-3-yl (C-attached). Further, a group derived from imidazole includes imidazol-1-yl or imidazol-3-yl (both N-attached) or imidazol-2-yl, imidazol-4-yl or imidazol-5-yl (all C-attached). The heterocyclic groups include benzo-fused ring systems. Non-aromatic heterocycles are optionally substituted with one or two oxo (═O) moieties, such as pyrrolidin-2-one. In some embodiments, at least one of the two rings of a bicyclic heterocycle is aromatic. In some embodiments, both rings of a bicyclic heterocycle are aromatic.

The terms “heteroaryl” or, alternatively, “heteroaromatic” refers to an aryl group that includes one or more ring heteroatoms selected from nitrogen, oxygen and sulfur. Illustrative examples of heteroaryl groups include monocyclic heteroaryls and bicycicic heteroaryls. Monocyclic heteroaryls include pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, pyridazinyl, triazinyl, oxadiazolyl, thiadiazolyl, and furazanyl. Bicyclic heteroaryls include indolizinyl, indolyl, benzofuranyl, benzothiophenyl, indazolyl, benzimidazolyl, purinyl, quinolizinyl, quinolinyl, isoquinolinyl, cinnolinyl, phthalazinyl, quinazolinyl, quinoxalinyl, 1,8-naphthyridinyl, and pteridinyl. In some embodiments, a heteroaryl contains 0-4 N atoms in the ring. In some embodiments, a heteroaryl contains 1-4 N atoms in the ring. In some embodiments, a heteroaryl contains 0-4 N atoms, 0-1 O atoms, and 0-1 S atoms in the ring. In some embodiments, a heteroaryl contains 1-4 N atoms, 0-1 O atoms, and 0-1 S atoms in the ring. In some embodiments, heteroaryl is a C₁-C₉heteroaryl. In some embodiments, monocyclic heteroaryl is a C₁-C₅heteroaryl. In some embodiments, monocyclic heteroaryl is a 5-membered or 6-membered heteroaryl. In some embodiments, bicyclic heteroaryl is a C₆-C₉heteroaryl.

A “heterocycloalkyl” or “heteroalicyclic” group refers to a cycloalkyl group that includes at least one heteroatom selected from nitrogen, oxygen and sulfur. In some embodiments, a heterocycloalkyl is fused with an aryl or heteroaryl. In some embodiments, the heterocycloalkyl is oxazolidinonyl, pyrrolidinyl, tetrahydrofuranyl, tetrahydrothienyl, tetrahydropyranyl, tetrahydrothiopyranyl, piperidinyl, morpholinyl, thiomorpholinyl, piperazinyl, piperidin-2-onyl, pyrrolidine-2,5-dithionyl, pyrrolidine-2,5-dionyl, pyrrolidinonyl, imidazolidinyl, imidazolidin-2-onyl, or thiazolidin-2-onyl. The term heteroalicyclic also includes all ring forms of the carbohydrates, including but not limited to the monosaccharides, the disaccharides and the oligosaccharides. In one aspect, a heterocycloalkyl is a C₂-C₁₀heterocycloalkyl. In another aspect, a heterocycloalkyl is a C₄-C₁₀heterocycloalkyl. In some embodiments, a heterocycloalkyl contains 0-2 N atoms in the ring. In some embodiments, a heterocycloalkyl contains 0-2 N atoms, 0-2 O atoms and 0-1 S atoms in the ring.

The term “bond” or “single bond” refers to a chemical bond between two atoms, or two moieties when the atoms joined by the bond are considered to be part of larger substructure. In one aspect, when a group described herein is a bond, the referenced group is absent thereby allowing a bond to be formed between the remaining identified groups.

The term “moiety” refers to a specific segment or functional group of a molecule. Chemical moieties are often recognized chemical entities embedded in or appended to a molecule.

The term “optionally substituted” or “substituted” means that the referenced group is optionally substituted with one or more additional group(s) individually and independently selected from halogen, —CN, —NH₂, —NH(alkyl), —N(alkyl)₂, —OH, —CO₂H, —CO₂alkyl, —C(═O)NH₂, —C(═O)NH(alkyl), —C(═O)N(alkyl)₂, —S(═O)₂NH₂, —S(═O)₂NH(alkyl), —S(═O)₂N(alkyl)₂, alkyl, cycloalkyl, fluoroalkyl, heteroalkyl, alkoxy, fluoroalkoxy, heterocycloalkyl, aryl, heteroaryl, aryloxy, alkylthio, arylthio, alkylsulfoxide, arylsulfoxide, alkylsulfone, and arylsulfone. In some other embodiments, optional substituents are independently selected from halogen, —CN, —NH₂, —NH(CH₃), —N(CH₃)₂, —OH, —CO₂H, —CO₂(C₁-C₄alkyl), —C(═O)NH₂, —C(═O)NH(C₁-C₄alkyl), —C(═O)N(C₁-C₄alkyl)₂, —S(═O)₂NH₂, —S(═O)₂NH(C₁-C₄alkyl), —S(═O)₂N(C₁-C₄alkyl)₂, C₁-C₄alkyl, C₃-C₆cycloalkyl, C₁-C₄fluoroalkyl, C₁-C₄heteroalkyl, C₁-C₄alkoxy, C₁-C₄fluoroalkoxy, —SC₁—C₄alkyl, —S(═O)C₁-C₄alkyl, and —S(═O)₂C₁-C₄alkyl. In some embodiments, optional substituents are independently selected from halogen, —CN, —NH₂, —OH, —NH(CH₃), —N(CH₃)₂, —CH₃, —CH₂CH₃, —CF₃, —OCH₃, and —OCF₃. In some embodiments, substituted groups are substituted with one or two of the preceding groups. In some embodiments, an optional substituent on an aliphatic carbon atom (acyclic or cyclic) includes oxo (═O).

The term “acceptable” with respect to a formulation, composition or ingredient, as used herein, means having no persistent detrimental effect on the general health of the subject being treated.

The term “modulate” as used herein, means to interact with a target either directly or indirectly so as to alter the activity of the target, including, by way of example only, to enhance the activity of the target, to inhibit the activity of the target, to limit the activity of the target, or to extend the activity of the target.

The term “modulator” as used herein, refers to a molecule that interacts with a target either directly or indirectly. The interactions include, but are not limited to, the interactions of an agonist, partial agonist, an inverse agonist, antagonist, degrader, or combinations thereof. In some embodiments, a modulator is an antagonist. In some embodiments, a modulator is a degrader.

The terms “administer,” “administering”, “administration,” and the like, as used herein, refer to the methods that may be used to enable delivery of compounds or compositions to the desired site of biological action. These methods include, but are not limited to oral routes, intraduodenal routes, parenteral injection (including intravenous, subcutaneous, intraperitoneal, intramuscular, intravascular or infusion), topical and rectal administration. Those of skill in the art are familiar with administration techniques that can be employed with the compounds and methods described herein. In some embodiments, the compounds and compositions described herein are administered orally.

The terms “co-administration” or the like, as used herein, are meant to encompass administration of the selected therapeutic agents to a single patient, and are intended to include treatment regimens in which the agents are administered by the same or different route of administration or at the same or different time.

The terms “effective amount” or “therapeutically effective amount,” as used herein, refer to a sufficient amount of an agent or a compound being administered, which will relieve to some extent one or more of the symptoms of the disease or condition being treated. The result includes reduction and/or alleviation of the signs, symptoms, or causes of a disease, or any other desired alteration of a biological system. For example, an “effective amount” for therapeutic uses is the amount of the composition comprising a compound as disclosed herein required to provide a clinically significant decrease in disease symptoms. An appropriate “effective” amount in any individual case is optionally determined using techniques, such as a dose escalation study.

The terms “enhance” or “enhancing,” as used herein, means to increase or prolong either in potency or duration a desired effect. Thus, in regard to enhancing the effect of therapeutic agents, the term “enhancing” refers to the ability to increase or prolong, either in potency or duration, the effect of other therapeutic agents on a system. An “enhancing-effective amount,” as used herein, refers to an amount adequate to enhance the effect of another therapeutic agent in a desired system.

The term “pharmaceutical combination” as used herein, means a product that results from the mixing or combining of more than one active ingredient and includes both fixed and non-fixed combinations of the active ingredients. The term “fixed combination” means that the active ingredients, e.g. a compound described herein, or a pharmaceutically acceptable salt thereof, and a co-agent, are both administered to a patient simultaneously in the form of a single entity or dosage. The term “non-fixed combination” means that the active ingredients, e.g. a compound described herein, or a pharmaceutically acceptable salt thereof, and a co-agent, are administered to a patient as separate entities either simultaneously, concurrently or sequentially with no specific intervening time limits, wherein such administration provides effective levels of the two compounds in the body of the patient. The latter also applies to cocktail therapy, e.g. the administration of three or more active ingredients.

The terms “kit” and “article of manufacture” are used as synonyms.

The term “subject” or “patient” encompasses mammals. Examples of mammals include, but are not limited to, any member of the Mammalian class: humans; non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. In one aspect, the mammal is a human.

The terms “treat,” “treating” or “treatment,” as used herein, include alleviating, abating or ameliorating at least one symptom of a disease or condition, preventing additional symptoms, inhibiting the disease or condition, e.g., arresting the development of the disease or condition, relieving the disease or condition, causing regression of the disease or condition, relieving a condition caused by the disease or condition, or stopping the symptoms of the disease or condition either prophylactically and/or therapeutically.

### Pharmaceutical Compositions

In some embodiments, the probe compound is formulated into a pharmaceutical composition. Pharmaceutical compositions are formulated in a conventional manner using one or more pharmaceutically acceptable inactive ingredients that facilitate processing of the compounds into preparations that are used pharmaceutically. Proper formulation is dependent upon the route of administration chosen. A summary of pharmaceutical compositions described herein is found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999), herein incorporated by reference for such disclosure.

In some embodiments, the compounds described herein are administered either alone or in combination with pharmaceutically acceptable carriers, excipients or diluents, in a pharmaceutical composition. Administration of the compounds and compositions described herein can be effected by any method that enables delivery of the compounds to the site of action. These methods include, though are not limited to delivery via enteral routes (including oral, gastric or duodenal feeding tube, rectal suppository and rectal enema), parenteral routes (injection or infusion, including intraarterial, intracardiac, intradermal, intraduodenal, intramedullary, intramuscular, intraosseous, intraperitoneal, intrathecal, intravascular, intravenous, intravitreal, epidural and subcutaneous), inhalational, transdermal, transmucosal, sublingual, buccal and topical (including epicutaneous, dermal, enema, eye drops, ear drops, intranasal, vaginal) administration, although the most suitable route may depend upon for example the condition and disorder of the recipient. By way of example only, compounds described herein can be administered locally to the area in need of treatment, by for example, local infusion, injection, catheter, or implant. The administration can also be by direct injection at the site of a diseased tissue or organ.

### Samples, Analytical Techniques, and Instrumentation

In certain embodiments, one or more of the methods disclosed herein comprise a sample. In some embodiments, the sample is a cell sample or a tissue sample. In some instances, the sample is a cell sample. In some embodiments, the sample for use with the methods described herein is obtained from cells of a mammal. In some instances, the mammalian cell is a primate, human, ape, equine, bovine, porcine, canine, feline, or rodent. In some instances, the mammal is a human, ape, dog, cat, rabbit, ferret, mouse, rat, hamster, gerbil, hamster, chinchilla, or guinea pig. In some instances, the mammal is a human.

In some embodiments, the sample for use with the methods described herein is obtained from a mammalian cell. In some instances, the mammalian cell is an epithelial cell, connective tissue cell, hormone secreting cell, a nerve cell, a skeletal muscle cell, a blood cell, or an immune system cell.

In some embodiments, the sample for use in the methods is from any tissue or fluid from an individual. Samples include, but are not limited to, tissue (e.g. connective tissue, muscle tissue, nervous tissue, or epithelial tissue), whole blood, dissociated bone marrow, bone marrow aspirate, pleural fluid, peritoneal fluid, central spinal fluid, abdominal fluid, pancreatic fluid, cerebrospinal fluid, brain fluid, ascites, pericardial fluid, urine, saliva, bronchial lavage, sweat, tears, ear flow, sputum, hydrocele fluid, semen, vaginal flow, milk, amniotic fluid, and secretions of respiratory, intestinal or genitourinary tract. In some embodiments, the sample is a tissue sample, such as a sample obtained from a biopsy or a tumor tissue sample. In some embodiments, the sample is a blood serum sample.

As used herein, “sample” refers to a composition containing a material to be detected.

For the purposes herein, sample refers to anything which can contain a biomolecule, such as but not limited to LOXL2. The sample can be a biological sample, such as a biological fluid or a biological tissue obtained from any organism or a cell of or from an organism. Examples of biological fluids include urine, blood, plasma, serum, saliva, semen, stool, sputum, cerebral spinal fluid, tears, mucus, sperm, amniotic fluid or the like. Biological tissues are aggregates of cells, usually of a particular kind together with their intercellular substance that form one of the structural materials of a human, animal, plant, bacterial, fungal or viral structure, including connective, epithelium, muscle and nerve tissues. Examples of biological tissues also include organs, tumors, lymph nodes, arteries and individual cell(s). Thus, samples include biological samples (e.g., any material obtained from a source originating from a living being (e.g., human, animal, plant, bacteria, fungi, protist, virus). The biological sample can be in any form, including solid materials (e.g., tissue, cell pellets and biopsies, tissues from cadavers) and biological fluids (e.g., urine, blood, saliva, amniotic fluid and mouth wash (containing buccal cells)). In certain embodiments, solid materials are mixed with a fluid. In embodiments herein, a sample for mass spectrometric analysis includes samples that contain a mixture of compound of Formula (I)-biomolecule complexes, such as Formula (I)-LOXL2 complexes.

In some embodiments, the samples are obtained from the individual by any suitable means of obtaining the sample using well-known and routine clinical methods. Procedures for obtaining tissue samples from an individual are well known. For example, procedures for drawing and processing tissue sample such as from a needle aspiration biopsy is well-known and is employed to obtain a sample for use in the methods provided. Typically, for collection of such a tissue sample, a thin hollow needle is inserted into a mass such as a tumor mass for sampling of cells that, after being stained, will be examined under a microscope.

### Sample Preparation and Analysis

In some embodiments, the sample is a solution. In some instances, the sample solution comprises a solution such as a buffer (e.g. phosphate buffered saline) or a media.

In some embodiments, the sample (e.g., cells or a cell solution) is incubated with a probe described herein for analysis of probe interactions with biomolecules in the sample, such as LOXL2. In some instances, the sample (e.g., cells or a cell solution) is further incubated in the presence of a LOXL2i prior to addition of the probe described herein. In some instances, the sample is compared with a control. In some instances, the control comprises the probe but not the LOXL2i. In some instances, a difference is observed between probe-protein interactions between the sample and the control. In some instances, the difference correlates to the interaction between the LOXL2i and the biomolecules in the sample, such as LOXL2.

In some instances, the sample is divided into a first cell solution and a second cell solution. In some cases, the first cell solution is incubated with a LOXL2i for an extended period of time prior to incubating the first cell solution with a probe described herein to generate a first group of cysteine-reactive probe-protein complexes. In some instances, the extended period of time is about 5, 10, 15, 20, 30, 60, 90, 120 minutes or longer. In some instances, the second cell solution comprises a second probe to generate a second group of probe-protein complexes. In some instances, the first probe and the second probe are the same. In some embodiments, the second cell solution further comprises a control.

In some embodiments, the probe-protein complex is further conjugated to a chromophore, such as a fluorophore. In some instances, the probe-protein complex is separated and visualized utilizing an electrophoresis system, such as through a gel electrophoresis, or a capillary electrophoresis. Exemplary gel electrophoresis includes agarose based gels, polyacrylamide based gels, or starch based gels. In some instances, the probe-protein is subjected to a native electrophoresis condition. In some instances, the probe-protein is subjected to a denaturing electrophoresis condition.

In some instances, the probe-protein complex after harvesting is further fragmentized to generate protein fragments. In some instances, fragmentation is generated through mechanical stress, pressure, or chemical means. In some instances, the protein from the probe-protein complexes is fragmented by a chemical means. In some embodiments, the chemical means is a protease.

In some instances, the fragmentation is a random fragmentation. In some instances, the fragmentation generates specific lengths of protein fragments, or the shearing occurs at particular sequence of amino acid regions.

In some instances, the protein fragments are further analyzed by a proteomic method such as by liquid chromatography (LC) (e.g. high performance liquid chromatography), liquid chromatography-mass spectrometry (LC-MS), matrix-assisted laser desorption/ionization (MALDI-TOF), gas chromatography-mass spectrometry (GC-MS), capillary electrophoresis-mass spectrometry (CE-MS), or nuclear magnetic resonance imaging (NMR).

In some embodiments, the LC method is any suitable LC method known in the art, for separation of a sample into its individual parts. This separation occurs based on the interaction of the sample with the mobile and stationary phases. Since there are many stationary/mobile phase combinations that are employed when separating a mixture, there are several different types of chromatography that are classified based on the physical states of those phases. In some embodiments, the LC is further classified as normal-phase chromatography, reverse-phase chromatography, size-exclusion chromatography, ion-exchange chromatography, affinity chromatography, displacement chromatography, partition chromatography, flash chromatography, chiral chromatography, or aqueous normal-phase chromatography.

In some embodiments, the LC method is a high performance liquid chromatography (HPLC) method. In some embodiments, the HPLC method is further categorized as normal-phase chromatography, reverse-phase chromatography, size-exclusion chromatography, ion-exchange chromatography, affinity chromatography, displacement chromatography, partition chromatography, chiral chromatography, or aqueous normal-phase chromatography.

In some embodiments, the HPLC method of the present disclosure is performed by any standard techniques well known in the art. Exemplary HPLC methods include hydrophilic interaction liquid chromatography (HILIC), electrostatic repulsion-hydrophilic interaction liquid chromatography (ERLIC) and reverse phase liquid chromatography (RPLC).

In some embodiments, the LC is coupled to a mass spectroscopy as a LC-MS method. In some embodiments, the LC-MS method includes ultra-performance liquid chromatography-electrospray ionization quadrupole time-of-flight mass spectrometry (UPLC-ESI-QTOF-MS), ultra-performance liquid chromatography-electrospray ionization tandem mass spectrometry (UPLC-ESI-MS/MS), reverse phase liquid chromatography-mass spectrometry (RPLC-MS), hydrophilic interaction liquid chromatography-mass spectrometry (HILIC-MS), hydrophilic interaction liquid chromatography-triple quadrupole tandem mass spectrometry (HILIC-QQQ), electrostatic repulsion-hydrophilic interaction liquid chromatography-mass spectrometry (ERLIC-MS), liquid chromatography time-of-flight mass spectrometry (LC-TOF-MS), liquid chromatography quadrupole time-of-flight mass spectrometry (LC-QTOF-MS), liquid chromatography-tandem mass spectrometry (LC-MS/MS), multidimensional liquid chromatography coupled with tandem mass spectrometry (LC/LC-MS/MS). In some instances, the LC-MS method is LC/LC-MS/MS. In some embodiments, the LC-MS methods of the present disclosure are performed by standard techniques well known in the art.

In some embodiments, the GC is coupled to a mass spectroscopy as a GC-MS method. In some embodiments, the GC-MS method includes two-dimensional gas chromatography time-of-flight mass spectrometry (GC*GC-TOFMS), gas chromatography time-of-flight mass spectrometry (GC-QTOF-MS) and gas chromatography-tandem mass spectrometry (GC-MS/MS).

In some embodiments, CE is coupled to a mass spectroscopy as a CE-MS method. In some embodiments, the CE-MS method includes capillary electrophoresis-negative electrospray ionization-mass spectrometry (CE-ESI-MS), capillary electrophoresis-negative electrospray ionization-quadrupole time of flight-mass spectrometry (CE-ESI-QTOF-MS) and capillary electrophoresis-quadrupole time of flight-mass spectrometry (CE-QTOF-MS).

In some embodiments, the nuclear magnetic resonance (NMR) method is any suitable method well known in the art for the detection of one or more cysteine binding proteins or protein fragments disclosed herein. In some embodiments, the NMR method includes one dimensional (1D) NMR methods, two dimensional (2D) NMR methods, solid state NMR methods and NMR chromatography. Exemplary 1D NMR methods include ¹Hydrogen, ¹³Carbon, ¹⁵Nitrogen, ¹⁷Oxygen, ¹⁹Fluorine, ³¹Phosphorus, ³⁹Potassium, ²³Sodium, ³³Sulfur, ⁸⁷Strontium, ²⁷Aluminium, ⁴³Calcium, ³⁵Chlorine, ³⁷Chlorine, ⁶³Copper, ⁶⁵Copper, ⁵⁷Iron, ²⁵Magnesium, ¹⁹⁹Mercury or ⁶⁷Zinc NMR method, distortionless enhancement by polarization transfer (DEPT) method, attached proton test (APT) method and 1D-incredible natural abundance double quantum transition experiment (INADEQUATE) method. Exemplary 2D NMR methods include correlation spectroscopy (COSY), total correlation spectroscopy (TOCSY), 2D-INADEQUATE, 2D-adequate double quantum transfer experiment (ADEQUATE), nuclear overhauser effect spectroscopy (NOSEY), rotating-frame NOE spectroscopy (ROESY), heteronuclear multiple-quantum correlation spectroscopy (HMQC), heteronuclear single quantum coherence spectroscopy (HSQC), short range coupling and long range coupling methods. Exemplary solid state NMR method include solid state ¹³Carbon NMR, high resolution magic angle spinning (HR-MAS) and cross polarization magic angle spinning (CP-MAS) NMR methods. Exemplary NMR techniques include diffusion ordered spectroscopy (DOSY), DOSY-TOCSY and DOSY-HSQC.

In some embodiments, the protein fragments are analyzed by method as described in Weerapana et al., “Quantitative reactivity profiling predicts functional cysteines in proteomes,” Nature, 468:790-795 (2010).

In some embodiments, the results from the mass spectroscopy method are analyzed by an algorithm for protein identification. In some embodiments, the algorithm combines the results from the mass spectroscopy method with a protein sequence database for protein identification. In some embodiments, the algorithm comprises ProLuCID algorithm, Probity, Scaffold, SEQUEST, or Mascot.

### Digital Processing Device

In some embodiments, the methods described herein include a digital processing device, or use of the same. In further embodiments, the digital processing device includes one or more hardware central processing units (CPU) that carry out the device's functions. In still further embodiments, the digital processing device further comprises an operating system configured to perform executable instructions. In some embodiments, the digital processing device is optionally connected to a computer network. In further embodiments, the digital processing device is optionally connected to the Internet such that it accesses the World Wide Web. In still further embodiments, the digital processing device is optionally connected to a cloud computing infrastructure. In other embodiments, the digital processing device is optionally connected to an intranet. In other embodiments, the digital processing device is optionally connected to a data storage device.

In accordance with the description herein, suitable digital processing devices include, by are not limited to, server computers, desktop computers, laptop computers, notebook computers, sub-notebook computers, netbook computers, netpad computers, set-top computers, media streaming devices, handheld computers, Internet appliances, mobile smartphones, tablet computers, personal digital assistants, video game consoles, and vehicles. Suitable tablet computers include those with booklet, slate, or convertible configurations.

In some embodiments, the digital processing device includes an operating system configured to perform executable instructions. The operating system is, for example, software, including programs and data, which manages the device's hardware and provides services for execution of applications.

In some embodiments, the device includes a storage and/or memory device. The storage and/or memory device is one or more physical apparatuses used to store data or programs on a temporary or permanent basis. In some embodiments, the device is volatile memory and requires power to maintain stored information. In some embodiments, the device is non-volatile memory and retains stored information when the digital processing device is not powered. In further embodiments, the non-volatile memory comprises flash memory. In some embodiments, the non-volatile memory comprises dynamic random-access memory (DRAM). In some embodiments, the non-volatile memory comprises ferroelectric random access memory (FRAM). In some embodiments, the non-volatile memory comprises phase-change random access memory (PRAM). In other embodiments, the device is a storage device including, by way of non-limiting examples, CD-ROMs, DVDs, flash memory devices, magnetic disk drives, magnetic tapes drives, optical disk drives, and cloud computing based storage. In further embodiments, the storage and/or memory device is a combination of devices such as those disclosed herein.

In some embodiments, the digital processing device includes a display to send visual information to a user. In some embodiments, the display includes a cathode ray tube (CRT), a liquid crystal display (LCD), a thin film transistor liquid crystal display (TFT-LCD), an organic light emitting diode (OLED) display, a plasma display, a video projector, or a combination thereof.

In some embodiments, the digital processing device includes an input device to receive information from a user. In some embodiments, the input device is a keyboard. In some embodiments, the input device is a pointing device including, by way of non-limiting examples, a mouse, trackball, track pad, joystick, game controller, or stylus. In some embodiments, the input device is a touch screen or a multi-touch screen. In other embodiments, the input device is a microphone to capture voice or other sound input. In other embodiments, the input device is a video camera or other sensor to capture motion or visual input. In still further embodiments, the input device is a combination of devices such as those disclosed herein.

In some embodiments, the systems and methods disclosed herein include one or more non-transitory computer readable storage media encoded with a program including instructions executable by the operating system of an optionally networked digital processing device. In further embodiments, a computer readable storage medium is a tangible component of a digital processing device. In still further embodiments, a computer readable storage medium is optionally removable from a digital processing device. In some embodiments, a computer readable storage medium includes, by way of non-limiting examples, CD-ROMs, DVDs, flash memory devices, solid state memory, magnetic disk drives, magnetic tape drives, optical disk drives, cloud computing systems and services, and the like. In some cases, the program and instructions are permanently, substantially permanently, semi-permanently, or non-transitorily encoded on the media.

In some embodiments, the systems and methods disclosed herein include at least one computer program, or use of the same. A computer program includes a sequence of instructions, executable in the digital processing device's CPU, written to perform a specified task. In some embodiments, computer readable instructions are implemented as program modules, such as functions, objects, Application Programming Interfaces (APIs), data structures, and the like, that perform particular tasks or implement particular abstract data types.

In some embodiments, the functionality of the computer readable instructions are combined or distributed as desired in various environments. In some embodiments, a computer program comprises one sequence of instructions. In some embodiments, a computer program comprises a plurality of sequences of instructions. In some embodiments, a computer program is provided from one location. In other embodiments, a computer program is provided from a plurality of locations. In various embodiments, a computer program includes one or more software modules. In various embodiments, a computer program includes, in part or in whole, one or more web applications, one or more mobile applications, one or more standalone applications, one or more web browser plug-ins, extensions, add-ins, or add-ons, or combinations thereof.

### Compositions, Process of Producing a Probe-Protein Composition

In some embodiments, disclosed herein are compositions of the probe-LOXL2 protein complex, and compositions that comprise a probe-LOXL2 protein complex and a sample.

In some embodiments, disclosed herein is a probe-protein composition which comprises a probe described herein and a LOXL2 protein. In some embodiments, also described herein is a probe-protein composition produced by a process described herein.

### Kits/Article of Manufacture

Disclosed herein, in certain embodiments, are kits and articles of manufacture for use with one or more methods described herein. In some embodiments, described herein is a kit for identifying and/or quantifying target engagement of a LOXL2i with LOXL2. In some instances, also described herein is a kit for mapping binding sites on LOXL2. In some cases, described herein is a kit for identifying proteins that interact with a LOXL2i.

In some embodiments, such kit includes LOXL2i probes such as the probes described herein, and reagents suitable for carrying out one or more of the methods described herein. In some instances, the kit further comprises samples, such as a cell sample, and suitable solutions such as buffers or media. In some embodiments, the kit further comprises recombinant proteins for use in one or more of the methods described herein. In some embodiments, additional components of the kit comprises a carrier, package, or container that is compartmentalized to receive one or more containers such as vials, tubes, and the like, each of the container(s) comprising one of the separate elements to be used in a method described herein. Suitable containers include, for example, bottles, vials, plates, syringes, and test tubes. In one embodiment, the containers are formed from a variety of materials such as glass or plastic.

The articles of manufacture provided herein contain packaging materials. Examples of pharmaceutical packaging materials include, but are not limited to, bottles, tubes, bags, containers, and any packaging material suitable for a selected formulation and intended mode of use.

For example, the container(s) include one or more of the probes described herein, and one or more reagents for use in a method disclosed herein. Such kits optionally include an identifying description or label or instructions relating to its use in the methods described herein.

A kit typically includes labels listing contents and/or instructions for use, and package inserts with instructions for use. A set of instructions will also typically be included.

In one embodiment, a label is on or associated with the container. In one embodiment, a label is on a container when letters, numbers or other characters forming the label are attached, molded or etched into the container itself; a label is associated with a container when it is present within a receptacle or carrier that also holds the container, e.g., as a package insert. In one embodiment, a label is used to indicate that the contents are to be used for a specific therapeutic application. The label also indicates directions for use of the contents, such as in the methods described herein.

### Services

In some embodiments, the methods provided herein also perform as a service. In some instances, a service provider obtains from the customer a sample for analysis with one or more of the probes described herein. In some embodiments, the service provider analyzes the sample using one or more of the methods described herein, and then provides the results to the customer. In some instances, the service provider provides the appropriate reagents to the customer for analysis utilizing one or more of the probes described herein and one or more of the methods described herein. In some cases, the customer performs one or more of the methods described herein and then provides the results to the service provider for analysis. In some embodiments, the service provider then analyzes the results and provides the results to the costumer. In some cases, the customer further analyzes the results by interacting with software installed locally (at the customer's location) or remotely (e.g., on a server reachable through a network). Exemplary customers include pharmaceutical companies, clinical laboratories, physicians, patients, and the like. In some instances, a customer is any suitable customer or party with a need or desire to use the methods, systems, compositions, and kits described herein.

### EXAMPLES

The following examples are provided for illustrative purposes only and not to limit the scope of the claims provided herein.

### Synthesis of Int-A

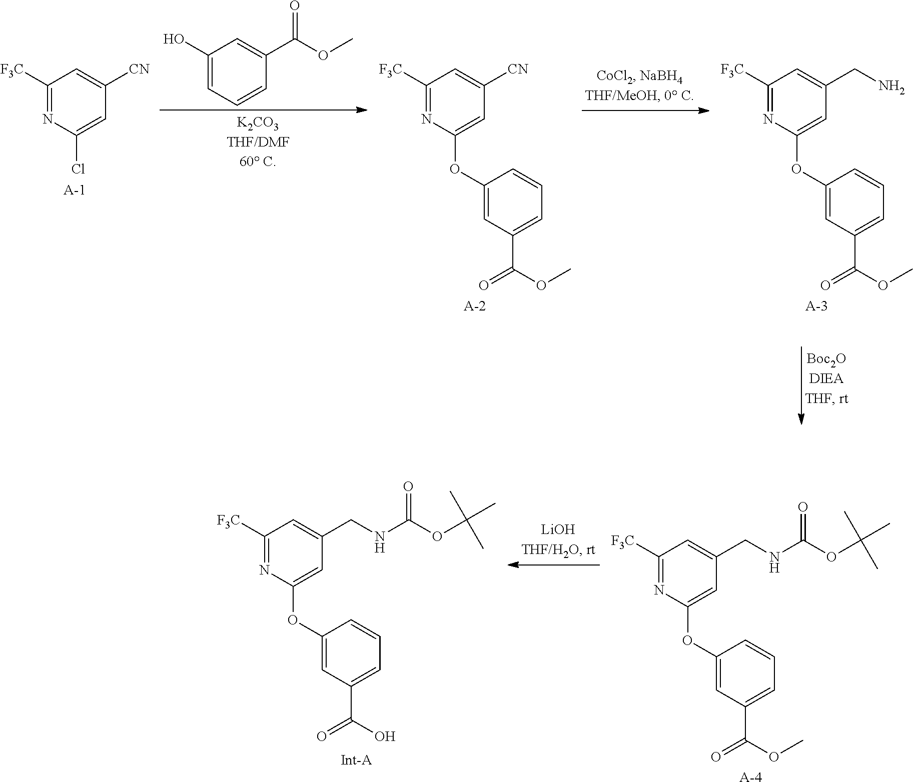

### Step 1: Methyl 3-((4-cyano-6-(trifluoromethyl)pyridin-2-yl)oxy)benzoate (A-2)

To a solution of 2-chloro-6-(trifluoromethyl)isonicotinonitrile A-1 (4.0 g, 19.4 mmol) and methyl 3-hydroxybenzoate (3.24 g, 21.3 mmol) in a mixture of THF/DMF (4:1, 55 ml), was added potassium carbonate (8.0 g, 58 mmol). The reaction mixture was heated at 60° C. for 2 h. The THF was evaporated under reduced pressure and the remaining reaction mixture was partitioned between water (200 mL) and ethyl acetate (100 mL). The organic layer was separated and the aqueous layer was re-extracted with EtOAc (1×100 ml). The combined organic layers were dried (Na₂SO₄), filtered, and then concentrated under reduced pressure. The crude residue was purified (silica gel; eluting with 0-50% EtOAc in hexanes), to afford compound A-2 as a light yellow solid (5.63 g, 91%). ¹H NMR (300 MHz, DMSO-d₆): δ 8.21 (m, 1H), 8.07 (m, 1H), 7.87 (m, 1H), 7.77 (m, 1H), 7.64 (m, 1H), 7.55 (m, 1H), 3.85 (s, 3H); LCMS Mass: 323.0 (M⁺+1).

### Step 2: Methyl 3-((4-(aminomethyl)-6-(trifluoromethyl)pyridin-2-yl)oxy)benzoate (A-3)

To a stirred solution of methyl 3-((4-cyano-6-(trifluoromethyl)pyridin-2-yl)oxy)benzoate A-2 (1.5 g, 4.65 mmol) in THF/MeOH (1:1, 140 mL) at 0° C., was added portion-wise CoCl₂ (1.8 g, 13.98 mmol) followed by NaBH₄ (1.77 g, 46.5 mmol). The reaction mixture was stirred at 0° C. for 20 minutes. The mixture was diluted with EtOAc (100 mL) and filtered through celite. The filtrate was concentrated and the resulting residue was partitioned between water (200 mL) and EtOAc (200 mL). The water-organic layer was filtered through celite and the organic layer was separated, dried (Na₂SO₄), filtered, and then concentrated under reduced pressure to obtain compound A-3 as an amber oil (1.38 g, 92%) which did not require further purification. ¹H NMR (300 MHz, DMSO-d₆): δ 7.83 (m, 1H), 7.67 (m, 1H), 7.65 (br m, 1H), 7.60 (m, 1H), 7.47 (m, 1H), 7.33 (br m, 1H), 3.80-3.83 (m, 5H); LCMS Mass: 327.0 (M⁺+1).

### Step 3: Methyl 3-((4-(((tert-butoxycarbonyl)amino)methyl)-6-(trifluoromethyl)pyridin-2-yl)oxy)benzoate (A-4)

To a stirred solution of ester A-3 (1.38 g, 4.24 mmol) in THF (25 mL) at 0° C., was added di-tert-butyl dicarbonate (1.29 g, 5.94 mmol) and DIEA (2.21 mL, 12.74 mmol). The mixture was warmed to RT and stirred for a further 4 h. The mixture was concentrated and the residue partitioned between EtOAc (50 mL) and water (50 mL). The organic layer was separated, dried (Na₂SO₄), filtered, and concentrated in vacuo. The residue was purified (silica gel; 0-60% EtOAc in hexanes), to afford compound A-4 as an amber oil (1.42 g, 78%). ¹H NMR (300 MHz, DMSO-d₆): δ 7.85 (m, 1H), 7.69 (m, 1H), 7.58-7.62 (m, 2H), 7.48-7.51 (m, 2H), 7.13 (br m, 1H), 4.20 (m, 2H), 3.84 (s, 3H), 1.36 (s, 9H); LCMS Mass: 427.0 (M⁺+1).

### Step 4: 3-((4-(((tert-Butoxycarbonyl)amino)methyl)-6-(trifluoromethyl)pyridin-2-yl)oxy)benzoic acid (Int-A)

To a stirred solution of ester A-4 (1.42 g, 3.34 mmol) in a mixture of THF/H₂O (6:1, 21 mL) was added aqueous 4M LiOH (17 mL, 68 mmol). The mixture was stirred at RT for 16 h, then diluted with water (30 ml) and acidified to pH 3-4 using aq. sat. citric acid. The mixture was extracted with EtOAc (2×50 mL), and the combined organic layers were dried (Na₂SO₄), filtered, and concentrated under reduced pressure to afford Int-A as an off white solid (1.2 g, 87%). ¹H NMR (300 MHz, DMSO-d₆): δ 13.17 (br s, 1H), 7.83 (m, 1H), 7.66 (br m, 1H), 7.53-7.62 (m, 2H), 7.44-7.51 (m, 2H), 7.12 (br m, 1H), 4.25 (m, 2H), 1.36 (s, 9H); LCMS Mass: 413.0 (M⁺+1).

### Synthesis of Int-B

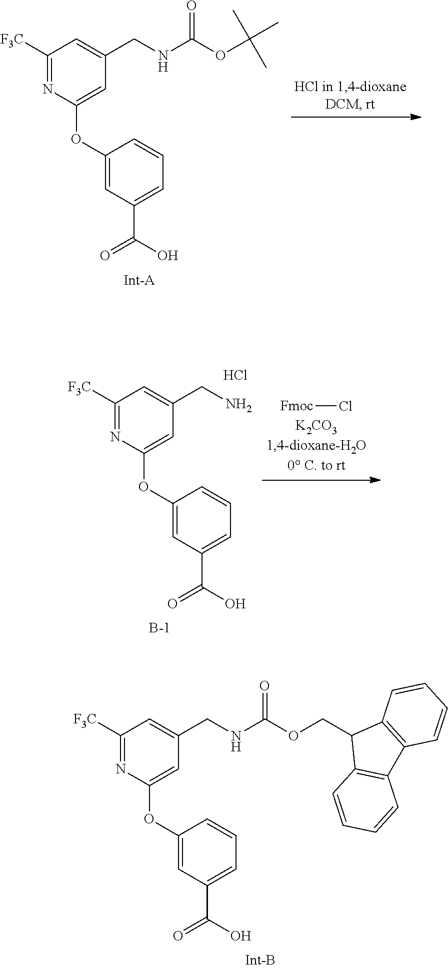

### Step 1: 3-((4-(Aminomethyl)-6-(trifluoromethyl)pyridin-2-yl)oxy)benzoic acid (B-1)

To a stirred solution of Int-A (3 g, 7.28 mmol) in DCM at RT, was added 4M HCl in 1,4-dioxane (36 mL, 144 mmol). The mixture was stirred at RT for 2 h. The mixture was concentrated under reduced pressure to afford compound B-1 (2.54 g, 100%) as a light yellow solid that did not require further purification. ¹H NMR (300 MHz, DMSO-d₆): δ 13.19 (br s, 1H), 8.63 (br s, 3H), 7.82-7.85 (m, 2H), 7.45-7.66 (m, 4H), 4.18-4.25 (m, 2H); LCMS Mass: 313.0 (M⁺+1).

### Step 2: 3-((4-(((((9H-Fluoren-9-yl)methoxy)carbonyl)amino)methyl)-6-(trifluoromethyl)pyridin-2-yl)oxy)benzoic acid (Int-B)

To a stirred mixture of amine B-1 (2.54 g, 7.28 mmol), K₂CO₃ (3.07 g, 22.2 mmol), 1,4-dioxane (74 mL) and water (74 mL) at 0° C., was added dropwise (9H-fluoren-9-yl)methyl chloroformate (2.11 g, 8.14 mmol). The mixture was allowed to warm to RT and stirred for 15 h. The reaction mixture was washed with Et₂O (2×50 mL) and the aqueous layer was separated and acidified to pH 3 using aq. citric acid. The aqueous layer was extracted with EtOAc (2×75 mL) and the combined organic layers were dried (Na₂SO₄), filtered, and then concentrated under reduced pressure. The crude residue was purified (silica gel; eluting with 0-100% EtOAc in hexanes, followed by 0-10% MeOH in DCM), to afford Int-B (2.7 g, 69%) as a light yellow solid. ¹H NMR (300 MHz, DMSO-d₆): δ 13.17 (br s, 1H), 7.66-8.02 (m, 15H), 4.20-4.40 (m, 5H); LCMS Mass: 535.0 (M⁺+1).

### Synthesis of Int-C

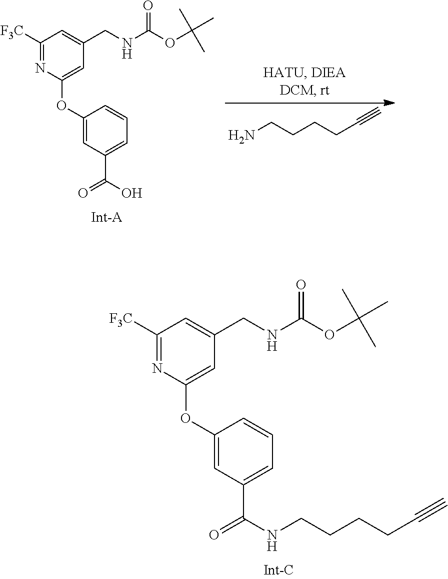

To a stirred solution of Int-A (600 mg, 1.45 mmol) in DCM (10 mL) at RT, was added HATU (633 mg, 1.67 mmol) and the mixture was stirred at RT for 20 min. Hex-5-yn-1-amine hydrochloride (214 μL, 1.6 mmol) and DIEA (430 mg, 3.33 mmol) were added, and the mixture stirred at RT for 3.2 h. The reaction mixture was diluted with a mixture of water and brine. The mixture was repeatedly extracted with EtOAc and the combined organic layers were dried (Na₂SO₄), filtered, and concentrated under reduced pressure. The crude residue was purified (silica gel; eluting with 0-100% EtOAc in hexanes), to afford compound Int-C (610 mg, 86%) as a white solid. ¹H NMR (300 MHz, DMSO-d₆): δ 8.52 (m, 1H), 7.74 (m, 1H), 7.50-7.63 (m, 4H), 7.34 (m, 1H), 7.08 (s, 1H), 4.20-4.28 (m, 2H), 3.20-3.28 (m, 2H), 2.74 (m, 1H), 2.10-2.20 (m, 2H), 1.40-1.62 (m, 4H), 1.35 (s, 9H).

### Example 1

### 3-((4-(Aminomethyl)-6-(trifluoromethyl)pyridin-2-yl)oxy)-N-(5-(5-((3aS,4S,6aR)-2-oxohexahydro-1H-thieno[3,4-d]imidazol-4-yl)pentanamido)pentyl)benzamide hydrochloride (Compound 1-1)

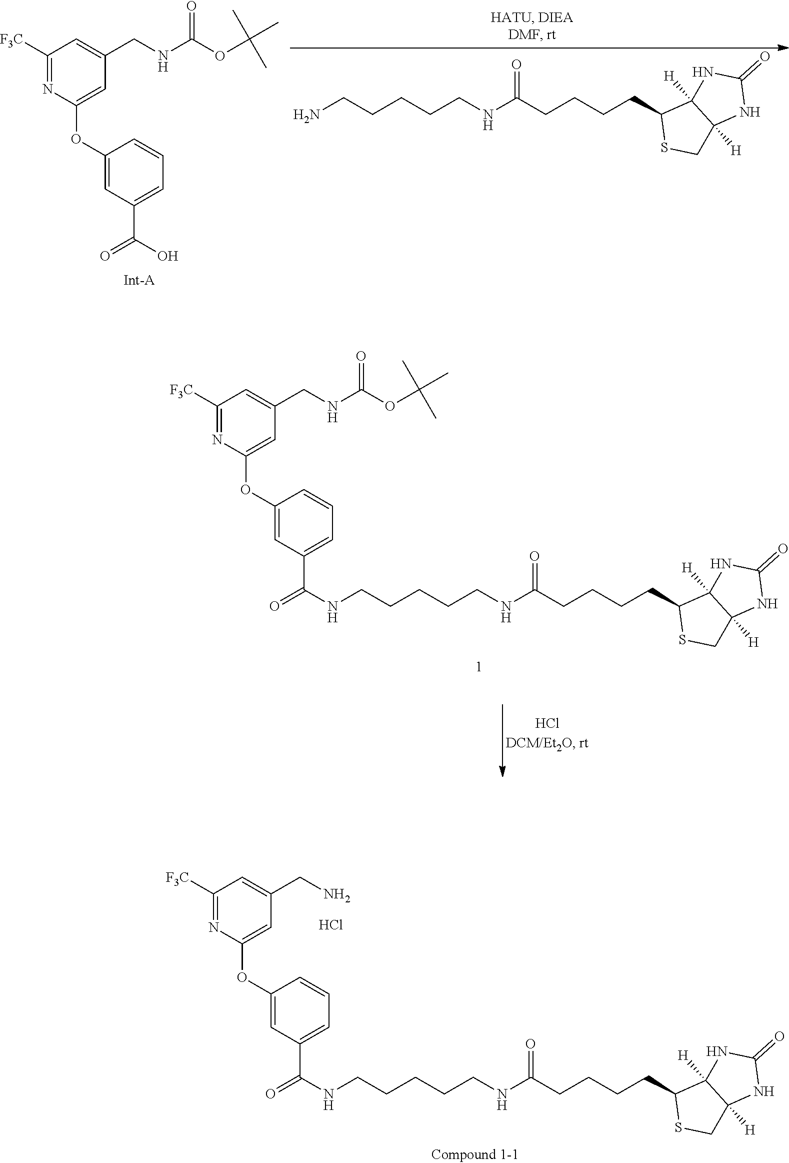 [SMILES: CC(C)(C)OC(=O)NCc1cc(Oc2cccc(C(=O)O)c2)nc(C(F)(F)F)c1 ; NCCCCCNC(=O)CCCC[C@@H]1SC[C@@H]2NC(=O)N[C@@H]21 ; CC(C)(C)OC(=O)NCc1cc(Oc2cccc(C(=O)NCCCCCNC(=O)CCCC[C@@H]3SC[C@@H]4NC(=O)N[C@@H]43)c2)nc(C(F)(F)F)c1 ; NCc1cc(Oc2cccc(C(=O)NCCCCCNC(=O)CCCC[C@@H]3SC[C@@H]4NC(=O)N[C@@H]43)c2)nc(C(F)(F)F)c1 ; Cl]

### Step 1: tert-Butyl((2-(3-((5-(5-((3aS,4S,6aR)-2-oxohexahydro-1H-thieno[3,4-d]imidazol-4-yl)pentanamido)pentyl)carbamoyl)phenoxy)-6-(trifluoromethyl)pyridin-4-yl)methyl)carbamate (1)

To a stirred solution of Int-A (207 mg, 0.50 mmol) in DMF (2 mL) at RT, was added HATU (174 mg, 0.46 mmol) and the mixture was stirred at RT for 20 min. N-(5-Aminopentyl)-5-((3aS,4S,6aR)-2-oxohexahydro-1H-thieno[3,4-d]imidazol-4-yl)pentanamide (EZ-Link™ Pentylamine-Biotin; ThermoFisher Scientific, USA; catalog number 21345) (150 mg, 0.46 mmol) and DIEA (239 μL, 1.37 mmol) were added, and the mixture stirred at RT for 6.5 h. The reaction mixture was diluted with a mixture of water, brine, and aq. 1M HCl. The mixture was repeatedly extracted with EtOAc and the combined organic layers were dried (Na₂SO₄), filtered, and concentrated under reduced pressure. The crude residue was purified (silica gel; eluting with 0-20% MeOH in DCM), to afford compound 1 (248 mg, 75%) as a white solid. LCMS Mass: 623.0 (MH⁺−C₅H₈O₂).

### Step 2: 3-((4-(Aminomethyl)-6-(trifluoromethyl)pyridin-2-yl)oxy)-N-(5-(5-((3aS,4S,6aR)-2-oxohexahydro-1H-thieno[3,4-d]imidazol-4-yl)pentanamido)pentyl)benzamide hydrochloride (Compound 1-1)

To a stirred mixture of 1 (248 mg, 0.34 mmol) in DCM (2 mL) at RT, was added 2 M HCl in Et₂O (2 mL, 4.0 mmol) and the mixture was stirred at RT for 18 h. The mixture was concentrated under reduced pressure to afford the title compound 1-1 (216 mg, 96%) as a white solid. ¹H NMR (300 MHz, DMSO-d₆): δ 8.50-8.70 (m, 4H), 7.83 (m, 1H), 7.73-7.77 (m, 3H), 7.62 (m, 1H), 7.40-7.60 (m, 3H), 7.34 (m, 1H), 4.10-4.30 (m, 4H), 3.20-3.30 (m, 2H), 2.90-3.20 (m, 3H), 2.79 (m, 1H), 2.50 (m, 1H), 1.99-2.05 (m, 2H), 1.20-1.60 (m, 12H).

### Example 2

### 3-((4-(Aminomethyl)-6-(trifluoromethyl)pyridin-2-yl)oxy)-N-(37-oxo-41-((3aS,4S,6aR)-2-oxohexahydro-1H-thieno[3,4-d]imidazol-4-yl)-3,6,9,12,15,18,21,24,27,30,33-undecaoxa-36-azahentetracontyl)benzamide hydrochloride (Compound 1-2)

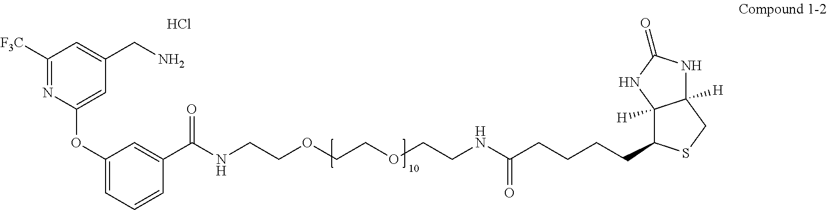

The title compound (1-2) was prepared using the procedure for Example 1, using N-(35-amino-3,6,9,12,15,18,21,24,27,30,33-undecaoxapentatriacontyl)-5-((3aS,4S,6aR)-2-oxohexahydro-1H-thieno[3,4-d]imidazol-4-yl)pentanamide (EZ-Link™ Amine-PEG11-Biotin; ThermoFisher Scientific, USA; catalog number 26136) in Step 1. ¹H NMR (300 MHz, DMSO-d₆): δ 8.60 (m, 1H), 8.51 (br s, 3H), 7.77-7.83 (m, 3H), 7.65 (m, 1H), 7.55 (m, 1H), 7.48 (s, 1H), 7.37 (m, 1H), 6.39 (br s, 2H), 4.30 (m, 1H), 4.21-4.24 (m, 2H), 4.12 (m, 1H), 3.65-3.90 (br m, 40H), 3.48-3.53 (m, 5H), 3.35-3.45 (m, 5H), 3.16-3.20 (m, 2H), 3.09 (m, 1H), 2.82 (m, 1H), 2.57 (m, 1H), 2.04-2.07 (m, 2H), 1.60 (m, 1H), 1.35-1.53 (m, 3H), 1.30 (m, 1H); LCMS Mass: 1066.0 (M⁺+1).

### Example 3

### 1-(6-((3-(4-(4-(3-((4-(Aminomethyl)-6-(trifluoromethyl)pyridin-2-yl)oxy)benzamido)butyl)-1H-1,2,3-triazol-1-yl)propyl)amino)-6-oxohexyl)-3,3-dimethyl-2-((1E,3E)-3-(1,3,3-trimethylindolin-2-ylidene)prop-1-en-1-yl)-3H-indol-1-ium chloride hydrochloride (Compound 1-3)

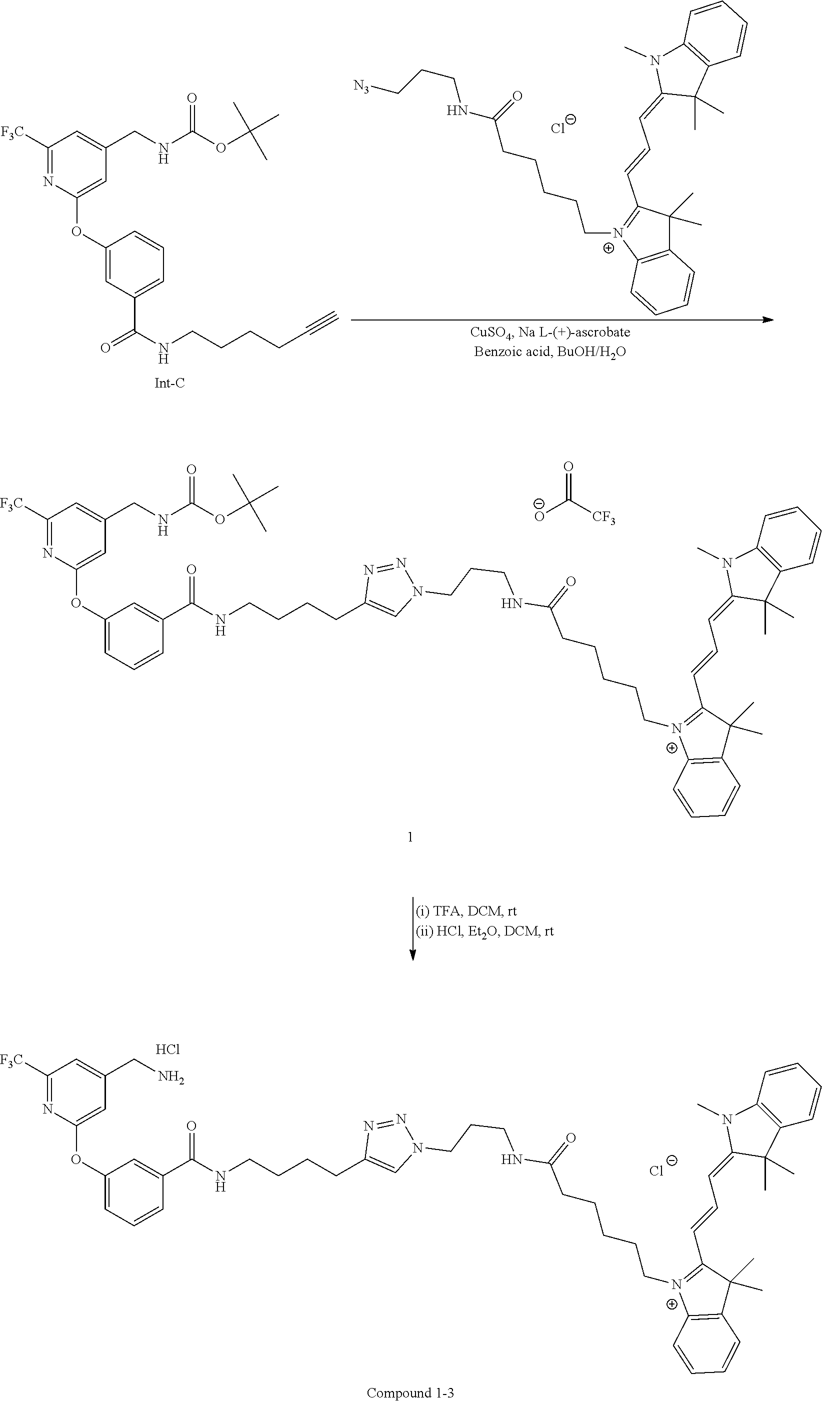

### Step 1: 1-(6-((3-(4-(4-(3-((4-(((tert-Butoxycarbonyl)amino)methyl)-6-(trifluoromethyl)pyridin-2-yl)oxy)benzamido)butyl)-1H-1,2,3-triazol-1-yl)propyl)amino)-6-oxohexyl)-3,3-dimethyl-2-((1E,3E)-3-(1,3,3-trimethylindolin-2-ylidene)prop-1-en-1-yl)-3H-indol-1-ium trifluoroacetate (1)

A mixture of Int-C (21 mg, 0.043 mmol), 1-(6-((3-azidopropyl)amino)-6-oxohexyl)-3,3-dimethyl-2-((1E,3E)-3-(1,3,3-trimethylindolin-2-ylidene)prop-1-en-1-yl)-3H-indol-1-ium chloride (Cyanine3 azide; Lumiprobe, Florida USA; catalog number D1030) (25 mg, 0.043 mmol), CuSO₄ (catalytic, 1.25 mol %), sodium L-(+)-ascorbate (catalytic, 25 mol %), benzoic acid (catalytic, 10 mol %), n-butanol (1.3 mL), and water (2.6 mL) was stirred at RT for 16 h. To the mixture was added DMSO (1.5 mL), CuSO₄ (catalytic, 1.25 mol %), and sodium L-(+)-ascorbate (catalytic, 25 mol %), and stirring was continued for additional 16 h. The reaction mixture was diluted with EtOAc (5 mL) and the organic layer was separated. The aq. layer was re-extracted with additional EtOAc, and the combined organic layers were concentrated under reduced pressure. The residue was dissolved in MeCN and purified via preparative reverse-phase HPLC (Waters XTerra® Prep MS C-18 OBD 5 μM 50×100 mm column; eluting with 0-95% MeCN/H₂O containing 0.1% TFA, over 18 min, followed by 95% MeCN/H₂O for 10 min) to afford compound 1 (21 mg, 48%) as a pink solid. ¹H NMR (300 MHz, DMSO-d₆): δ 8.51 (m, 1H), 8.31 (m, 1H), 7.82-7.88 (m, 2H), 7.72 (m, 1H), 7.56-7.62 (m, 4H), 7.48-7.55 (m, 2H), 7.21-7.45 (m, 7H), 7.08 (m, 1H), 6.40-6.50 (m, 2H), 4.20-4.30 (m, 4H), 4.00-4.15 (m, 2H), 3.62 (s, 3H), 3.20-3.30 (m, 2H), 2.92-3.02 (m, 2H), 2.55-2.65 (m, 2H), 2.03-2.10 (m, 2H), 1.80-1.90 (m, 2H), 1.50-1.75 (m, 20H), 1.30-1.40 (m, 11H); LCMS Mass: 1031.0 (M⁺).

### Step 2: 1-(6-((3-(4-(4-(3-((4-(Aminomethyl)-6-(trifluoromethyl)pyridin-2-yl)oxy)benzamido)butyl)-1H-1,2,3-triazol-1-yl)propyl)amino)-6-oxohexyl)-3,3-dimethyl-2-((1E,3E)-3-(1,3,3-trimethylindolin-2-ylidene)prop-1-en-1-yl)-3H-indol-1-ium chloride hydrochloride (Compound 1-3)

To a stirred solution of 1 (19 mg, 0.018 mmol) in DCM (3 mL) at RT, was added 20% TFA in DCM (0.5 mL) and the mixture was stirred at RT for 16 h. The mixture was concentrated under reduced pressure and dried under high vacuum. The residue was dissolved in DCM (1.5 mL) and to this was added 2M HCl in Et₂O (52 μL, 0.1 mmol) and the mixture was stirred at RT for 25 min. The reaction was concentrated under reduced pressure to afford compound 1-3 (18 mg, 100%) as a pink solid. ¹H NMR (300 MHz, DMSO-d₆): δ 8.55-8.70 (m, 4H), 8.32 (m, 1H), 7.96 (m, 1H), 7.82-7.88 (m, 2H), 7.75 (m, 1H), 7.20-7.65 (m, 12H), 6.42-6.50 (m, 2H), 4.15-4.25 (m, 4H), 4.02-4.12 (m, 2H), 3.62 (s, 3H), 3.20-3.30 (m, 2H), 2.92-3.00 (m, 2H), 2.55-2.62 (m, 2H), 2.03-2.10 (m, 2H), 1.80-1.90 (m, 2H), 1.50-1.75 (m, 20H), 1.30-1.40 (m, 2H); LCMS Mass: 931.0 (M⁺).

### Example 4

### 1-(6-((3-(4-(4-(3-((4-(Aminomethyl)-6-(trifluoromethyl)pyridin-2-yl)oxy)benzamido)butyl)-1H-1,2,3-triazol-1-yl)propyl)amino)-6-oxohexyl)-3,3-dimethyl-2-((1E,3E,5E)-5-(1,3,3-trimethylindolin-2-ylidene)penta-1,3-dien-1-yl)-3H-indol-1-ium chloride hydrochloride (Compound 1-4)

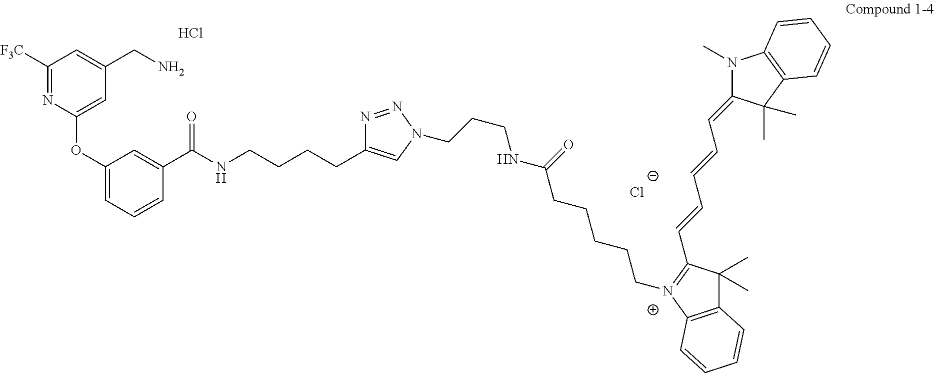

The title compound (1-4) was prepared using the procedure for Example 3, using 1-(6-((3-azidopropyl)amino)-6-oxohexyl)-3,3-dimethyl-2-((1E,3E,5E)-5-(1,3,3-trimethylindolin-2-ylidene)penta-1,3-dien-1-yl)-3H-indol-1-ium chloride (Cyanine5 azide; Lumiprobe, Florida USA; catalog number D3030) in Step 1. ¹H NMR (300 MHz, DMSO-d₆): δ 8.50-8.60 (m, 5H), 8.25-8.35 (m, 2H), 7.80-7.90 (m, 3H), 7.75 (m, 1H), 7.47-7.63 (m, 5H), 7.30-7.40 (m, 4H), 7.20-7.28 (m, 2H), 6.53 (m, 1H), 6.20-6.30 (m, 2H), 4.15-4.25 (m, 4H), 4.02-4.12 (m, 2H), 3.57 (s, 3H), 3.20-3.30 (m, 2H), 2.92-3.00 (m, 2H), 2.55-2.62 (m, 2H), 2.03-2.10 (m, 2H), 1.80-1.90 (m, 2H), 1.50-1.75 (m, 20H), 1.30-1.40 (m, 2H); LCMS Mass: 957.0 (M⁺).

### Example 5

### 3-((4-(Aminomethyl)-6-(trifluoromethyl)pyridin-2-yl)oxy)-N-(4-methyl-2-oxo-2H-chromen-7-yl)benzamide hydrochloride (Compound 1-5)

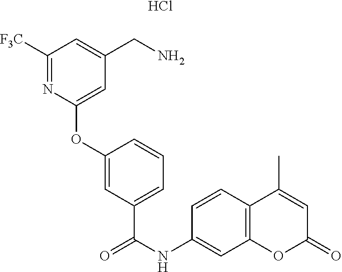 [SMILES: Cl ; Cc1cc(=O)oc2cc(NC(=O)c3cccc(Oc4cc(CN)cc(C(F)(F)F)n4)c3)ccc12]

The title compound (1-5) was prepared using the procedure for Example 1, using 7-amino-4-methylcoumarin in Step 1. ¹H NMR (300 MHz, DMSO-d₆): δ 10.76 (s, 1H), 8.50-8.75 (br s, 3H), 7.70-7.95 (m, 6H), 7.66 (m, 1H), 7.45-7.55 (m, 2H), 6.23 (s, 1H), 4.15-4.25 (m, 2H), 2.41 (s, 3H); LCMS Mass: 470.0 (M⁻+1).

### Example 6

### N-(3-((4-(Aminomethyl)-6-(trifluoromethyl)pyridin-2-yl)oxy)phenyl)-5-(dimethylamino)naphthalene-1-sulfonamide hydrochloride (Compound 1-6)

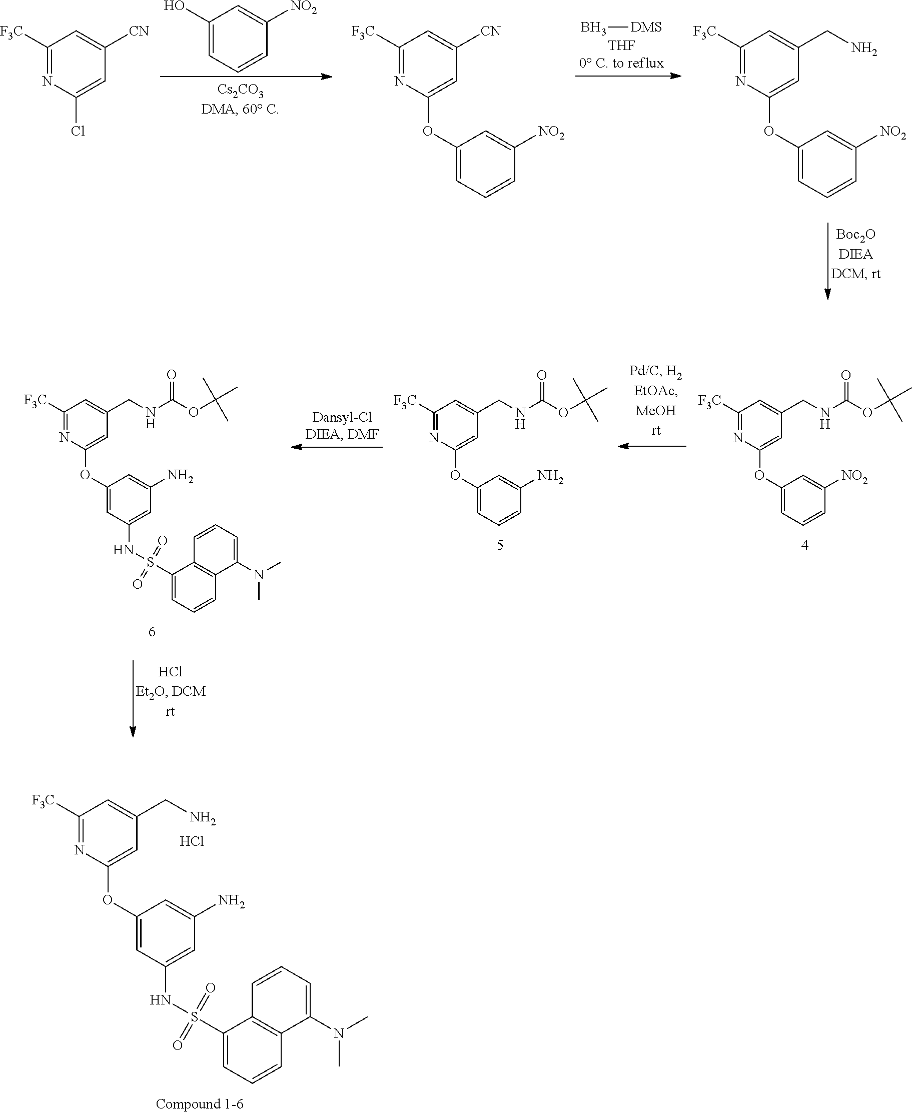

### Step 1: 2-(3-Nitrophenoxy)-6-(trifluoromethyl)isonicotinonitrile (2)

A stirred mixture of 2-chloro-6-(trifluoromethyl)isonicotinonitrile 1 (1 g, 4.84 mmol), 3-nitrophenol (1.35 g, 9.7 mmol), Cs₂CO₃ (4.73 g, 14.5 mmol), and DMA (24 mL), was heated at 60° C. for 1.5 h. The reaction mixture was diluted with water and brine, then extracted with EtOAc (4×30 mL). The combined organic layers were washed with water, then brine, dried (Na₂SO₄), filtered and concentrated under reduced pressure. The residue was purified (silica gel; eluting with 0-40% EtOAc in hexanes) to afford compound 2 (750 mg, 50%) as a solid.

### Step 2: (2-(3-Nitrophenoxy)-6-(trifluoromethyl)pyridin-4-yl)methanamine (3)

To a stirred solution of nitrile 2 (744 mg, 2.41 mmol) in THF (7 mL) at 0° C., was added drop-wise borane-dimethylsulfide (2M solution in THF, 2.77 mL, 5.54 mmol). The mixture was allowed to warm to RT then heated at 60° C. for 1.5 h. The mixture was cooled to RT then carefully quenched with MeOH (3 mL). The mixture was concentrated under reduced pressure and dried under high vacuum to afford compound 3 (763 mg) as an orange oil, that was not purified further. LCMS Mass: 314.0 (M⁺+1).

### Step 3: tert-Butyl((2-(3-nitrophenoxy)-6-(trifluoromethyl)pyridin-4-yl)methyl)carbamate (4)

To a stirred mixture of amine 3 (760 mg), di-tert butyl dicarbonate (622 mg, 4.85 mmol), and DCM (12 mL) at 0° C., was added DIEA (1.27 mL, 7.30 mmol). The mixture was allowed to warm to RT and stirred for 16 h. The mixture was diluted with water and brine, and then extracted with DCM (3×30 mL). The combined organic layers were washed with aq. 1M HCl (2×20 mL), 1:1 water/brine (5×20 mL), then dried (Na₂SO₄) and concentrated under reduced pressure. The residue was purified (silica gel; eluting with 0-40% EtOAc in hexanes) to afford compound 4 (505 mg, 51% over two steps) as an oil. ¹H NMR (300 MHz, DMSO-d₆): δ 8.11-8.13 (m, 2H), 7.70-7.80 (m, 2H), 7.62 (m, 1H), 7.54 (m, 1H), 7.21 (m, 1H), 4.20-4.30 (m, 2H), 1.38 (s, 9H); LCMS Mass: 414.0 (M⁺+1).

### Step 4: tert-Butyl((2-(3-aminophenoxy)-6-(trifluoromethyl)pyridin-4-yl)methyl)carbamate (5)

A mixture of compound 4 (459 mg, 1.11 mmol), 10 wt % Pd on carbon (catalytic, 10 mol %), and EtOAc: MeOH (1:1, 12 mL), was stirred at RT under 1 atmosphere of H₂ gas. After 3 h the mixture was filtered through celite and the celite pad was washed with additional EtOAc:MeOH (1:1, 100 mL). The obtained filtrate was concentrated under reduced pressure to afford compound 5 (511 mg) as a yellow oil, that was not purified further. LCMS Mass: 328.0 (MH⁺−C₄H₈).

### Step 5: tert-Butyl((2-(3-(5-(dimethylamino)naphthalene-1-sulfonamido)phenoxy)-6-(trifluoromethyl)pyridin-4-yl)methyl)carbamate (6)

To a stirred mixture of compound 5 (430 mg), dansyl chloride (393 mg, 1.46 mmol), and DMF at 0° C., was added DIEA (390 μL, 2.24 mmol). The mixture was warmed to RT and stirred for 16 h. The mixture was diluted with water and brine, and then repeatedly extracted with EtOAc. The combined organic layers were dried (Na₂SO₄) and concentrated under reduced pressure. The residue was purified (silica gel; eluting with 0-40% EtOAc in hexanes, followed by 0-10% EtOAc in DCM) to afford compound 6 (378 mg, 56% over two steps) as a yellow oil. ¹H NMR (300 MHz, DMSO-d₆): δ 10.86 (br s, 1H), 8.43 (m, 1H), 8.32 (m, 1H), 8.18 (m, 1H), 7.50-7.62 (m, 3H), 7.47 (m, 1H), 7.20-7.30 (m, 2H), 6.90 (m, 1H), 6.68-6.94 (m, 3H), 4.15-4.25 (m, 2H), 2.79 (s, 6H), 1.35 (s, 9H); LCMS Mass: 617.0 (M⁺+1).

### Step 6: N-(3-((4-(Aminomethyl)-6-(trifluoromethyl)pyridin-2-yl)oxy)phenyl)-5-(dimethylamino)naphthalene-1-sulfonamide hydrochloride (Compound 1-6)

To a stirred mixture of 6 (375 mg, 0.608 mmol) in DCM (6 mL) at RT, was added 2 M HCl in Et₂O (6 mL, 12.0 mmol) and the mixture was stirred at RT for 4 h. The mixture was concentrated under reduced pressure to afford the title compound 1-6 (274 mg, 82%) as a white solid. ¹H NMR (300 MHz, DMSO-d₆): δ 10.96 (br s, 1H), 8.60-8.75 (br s, 3H), 8.50 (m, 1H), 8.40 (m, 1H), 8.20 (m, 1H), 7.82 (m, 1H), 7.50-7.68 (m, 2H), 7.28-7.40 (m, 2H), 7.20 (m, 1H), 6.80-6.93 (m, 2H), 6.74 (m, 1H), 4.10-4.20 (m, 2H), 2.85 (br s, 6H); LCMS Mass: 517.0 (M⁺+1).

### Example 7

### 3-((4-(Aminomethyl)-6-(trifluoromethyl)pyridin-2-yl)oxy)-N-(21-(FG-bead)-10,17,21-trihydroxy-7-oxo-12,15-dioxa-3,4-dithia-8,19-diazahenicosyl)benzamide (Compound 1-7)

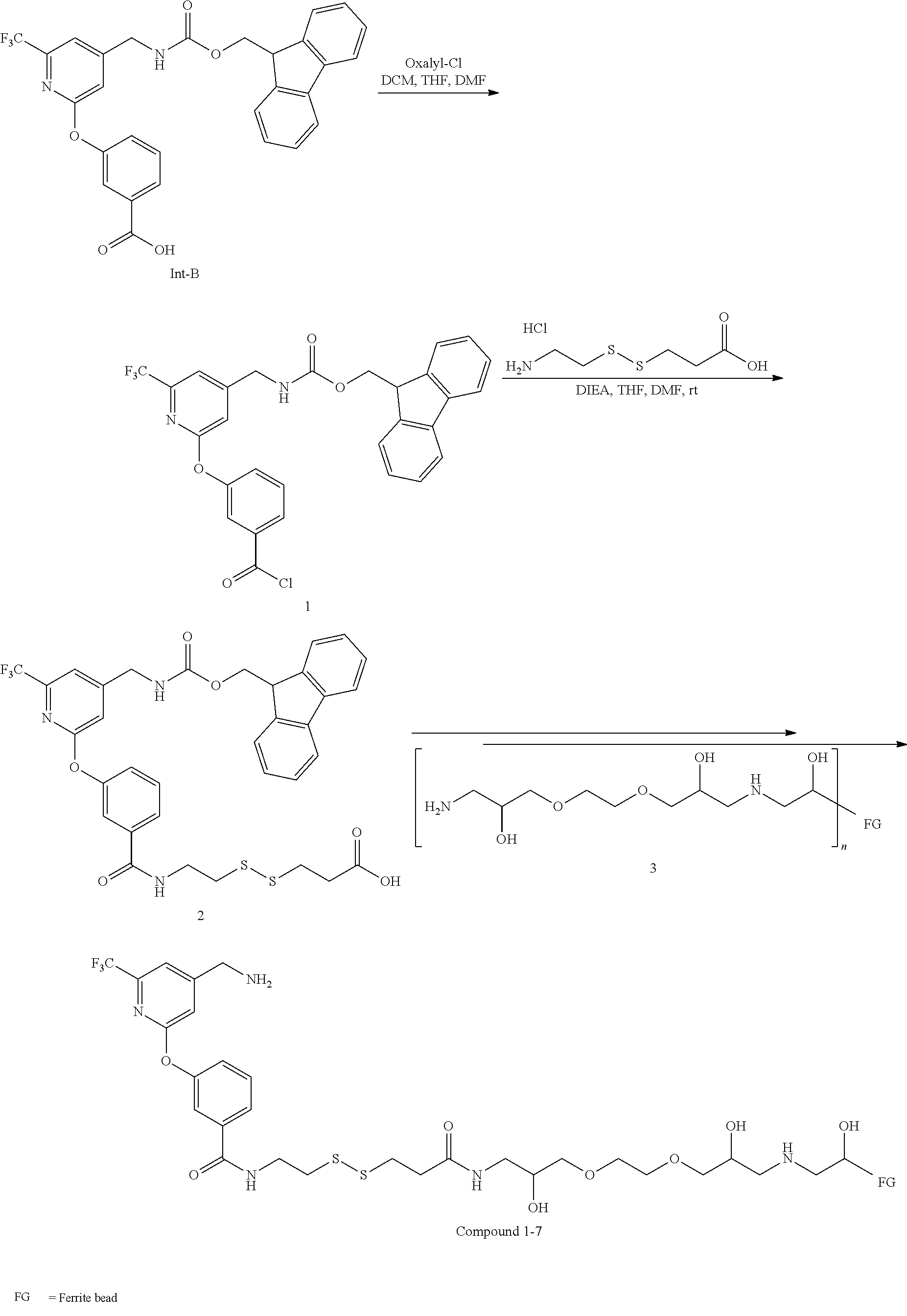

### Step 1: (9H-Fluoren-9-yl)methyl ((2-(3-(chlorocarbonyl)phenoxy)-6-(trifluoromethyl)pyridin-4-yl)methyl)carbamate (1)

To a stirred solution of Int-B (500 mg, 0.935 mmol), DCM (6 mL), and THF (6 mL) at RT under an inert atmosphere, were added DMF (catalytic, 3 drops) and oxalyl chloride (158 μL, 1.87 mmol). The mixture was stirred at RT for 40 min. The mixture was concentrated under reduced pressure. The mixture was diluted with water and brine, and then extracted with EtOAc (3×25 mL). The combined organic layers were washed with aq. sat NaHCO₃ (2×20 mL), brine, then dried (Na₂SO₄), filtered, and concentrated under reduced pressure. The obtained solid was purified via trituration with Et₂O (3×15 mL) to afford compound 1 (300 mg, 58%) as a white solid. ¹H NMR (300 MHz, DMSO-d₆): δ 8.03 (m, 1H), 7.80-7.90 (m, 3H), 7.63-7.70 (m, 3H), 7.50-7.60 (m, 2H), 7.25-7.50 (m, 5H), 7.16 (m, 1H), 4.10-4.40 (m, 5H).

### Step 2: 3-((2-(3-((4-(((((9H-Fluoren-9-yl)methoxy)carbonyl)amino)methyl)-6-(trifluoromethyl)pyridin-2-yl)oxy)benzamido)ethyl)disulfanyl)propanoic acid (2)

To a stirred solution of compound 1 (295 mg, 0.534 mmol), 3-[(2-aminoethyl)dithio]propionic acid hydrochloride (149 mg, 0.684 mmol), THF (12 mL), and DMF (0.5 mL) at RT, was added dropwise DIEA (186 μL, 1.07 mmol) and the mixture was stirred at RT for 25 min. The mixture was concentrated under reduced pressure, then diluted with water and brine, and then extracted with EtOAc (3×10 mL). The combined organic layers were washed with aq. 1M HCl (2×4 mL), brine, dried (Na₂SO₄), filtered, and concentrated under reduced pressure. The residue was purified (silica gel; eluting with 0-100% EtOAc in hexanes) to afford compound 2 (214 mg, 57%) as a white solid. ¹H NMR (300 MHz, DMSO-d₆): δ 12.35 (br s, 1H), 8.70 (br s, 1H), 8.03 (m, 1H), 7.80-7.95 (m, 2H), 7.60-7.78 (m, 4H), 7.51 (m, 1H), 7.23-7.43 (m, 6H), 7.14 (m, 1H), 4.20-4.40 (m, 5H), 3.45-3.55 (m, 2H), 2.80-2.90 (m, 4H), 2.55-2.65 (m, 2H); LCMS Mass: 696.0 (M⁺−1).

### Step 3: 3-((4-(Aminomethyl)-6-(trifluoromethyl)pyridin-2-yl)oxy)-N-(21-(FG-bead)-10,17,21-trihydroxy-7-oxo-12,15-dioxa-3,4-dithia-8,19-diazahenicosyl)benzamide (Compound 1-7)

Compound 1-7 was prepared in the following manner, and was used as is; NH2-derivatized high performance magnetic nanoparticle beads (FG-beads; containing 200 to 300 nmol/mg NH2) 3 were obtained from Nacalai USA, San Diego, Calif. (catalog number: TAS8848N1130; 1 mL of a 20 mg/mL suspension in deionized water).

NH2-derivatized FG-beads 3 (5 mg in 0.25 mL deionized water) were placed in a 1.5 mL microtube. The mixture was centrifuged at 15,000×g for 5 min at RT. The tube was placed on a magnet, and the supernatant was removed and discarded. The following washing procedure was carried out 3 times: DMF (1 mL) was added to disperse the beads and the mixture was centrifuged at 15,000×g for 5 min at RT. The tube was placed on a magnet and the supernatant was removed and discarded.

To the tube containing 5 mg FG-bead 3 at RT, was added DMF (400 μL) followed by compound 2 (200 μL of a 0.1M solution in DMF, 0.02 mmol). 1-Hydroxybenzotriazole hydrate (HOBt) (200 μL of a 0.5M solution in DMF, 0.1 mmol) was added, followed by 1-ethyl-3-(3-dimethylaminopropyl)carbodiimide (EDC) (200 μL of a 0.5M solution in DMF, 0.1 mmol). The tube was sealed and gently rotated at RT overnight. The mixture was centrifuged at 15,000×g for 5 min, then the tube was placed on a magnet and the supernatant was removed and discarded. The following washing procedure was carried out 3 times: DMF (1 mL) was added to disperse the beads and the mixture was centrifuged at 15,000×g for 5 min at RT. The tube was placed on a magnet and the supernatant was removed and discarded.

To acetylate (cap) any unreacted NH₂ groups attached to the FG-beads, the obtained derivatized FG-beads were resuspended in 20% acetic anhydride in DMF (1 mL) at RT, and the tube sealed and gently rotated for 1 h. The mixture was centrifuged at 15,000×g for 5 min, then the tube was placed on a magnet and the supernatant was removed and discarded. The following washing procedure was carried out 3 times: DMF (1 mL) was added to disperse the beads and the mixture was centrifuged at 15,000×g for 5 min at RT. The tube was placed on a magnet and the supernatant was removed and discarded.

The obtained derivatized FG-beads were resuspended in 30% piperidine in DMF (1 mL) at RT, and the tube sealed and gently rotated for 30 min. The mixture was centrifuged at 15,000×g for 5 min, then the tube was placed on a magnet and the supernatant was removed and discarded. The following washing procedure was carried out 3 times: DMF (1 mL) was added to disperse the beads. The mixture was centrifuged at 15,000×g for 5 min at RT. The tube was placed on a magnet and the supernatant was removed and discarded. An additional washing procedure was carried out 3 times: 50% methanol in water (1 mL) was added to disperse the beads and the mixture was centrifuged at 15,000×g for 5 min at RT. The tube was placed on a magnet and the supernatant was removed and discarded.

The obtained compound 1-7 was resuspended in 50% methanol (0.5 mL) and stored at 4° C. for up to one month prior to use.

### Example 8

### (E)-3-((4-(Aminomethyl)-6-(trifluoromethyl)pyridin-2-yl)oxy)-N-(2-(4-((4-(3-(4-(3-((3-(2-(3-((2-(FG-bead)-2-hydroxyethyl)amino)-2-hydroxypropoxy)ethoxy)-2-hydroxypropyl)carbamoyl)phenyl)-1H-1,2,3-triazol-1-yl)propoxy)phenyl)diazenyl)benzamido)ethyl)benzamide (Compound 1-8)

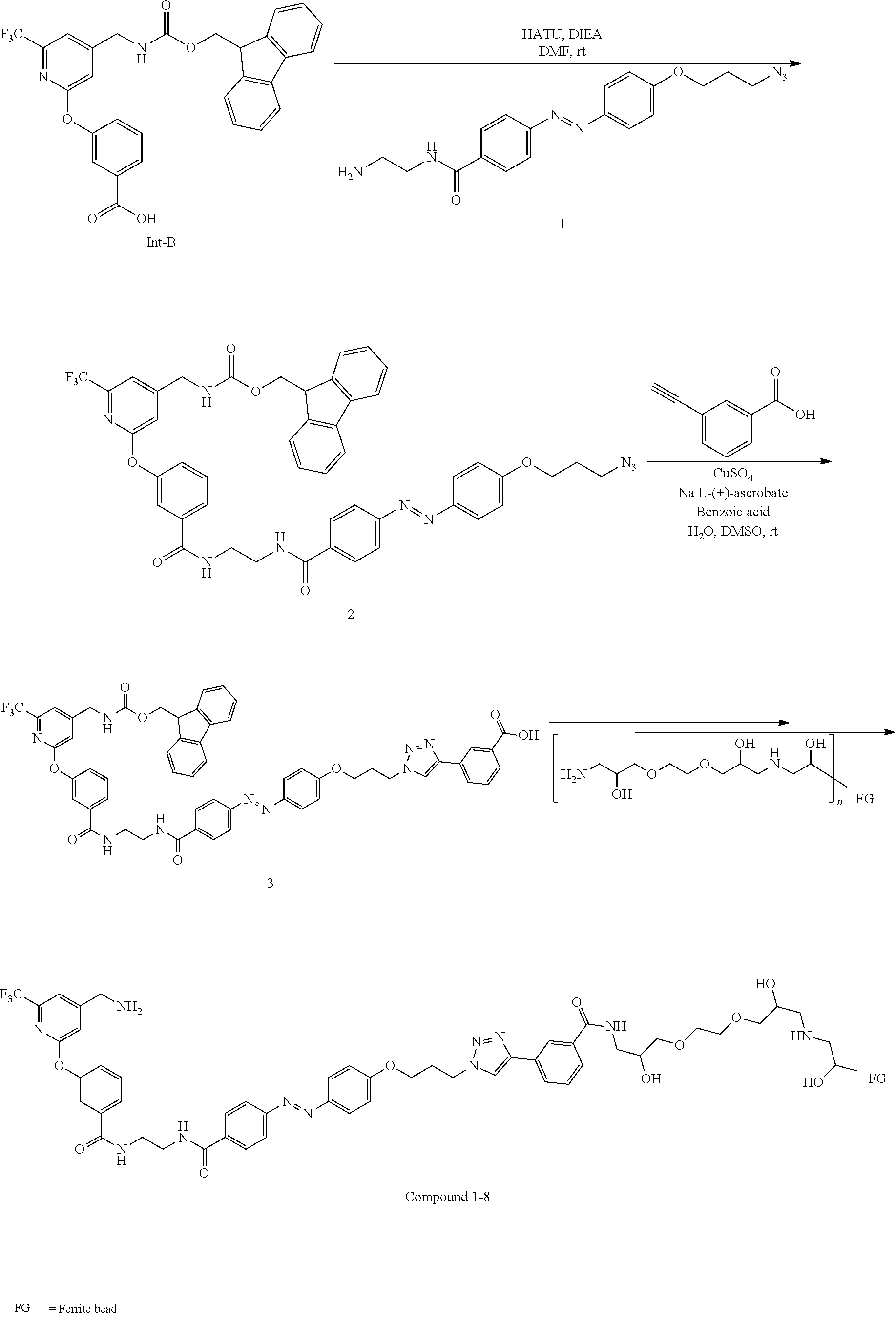

### Step 1: (E)-(9H-Fluoren-9-yl)methyl((2-(3-((2-(4-((4-(3-azidopropoxy)phenyl)diazenyl)benzamido)ethyl)carbamoyl)phenoxy)-6-(trifluoromethyl)pyridin-4-yl)methyl)carbamate (2)

To a stirred solution of Int-B (500 mg, 0.935 mmol) in DMF (6 mL) at RT, was added HATU (380 mg, 1.0 mmol) and the mixture was stirred for 20 min. (E)-N-(2-Aminoethyl)-4-{2-[4-(3-azidopropoxy)phenyl]diazenyl}benzamide hydrochloride 1 (Sigma-Aldrich, USA; catalog number 771139) (378 mg, 0.935 mmol) and DIEA (488 μL, 2.80 mmol) were added, and the mixture stirred at RT for 16 h. The reaction mixture was diluted with water, and repeatedly extracted with EtOAc. The combined organic layers were dried (Na₂SO₄), filtered, and concentrated under reduced pressure. The crude residue was purified via trituration with 4:1 MeOH:EtOAc to afford compound 2 (610 mg, 74%) as a yellow solid.¹H NMR (300 MHz, DMSO-d₆): δ 8.74 (br s, 1H), 8.72 (br s, 1H), 7.97-8.03 (m, 3H), 7.84-7.92 (m, 6H), 7.76 (m, 1H), 7.65-7.70 (m, 3H), 7.50-7.55 (m, 2H), 7.25-7.42 (m, 5H), 7.12-7.18 (m, 3H), 4.27-4.40 (m, 4H), 4.10-4.26 (m, 4H), 3.53 (m, 1H), 3.40-3.50 (m, 4H), 1.96-2.05 (m, 2H); LCMS Mass: 884.0 (M⁺+1).

### Step 2: (E)-3-(1-(3-(4-((4-((2-(3-((4-(((((9H-Fluoren-9-yl)methoxy)carbonyl)amino)methyl)-6-(trifluoromethyl)pyridin-2-yl)oxy)benzamido)ethyl)carbamoyl)phenyl)diazenyl)phenoxy)propyl)-1H-1,2,3-triazol-4-yl)benzoic acid (3)

A mixture of compound 2 (200 mg, 0.226 mmol), 3-ethynylbenzoic acid (35 mg, 0.242 mmol), CuSO₄ (catalytic, 1.25 mol %), sodium L-(+)-ascorbate (catalytic, 25 mol %), benzoic acid (catalytic, 10 mol %), DMSO (3 mL), and water (1 mL) was stirred at RT for 16 h. To the mixture was added 3-ethynylbenzoic acid (30 mg, 0.205 mmol) and additional CuSO₄ (catalytic, 1.25 mol %), sodium L-(+)-ascorbate (catalytic, 25 mol %), and benzoic acid (catalytic, 10 mol %). The reaction mixture was heated at 45° C. for 2 h. The mixture was concentrated under reduced pressure, then diluted with DMSO (4 mL). The mixture was purified via preparative reverse-phase HPLC (Waters XTerra® Prep MS C-18 OBD 5 μM 50×100 mm column; eluting with 25-100% MeCN/H₂O containing 0.1% TFA, over 18 min, followed by 100% MeCN/H₂O for 8 min) to afford compound 3 (58 mg, 25%) as a yellow solid. ¹H NMR (300 MHz, DMSO-d₆): δ 8.80 (m, 1H), 8.75 (br s, 1H), 8.67 (br s, 1H), 8.40 (m, 1H), 7.97-8.10 (m, 4H), 7.83-7.90 (m, 7H), 7.75 (m, 1H), 7.63-7.70 (m, 3H), 7.50-7.60 (m, 3H), 7.25-7.43 (m, 5H), 7.10-7.15 (m, 3H), 4.56-4.62 (m, 2H), 4.26-4.38 (m, 4H), 4.10-4.24 (m, 3H), 3.41-3.48 (m, 4H), 2.35-2.44 (m, 2H); LCMS Mass: 1031.0 (M⁺+1).

### Step 3: (E)-3-((4-(Aminomethyl)-6-(trifluoromethyl)pyridin-2-yl)oxy)-N-(2-(4-((4-(3-(4-(3-((3-(2-(3-((2-(FG-bead)-2-hydroxyethyl)amino)-2-hydroxypropoxy)ethoxy)-2-hydroxypropyl)carbamoyl)phenyl)-1H-1,2,3-triazol-1-yl)propoxy)phenyl)diazenyl)benzamido)ethyl)benzamide (Compound 1-8)

The title compound (1-8) was prepared from compound 3 using the procedure for Example 7, Step 3. The prepared compound 1-8 was used as is.

### Example 9

### 1-(4-(4-((2-(2-(4-(4-(3-((4-(Aminomethyl)-6-(trifluoromethyl)pyridin-2-yl)oxy)benzamido)butyl)-1H-1,2,3-triazol-1-yl)ethoxy)ethyl)amino)-4-oxobutoxy)-5-methoxy-2-nitrophenyl)ethyl (2-hydroxy-3-(2-(2-hydroxy-3-((2-hydroxy-2-(FG-bead)-ethyl)amino)propoxy)ethoxy)propyl)carbamate (Compound 1-9)

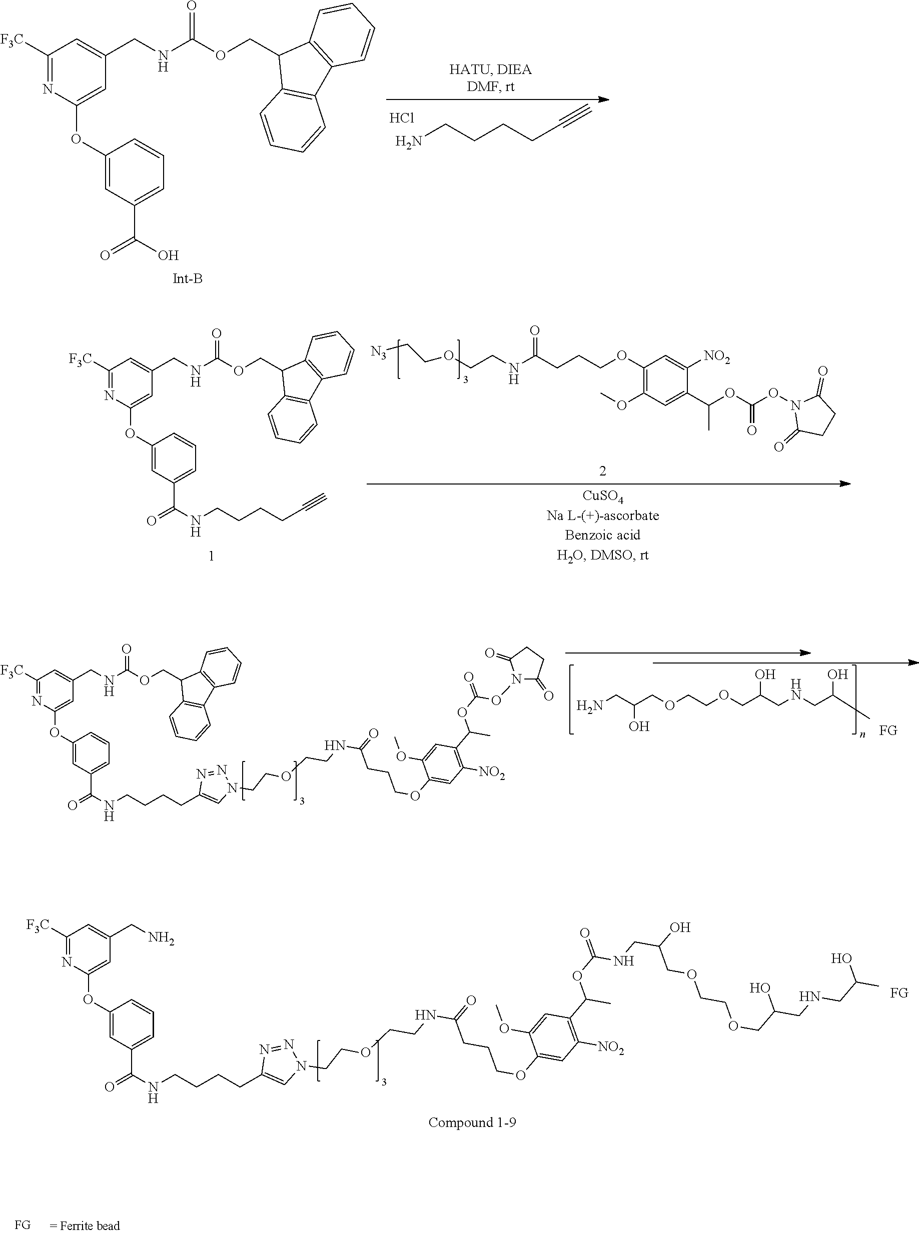

### Step 1: (9H-Fluoren-9-yl)methyl((2-(3-(hex-5-yn-1-ylcarbamoyl)phenoxy)-6-(trifluoromethyl)pyridin-4-yl)methyl)carbamate (1)

The title compound (1) (104 mg, 82%) was prepared using the procedure for Example 8, Step 1, using hex-5-yn-1-amine hydrochloride. ¹H NMR (300 MHz, DMSO-d₆): δ 8.52 (m, 1H), 8.03 (m, 1H), 7.85-7.90 (m, 2H), 7.62-7.74 (m, 4H), 7.48-7.54 (m, 2H), 7.26-7.43 (m, 5H), 7.14 (m, 1H), 4.28-4.40 (m, 4H), 4.23 (m, 1H), 3.20-3.28 (m, 2H), 2.75 (m, 1H), 2.12-2.20 (m, 2H), 1.51-1.62 (m, 2H), 1.40-1.50 (m, 2H); LCMS Mass: 614.0 (M⁺+1).

### Step 2: (9H-Fluoren-9-yl)methyl ((2-(3-((4-(1-(16-(4-(1-((((2,5-dioxopyrrolidin-1-yl)oxy)carbonyl)oxy)ethyl)-2-methoxy-5-nitrophenoxy)-13-oxo-3,6,9-trioxa-12-azahexadecyl)-1H-1,2,3-triazol-4-yl)butyl)carbamoyl)phenoxy)-6-(trifluoromethyl)pyridin-4-yl)methyl)carbamate (3)

A mixture of compound 1 (25 mg, 0.041 mmol), 1-(4-((1-azido-13-oxo-3,6,9-trioxa-12-azahexadecan-16-yl)oxy)-5-methoxy-2-nitrophenyl)ethyl (2,5-dioxopyrrolidin-1-yl) carbonate (PC Azido-NHS Ester; Click Chemistry Tools, Scottsdale, Ariz., USA; catalog number 1161) (26 mg, 0.041 mmol), CuSO₄ (catalytic, 1.25 mol %), sodium L-(+)-ascorbate (catalytic, 25 mol %), benzoic acid (catalytic, 10 mol %), DMSO (2 mL), and water (0.4 mL) was stirred at RT for 4 h. The mixture was concentrated under reduced pressure, then diluted with DMSO. The mixture was purified via preparative reverse-phase HPLC (Waters XTerra® Prep MS C-18 OBD 5 μM 50×100 mm column; eluting with 0-100% MeCN/H₂O containing 0.1% TFA) to afford compound 3 (21 mg, 41%) as a solid. ¹H NMR (300 MHz, DMSO-d₆): δ 8.51 (br m, 1H), 8.03 (br m, 1H), 7.85-7.94 (m, 3H), 7.78 (m, 1H), 7.60-7.75 (m, 4H), 7.57 (m, 1H), 7.47-7.53 (m, 2H), 7.26-7.42 (m, 5H), 7.12-7.15 (m, 2H), 6.27 (m, 1H), 4.32-4.42 (m, 4H), 4.28-4.31 (m, 2H), 4.21 (m, 1H), 4.00-4.07 (m, 2H), 3.92-3.96 (m, 3H), 3.70-3.90 (m, 5H), 3.39-3.44 (m, 6H), 3.32-3.37 (m, 2H), 3.20-3.30 (m, 2H), 3.12-3.19 (m, 2H), 2.74 (s, 3H), 2.55-2.64 (m, 2H), 2.19-2.24 (m, 2H), 1.90-1.98 (m, 2H), 1.67-1.71 (m, 3H), 1.50-1.63 (m, 4H); LCMS Mass: 1254.0 (M⁺).

### Step 3: 1-(4-(4-((2-(2-(4-(4-(3-((4-(Aminomethyl)-6-(trifluoromethyl)pyridin-2-yl)oxy)benzamido)butyl)-1H-1,2,3-triazol-1-yl)ethoxy)ethyl)amino)-4-oxobutoxy)-5-methoxy-2-nitrophenyl)ethyl (2-hydroxy-3-(2-(2-hydroxy-3-((2-hydroxy-2-(FG-bead)-ethyl)amino)propoxy)ethoxy)propyl)carbamate (Compound 1-9)

The title compound (1-9) may be prepared from compound 3 using the procedure for Example 7, Step 3, with the following key modification to that procedure; in place of the described HOBt/EDC mediated amide coupling, compound 3 may instead be treated directly with the NH2-derivatized FG-beads in the presence of a solvent such as DMF, with or without heating. The prepared compound 1-9 may be used as is.

### Example 10

### 3-((4-(Aminomethyl)-6-(trifluoromethyl)pyridin-2-yl)oxy)-N-(3-(1,1,2,2-tetra-³H-ethyl)phenyl)benzamide (Compound 1-12)

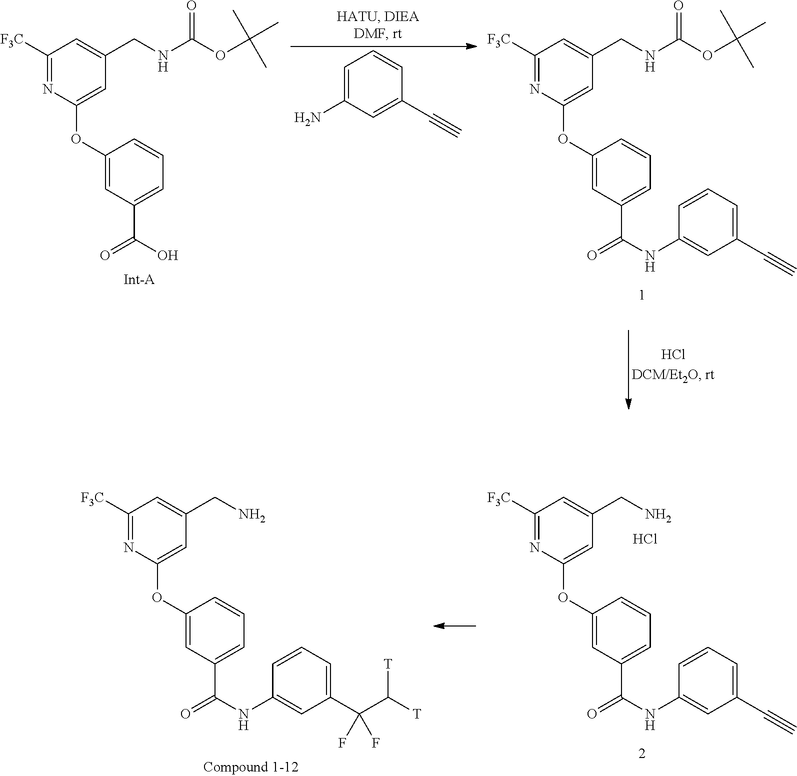

### Step 1: tert-Butyl((2-(3-((3-ethynylphenyl)carbamoyl)phenoxy)-6-(trifluoromethyl)pyridin-4-yl)methyl)carbamate (1)

To a stirred solution of Int-A (1 g, 2.43 mmol) in DMF (13 mL) at RT, was added HATU (1.85 g, 4.86 mmol) and the mixture was stirred at RT for 20 min. The mixture was cooled to 0° C. and 3-ethynylaniline (341 mg, 2.91 mmol) and DIEA (1.27 mL, 7.29 mmol) were added. The mixture was warmed to RT and stirred for 1 h. The reaction mixture was diluted with a mixture of water and brine. The mixture was repeatedly extracted with EtOAc and the combined organic layers were dried (Na₂SO₄), filtered, and concentrated under reduced pressure. The crude residue was purified (silica gel; eluting with 0-65% EtOAc in hexanes) to afford compound 1 (1 g, 81%) as a light yellow oil. ¹H NMR (300 MHz, DMSO-d₆): δ 10.35 (s, 1H), 7.10-8.00 (m, 11H), 4.18-4.27 (m, 3H), 1.36 (s, 9H).

### Step 2: 3-((4-(Aminomethyl)-6-(trifluoromethyl)pyridin-2-yl)oxy)-N-(3-ethynylphenyl)benzamide hydrochloride (2)

To a stirred mixture of 1 (278 mg, 0.54 mmol) in DCM (5.4 mL) at RT, was added 2 M HCl in Et₂O (5.4 mL, 10.8 mmol) and the mixture was stirred at RT for 16 h. Additional 2 M HCl in Et₂O (1.35 mL, 2.70 mmol) was added and the mixture stirred for a further 2 h. The mixture was concentrated under reduced pressure to afford compound 2 (230 mg, 95%) as a white solid. ¹H NMR (300 MHz, DMSO-d₆): δ 10.45 (s, 1H), 8.67 (br s, 3H), 7.20-8.00 (m, 10H), 4.20-4.40 (m, 3H); LCMS Mass: 412.0 (M⁺+1).

### Step 3: 3-((4-(Aminomethyl)-6-(trifluoromethyl)pyridin-2-yl)oxy)-N-(3-(1,1,2,2-tetra-³H-ethyl)phenyl)benzamide (Compound 1-12)

10% Palladium on carbon (0.4 mg) was added to a tritium reaction vessel, followed by a solution of compound 2 (0.4 mg) in DMF (0.3 mL). The vessel was attached to the tritium line and pressurized to 0.5 atmosphere with tritium gas at −200° C. The solution was stirred at RT for 30 mins, then cooled to −200° C. and excess gas removed. The reaction flask was rinsed several times with MeOH, passing each of the MeOH washes through a celite pad. The combined MeOH solutions were concentrated under reduced pressure. The crude material (21 mCi) was purified via semi-preparative reverse-phase HPLC to afford compound 1-12 (8 mCi, >99% pure) which was dissolved in EtOH. The specific activity was determined to be 58 Ci/mmol as measured by mass spectroscopy.

### Example 11

### (3-((4-(Aminomethyl)-6-(trifluoromethyl)pyridin-2-yl)oxy)phenyl)((3R,4R)-3-¹⁸fluoro-4-hydroxypyrrolidin-1-yl)methanone hydrochloride (Compound 1-13)

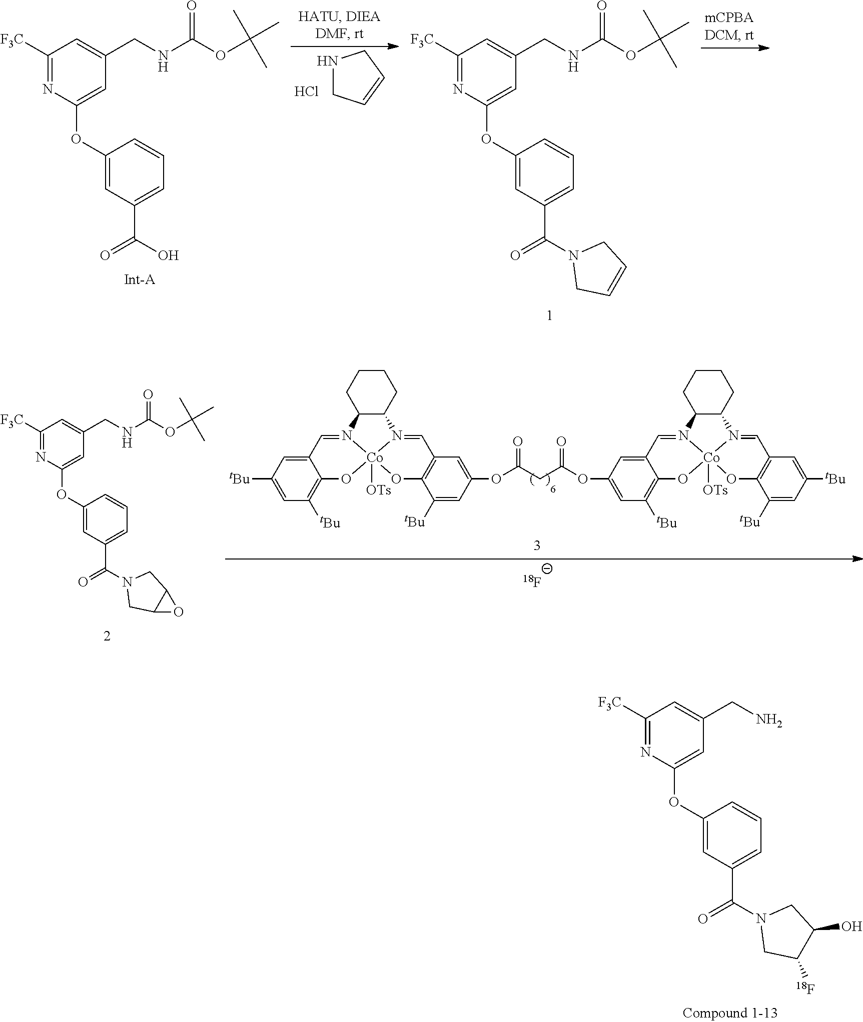

### Step 1: tert-Butyl((2-(3-(2,5-dihydro-1H-pyrrole-1-carbonyl)phenoxy)-6-(trifluoromethyl)pyridin-4-yl)methyl)carbamate (1)

To a stirred solution of Int-A (500 mg, 1.21 mmol) in DMF (4 mL) at RT, was added HATU (460 mg, 1.21 mmol) and the mixture was stirred at RT for 20 min. 2,5-Dihydro-1H-pyrrole hydrochloride (141 mg, 1.33 mmol) and DIEA (632 μL, 3.63 mmol) were added, and the mixture was stirred at RT for 1.5 h. The reaction mixture was diluted with a mixture of water and brine. The mixture was repeatedly extracted with EtOAc and the combined organic layers were dried (Na₂SO₄), filtered, and concentrated under reduced pressure. The crude residue was purified (silica gel; eluting with 0-80% EtOAc in hexanes), to afford compound 1 (443 mg, 79%) as a colorless oil. ¹H NMR (300 MHz, DMSO-d₆): δ 7.59 (m, 1H), 7.49-7.54 (m, 2H), 7.39-7.42 (m, 2H), 7.28 (m, 1H), 7.12 (m, 1H), 5.92 (m, 1H), 5.79 (m, 1H), 4.22-4.26 (m, 4H), 4.17-4.20 (m, 2H), 1.37 (s, 9H); LCMS Mass: 408.0 (MH⁺−C₄H₈).

### Step 2: tert-Butyl((2-(3-(6-oxa-3-azabicyclo[3.1.0]hexane-3-carbonyl)phenoxy)-6-(trifluoromethyl)pyridin-4-yl)methyl)carbamate (2)

To a stirred solution of compound 1 (50 mg, 0.108 mmol) in DCM at RT, was added mCPBA (70%; 330 mg, 1.35 mmol). The mixture was stirred at RT for 16 h. The mixture was diluted with aq. Na₂SO₃ then washed with sat. aq. NaHCO₃ solution. The mixture was concentrated under reduced pressure. The residue was washed with aq. Na₂SO₃ followed by sat. aq. NaHCO₃ solution. The organic layer was dried (Na₂SO₄), filtered, and concentrated under reduced pressure. The residue was purified (silica gel; eluting with 0-80% EtOAc in hexanes), to afford compound 2 (30 mg, 59%) as a white solid. LCMS Mass: 424.0 (MH⁺−C₄H₈). Step 3: (3-((4-(Aminomethyl)-6-(trifluoromethyl)pyridin-2-yl)oxy)phenyl)((3R,4R)-3-¹⁸fluoro-4-hydroxypyrrolidin-1-yl)methanone (Compound 1-13)

A Chromafix PS-HCO₃ cartridge was preconditioned with H₂O (10 mL), Na₂CO₃ (260 mg in 10 mL H₂O), followed by H₂O (10 mL). The cartridge was then loaded with aq. [¹⁸F]fluoride (in 2.5 mL H₂O) (produced by Siemens Eclipse cyclotron) and eluted with a solution of (S,S,S,S)-(linked salen)Co₂(OTs)₂ complex 3 (20 mg) (prepared using procedures described in T. J. A. Graham et al, J. Am. Chem. Soc. 2014, 136, 5291-4) in MeOH (1 mL) prepared immediately before use. The mixture was concentrated to dryness at 40° C. under a stream of N₂. The dry (S,S,S,S)-(linked salen)Co₂(OTs)¹⁸F complex was cooled to RT, then anhydrous MeCN (0.5 mL×2) was added and heated at 60° C. to dryness. The dry (S,S,S,S)-(linked salen)Co₂(OTs)¹⁸F complex was cooled to RT and a solution of compound 2 (12 mg) in MeCN (0.5 mL) was added, and the mixture was heated at 60° C. for 25 min. The mixture was quenched with H₂O (1 mL), filtered, and purified via semi-preparative reverse-phase HPLC (Agilent Eclipse XDB-Phenyl reverse-phase column, 9.4×250 mm, 5 μm, flow rate of 5 mL/min and eluting with 40% MeCN in H₂O over 30 min, Rₜ 17.9 min). The obtained N-Boc protected ¹⁸F-labeled product was diluted with 1M ammonium formate solution (20 mL), loaded onto a light C-18 cartridge and eluted with EtOH (1 mL). The obtained solution was concentrated at 50° C. 4M HCl (0.1 mL) was added and the mixture stirred for 5 minutes. Aq. 2M NaOH was used to neutralize the pH. An Agilent Eclipse XDB-Phenyl reverse-phase column (4.6×150 mm, 5 μm) was used for analysis, eluting with MeCN/H₂O (flow rate: 1 mL/min; t=0-2 min: 5% MeCN in H₂O, t=2-13 min: 5-95% MeCN in H₂O, t=12-13 min: 95-5% MeCN in H₂O, and t=13-14 min: 5% MeCN in H₂O; Rₜ 7.1 min). Tracers matched the retention time of the respective standards and exceeded 95% radiochemical purity, 0.75-3.2 mCi was obtained (0.3-1.3% radiochemical yield, decay corrected at TOI, 110 min synthesis time).

### Example 12

### (R,R)-trans-(3-((4-(Aminomethyl)-6-(trifluoromethyl)pyridin-2-yl)oxy)phenyl)(3-fluoro-4-hydroxypyrrolidin-1-yl)methanone hydrochloride (1-13a)

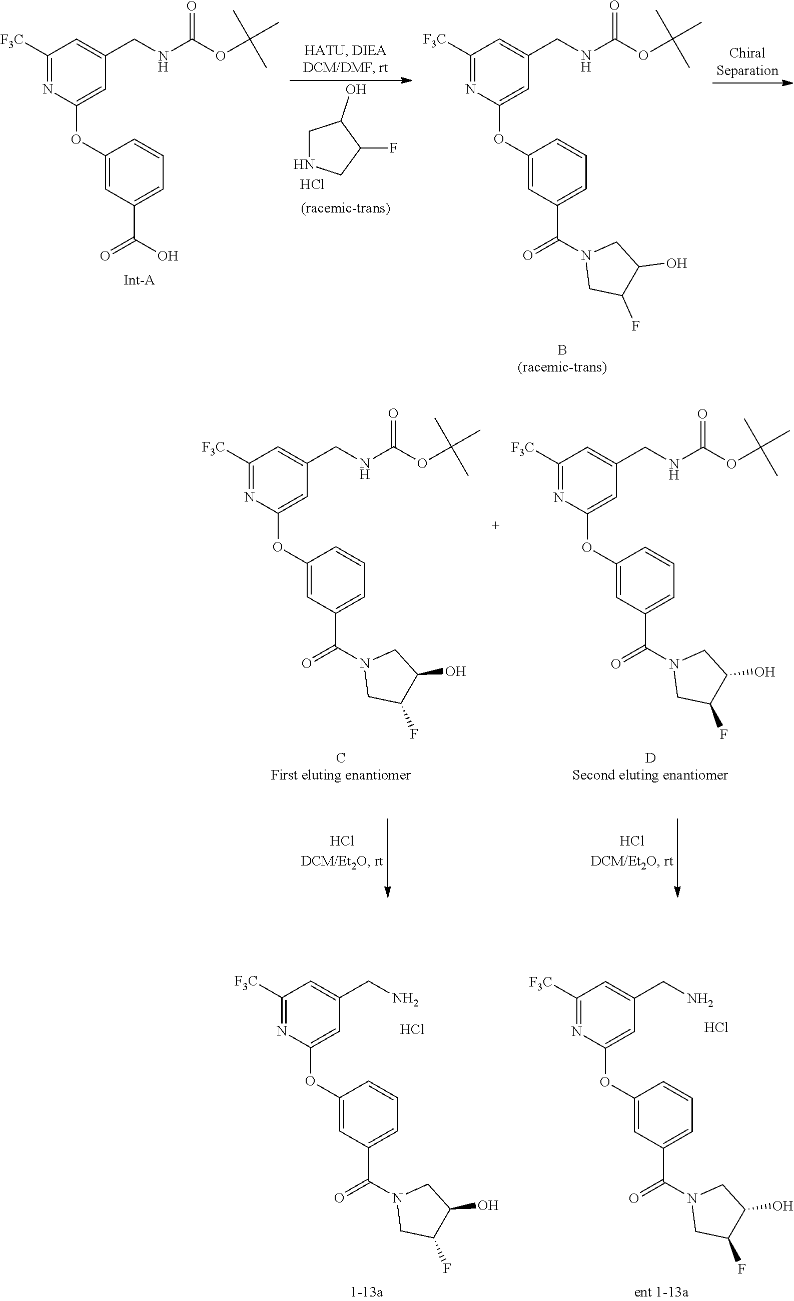

### Step 1: Racemic-trans-tert-butyl((2-(3-(3-fluoro-4-hydroxypyrrolidine-1-carbonyl)phenoxy)-6-(trifluoromethyl)pyridin-4-yl)methyl)carbamate (B)

Two separate equal reaction batches were set up as follows: To a stirred solution of Int-A (750 mg, 1.82 mmol) in a mixture of DCM/DMF (3:1, 11 mL), was added HATU (1.0 g, 2.63 mmol) and the mixture was stirred at RT for 20 min. Racemic-trans-4-fluoro-3-hydroxypyrrolidine hydrochloride (Synthonix; 304 mg, 2.14 mmol) and DIEA (938 mg, 7.27 mmol) were added and the mixture stirred at RT for 2.5 h. At this point both reaction batches were combined and the DCM was evaporated under reduced pressure. The remaining reaction mixture was partitioned between water (200 mL) and EtOAc (200 mL). The organic layer was separated, dried (Na₂SO₄), filtered, and then concentrated under reduced pressure. The crude residue was purified (silica gel; eluting with 10-100% EtOAc in hexanes) to afford compound B (1.58 g, 87%) as a white solid. ¹H NMR (300 MHz, DMSO-d₆): δ 7.60 (m, 1H), 7.47-7.56 (m, 2H), 7.36-7.44 (m, 2H), 7.31 (m, 1H), 7.14 (s, 1H), 5.56 (m, 1H), 4.93 (m, 1H), 4.10-4.30 (m, 3H), 3.45-3.90 (m, 4H), 1.38 (s, 9H); LCMS Mass: 522.0 (M⁻+Na).

### Step 2: (R,R)-trans-tert-butyl((2-(3-(3-fluoro-4-hydroxypyrrolidine-1-carbonyl)phenoxy)-6-(trifluoromethyl)pyridin-4-yl)methyl)carbamate (C) and (S,S)-trans-tert-butyl((2-(3-(3-fluoro-4-hydroxypyrrolidine-1-carbonyl)phenoxy)-6-(trifluoromethyl)pyridin-4-yl)methyl)carbamate (D)

Compound C (102 mg) and compound D (88 mg) were both obtained from compound B (300 mg, 0.60 mmol) via chiral HPLC separation (Chiral Pak ADH, 250×20 mm, 5 column, eluting isocratically with 10% MeOH:isopropanol (1:1) and 90% hexanes (containing 0.1% DEA), flow rate 18 mL/min), wherein compound C was the first to elute and compound D was the second to elute.

Compound C: ¹H NMR (400 MHz, DMSO-d₆): δ 7.59 (m, 1H), 7.47-7.56 (m, 2H), 7.35-7.45 (m, 2H), 7.31 (m, 1H), 7.16 (s, 1H), 5.56 (m, 1H), 4.94 (m, 1H), 4.25-4.30 (m, 2H), 4.17 (m, 1H), 3.45-3.90 (m, 4H), 1.39 (s, 9H); LCMS Mass: 500.0 (M⁺+1). Chiral HPLC analysis: Rₜ=11.84 min (Chiral Pak ADH, 250×4.6 mm, 5 μm column, eluting isocratically with 10% MeOH:EtOH (1:1) and 90% hexanes (containing 0.1% DEA) over 25 mins; flow rate 1.0 mL/min).

Compound D: ¹H NMR (400 MHz, DMSO-d₆): δ 7.59 (m, 1H), 7.47-7.56 (m, 2H), 7.35-7.45 (m, 2H), 7.31 (m, 1H), 7.16 (s, 1H), 5.56 (m, 1H), 4.95 (m, 1H), 4.25-4.30 (m, 2H), 4.17 (m, 1H), 3.45-3.90 (m, 4H), 1.39 (s, 9H); LCMS Mass: 500.0 (M⁺+1). Chiral HPLC analysis: Rₜ=14.71 min (Chiral Pak ADH, 250×4.6 mm, 5 μm column, eluting isocratically with 10% MeOH:EtOH (1:1) and 90% hexanes (containing 0.1% DEA) over 25 mins; flow rate 1.0 mL/min).

### Step 3: (S,S)-trans-(3-((4-(Aminomethyl)-6-(trifluoromethyl)pyridin-2-yl)oxy)phenyl)(3-fluoro-4-hydroxypyrrolidin-1-yl)methanone hydrochloride (Compound ent-13a)

To a stirred solution of compound D (87 mg, 0.174 mmol) in DCM (2 mL) at RT, Was added 2 M HCl in Et₂O (2.0 mL, 4.0 mmol) and the mixture was stirred at RT for 18 h. The mixture was concentrated under reduced pressure to afford the title compound (77 mg, 100% mmol) as a white solid. ¹H NMR (300 MHz, DMSO-d₆): δ 8.61 (br s, 3H), 7.84 (s, 1H), 7.51-7.57 (m, 2H), 7.43 (m, 1H), 7.28-7.37 (m, 2H), 5.57 (br m, 1H), 4.95 (m, 1H), 4.12-4.30 (br m, 3H), 3.30-3.92 (m, 4H); LCMS Mass: 400.0 (M⁺+1).

### Step 4: (R,R)-trans-(3-((4-(Aminomethyl)-6-(trifluoromethyl)pyridin-2-yl)oxy)phenyl)(3-fluoro-4-hydroxypyrrolidin-1-yl)methanone hydrochloride (1-13a)

To a stirred solution of compound C (102 mg, 0.204 mmol) in DCM (2 mL) at RT, Was added 2 M HCl in Et₂O (2.0 mL, 4.0 mmol) and the mixture was stirred at RT for 18 h. The mixture was concentrated under reduced pressure to afford the title compound (102 mg, 100%) as a white solid. ¹H NMR (300 MHz, DMSO-d₆): δ 8.61 (br s, 3H), 7.84 (s, 1H), 7.51-7.57 (m, 2H), 7.43 (m, 1H), 7.28-7.37 (m, 2H), 5.62 (br m, 1H), 4.95 (m, 1H), 4.12-4.30 (br m, 3H), 3.30-3.92 (m, 4H); LCMS Mass: 400.0 (M⁺+1).

### Example 13

### 3-((4-(Aminomethyl)-6-(trifluoromethyl)pyridin-2-yl)oxy)-N-(13-oxo-17-((3aS,4S,6aR)-2-oxohexahydro-1H-thieno[3,4-d]imidazol-4-yl)-3,6,9-trioxa-12-azaheptadecyl)benzamide hydrochloride (Compound 1-19)

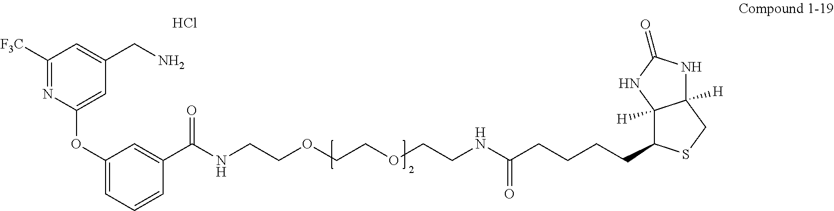

The title compound (1-19) was prepared using the procedure for Example 1, using N-(2-(2-(2-(2-aminoethoxy)ethoxy)ethoxy)ethyl)-5-((3aS,4S,6aR)-2-oxohexahydro-1H-thieno[3,4-d]imidazol-4-yl)pentanamide (EZ-Link™ Amine-PEG3-Biotin; ThermoFisher Scientific, USA; catalog number 21347) in Step 1. ¹H NMR (300 MHz, DMSO-d₆): δ 8.64 (m, 1H), 8.61 (br s, 3H), 7.80-7.90 (m, 2H), 7.78 (m, 1H), 7.65 (m, 1H), 7.56 (m, 1H), 7.50 (s, 1H), 7.37 (m, 1H), 6.39 (br s, 2H), 4.30 (m, 1H), 4.21-4.24 (m, 2H), 4.12 (m, 1H), 3.65-3.90 (br m, 8H), 3.34-3.41 (m, 5H), 3.04-3.18 (m, 4H), 2.82 (m, 1H), 2.57 (m, 1H), 2.04-2.07 (m, 2H), 1.60 (m, 1H), 1.35-1.53 (m, 3H), 1.20-1.30 (m, 2H); LCMS Mass: 713.0 (M⁺+1).

### Example 14

### 3-((4-(Aminomethyl)-6-(trifluoromethyl)pyridin-2-yl)oxy)-N-(2-(6-(6-(5-((3aS,4S,6aR)-2-oxohexahydro-1H-thieno[3,4-d]imidazol-4-yl)pentanamido)hexanamido)hexanamido)ethyl)benzamide hydrochloride (Compound 1-20)

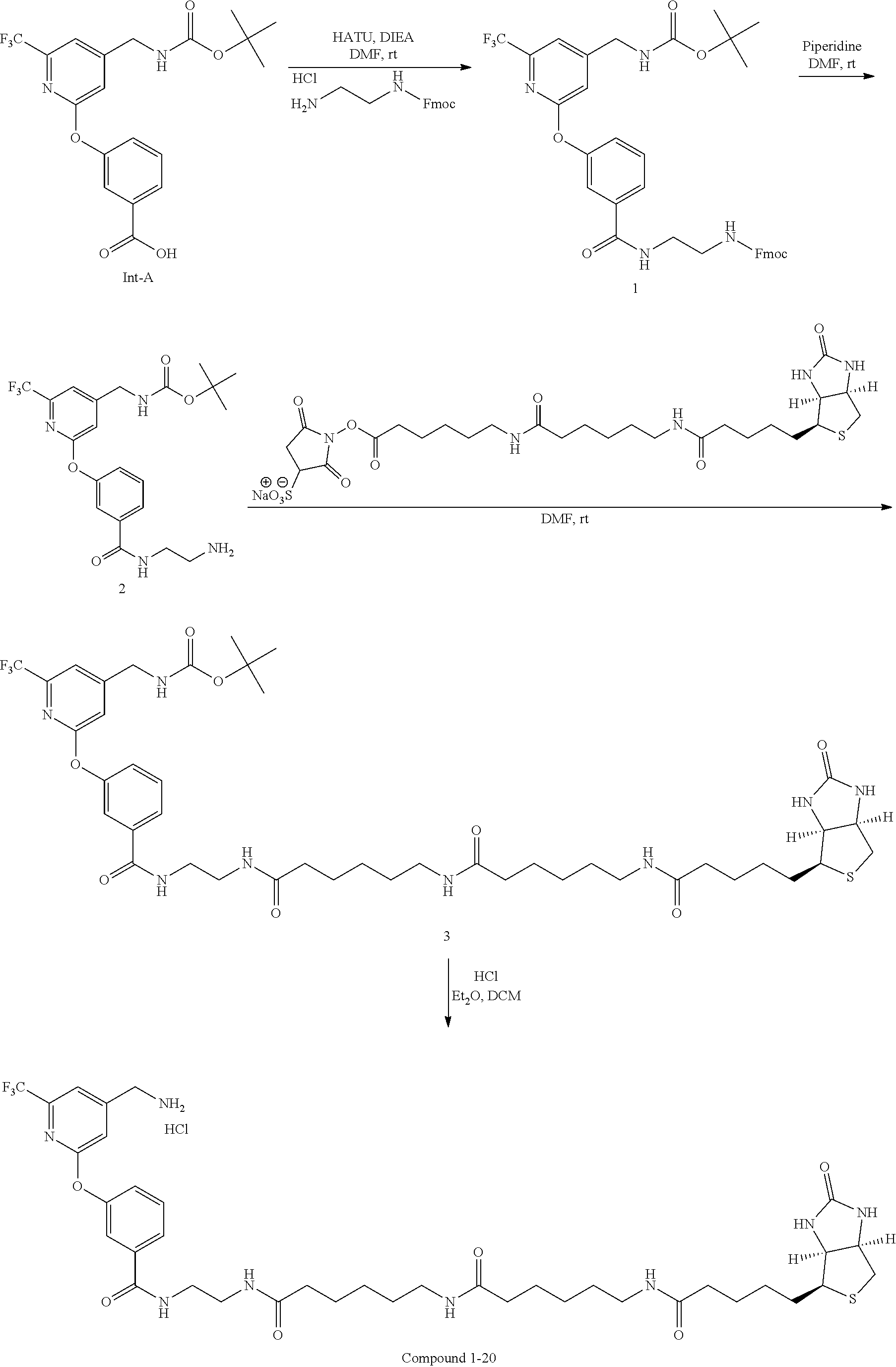

### Step 1: tert-Butyl((2-(3-((2-(3-((9H-fluoren-9-yl)methyl)carbamoyl)ethyl)phenoxy)-6-(trifluoromethyl)pyridin-4-yl)methyl)carbamate (1)

To a stirred solution of Int-A (500 mg, 1.21 mmol) in a mixture of DCM (5 mL) and DMF (4 mL) at RT, was added HATU (920 mg, 2.42 mmol) and the mixture was stirred at RT for 20 min. (9H-Fluoren-9-yl)methyl 2-aminoethylcarbamate hydrochloride (463 mg, 1.45 mmol) and DIEA (632 μL, 3.63 mmol) were added, and the mixture was stirred at RT for 1 h. The reaction mixture was diluted with a mixture of water, sat. aq. NaHCO₃, and brine. The mixture was repeatedly extracted with DCM. The combined organic layers were washed with a mixture of water and aq. 2M HCl, then separated, dried (Na₂SO₄), filtered, and concentrated under reduced pressure. The crude residue was purified via trituration with DCM to afford compound 1 (320 mg, 39%) as a white solid. LCMS Mass: 677.0 (M⁺+1).

### Step 2: tert-Butyl((2-(3-((2-aminoethyl)carbamoyl)phenoxy)-6-(trifluoromethyl)pyridin-4-yl)methyl)carbamate (2)

A mixture of compound 1 (256 mg, 0.378 mmol) in piperidine (2.26 mL) and DMF (5.30 mL) was stirred at RT for 10 min. The reaction mixture was diluted with a mixture of water and brine. The mixture was repeatedly extracted with EtOAc and the combined organic layers were dried (Na₂SO₄), filtered, and concentrated under reduced pressure. The crude residue was purified via trituration with a mixture of Et₂O and DCM to afford compound 2 (43 mg, 25%) as a yellow solid. LCMS Mass: 455.0 (M⁺+1).

### Step 3: tert-Butyl((2-(3-((2-(6-(6-(5-((3aS,4S,6aR)-2-oxohexahydro-1H-thieno[3,4-d]imidazol-4-yl)pentanamido)hexanamido)hexanamido)ethyl)carbamoyl)phenoxy)-6-(trifluoromethyl)pyridin-4-yl)methyl)carbamate (3)

A mixture of compound 2 (25 mg, 0.055 mmol), EZ-Link® Sulfo-NHS-LC-LC-Biotin (ThermoFisher Scientific, USA; catalog number 21338) (72 mg, 0.108 mmol) and DMF (0.5 mL) was stirred at RT for 2.5 h. The reaction mixture was diluted with a mixture of water, brine, and Et₂O. The observed solid was collected via filtration and dried to afford compound 3 (16 mg, 32%) as a white solid. LCMS Mass: 907.0 (M⁺).

### Step 4: 3-((4-(Aminomethyl)-6-(trifluoromethyl)pyridin-2-yl)oxy)-N-(2-(6-(6-(5-((3aS,4S,6aR)-2-oxohexahydro-1H-thieno[3,4-d]imidazol-4-yl)pentanamido)hexanamido)hexanamido)ethyl)benzamide hydrochloride (Compound 1-20)

To a stirred mixture of compound 3 (16 mg, 0.0176 mmol) in DCM (0.35 mL) at RT, was added 2 M HCl in Et₂O (0.35 mL, 0.704 mmol) and the mixture was stirred at RT for 10 min. The mixture was concentrated under reduced pressure. The crude residue was purified via trituration with Et₂O to afford the title compound 1-20 (10 mg, 68%) as a yellow solid. LCMS Mass: 807.0 (M⁺+1).

### Example 15

### 3-(2-((1E,3Z,5Z)-3-(3-(5-((6-(3-((4-(Aminomethyl)-6-(trifluoromethyl)pyridin-2-yl)oxy)benzamido)hexyl)amino)-5-oxopentyl)phenyl)-5-(1,1-dimethyl-6,8-disulfo-3-(3-sulfopropyl)-1H-benzo[e]indol-2(3H)-ylidene)penta-1,3-dien-1-yl)-1,1-dimethyl-6,8-disulfo-1H-benzo[e]indol-3-ium-3-yl)propane-1-sulfonate trifluoroacetate (Compound 1-22)

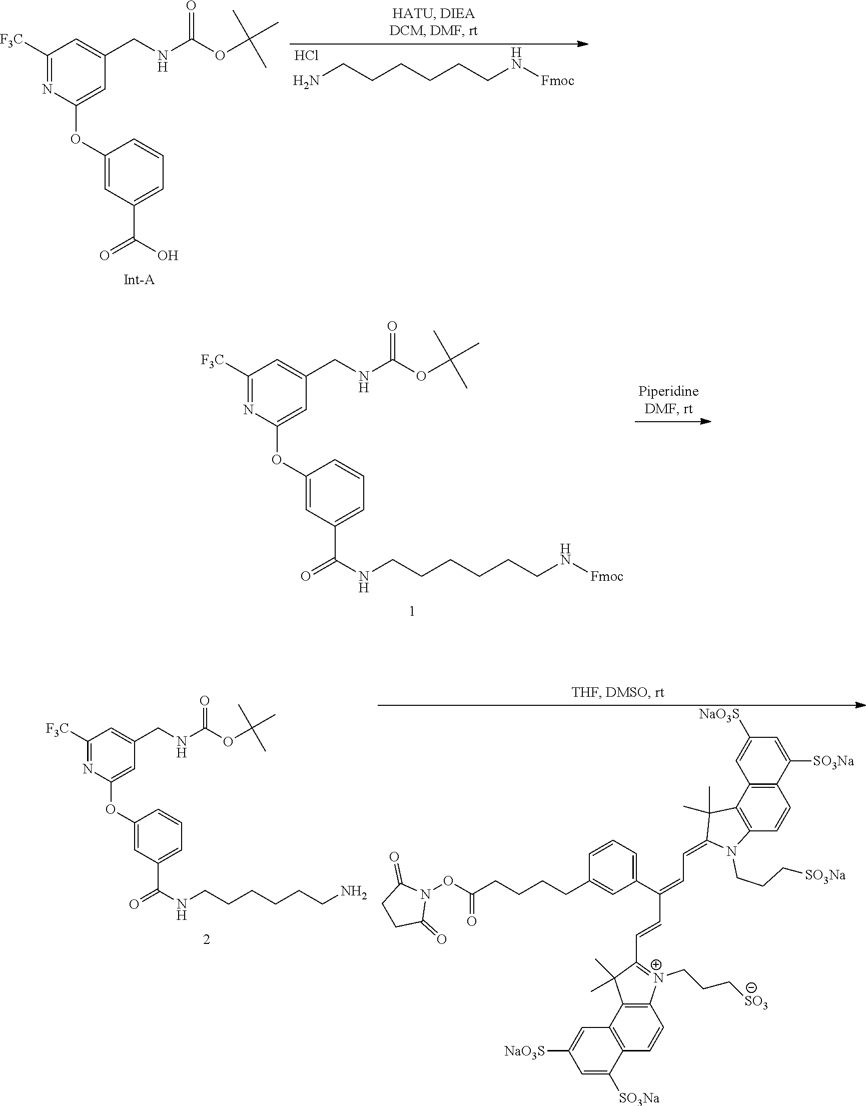

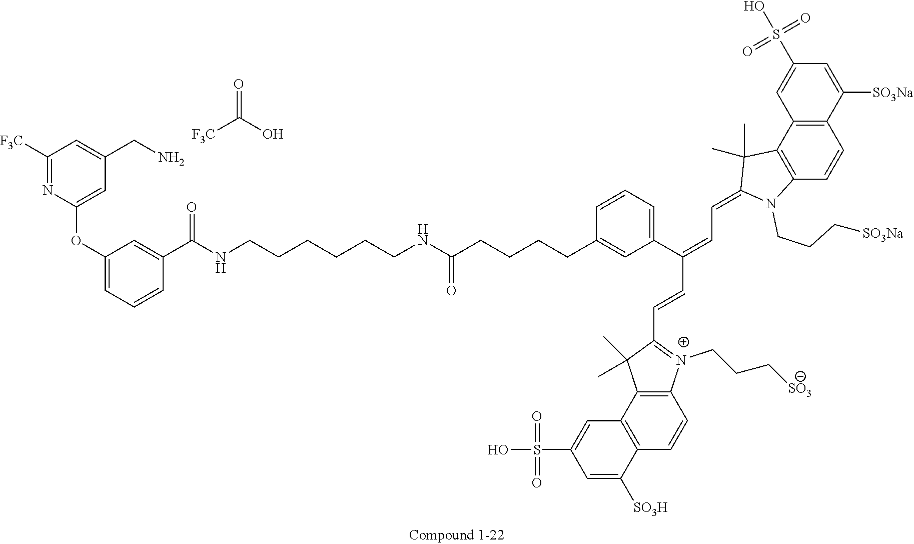

### Step 1: tert-Butyl((2-(3-((6-(Fmoc-amino)hexyl)carbamoyl)phenoxy)-6-(trifluoromethyl)pyridin-4-yl)methyl)carbamate (1)

To a stirred solution of Int-A (700 mg, 1.69 mmol) in DCM (10 mL) and DMF (1 mL) at RT, was added HATU (742 mg, 1.95 mmol) and the mixture stirred at rt for 10 min. DIEA (886 μL, 5.09 mmol) and Fmoc-1,6-diaminohexane hydrochloride (764 mg, 2.03 mmol) were added and the mixture stirred at rt for a further 3 h. The mixture was partitioned between water and DCM and the aqueous layer separated and extracted further with DCM. The combined organic layers were dried (MgSO₄), filtered, and concentrated under reduced pressure. The residue was purified (silica gel; eluting with 10-100% EtOAc in hexanes) to afford compound 1 (1.05 g, 85%) as an off-white solid. LCMS Mass: 733.0 (M⁺+1).

### Step 2: tert-Butyl((2-(3-((6-aminohexyl)carbamoyl)phenoxy)-6-(trifluoromethyl)pyridin-4-yl)methyl)carbamate (2)

A mixture of compound 1 (250 mg, 0.341 mmol) in piperidine (0.5 mL) and DMF (2 mL) was stirred at RT for 15 min. The reaction mixture was concentrated under reduced pressure. The crude residue was purified (silica gel; eluting with 10-100% EtOAc in hexanes followed by MeOH in DCM) to afford compound 2 (62 mg, 36%) as a white solid. LCMS Mass: 511.0 (M⁺+1).

### Step 3: 3-(2-((1E,3Z,5Z)-3-(3-(5-((6-(3-((4-(Aminomethyl)-6-(trifluoromethyl)pyridin-2-yl)oxy)benzamido)hexyl)amino)-5-oxopentyl)phenyl)-5-(1,1-dimethyl-6,8-disulfo-3-(3-sulfopropyl)-1H-benzo[e]indol-2(3H)-ylidene)penta-1,3-dien-1-yl)-1,1-dimethyl-6,8-disulfo-1H-benzo[e]indol-3-ium-3-yl)propane-1-sulfonate trifluoroacetate (Compound 1-22)

To a stirred solution of compound 2 (3.8 mg, 0.0074 mmol) in THF (0.3 mL) at rt, was added a solution of IRDye® 680LT NHS ester (Li-Cor; catalog number P/N 929-71500) (10 mg, 0.007 mmol) in DMSO (1 mL). The mixture was stirred at RT for 16 h. The mixture was concentrated under reduced pressure. The residue was purified via preparative reverse-phase HPLC (Waters XTerra® Prep MS C-18 OBD 5 μM 50×100 mm column; eluting with 10-70% MeCN/H₂O containing 0.1% TFA) to afford directly the N-Boc deprotected compound 1-22 (12 mg, 100%) as a solid. ¹H NMR (300 MHz, DMSO-d₆): δ 8.96-8.98 (m, 2H), 8.60-8.62 (m, 2H), 8.30-8.50 (m, 5H), 8.21 (s, 2H), 7.95 (m, 1H), 7.70-7.80 (m, 4H), 7.58 (s, 1H), 7.40-7.56 (m, 2H), 7.30 (m, 1H), 7.20-7.28 (m, 2H), 7.10-7.15 (m, 2H), 5.70-5.75 (m, 2H), 3.90-4.30 (m, 11H), 3.10-3.20 (m, 2H), 2.95-3.05 (m, 4H), 2.65-2.70 (m, 2H), 2.40-2.60 (m, 6H), 2.10-2.15 (m, 2H), 1.85-2.05 (m, 14H), 1.50-1.55 (m, 4H), 1.10-1.45 (m, 4H), 1.15-1.20 (m, 4H); LCMS Mass: 1589.0 (M⁺+1).

### Example 16

### 3-(2-((1E,3Z,5Z)-3-(3-(1-(3-((4-(Aminomethyl)-6-(trifluoromethyl)pyridin-2-yl)oxy)phenyl)-1,27-dioxo-5,8,11,14,17,20,23-heptaoxa-2,26-diazahentriacontan-31-yl)phenyl)-5-(1,1-dimethyl-6,8-disulfo-3-(3-sulfopropyl)-1H-benzo[e]indol-2(3H)-ylidene)penta-1,3-dien-1-yl)-1,1-dimethyl-6,8-disulfo-1H-benzo[e]indol-3-ium-3-yl)propane-1-sulfonate trifluoroacetate (Compound 1-25)

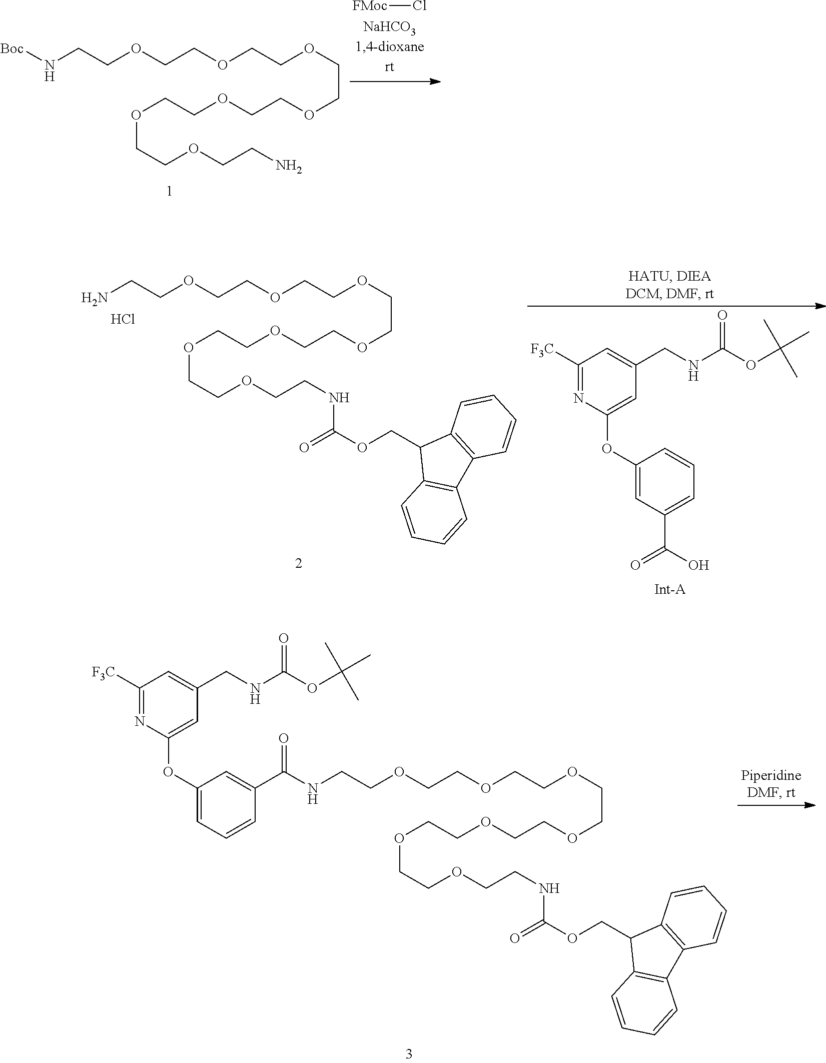

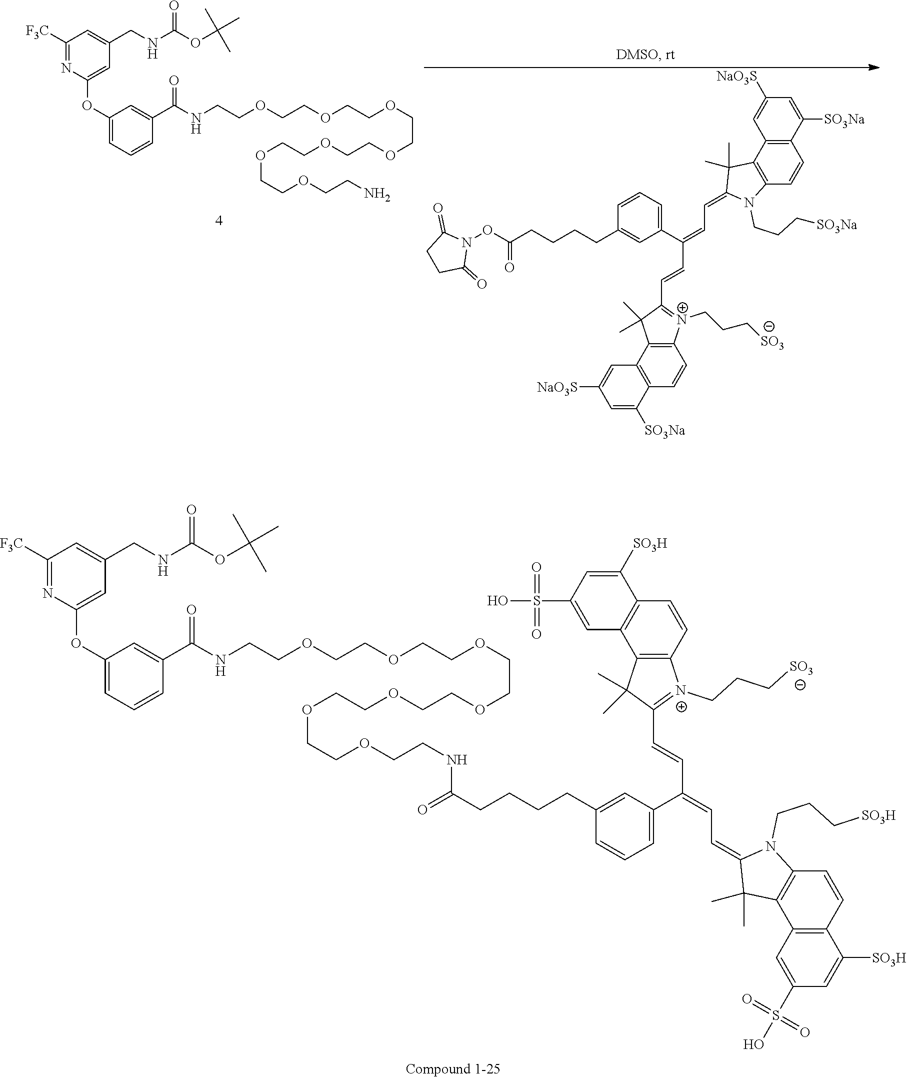

### Step 1: (9H-Fluoren-9-yl)methyl (23-amino-3,6,9,12,15,18,21-heptaoxatricosyl)carbamate hydrochloride (2)

To a stirred mixture of tert-butyl (23-amino-3,6,9,12,15,18,21-heptaoxatricosyl)carbamate 1 (500 mg, 1.07 mmol) and NaHCO₃ (269 mg, 3.2 mmol) in 1,4-dioxane (5 mL) at 0° C., was added a solution of 9-fluorenylmethyl chloroformate (304 mg, 1.17 mmol) in 1,4-dioxane (2 mL). The mixture was warmed to RT and stirred for 16 h. DCM and Et₂O were added and the mixture filtered. The obtained filtrate was concentrated under reduced pressure. The residue was purified (silica gel; eluting with 100% EtOAc in hexanes followed by 10% MeOH in DCM) to afford a colorless oil. The obtained oil was dissolved in DCM (7 mL) and to this was added 2M HCl in ether (9 mL, 18 mmol). The mixture was stirred at RT for 4 h. The mixture was concentrated under reduced pressure to afford compound 2 (479 mg, 81%) as a semi-solid. LCMS Mass: 591.0 (M⁺+1).

### Step 2: tert-Butyl((2-(3-((23-Fmoc-amino-3,6,9,12,15,18,21-heptaoxatricosyl)carbamoyl)phenoxy)-6-(trifluoromethyl)pyridin-4-yl)methyl)carbamate (3)

To a stirred solution of Int-A (171 mg, 0.40 mmol) in DCM (1 mL) and DMF (1 mL) at RT, was added HATU (180 mg, 0.40 mmol) and the mixture stirred at rt for 15 min. DIEA (216 μL, 2.2 mmol) and compound 2 (260 mg, 0.40 mmol) were added and the mixture stirred at rt for a further 3 h. The mixture was partitioned between water and DCM and the aqueous layer separated and extracted further with DCM. The combined organic layers were dried (MgSO₄), filtered, and concentrated under reduced pressure. The residue was purified (silica gel; eluting with 100% EtOAc in hexanes followed by 1-20% MeOH in DCM) to afford compound 3 (186 mg, 47%) as an oil. LCMS Mass: 986.0 (M⁺+1).

### Step 3: tert-Butyl((2-(3-((23-amino-3,6,9,12,15,18,21-heptaoxatricosyl)carbamoyl)phenoxy)-6-(trifluoromethyl)pyridin-4-yl)methyl)carbamate (4)

A mixture of compound 3 (180 mg, 0.183 mmol) in piperidine (0.3 mL) and DMF (1.2 mL) was stirred at RT for 1 h. The reaction mixture was concentrated under reduced pressure. The residue was purified via preparative reverse-phase HPLC (Waters XTerra® Prep MS C-18 OBD 5 μM 50×100 mm column; eluting with 15-65% MeCN/H₂O containing 0.1% TFA). The obtained pure fractions were partially concentrated and basified with aq. sat. NaHCO₃. The mixture was repeatedly extracted with EtOAc, and the combined organic layers were dried (MgSO₄), filtered, and concentrated under reduced pressure to afford compound 4 (75 mg, 54%) as a yellow oil. LCMS Mass: 763.0 (M⁺+1).

### Step 4: 3-(2-((1E,3Z,5Z)-3-(3-(1-(3-((4-(Aminomethyl)-6-(trifluoromethyl)pyridin-2-yl)oxy)phenyl)-1,27-dioxo-5,8,11,14,17,20,23-heptaoxa-2,26-diazahentriacontan-31-yl)phenyl)-5-(1,1-dimethyl-6,8-disulfo-3-(3-sulfopropyl)-1H-benzo[e]indol-2(3H)-ylidene)penta-1,3-dien-1-yl)-1,1-dimethyl-6,8-disulfo-1H-benzo[e]indol-3-ium-3-yl)propane-1-sulfonate trifluoroacetate (Compound 1-25)

To a stirred solution of compound 4 (18 mg, 0.0235 mmol) in DMSO (0.2 mL) at rt, was added a solution of IRDye® 680LT NHS ester (Li-Cor; catalog number P/N 929-71500) (20 mg, 0.014 mmol) in DMSO (0.8 mL). The mixture was stirred at RT for 16 h, followed by heating at 40° C. for 5 h, followed by RT for an additional 16 h. The mixture was directly purified via preparative reverse-phase HPLC (Waters XTerra® Prep MS C-18 OBD 5 μM 50×100 mm column; eluting with 10-95% MeCN/H₂O containing 0.1% TFA) to afford directly the N-Boc deprotected compound 1-25 (14 mg, 31%) as a solid. LCMS Mass: 1841.0 (M⁺+1).

### Example A-1

### Parenteral Pharmaceutical Composition

To prepare a parenteral pharmaceutical composition suitable for administration by injection (subcutaneous, intravenous), 1-1000 mg of a compound described herein, or a pharmaceutically acceptable salt or solvate thereof, is dissolved in sterile water and then mixed with 10 mL of 0.9% sterile saline. A suitable buffer is optionally added as well as optional acid or base to adjust the pH. The mixture is incorporated into a dosage unit form suitable for administration by injection

### Example A-2

### Oral Solution

To prepare a pharmaceutical composition for oral delivery, a sufficient amount of a compound described herein, or a pharmaceutically acceptable salt thereof, is added to water (with optional solubilizer(s),optional buffer(s) and taste masking excipients) to provide a 20 mg/mL solution.

### Example A-3

### Oral Tablet

A tablet is prepared by mixing 20-50% by weight of a compound described herein, or a pharmaceutically acceptable salt thereof, 20-50% by weight of microcrystalline cellulose, 1-10% by weight of low-substituted hydroxypropyl cellulose, 1-10% by weight of magnesium stearate, and other appropriate excipients. Tablets are prepared by direct compression. The total weight of the compressed tablets is maintained at 100-500 mg.

### Example A-4

### Oral Capsule

To prepare a pharmaceutical composition for oral delivery, 10-500 mg of a compound described herein, or a pharmaceutically acceptable salt thereof, is mixed with starch or other suitable powder blend. The mixture is incorporated into an oral dosage unit such as a hard gelatin capsule, which is suitable for oral administration.

In another embodiment, 10-500 mg of a compound described herein, or a pharmaceutically acceptable salt thereof, is placed into Size 4 capsule, or Size 1 capsule (hypromellose or hard gelatin) and the capsule is closed.

### Example B-1

### Human LOXL2 Amine Oxidase Activity Assay

LOXL2 amine oxidase activity is evaluated by measuring Amplex Red fluorescence using 10-20× concentrated conditioned media from CHO cells stably expressing human LOXL2. To assay for amine oxidase activity, 10 μL of the concentrated conditioned media is incubated with 2 μL of test compound in DMSO and 73 μL Assay Buffer (50 mM Borate Buffer, pH8) for 2 h at 37° C. After the 2 h incubation, 5 μL of 10 mM 1,5-diaminopentane (DAP) diluted in Assay Buffer and 10 μL of Amplex Red Mix (8.5 μL Assay Buffer+0.5 μL of 10 mM Amplex Red+1 μL of 500 U/mL Horseradish Peroxidase) are added and the plate mixed and immediately placed on the FlexStaion for fluorescence measurements. Fluorescence is read in kinetic mode every 2 min for 1 hour at excitation=544 nm and emission=590 nm. The amine oxidase activity is calculated from the slope of the linear portion of the curve.

TABLE 2

| Example | Compound | IC₅₀ |
|---|---|---|
| 1 | 1-1 | A |
| 2 | 1-2 | A |
| 3 | 1-3 | A |
| 4 | 1-4 | A |
| 5 | 1-5 | A |
| 6 | 1-6 | B |
| 12 | 1-13a | A |
| 13 | 1-19 | A |
| 14 | 1-20 | A |
| 15 | 1-22 | C |

A is < 300 nM;
B is 300 nM to 1000 nM;
C is > 1000 nM

### Example B-2

### LOXL2 Human Blood Amine Oxidase Activity Assay

The amine oxidase activity of human LOXL2 in the context of human whole blood is measured using an Amplex Red assay. Briefly, 0.5-2 μg recombinant, human LOXL2 (Sino Biologicals, Beijing, China) reconstituted in water is added to 192 μL human blood that was collected in heparin vacutainer tubes followed by the addition of 2 μL test compound in DMSO. Samples are mixed and incubated at 37° C. for 2 h. After the 2 h incubation, the blood is centrifuged at 2000×g for 15 min at room temperature to isolate the plasma. 50 μL of plasma is removed and mixed with 25 μL of 40 mM DAP (diluted in water) and 25 μL Amplex Red Mix (23.5 μL 50 mM Borate Buffer, pH8+0.5 μL 10 mM Amplex Red+1 μL 500 U/mL Horseradish Peroxidase). Samples are mixed and immediately placed on the FlexStaion for fluorescence measurements. Fluorescence is read in kinetic mode every 2 min for 1 hour at excitation=544 nm and emission=590 nm. The amine oxidase activity is calculated from the slope of the linear portion of the curve.

### Example B-3

### ELISA-Based Quantification of LOXL2 Using Biotinylated Labeled LOXL2 Inhibitor (Compound 1-1)

To quantify LOXL2 using an ELISA-based assay, the biotinylated LOXL2 inhibitor (Compound 1-1) was incubated with recombinant mouse or human LOXL2 for an appropriate amount of time and temperature to allow for suitable binding, such as 2 hours at 37° C. After incubation, the mixture was then transferred to a 96-well Streptavidin-coated plate and incubated for 1-2 hours at room temperature to capture the biotinylated labeled LOXL2 inhibitor (Compound 1-1)/LOXL2 complex. Each well was then washed to remove the excess/unbound LOXL2 and then incubated with a goat anti-LOXL2 antibody for ELISA detection, such as the commercially available antibody from R&D Systems cat #AF2639. Detection was performed using a horseradish peroxidase (HRP) conjugated anti-goat antibody.

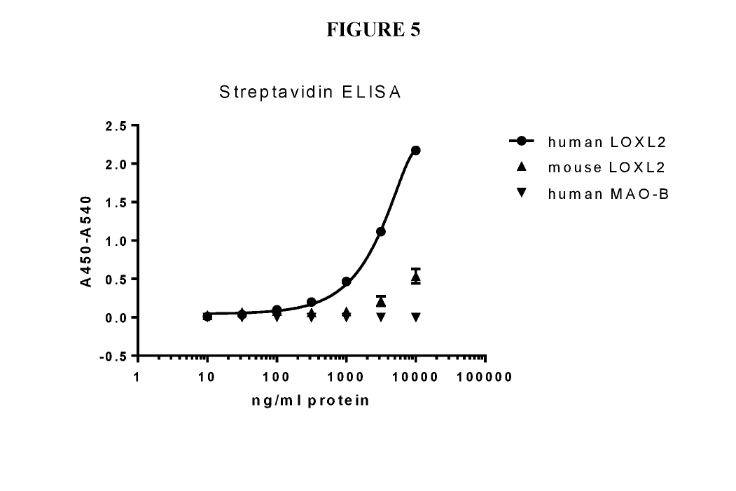

FIG. 5 shows the results of the ELISA-based quantification of human or mouse LOXL2 bound to a biotinylated LOXL2 inhibitor (Compound 1-1). A concentration range of human and mouse LOXL2 was tested. Purified recombinant human MAO-B was used as the negative control.

### Example B-4

### Western-Based Quantification of LOXL2 Using Biotinylated Labeled LOXL2 Inhibitor (Compound 1-1)

To quantify LOXL2 using a Western-based assay, a suitable amount of the LOXL2 inhibitor 1-13a, such as 10 or DMSO was pre-incubated with a suitable amount of LOXL2, such as 1 ng, 10 ng, or 100 ng, for a suitable amount of time at an appropriate temperature, such as 1-2 hrs at 37° C. Then an appropriate amount of the biotinylated labeled LOXL2 inhibitor (Compound 1-1), such as 5, was added and was allowed to incubate with the LOXL2 for a suitable amount of time at an appropriate temperature, such as 1 hr at 37° C. Then the biotinylated labeled LOXL2 inhibitor (Compound 1-1)/LOXL2 complex was isolated with Streptavidin coated magnetic beads. After incubation with the streptavidin magnetic beads for an appropriate amount of time, a magnet (for example, DynaMag™-2 Magnet) was used to capture the biotinylated labeled LOXL2 inhibitor (Compound 1-1)/LOXL2 bead bound complex and the supernatant was removed. The biotinylated labeled LOXL2 inhibitor (Compound 1-1)/LOXL2 bead bound complex is then washed with the appropriate solutions and the biotinylated labeled LOXL2 inhibitor (Compound 1-1)/LOXL2 complex was then eluted from the beads using 1× BOLT® LDS Sample Buffer (Thermo Fisher, Waltham, Mass.) containing 1× BOLT® Sample Reducing Agent (Thermo Fisher, Waltham, Mass.) and incubation at 90° C. for 10 minutes.

A Western Blot is then performed with an anti-LOXL2 antibody to quantify the amount of LOXL2 bound to the biotinylated LOXL2 inhibitor (Compound 1-1).

Pre-incubation with the LOXL2 inhibitor 1-13a was observed to reduce the amount of unbound/free LOXL2 captured by the biotinylated LOXL2 inhibitor by 70-100%.

### Example B-5

### LOXL2 Detection in Biological Fluids with LOXL2 Inhibitor-Coupled Ferrite Beads

### Method 1: Western-Based Magnetic Bead Assay for Determination of Free/Unbound LOXL2.

Coupled FG-beads 3-((4-(Aminomethyl)-6-(trifluoromethyl)pyridin-2-yl)oxy)-N-(21-(FG-bead)-10,17,21-trihydroxy-7-oxo-12,15-dioxa-3,4-dithia-8,19-diazahenicosyl)benzamide (Example 7; Compound 1-7) at 10 mg/mL in 50% methanol are centrifuged at 15,000×g for 5 minutes at room temperature and the water aspirated. Beads are resuspended to 10 mg/mL in Wash Buffer (20 mM Tris pH 8, 0.2% NP-40, 120 mM NaCl) and 1 mg beads (100 μL) are added to 1-10 mL of plasma, serum or other biological fluid containing LOXL2 and incubated at 37° C. for 0.5-2 hours with rotation. Beads are separated from plasma or other matrix using magnetic separation (for example, DynaMag™-15 Magnet) for 10 minutes at room temperature then the plasma/matrix carefully removed. The beads are resuspended in 1 mL Wash Buffer, vortexed and centrifuged at 4500×g for 5 minutes at room temperature to move all beads to the bottom of the tube. Beads are transferred to a 1.5 mL microfuge tube and washed 3 times with Wash Buffer using magnetic separation (for example, DynaMag™-2 Magnet). After the final wash, beads are resuspended in 40 μL of 1× BOLT® LDS Sample Buffer (Thermo Fisher, Waltham, Mass.) containing 1× BOLT® Sample Reducing Agent (Thermo Fisher, Waltham, Mass.) and incubated for 10 minutes at 90° C. Samples are centrifuged at 10,000×g for 1 minute then magnetically separated using a DynaMag™-2 Magnet for 2 minutes. 40 μL of each supernatant is loaded into one well of a 10 well 4-12% Bis-Tris Plus gel (Thermo Fisher, Waltham, Mass.) and the gels run for 45 minutes at 164 volts in 1×MES SDS Running Buffer (Thermo Fisher, Waltham, Mass.). Gels are transferred to nitrocellulose using iBlot (Thermo Fisher, Waltham, Mass.) according to the manufacturer's recommendation. Blots are incubated in Bullet Blocking One (Nacalai USA, Inc) for 5 minutes at room temperature then incubated overnight at 4° C. with an anti-LOXL2 antibody (for example, R&D Systems goat polyclonal) diluted in Signal Enhancer Solution 1 (Nacalai USA, Inc). Blots are rinsed quickly twice in PBS/0.1% tween-20 then washed 3 times for 10 minutes each in the PBS/tween solution. A secondary infrared dye (IRdye)-conjugated secondary antibody (LI-COR Biosciences, Lincoln, Neb.) diluted in Signal Enhancer 2 (Nacalai USA, Inc) is added to the blot and incubated for 45 minutes-1 hour at room temperature in the dark. Blots are washed as above with PBS/tween then rinsed in PBS before imaging with a LI-COR Odyssey (LI-COR, Lincoln, Neb.).

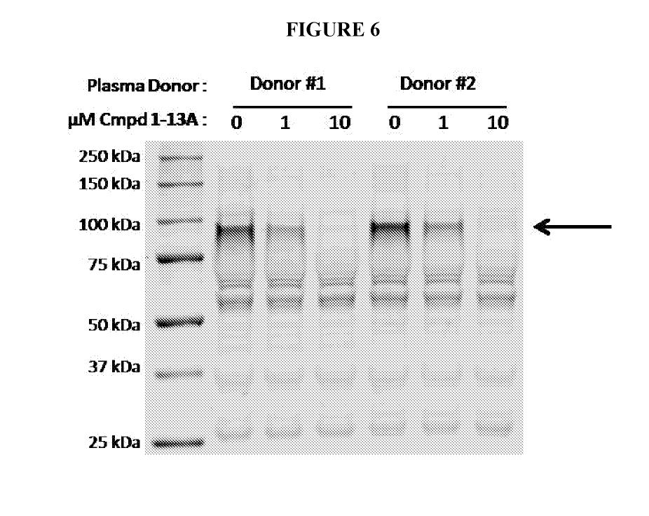

When 10 mL of plasma from two different human donors was pre-incubated with either 1 μM or 10 μM Compound 1-13a for 2 hours at 37° C. prior to incubation with LOXL2 inhibitor-coupled FG beads, the amount of free/unbound LOXL2 was decreased approximately 63% and 100%, respectively, relative to the vehicle control (see FIG. 6).

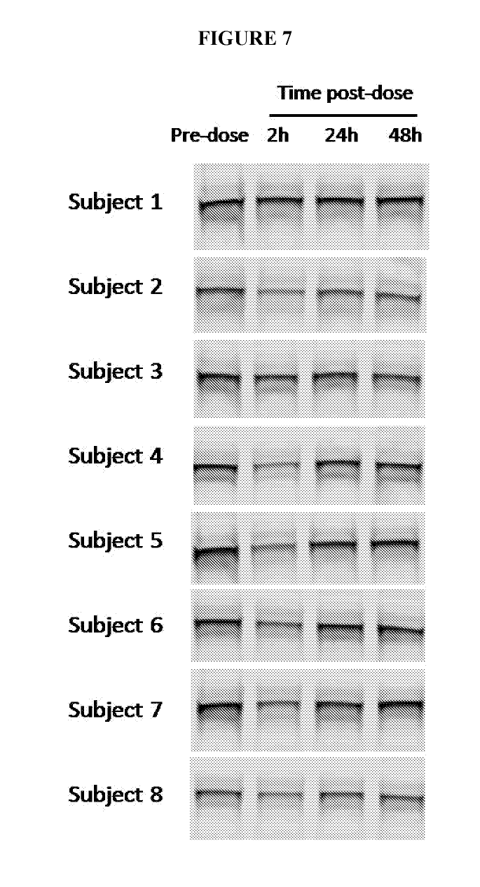

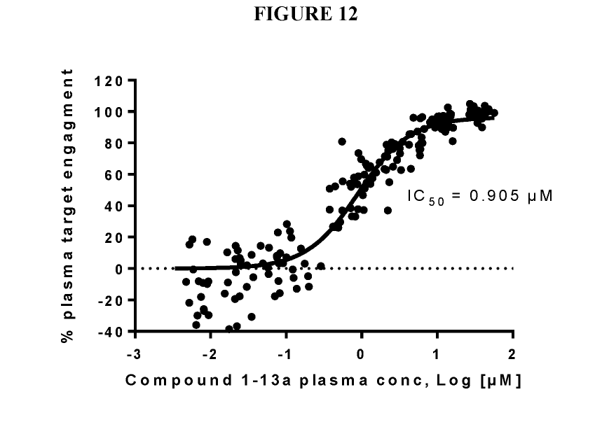

The amount of free/unbound LOXL2 was evaluated in 8 mL plasma from six healthy subjects at pre-dose or at 2, 24 or 48 hours after a single oral dose of Compound 1-13A and in two healthy subjects dosed with placebo. On average, the amount of free/unbound LOXL2 was decreased by approximately 60% at 2 hours post-dosing in the subjects receiving 150 mg of Compound 1-13a (see FIG. 7). Table 3 shows the relationship between the plasma concentration of compound 1-13a and target engagement at 2 and 24 hour following single rising doses of compound 1-13a administered to healthy volunteers. FIG. 12 shows the % target engagement versus plasma concentration relationship measured from plasma samples obtained following the administration of compound 1-13a to healthy volunteers. The IC₅₀ was determined to be 0.905 μM.

TABLE 3
Average human plasma drug concentrations and percent target engagement (TE) after a
single oral dose of Compound 1-13a.

| | Dose | | | | | | | |
|---|---|---|---|---|---|---|---|---|
| | 150 mg | | 450 mg | | 1000 mg | | Placebo | |
| | Time post-dose | | | | | | | |
| | 2 hour | 24 hour | 2 hour | 24 hour | 2 hour | 24 hour | 2 hour | 24 hour |
| Ave plasma | 966 | BLOQ | 4382 | 3.5 | 12855 | 8.0 | 0 | 0 |
| Cmpd 2 conc | | | | | | | | |
| (nM) | | | | | | | | |
| Ave plasma | 62% | 20% | 78% | −13% | 92% | 1% | 15% | 5% |
| % TE | | | | | | | | |

Healthy human subjects (male and female) were dosed with compound 2 in solution. Blood samples were drawn at regular intervals and the plasma isolated and analyzed for compound 1-13a concentration as well as LOXL2 target engagement. Placebo data is the average of all the placebo subjects in the single dose study (n=10).

### Method 2: Erenna-Based Fluorescence Magnetic Bead Assay for Determination of Free/Unbound LOXL2.

Plasma, serum or another biological fluid containing LOXL2 is incubated with 15-35 nM Compound 1-2 in a 96-well polypropylene V-bottom plate for 2-15 hours at room temperature with shaking. Following the incubation, 10 μg of streptavidin-coated magnetic microparticles (MPs) are added to each well and incubated for 30 min-2 hours at room temperature with shaking. The MPs are washed once using magnetic separation and 20 μL of a fluorescently conjugated anti-LOXL2 antibody is added to each well (for example, R&D Systems anti-LOXL2 Goat IgG polyclonal antibody conjugated to AlexaFluor 647) and incubated for 30 min-2 hours at room temperature with shaking. The MPs are washed four times with magnetic separation and transferred to a new plate. All liquid is aspirated from the plate and elution buffer added and incubated for 10 minutes at room temperature with shaking. The eluate is transferred to a 384 well assay plate containing neutralization buffer, sealed and read on Erenna®.

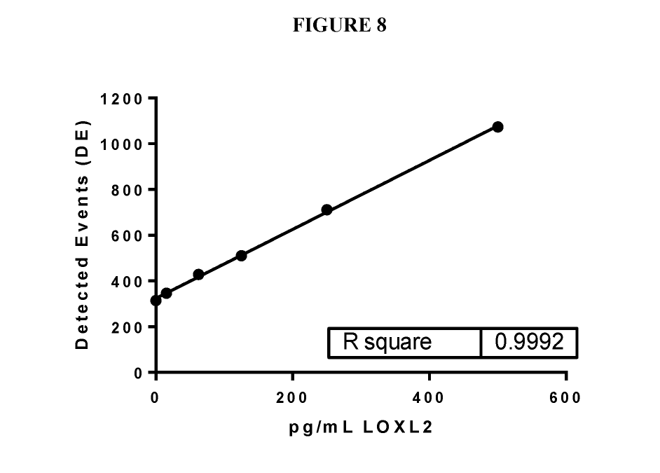

Various concentrations of LOXL2 were incubated with 34 nM Compound 1-2 for 15 hours at room temperature followed by incubation with streptavidin-coated magnetic microparticles and an AlexaFluor 647-coupled anti-LOXL2 antibody. There was a linear increase in signal of free/unbound LOXL2 up to 500 pg/mL (see FIG. 8).

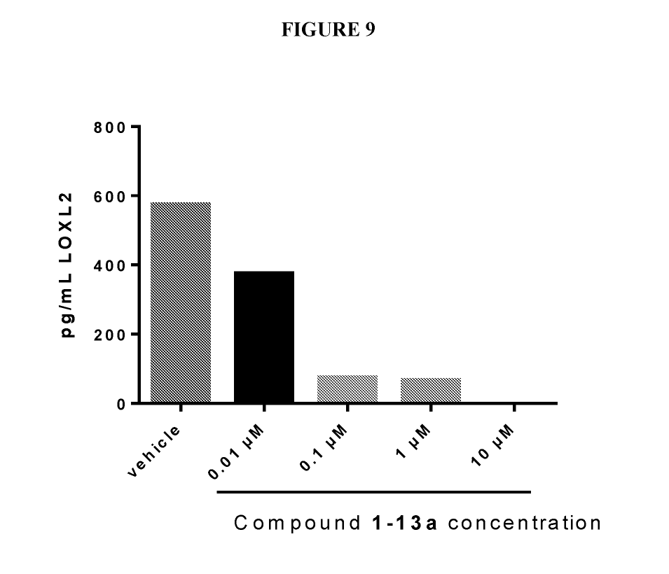

When 100 μL of a 500 pg/mL solution of LOXL2 was pre-incubated with increasing concentrations of Compound 1-13a, there was a concentration dependent decrease in the amount of free/unbound LOXL2 detected using Compound 1-2 combined with streptavidin-coated magnetic microparticles and an AlexaFluor 647-coupled anti-LOXL2 antibody (see FIG. 9).

### Example B-6

### Compound Tissue Distribution Studies with ¹⁸F-PET Imaging

In these studies, naïve or bleomycin-instilled mice were injected with 30-50 μCi of Compound 1-13 (¹⁸F-labeled) intravenously and were PET imaged for 90-120 minutes following injection of the radiolabel. These animals were then sacrificed at 2.5 hours post-administration of the ¹⁸F-labeled Compound 1-13 and tissues and blood were analyzed by scintillation counting to determine concentration of radioactivity remaining.

To determine specific binding, a subset of mice were administered a blocking dose of Compound 1-13a or vehicle (delivered at 60 mg/kg PO) at a suitable time point prior to the PET-ligand injection, such as 2.5-4 h prior.

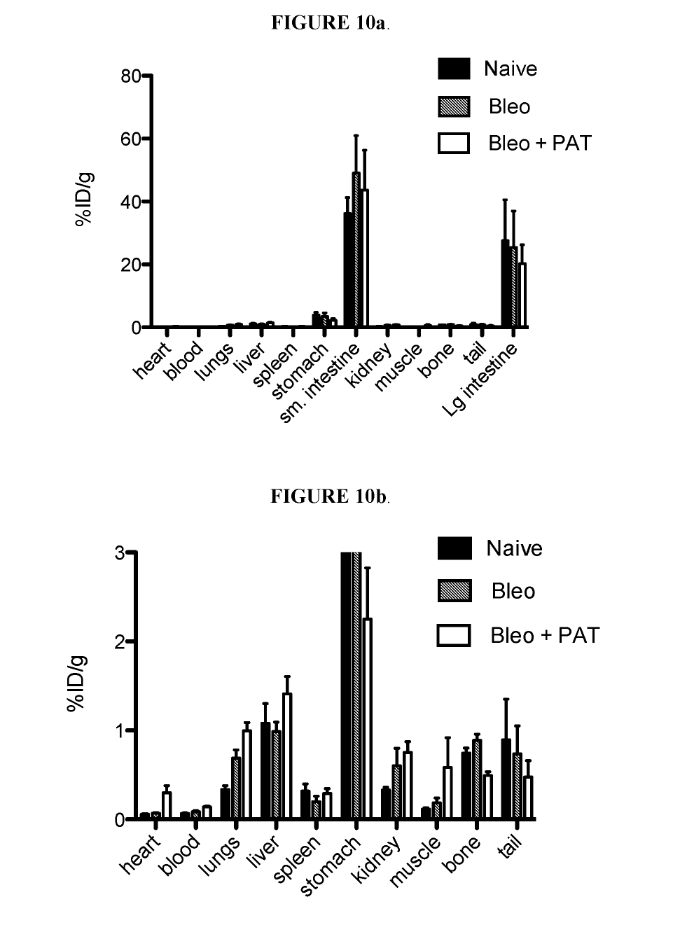

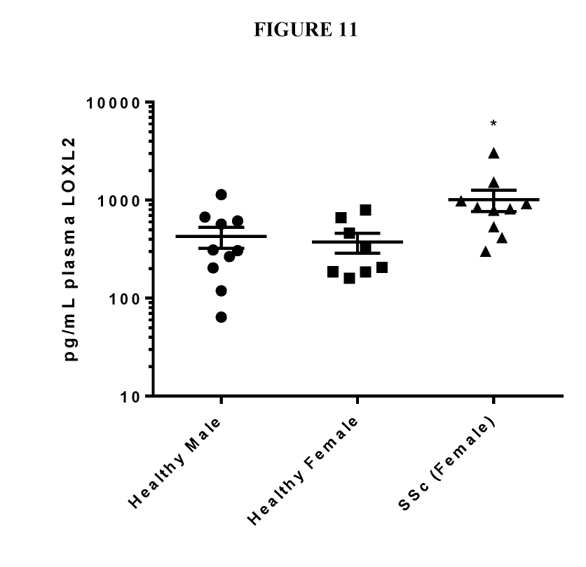

FIG. 10a shows the overall % injected dose (% ID)/g biodistribution data for all organs. FIG. 10b shows a closer view of the % injected dose (% ID)/g biodistribution data from FIG. 10a for specific organs.

Highest initial concentrations of ligand were observed in liver, kidney and lung.

The examples and embodiments described herein are for illustrative purposes only and various modifications or changes suggested to persons skilled in the art are to be included within the spirit and purview of this application and scope of the appended claims.

What is claimed is:

1. A probe compound comprising:
(a) a small molecule lysyl oxidase like-2 (LOXL2) inhibitor (LOXL2i);
(b) a tag moiety (Q) for the detection, isolation, or detection and isolation of the small molecule LOXL2i bound to LOXL2; and
(c) an optional linker (L) that separates the LOXL2 inhibitor from the tag moiety.

2. The probe compound of claim 1, wherein:
the small molecule LOXL2i is selective for LOXL2 versus lysyl oxidase (LOX).

3. The probe compound of claim 1 or claim 2, wherein:
the small molecule LOXL2i binds to the lysine tyrosylquinone (LTQ)-dependent amine oxidase of LOXL2.

4. The probe compound of any one of claims 1-3, wherein:
the small molecule LOXL2i is a substituted or unsubstituted heterocyclylmethylamine compound, or a substituted or unsubstituted arylmethylamine compound.

5. The probe compound of any one of claims 1-4, wherein:
the small molecule LOXL2i is a substituted or unsubstituted heteroarylmethylamine.

6. The probe compound of any one of claims 1-5, wherein:
the small molecule LOXL2i is a substituted or unsubstituted (monocyclic heteroaryl)methylamine.

7. The probe compound of any one of claims 1-6, wherein:
the small molecule LOXL2i is a substituted or unsubstituted (6-membered monocyclic heteroaryl)methylamine.

8. The probe compound of any one of claims 1-7, wherein:
the small molecule LOXL2i is a substituted or unsubstituted pyridinylmethylamine, substituted or unsubstituted pyrimidinylmethylamine, substituted or unsubstituted pyrazinylmethylamine, substituted or unsubstituted pyridazinylmethylamine, or substituted or unsubstituted triazinylmethylamine.

9. The probe compound of any one of claims 1-6, wherein:
the small molecule LOXL2i is a substituted or unsubstituted (5-membered monocyclic heteroaryl)methylamine.

10. The probe compound of any one of claims 1-6, wherein:
the small molecule LOXL2i is a substituted or unsubstituted (5-membered monocyclic heteroaryl)methylamine that is a substituted or unsubstituted imidazolylmethylamine, substituted or unsubstituted pyrazolylmethylamine, substituted or unsubstituted triazolylmethylamine, substituted or unsubstituted furylmethylamine, substituted or unsubstituted thienylmethylamine, substituted or unsubstituted isoxazolylmethylamine, substituted or unsubstituted thiazolylmethylamine, substituted or unsubstituted oxazolylmethylamine, substituted or unsubstituted isothiazolyl methylamine, substituted or unsubstituted pyrrolylmethylamine, substituted or unsubstituted oxadiazolylmethylamine, substituted or unsubstituted thiadiazolyl methylamine, or substituted or unsubstituted furazanylmethylamine.

11. The probe compound of any one of claims 1-5, wherein:
the small molecule LOXL2i is a substituted or unsubstituted (bicyclic heteroaryl)methylamine.

12. The probe compound of any one of claims 1-5, wherein:
the small molecule LOXL2i is a substituted or unsubstituted (bicyclic heteroaryl)methylamine that is a is substituted or unsubstituted indolizinylmethylamine, substituted or unsubstituted indolylmethylamine, substituted or unsubstituted benzofuranylmethylamine, substituted or unsubstituted benzothiophenylmethylamine, substituted or unsubstituted indazolylmethylamine, benzimidazolylmethylamine, substituted or unsubstituted purinylmethylamine, substituted or unsubstituted quinolizinylmethylamine, substituted or unsubstituted quinolinylmethylamine, substituted or unsubstituted isoquinolinylmethylamine, substituted or unsubstituted cinnolinylmethylamine, substituted or unsubstituted phthalazinylmethylamine, substituted or unsubstituted quinazolinylmethylamine, substituted or unsubstituted quinoxalinylmethylamine, substituted or unsubstituted 1,8-naphthyridinylmethylamine, or substituted or unsubstituted pteridinylmethylamine.

13. The probe compound of any one of claims 1-4, wherein:
the small molecule LOXL2i is a substituted or unsubstituted bicyclic heterocyclylmethylamine that is a substituted or unsubstituted quinolinonylmethylamine, substituted or unsubstituted isoquinolinonylmethylamine, substituted or unsubstituted chromonylmethylamine, or substituted or unsubstituted coumarinylmethylamine.

14. The probe compound of any one of claims 1-13, wherein:
the tag moiety (Q) for the detection, isolation, or detection and isolation of the small molecule LOXL2i bound to LOXL2 is: a solid support, a reporter group, a tag used for affinity purification, a tag used for sorting or immobilizing the compound of Formula (I) on a solid support, a hapten, a fluorescent moiety, radioactive moiety, magnetic resonance imaging (MRI) moiety, colorometric moiety, luminescent moiety, bioluminescent moiety, chemiluminescent moiety, oligonucleotide or combination thereof.

15. A compound that has the following structure of Formula (I): 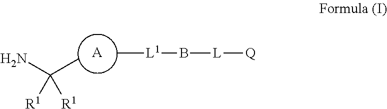
wherein,
Q is a tag moiety for the detection, isolation, or detection and isolation of the compound of Formula (I) in a biological sample;
or Q is absent provided that the compound of Formula (I) comprises a radioactive or an isotopic variant of any atom in the compound of Formula (I);
L is absent or a linker;
each R¹ is independently H, D, or F;
ring A is an unsubstituted or substituted aryl, or an unsubstituted or substituted heterocycle, wherein if ring A is substituted then ring A is substituted with 1, 2, or 3 R^{a} groups;
L¹ is X¹—Y¹—, —Y¹—X¹—, or Y¹;
X¹ is —O—, —S—, —S(═O)—, —S(═O)₂—, —C(═O)—, —C(═O)O—, —C(═O)NR²—, —NR²C(═O)—, or —NR²—;
R² is H, C₁-C₆alkyl, C₁-C₆fluoroalkyl, or C₁-C₆deuteroalkyl;
Y¹ is absent, or C₁-C₆alkylene;
B is absent or an unsubstituted or substituted monocyclic carbocycle, unsubstituted or substituted bicyclic carbocycle, unsubstituted or substituted monocyclic heterocycle, or unsubstituted or substituted bicyclic heterocycle, wherein if B is substituted then B is substituted with one or more R^{b};
each R^{a}, and R^{b} is independently selected from the group consisting of H, D, halogen, CN, —OR⁵, —SR⁵, —S(═O)R⁴, —S(═O)₂R⁴, —S(═O)₂N(R⁵)₂, —NR⁵S(═O)₂R⁴, —C(═O)R⁴, —OC(═O)R⁴, —CO₂R⁵, —OCO₂R⁴, N(R⁴)₂, —OC(═O)N(R⁵)₂, —C(═O)N(R⁵)₂, —NHC(═O)R⁴, —NHC(═O)OR⁴, C₁-C₆alkyl, C₁-C₆fluoroalkyl, C₁-C₆deuteroalkyl, C₁-C₆heteroalkyl, substituted or unsubstituted C₃-C₁₀cycloalkyl, substituted or unsubstituted C₂-C₁₀heterocycloalkyl, substituted or unsubstituted aryl, and substituted or unsubstituted heteroaryl;
each R⁴ is independently selected from C₁-C₆alkyl, C₁-C₆fluoroalkyl, C₁-C₆deuteroalkyl, C₁-C₆heteroalkyl, substituted or unsubstituted C₃-C₁₀cycloalkyl, substituted or unsubstituted C₂-C₁₀heterocycloalkyl, substituted or unsubstituted aryl, and substituted or unsubstituted heteroaryl;
each R⁵ is independently selected from H, C₁-C₆alkyl, C₁-C₆fluoroalkyl, C₁-C₆deuteroalkyl, C₁-C₆heteroalkyl, substituted or unsubstituted C₃-C₁₀cycloalkyl, substituted or unsubstituted C₂-C₁₀heterocycloalkyl, substituted or unsubstituted aryl, substituted or unsubstituted heteroaryl, —C₁-C₄alkylene-(substituted or unsubstituted C₃-C₈cycloalkyl), substituted or unsubstituted C₂-C₈heterocycloalkyl, —C₁-C₄alkylene-(substituted or unsubstituted C₂-C₈heterocycloalkyl), substituted or unsubstituted aryl, —C₁-C₄alkylene-(substituted or unsubstituted aryl), substituted or unsubstituted heteroaryl, and —C₁-C₄alkylene-(substituted or unsubstituted heteroaryl);
or two R⁵ on the same N atom are taken together with the N atom to which they are attached to a substituted or unsubstituted N-containing heterocycle.

16. The compound of claim 15, wherein:
L is absent or a linker with the formula -L²-C-L³-;
L² is X²—Y²—, —Y²—X²—, or Y²;
X² is —O—, —S—, —S(═O)—, —S(═O)₂—, —S(═O)₂NR³—, —C(═O)—, —C(═O)O—, —OC(═O)—, —C(═O)NR³—, —NR³C(═O)—, —NR³S(═O)₂—, or —NR³—;
R³ is H, C₁-C₆alkyl, C₁-C₆fluoroalkyl, or C₁-C₆deuteroalkyl;
Y² is absent, or C₁-C₆alkylene;
C is absent, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₈cycloalkyl, —C₁-C₄alkylene-(substituted or unsubstituted C₃-C₈cycloalkyl), substituted or unsubstituted C₂-C₈heterocycloalkyl, —C₁-C₄alkylene-(substituted or unsubstituted C₂-C₈heterocycloalkyl), substituted or unsubstituted aryl, —C₁-C₄alkylene-(substituted or unsubstituted aryl), substituted or unsubstituted heteroaryl, or —C₁-C₄alkylene-(substituted or unsubstituted heteroaryl), wherein if C is substituted then C is substituted with one or more R^{c};
or when C and R³ are attached to the same N-atom then C and R³ are taken together with the N atom to which they are attached to form ring D, wherein ring D is a substituted or unsubstituted N-containing heterocycle, wherein if ring D is substituted then ring D is substituted with 1, 2, or 3 R^{d};
each R^{c}, and R^{d}, is independently selected from the group consisting of D, halogen, CN, —OR⁵, —SR⁵, —S(═O)R⁴, —S(═O)₂R⁴, —S(═O)₂N(R⁵)₂, NR⁵S(═O)₂R⁴, —C(═O)R⁴, —OC(═O)R⁴, —CO₂R⁵, —OCO₂R⁴, —N(R⁴)₂, —OC(═O)N(R⁵)₂, —C(═O)N(R⁵)₂, —NHC(═O)R⁴, —NHC(═O)OR⁴, C₁-C₆alkyl, C₁-C₆fluoroalkyl, C₁-C₆deuteroalkyl, C₁-C₆heteroalkyl, substituted or unsubstituted C₃-C₁₀cycloalkyl, substituted or unsubstituted C₂-C₁₀heterocycloalkyl, substituted or unsubstituted aryl, and substituted or unsubstituted heteroaryl;
or two R^{d} groups attached to the same carbon atom are taken together with the carbon atom to which they are attached to form either a substituted or unsubstituted carbocycle or substituted or unsubstituted heterocycle;
L³ is absent or -L⁴-L⁵-L⁶-L⁷-;
L⁴ is absent, —O—, —S—, —S(═O)—, —S(═O)_{2—}, —NR⁴—, —CH(OH)—, —C(═O)—, —C(═O)NH—, —NHC(═O)—, —C(═O)O—, —OC(═O)—, —CH(═N)—, —CH(═N—NH)—, —CCH₃(═N)—, —CCH₃(═N—NH)—, —OC(═O)NH—, —NHC(═O)NH—, —NHC(═O)O—, —(CH₂)ₚ—, —(OCH₂CH₂)ₚ—, or —(OCH₂CH₂)ₚ—, p is 1, 2, 3, 4, 5, or 6;
L⁵ is absent, unsubstituted or substituted alkylene, unsubstituted or substituted heteroalkylene, unsubstituted or substituted alkenylene, unsubstituted or substituted alkynylene, unsubstituted or substituted cycloalkylene, unsubstituted or substituted heterocycloalkylene, unsubstituted or substituted arylene, unsubstituted or substituted heteroarylene, —(OCH₂CH₂)ₚ—, or —(OCH₂CH₂)ₚ—, p is 1, 2, 3, 4, 5, 6, 7, 8, 9, 10, 11, or 12;
L⁶ is absent, —O—, —S—, —S(O)—, —S(O)_{2—}, —NR⁴—, —CH(OH)—, —C(═O)—, —C(═O)NH—, —NHC(═O)—, —C(═O)O—, —OC(═O)—, —OC(═O)NH—, —NHC(═O)NH—, or —NHC(═O)O—;
L⁷ is absent, unsubstituted or substituted alkylene, unsubstituted or substituted heteroalkylene.

17. The compound of claim 15 or claim 16, wherein:
each R¹ is H;
each R^{a} is independently selected from the group consisting of H, D, F, Cl, Br, —CN, —OR⁵, —CO₂R⁵, —N(R⁵)₂, —NR²C(═O)R⁴, —CH₃, —CH₂CH₃, —CH(CH₃)₂, —C(CH₃)₃, —CH₂F, —CHF₂, —CF₃, cyclopropyl, cyclobutyl, cyclopentyl, cyclohexyl, substituted or unsubstituted monocyclic C₂-C₆heterocycloalkyl, substituted or unsubstituted phenyl, and substituted or unsubstituted monocyclic heteroaryl.

18. The compound of any one of claims 15-17, wherein:
each R^{a} is independently selected from the group consisting of H, D, F, Cl, Br, —CN, —OCH₃, —OCF₃, —CH₃, —CH₂F, —CHF₂, —CF₃, cyclopropyl, cyclobutyl, cyclopentyl, cyclohexyl, substituted or unsubstituted monocyclic C₂-C₆heterocycloalkyl, substituted or unsubstituted phenyl, or substituted or unsubstituted monocyclic heteroaryl.

19. The compound of any one of claims 15-18, wherein:
each R^{a} is independently selected from the group consisting of H, D, F, Cl, Br, —CN, —OCH₃, —OCF₃, —CH₃, —CH₂F, —CHF₂, and —CF₃.

20. The compound of any one of claims 15-19, wherein:
each R^{a} is independently selected from the group consisting of H, D, and —CF₃.

21. The compound of any one of claims 15-20, wherein:
ring A is an unsubstituted or substituted monocyclic aromatic heterocycle, wherein if ring A is substituted then ring A is substituted with 1, 2, or 3 R^{a} groups.

22. The compound of any one of claims 15-21, wherein:
ring A is an unsubstituted or substituted monocyclic aromatic 6-membered heterocycle or an unsubstituted or substituted monocyclic aromatic 5-membered heterocycle, wherein if ring A is substituted then ring A is substituted with 1, 2, or 3 R^{a} groups.

23. The compound of any one of claims 15-22, wherein:
ring A is an unsubstituted or substituted pyridinyl, an unsubstituted or substituted pyrimidinyl, an unsubstituted or substituted pyrazinyl, an unsubstituted or substituted pyridazinyl, or an unsubstituted or substituted triazinyl, wherein if ring A is substituted then ring A is substituted with 1, 2, or 3 R^{a} groups.

24. The compound of any one of claims 15-23, wherein the compound of Formula (I) has the structure of Formula (II) or Formula (III):

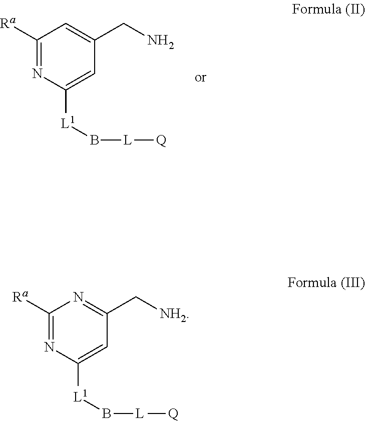

25. The compound of any one of claims 15-24, wherein the compound of Formula (I) has the structure of Formula (IIa):

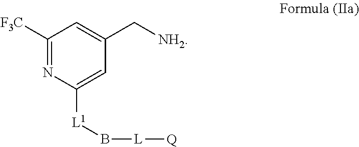

26. The compound of any one of claims 15-22, wherein:
ring A is an unsubstituted or substituted monocyclic aromatic 5-membered heterocycle that is an unsubstituted or substituted imidazolyl, an unsubstituted or substituted pyrazolyl, an unsubstituted or substituted triazolyl, an unsubstituted or substituted tetrazolyl, an unsubstituted or substituted furyl, an unsubstituted or substituted thienyl, an unsubstituted or substituted isoxazolyl, an unsubstituted or substituted thiazolyl, an unsubstituted or substituted oxazolyl, an unsubstituted or substituted isothiazolyl, an unsubstituted or substituted pyrrolyl, an unsubstituted or substituted oxadiazolyl, an unsubstituted or substituted thiadiazolyl, or an unsubstituted or substituted furazanyl.

27. The compound of any one of claims 15-20, wherein:
ring A is an unsubstituted or substituted bicyclic heterocycle.

28. The compound of any one of claims 15-20, wherein:
ring A is an unsubstituted or substituted quinolinone, unsubstituted or substituted isoquinolinone, unsubstituted or substituted chromone, or unsubstituted or substituted coumarin.

29. The compound of any one of claims 15-20, wherein the compound of Formula (I) has the structure of Formula (IV), Formula (V), Formula (VI), Formula (VII), or Formula (VIII):

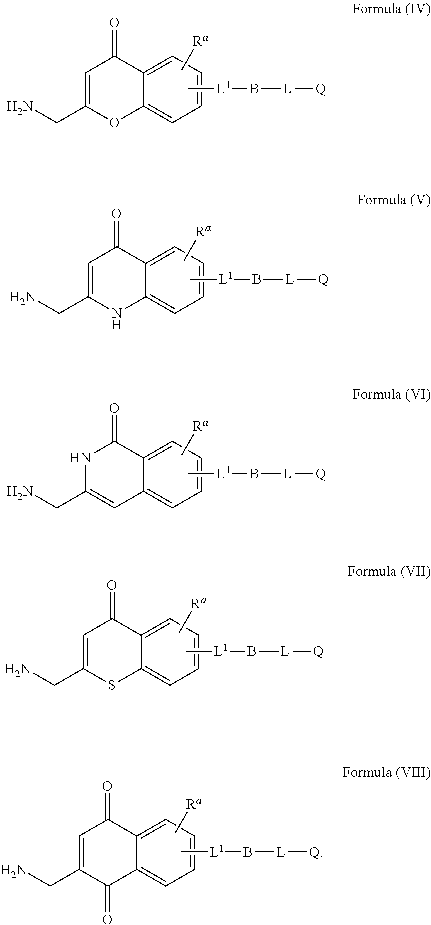

30. The compound of any one of claims 15-20, wherein:
ring A is an unsubstituted or substituted indolizinyl, unsubstituted or substituted indolyl, unsubstituted or substituted benzofuranyl, unsubstituted or substituted benzothiophenyl, unsubstituted or substituted indazolyl, unsubstituted or substituted benzimidazolyl, unsubstituted or substituted purinyl, unsubstituted or substituted quinolizinyl, unsubstituted or substituted quinolinyl, unsubstituted or substituted isoquinolinyl, unsubstituted or substituted cinnolinyl, unsubstituted or substituted phthalazinyl, unsubstituted or substituted quinazolinyl, unsubstituted or substituted quinoxalinyl, unsubstituted or substituted 1,8-naphthyridinyl, or unsubstituted or substituted pteridinyl.

31. The compound of any one of claims 15-20, wherein the compound of Formula (I) has the structure of Formula (IX), Formula (X), Formula (XI), or Formula (XII):

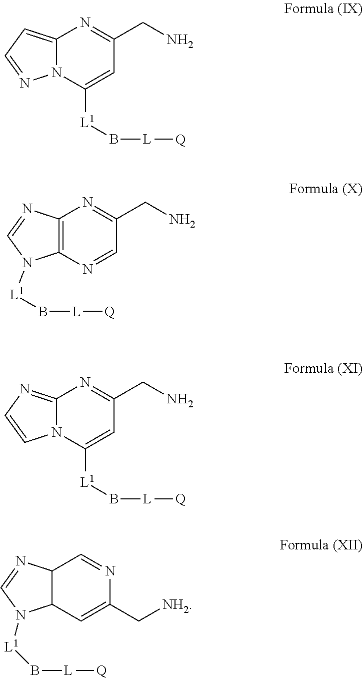

32. The compound of any one of claims 15-20, wherein:
ring A is an unsubstituted or substituted phenyl, or an unsubstituted or substituted naphthyl.

33. The compound of any one of claims 15-32, wherein:
L¹ is absent, X¹, or X¹—C₁-C₆alkylene.

34. The compound of any one of claims 15-33, wherein:
L¹ is absent, —O—, —O—CH₂—, —C(═O)—, —C(═O)NHCH₂—, —NHC(═O)—, —NHC(═O)CH₂—, or —NH—.

35. The compound of any one of claims 15-34, wherein:
L¹ is —O—, or —O—CH₂—.

36. The compound of any one of claims 15-35, wherein:
L¹ is —O—.

37. The compound of any one of claims 15-36, wherein:
B is monocyclic C₃-C₆carbocycle, bicyclic C₆-C₁₂carbocycle, monocyclic C₁-C₅heterocycle, bicyclic C₅-C₁₀heterocycle.

38. The compound of any one of claims 15-37, wherein:
B is phenyl.

39. The compound of any one of claims 15-38, wherein:
B is

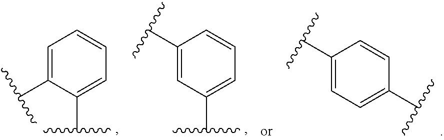

40. The compound of any one of claims 15-37, wherein:
B is bicyclic C₉-C₁₀carbocycle that is naphthyl, indanyl, indenyl, or tetrahyodronaphthyl.

41. The compound of any one of claims 15-37, wherein:
B is a monocyclic heterocycle containing 1-4 N atoms and 0 or 1 O or S atom, monocyclic heterocycle containing 0-4 N atoms and 1 O or S atoms, bicyclic heterocycle containing 1-4 N atoms and 0 or 1 O or S atoms, or bicyclic heterocycle containing 0-4 N atoms and 1 O or S atoms.

42. The compound of any one of claims 15-37, wherein:
B is pyrrolidinyl, pyrrolidinonyl, tetrahydrofuranyl, tetrahydrofuranonyl, dihydrofuranonyl, dihydrofuranyl, tetrahydrothienyl, oxazolidinonyl, tetrahydropyranyl, dihydropyranyl, tetrahydrothiopyranyl, piperidinyl, morpholinyl, thiomorpholinyl, piperazinyl, aziridinyl, azetidinyl, oxetanyl, thietanyl, homopiperidinyl, oxepanyl, thiepanyl, oxazepinyl, diazepinyl, thiazepinyl, 1,2,3,6-tetrahydropyridinyl, indolinyl, indolinonyl, 1,2,3,4-tetrahydroquinolinyl, 1,2,3,4-tetrahydroisoquinolinyl, 3,4-dihydro-2(1H)-quinolinonyl, furanyl, pyrrolyl, oxazolyl, thiazolyl, imidazolyl, pyrazolyl, triazolyl, tetrazolyl, isoxazolyl, isothiazolyl, oxadiazolyl, thiadiazolyl, pyridinyl, pyrimidinyl, pyrazinyl, pyridazinyl, triazinyl, quinolinyl, isoquinolinyl, quinazolinyl, quinoxalinyl, naphthyridinyl, indolyl, indazolyl, benzoxazolyl, benzisoxazolyl, benzofuranyl, benzothienyl, benzothiazolyl, benzimidazolyl, purinyl, cinnolinyl, phthalazinyl, pteridinyl, pyridopyrimidinyl, pyrazolopyrimidinyl, or azaindolyl.

43. The compound of any one of claims 15-37, wherein:
B is

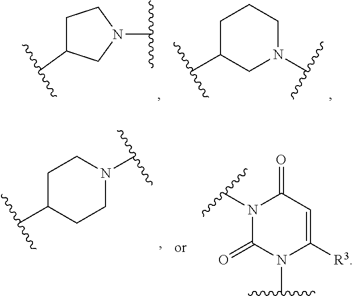

44. The compound of any one of claims 15-37, wherein:
B is

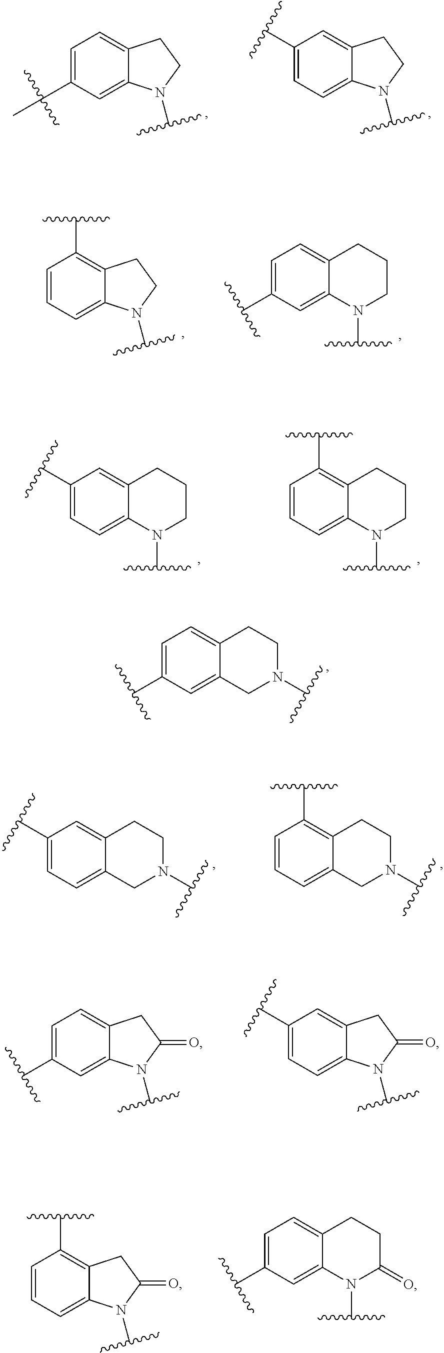

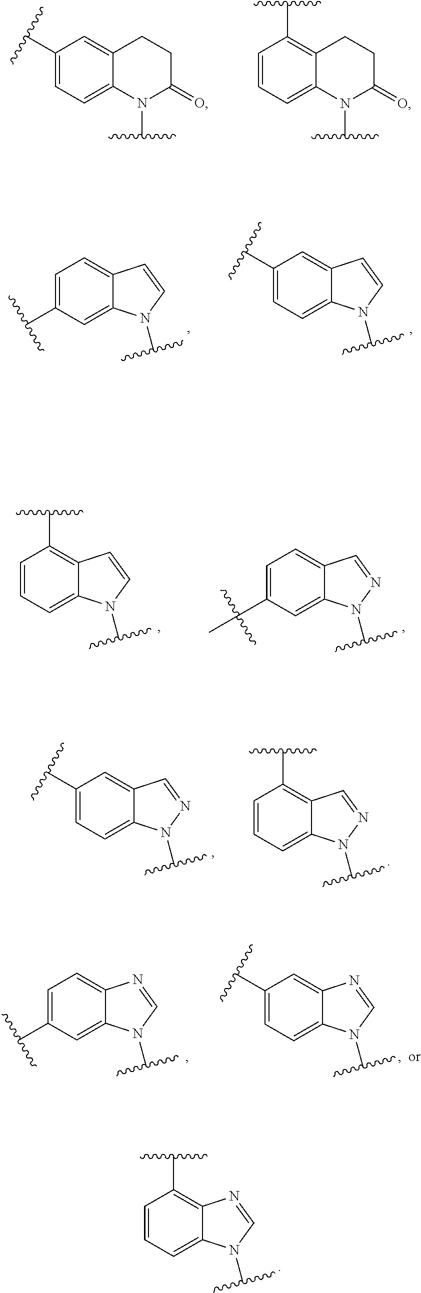

45. The compound of any one of claims 15-38, wherein the compound of Formula (I) has the structure of Formula (IIb):

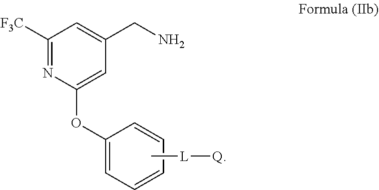

46. The compound of claim 45, wherein the compound of Formula (I) has the following structure of Formula (IIc):

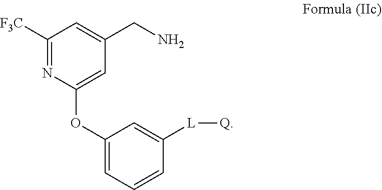

47. The compound of any one of claims 16-46, wherein:
L is -L²-C-L³-;
L² is —X²—Y²—, —Y²—X²—, or Y²;
X² is —O—, —S(═O)₂NR³—, —C(═O)—, —C(═O)NR³—, —NR³C(═O)—, —NR³S(═O)₂—, or —NR³—;
R³ is H, C₁-C₆alkyl, C₁-C₆fluoroalkyl, or C₁-C₆deuteroalkyl;
Y² is absent, or C₁-C₆alkylene;
C is absent, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₈cycloalkyl, —C₁-C₄alkylene-(substituted or unsubstituted C₃-C₈cycloalkyl), substituted or unsubstituted C₂-C₈heterocycloalkyl, —C₁-C₄alkylene-(substituted or unsubstituted C₂-C₈heterocycloalkyl), substituted or unsubstituted aryl, —C₁-C₄alkylene-(substituted or unsubstituted aryl), substituted or unsubstituted heteroaryl, or —C₁-C₄alkylene-(substituted or unsubstituted heteroaryl), wherein if C is substituted then C is substituted with one or more R^{c};
or when C and R³ are on the same N atom then C and R³ are taken together with the N atom to which they are attached to form ring D, wherein ring D is a substituted or unsubstituted monocyclic N-containing heterocycle, or a substituted or unsubstituted bicyclic N-containing heterocycle, wherein if ring D is substituted then ring D is substituted with 1, 2, or 3 R^{d}.

48. The compound of any one of claims 16-47, wherein:
L² is —X²—Y²—;
X² is —C(═O)NR³—;
Y² is absent, or C₁-C₆alkylene.

49. The compound of any one of claims 16-48, wherein:
C is absent, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₈cycloalkyl, —C₁-C₄alkylene-(substituted or unsubstituted C₃-C₈cycloalkyl), substituted or unsubstituted C₂-C₈heterocycloalkyl, —C₁-C₄alkylene-(substituted or unsubstituted C₂-C₈heterocycloalkyl), substituted or unsubstituted aryl, —C₁-C₄alkylene-(substituted or unsubstituted aryl), substituted or unsubstituted heteroaryl, or —C₁-C₄alkylene-(substituted or unsubstituted heteroaryl), wherein if C is substituted then C is substituted with one or more R^{c};
or when C and R³ are on the same N atom then C and R³ are taken together with the N atom to which they are attached to form ring D, wherein ring D is a substituted or unsubstituted aziridinyl, substituted or unsubstituted azetidinyl, substituted or unsubstituted pyrrolidinyl, substituted or unsubstituted pyrrolidinonyl, substituted or unsubstituted piperidinyl, substituted or unsubstituted piperidinonyl, substituted or unsubstituted morpholinyl, substituted or unsubstituted thiomorpholinyl, substituted or unsubstituted piperazinyl, substituted or unsubstituted piperazinonyl, substituted or unsubstituted indolinyl, substituted or unsubstituted indolinonyl, substituted or unsubstituted 1,2,3,4-tetrahydroquinolinyl, substituted or unsubstituted 1,2,3,4-tetrahydroisoquinolinyl, or substituted or unsubstituted 3,4-dihydro-2(1H)-quinolinonyl, wherein if ring D is substituted then ring D is substituted with 1, 2, or 3 R^{D}.

50. The compound of any one of claims 16-49, wherein the compound of Formula (I) has the following structure of Formula (IId):

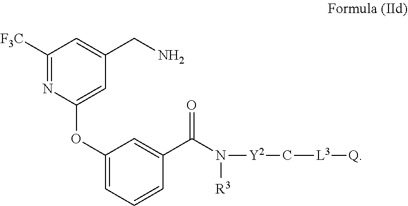

51. The compound of any one of claims 16-50, wherein:
L³ is absent or a linker that is -L⁴-L⁵-L⁶-L⁷-;
L⁴ is absent, —O—, —S—, —S(═O)—, —S(═O)_{2—}, —NR⁴—, —CH(OH)—, —C(═O)—, —C(═O)NH—, —NHC(═O)—, —C(═O)O—, —OC(═O)—, —CH(═N)—, —CH(═N—NH)—, —CCH₃(═N)—, —CCH₃(═N—NH)—, —OC(═O)NH—, —NHC(═O)NH—, —NHC(═O)O—, —(CH₂)ₚ—, —(OCH₂CH₂)ₚ—, or —(OCH₂CH₂)ₚ—, p is 1, 2, 3, 4, 5, or 6;
L⁵ is absent, unsubstituted or substituted alkylene, unsubstituted or substituted heteroalkylene, unsubstituted or substituted alkenylene, unsubstituted or substituted alkynylene, unsubstituted or substituted cycloalkylene, unsubstituted or substituted heterocycloalkylene, unsubstituted or substituted arylene, unsubstituted or substituted heteroarylene, —(OCH₂CH₂)ₚ—, or —(OCH₂CH₂)ₚ—, p is 1, 2, 3, 4, 5, 6, 7, 8, 9, 10, 11, or 12;
L⁶ is absent, —O—, —S—, —S(O)—, —S(O)_{2—}, —NR⁴—, —CH(OH)—, —C(═O)—, —C(═O)NH—, —NHC(═O)—, —C(═O)O—, —OC(═O)—, —OC(═O)NH—, —NHC(═O)NH—, or —NHC(═O)O—;
L⁷ is absent, unsubstituted or substituted alkylene, unsubstituted or substituted heteroalkylene.

52. The compound of any one of claims 15-51, wherein:
Q is a tag moiety for the detection, isolation, or detection and isolation of the compound of Formula (I) in a biological sample that is: a solid support, a reporter group, a tag used for affinity purification, a tag used for sorting or immobilizing the compound of Formula (I) on a solid support, a hapten, a fluorescent moiety, radioactive moiety, magnetic resonance imaging (MRI) moiety, colorometric moiety, luminescent moiety, bioluminescent moiety, chemiluminescent moiety, oligonucleotide or combination thereof;
or Q is absent provided that the compound of Formula (I) comprises a radioactive or an isotopic variant of any atom in the compound of Formula (I).

53. The compound of any one of claims 15-52, wherein:
Q is a tag used for affinity purification that is capable of specific binding to a protein to produce a tightly bound complex.

54. The compound of any one of claims 15-53, wherein:
Q is a tag that is capable of specific binding to avidin or streptavidin.

55. The compound of any one of claims 15-54, wherein:
Q is biotin or desthiobiotin.

56. The compound of any one of claims 15-55, wherein:
L is

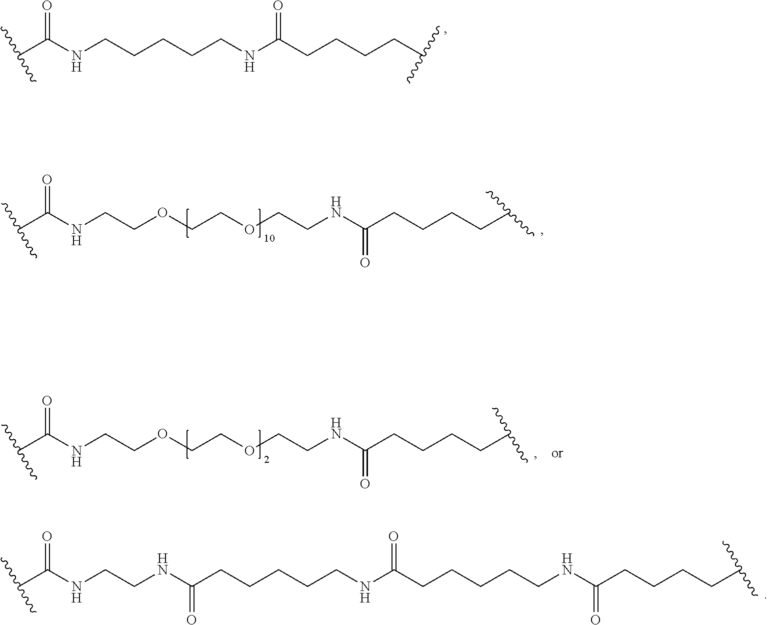

57. The compound of claim 55 or 56, wherein:
-L-Q is

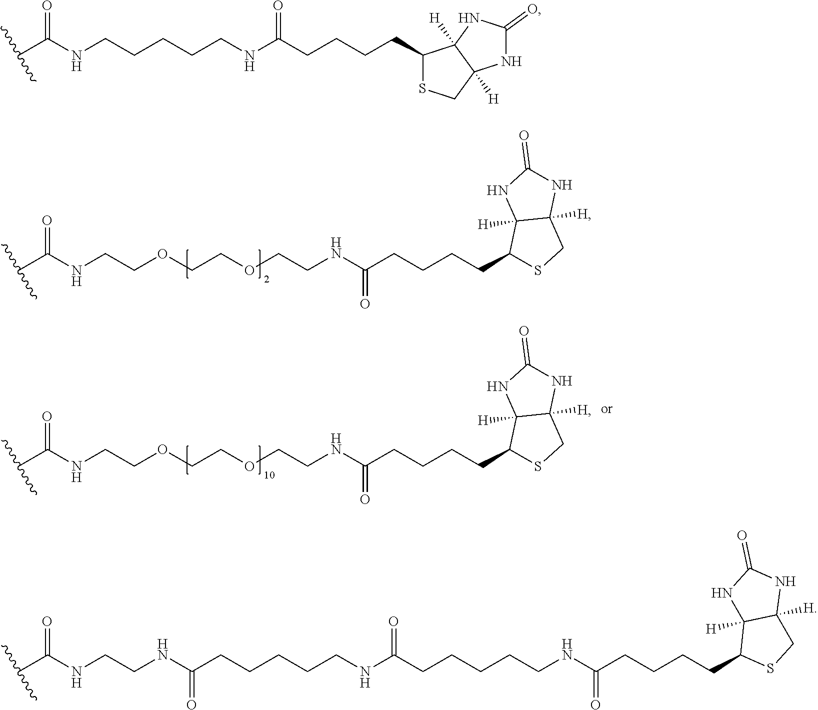

58. The compound of any one of claims 15-52, wherein:
Q is a hapten selected from biotin, a coumarin dye, a rhodamine dye, a xanthene dye, a cyanine dye, a BODIPY dye, a Lucifer yellow dye, digoxigenin, dansyl, or dintrophenyl.

59. The compound of any one of claims 15-52, wherein:
Q is a tag moiety that is: a fluorescent moiety, radioactive moiety, colorometric moiety, luminescent moiety, chemiluminescent moiety, or combination thereof.

60. The compound of any one of claims 15-52, wherein:
Q is a tag moiety that is a fluorescent moiety.

61. The compound of any one of claims 15-52, wherein:
Q is a tag moiety that is a fluorescent moiety selected from the group consisting of xanthene dyes, cyanine dyes, squaraine dyes, ring-substituted squaraine dyes, naphthalene dyes, coumarin dyes, oxadiazole dyes, anthracene dyes, oxazine dyes, acridine dyes, arylmethine dyes, BODIPY dyes, and tetrapyrrole dyes.

62. The compound of any one of claims 15-52, wherein:
Q is a fluorescent moiety selected from the group consisting fluorescein dyes, rhodamine dyes, Oregon green dyes, eosin dyes, Texas red dyes, cyanine dyes, indocarbocyanine dyes, oxacarbocyanine dyes, thiacarbocyanine dyes, merocyanine dyes, Seta, SeTau, Square dyes, dansyl dyes, prodan dyes, coumarin dyes, BODIPY dyes, pyridyloxazole dyes, nitrobenzoxadiazole dyes, benzoxadiazole dyes, DRAQ5, DRAQ7, CyTRAK Orange cascade blue, Nile red, Nile blue, cresyl violet, oxazine 170, proflavin dyes, acridine orange dyes, acridine yellow dyes, auramine dyes, crystal violet dyes, malachite green dyes, porphin dyes, phthalocyanine dyes, and bilirubin dyes.

63. The compound of any one of claims 15-52, wherein:
Q is xanthene, cyanine, squaraine, naphthalene, coumarin, oxadiazole, anthracene, pyrene, oxazine, acridine, arylmethine, tetrapyrrole, dansyl, BODIPY.

64. The compound of any one of claims 15-52, wherein:
Q is cyanine, coumarin, or dansyl.

65. The compound of any one of claims 15-52, wherein:
Q is xanthene, cyanine 2, cyanine 3, cyanine 3B, cyanine 3.5, cyanine 5, cyanine 5.5, cyanine7, squaraine, naphthalene, coumarin, oxadiazole, anthracene, pyrene, oxazine, acridine, arylmethine, tetrapyrrole, dansyl, BODIPY FL, BODIPY R6G, BODIPY TMR, BODIPY 581/591, BODIPY TR, BODIPY 630/650, or BODIPY 650/665.

66. The compound of any one of claims 58-65, wherein:
-L- is —NH—, —C(═O)NH—,

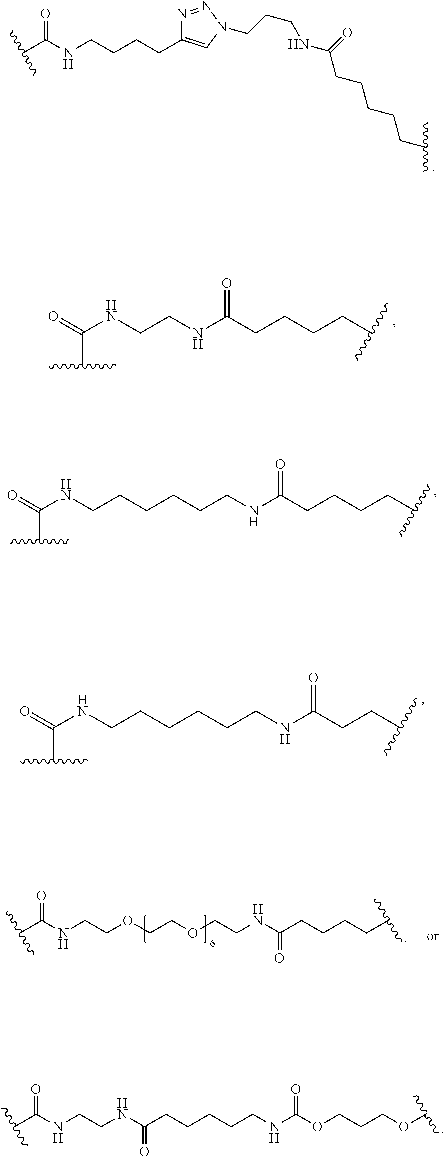

67. The compound of claim 66, wherein:
Q is

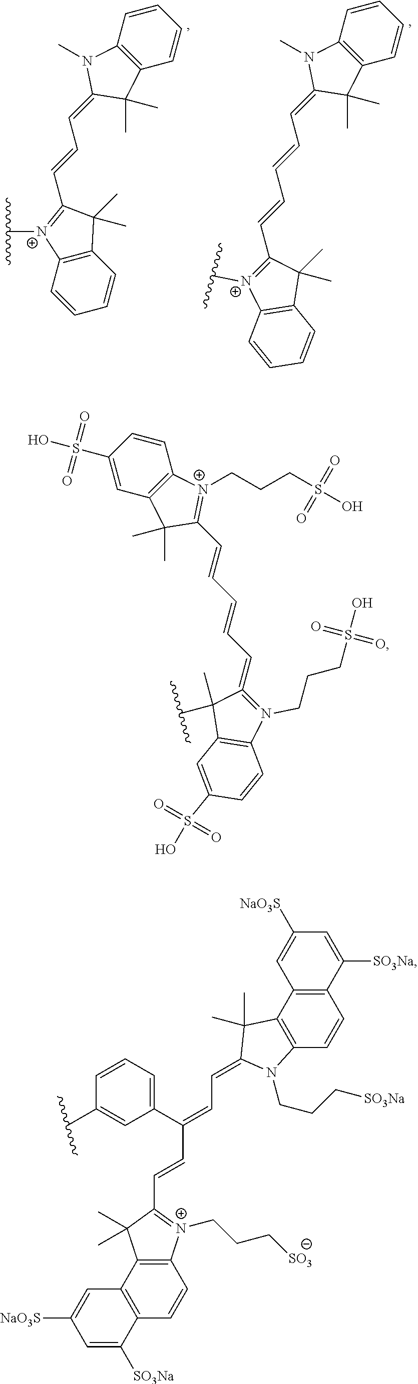

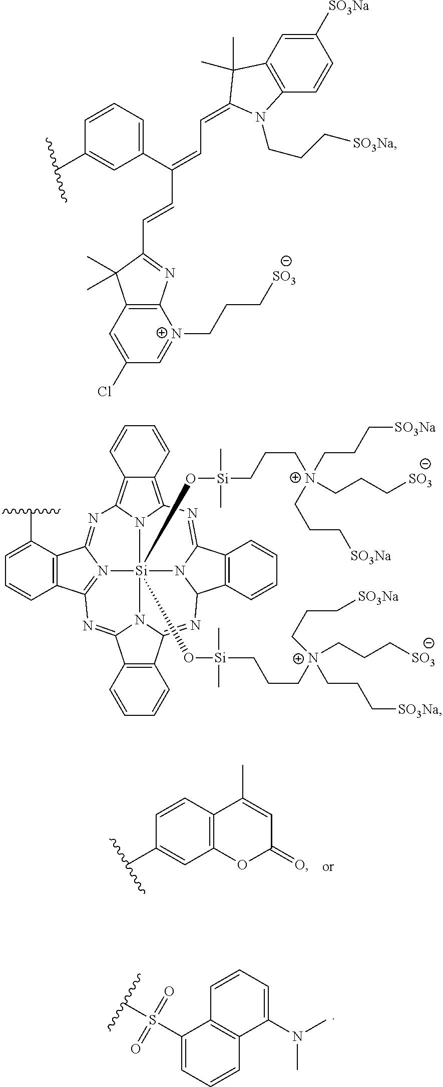

68. The compound of claim 66 or 67, wherein:
-L-Q is

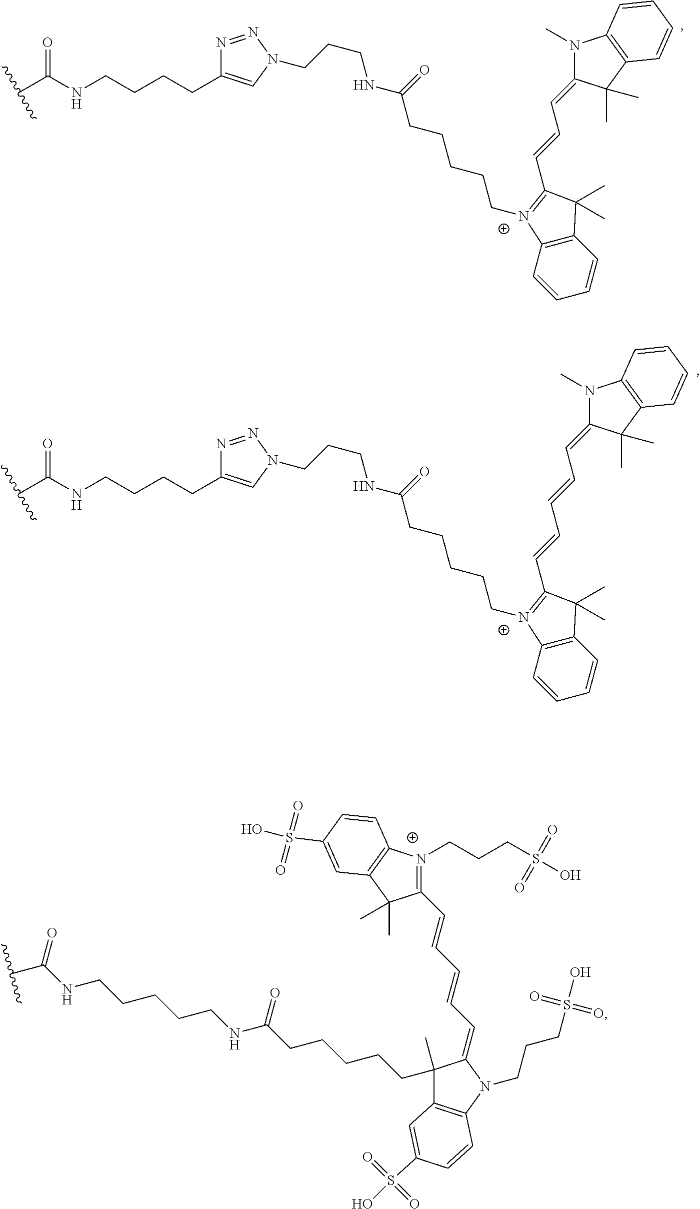

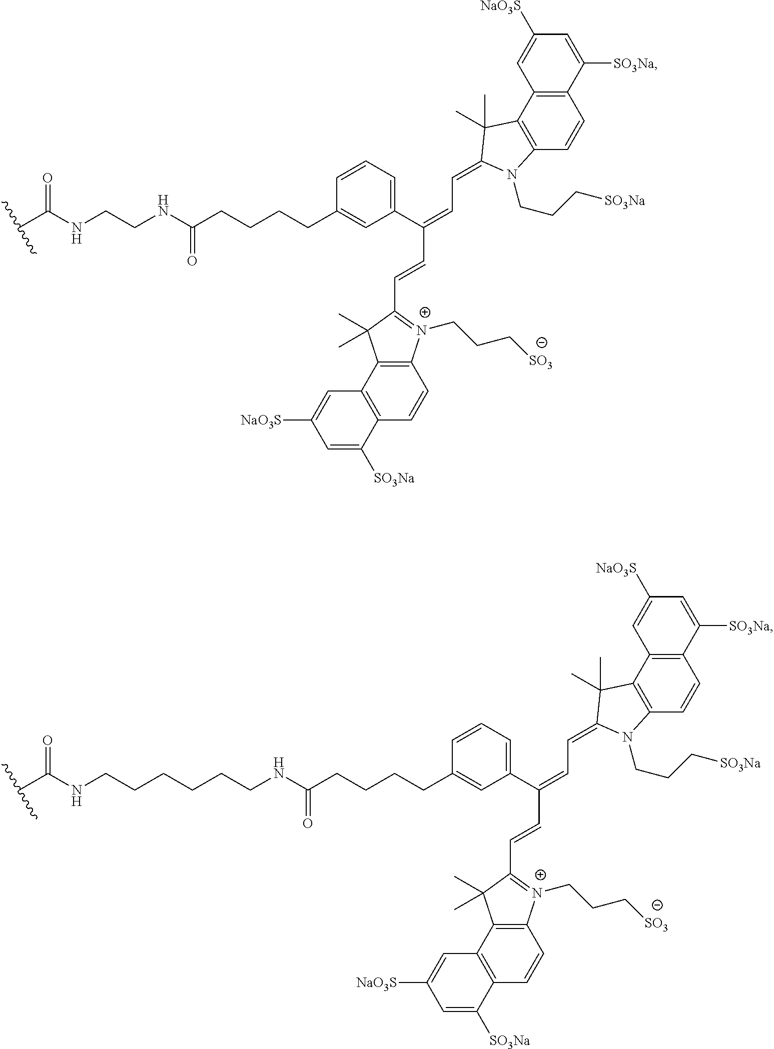

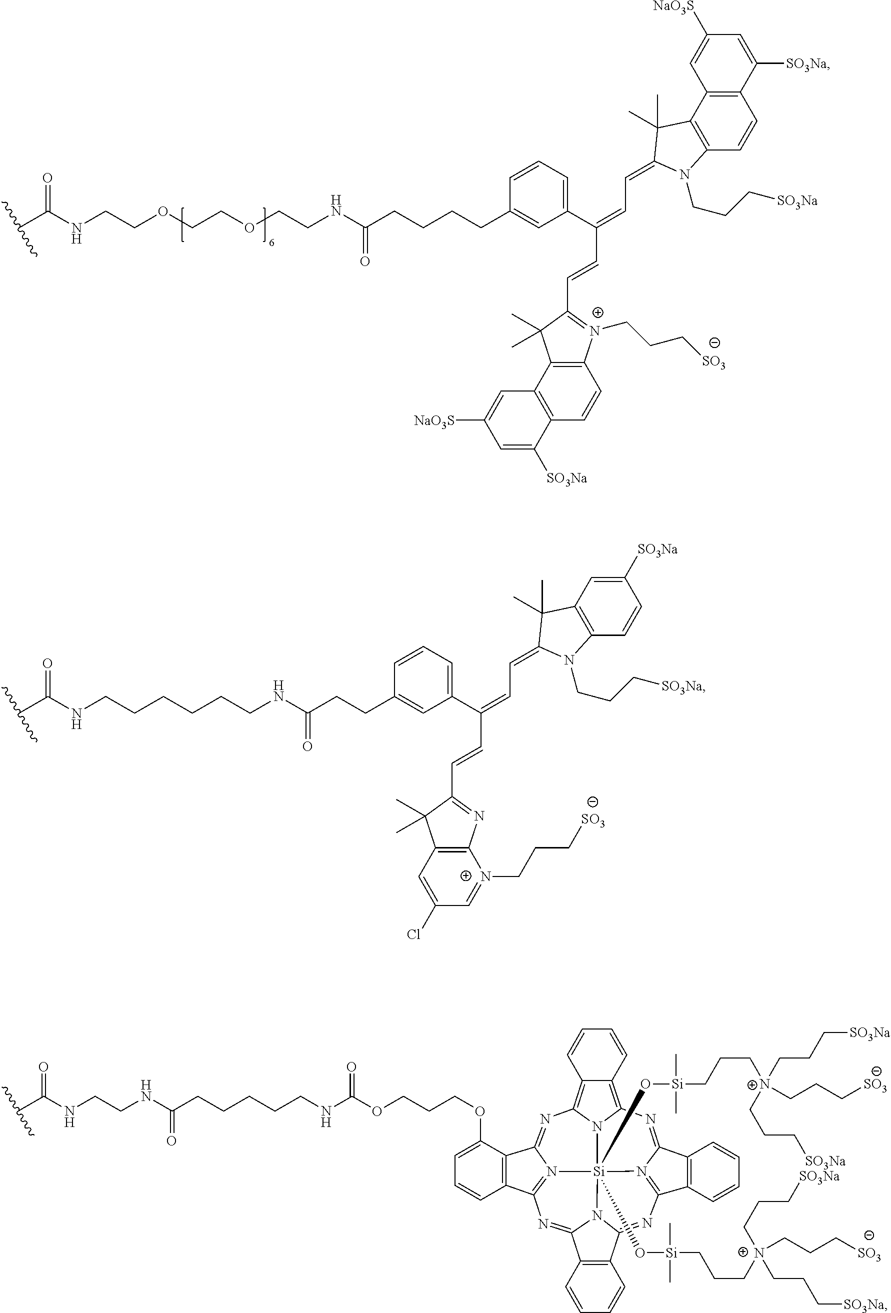

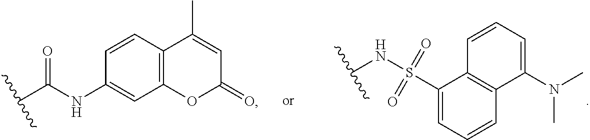 [SMILES: *C(=O)Nc1ccc2c(C)cc(=O)oc2c1 ; *NS(=O)(=O)c1cccc2c(N(C)C)cccc12]

69. The compound of any one of claims 15-52, wherein:
Q is a chemiluminescent moiety.

70. The compound of any one of claims 15-52, wherein:
Q is a chemiluminescent moiety that generates light or a colored product upon treatment with peroxide or a peroxidase.

71. The compound of any one of claim 15-52 or 69, wherein:
Q is luminol, isoluminol, N-(4-aminobutyl)-N-ethyl isoluminol (ABEI), N-(4-aminobutyl)-N-methyl isoluminol (ABMI), 2,2′-azino-bis(3-ethylbenzothiazoline-6-sulphonic acid) (ABTS), 3,3′,5,5′-Tetramethylbenzidine (TMB), 3,3′-diaminobenzidine (DAB), o-phenylenediamine dihydrochloride (OPD), AmplexRed, AEC, or homovanillic acid.

72. The compound of any one of claim 15-52 or 69, wherein:
Q is a chemiluminescent moiety that generates light or a colored product upon treatment with horseradish peroxidase (HRP).

73. The compound of claim 69, wherein:
Q is 3,3′-diaminobenzidine (DAB), 3,3′,5,5′-tetramethylbenzidine (TMB), 2,2′-Azinobis [3-ethylbenzothiazoline-6-sulfonic acid] (ABTS), o-phenylenediamine or dihydrochloride (OPD).

74. The compound of any one of claims 15-52, wherein:
Q is a substrate for a luciferase enzyme.

75. The compound of claim 74, wherein:
Q is D-luciferin, or coelenterazine.

76. The compound of any one of claims 15-52, wherein:
Q is a chemiluminescent moiety that generates light or a colored product upon treatment with alkaline phosphatase (AP).

77. The compound of claim 76, wherein:
Q is nitro blue tetrazolium chloride (NBT), 5-bromo-4-chloro-3-indolyl phosphate (BCIP), or p-Nitrophenyl Phosphate (PNPP).

78. The compound of any one of claims 15-52, wherein:
Q is a chemiluminescent moiety that generates light or a colored product upon treatment with glucose oxidase.

79. The compound of claim 78, wherein:
Q is nitro blue tetrazolium chloride (NBT).

80. The compound of any one of claims 15-52, wherein:
Q is a chemiluminescent moiety that generates light or a colored product upon treatment with β-galactosidase.

81. The compound of claim 80, wherein:
Q is 5-bromo-4-chloro-3-indoyl-β-D-galactopyranoside (BCIG or X-Gal)

82. The compound of any one of claims 15-52, wherein:
Q is absent and the compound of Formula (I) comprises a radioactive or an isotopic variant of any atom in the compound of Formula (I).

83. The compound of any one of claims 15-82, wherein:
the compound of Formula (I) comprises a radioactive or an isotopic variant of any atom in the compound of Formula (I) and is suitable for use in positron emission tomography (PET) analysis.

84. The compound of any one of claims 15-83, wherein:
Q is absent and the compound of Formula (I) comprises one or more atoms selected from tritium (³H), fluorine-18 (¹⁸F), carbon-11 (¹¹C), carbon-14 (¹⁴C), nitrogen-13 (¹³N), oxygen-15 (¹⁵O), or sulfur-35 (³⁵S).

85. The compound of any one of claims 15-52, wherein:
-L-Q is

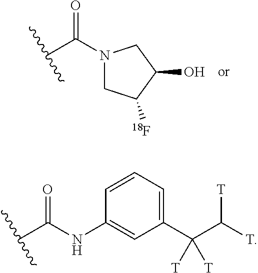

86. The compound of any one of claims 15-52, wherein:
Q comprises a chelated radioactive isotope.

87. The compound of any one of claims 15-52, wherein:
Q comprises a chelated radioactive isotope that is suitable for positron emission tomography (PET) analysis.

88. The compound of any one of claims 15-52, wherein:
Q comprises a chelated radioactive isotope, wherein Q is a diethylenetriaminepentaacetic acid (DTPA) chelate, 1,4,7,10-tetraazacyclododecane-1,4,7,10-tetraacetic acid (DOTA) chelate, or 1,4,7-triazacyclononane-1,4,7-trisacetic acid (NOTA) chelate or 1,4,7,10-tetraazacyclododecane-1,4,7,10-tetramethyl-1,4,7,10-tetraacetic acid (DOTMA) chelate or a radioactive isotope.

89. The compound of claim 88, wherein:
L is

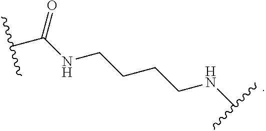 [SMILES: *NCCCCNC(*)=O]

90. The compound of any one of claims 86-89, wherein:
Q is 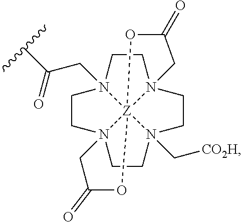
where Z is a radioactive isotope.

91. The compound of any one of claims 86-90, wherein:
Q comprises a chelated radioactive isotope that is copper-64 (⁶⁴Cu), gallium-68 (⁶⁸Ga), or technetium-99m (^{99m}Tc).

92. The compound of any one of claims 86-91, wherein:
-L-Q is

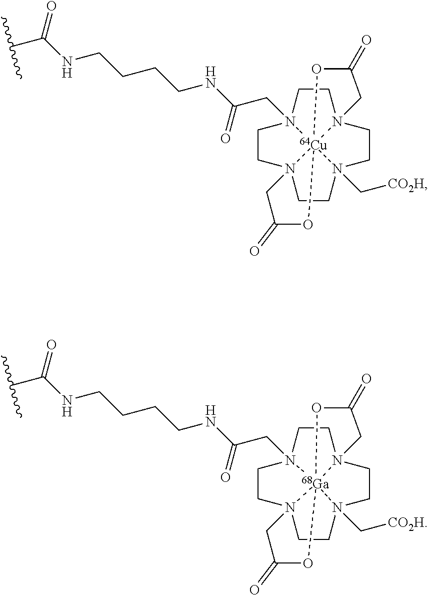

93. The compound of any one of claims 15-52, wherein:
Q is a magnetic resonance imaging (MRI) moiety.

94. The compound of any one of claims 15-52, wherein:
Q comprises a chelate of an atom that is suitable for magnetic resonance imaging (MRI).

95. The compound of any one of claims 15-52, wherein:
Q comprises a chelate of an atom that is suitable for magnetic resonance imaging (MRI) where the chelate is a diethylenetriaminepentaacetic acid (DTPA) chelate, 1,4,7,10-tetraazacyclododecane-1,4,7,10-tetraacetic acid (DOTA) chelate, 1,4,7-triazacyclononane-1,4,7-trisacetic acid (NOTA) chelate, or 1,4,7,10-tetraazacyclododecane-1,4,7,10-tetramethyl-1,4,7,10-tetraacetic acid (DOTMA) chelate.

96. The compound of any one of claims 15-52, wherein:
Q comprises a chelate of copper, gallium, thulium, europium, gadolinium, or manganese.

97. The compound of any one of claims 15-52, wherein:
Q comprises a chelate of gadolinium that is selected from gadoterate, gadodiamide, gadobenate, gadopentetate, gadoteridol, gadoversetamide, gadoxetate, gadobutrol, and gadofosveset.

98. The compound of any one of claims 15-52, wherein:
-L-Q is

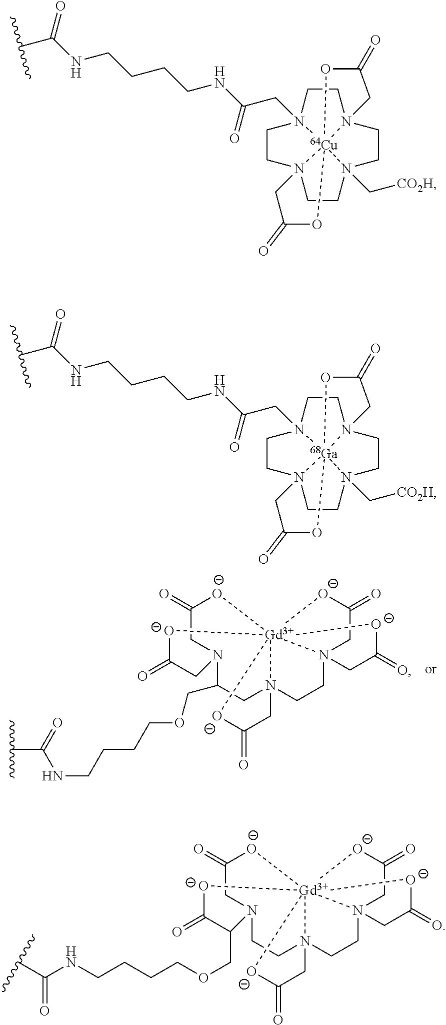

99. The compound of any one of claims 15-52, wherein:
Q is a solid support.

100. The compound of any one of claims 15-52, wherein:
Q is a solid support that is a nanoparticle, bead, or resin.

101. The compound of claim 100, wherein:
Q is a nanoparticle or bead comprising one or more metals selected from iron, cobalt, nickel, gadolium, chromium, manganese and gold.

102. The compound of any one of claims 100-101, wherein:
Q is a nanoparticle or bead that is magnetic or paramagnetic.

103. The compound of claim 102, wherein:
the magnetic moiety is a ferrite magnetic bead.

104. The compound of any one of claims 99-103, wherein:
L³ is a linker that is non-cleavable linker.

105. The compound of any one of claims 99-104, wherein:
-L-Q is 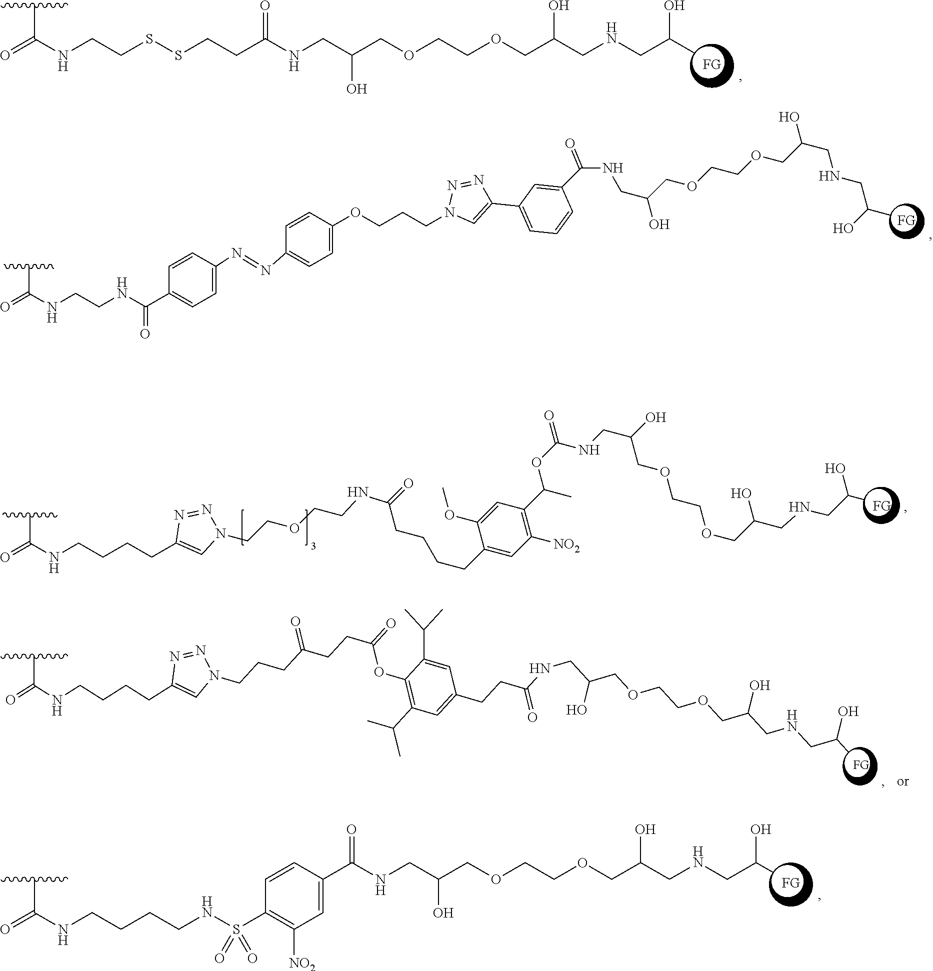
where FG is a ferrite bead.

106. A composition comprising LOXL2 labelled with a compound, wherein the compound is described in any one of claims 1-105.

107. A method for the quantification of LOXL2 in a biological sample:
(i) contacting a probe compound comprising:
(a) a small molecule lysyl oxidase like-2 (LOXL2) inhibitor (LOXL2i);
(b) a tag moiety (Q) for the detection, isolation, or detection and isolation of the small molecule LOXL2i bound to LOXL2; and
(c) an optional linker (L) that separates the LOXL2 inhibitor from the tag moiety;
with a biological sample comprising one or more proteins, wherein the contacting is effected under conditions and a duration sufficient for interaction between the probe compound and proteins in the biological sample to reach equilibrium; and
(ii) identifying and quantifying proteins labelled with the probe compound.

108. A method for the evaluating engagement of a lysyl oxidase like-2 (LOXL2) inhibitor (LOXL2i) with LOXL2 in a biological sample comprising:
(i) contacting a small molecule lysyl oxidase like-2 (LOXL2) inhibitor (LOXL2i) with a biological sample comprising one or more proteins, wherein the contacting is effected under conditions and a duration sufficient for interaction between the LOXL2i and proteins in the biological sample to reach equilibrium;
(ii) adding a probe compound comprising:
(a) a small molecule lysyl oxidase like-2 (LOXL2) inhibitor (LOXL2i);
(b) a tag moiety (Q) for the detection, isolation, or detection and isolation of the small molecule LOXL2i bound to LOXL2; and
(c) an optional linker (L) that separates the LOXL2 inhibitor from the tag moiety;
to the biological sample of step (i) under conditions and a duration sufficient for interaction between the probe compound and the proteins in the biological sample to reach equilibrium;
and
(iii) identifying and quantifying the proteins labelled with the probe compound.

109. A method for assessing interactions of a LOXL2 inhibitor (LOXL2i) with target proteins and non-target proteins, comprising:
(i) contacting a small molecule lysyl oxidase like-2 (LOXL2) inhibitor (LOXL2i) with a biological sample comprising one or more proteins, wherein contacting is effected under conditions and a duration sufficient for interaction between the LOXL2i and proteins in the biological sample to reach equilibrium;
(ii) adding a probe compound comprising:
(a) a small molecule lysyl oxidase like-2 (LOXL2) inhibitor (LOXL2i);
(b) a tag moiety (Q) for the detection, isolation, or detection and isolation of the small molecule LOXL2i bound to LOXL2; and
(c) an optional linker (L) that separates the LOXL2 inhibitor from the tag moiety;
to the biological sample of step (i) under conditions and a duration sufficient for interaction between the probe compound and proteins in the biological sample to reach equilibrium; and
(iii) identifying and quantifying proteins labelled with the probe compound.

110. The method of claim 109, wherein:
the method further comprises redesigning the LOXL2i in order to eliminate or alter the LOXL2 proteins captured with the probe compound when steps (i)-(iii) are repeated with the redesigned LOXL2i.

111. The method of any one of claims 107-110, wherein:
the biological sample is a cell lysate or tissue containing proteins.

112. The method of any one of claims 107-111, wherein:
the biological sample comprises LOXL2.

113. The method of any one of claims 107-112, wherein:
the probe compound is a compound described in any one of claims 1-105.

114. A method for in vivo imaging a tissue or organ that expresses LOXL2 in a mammal comprising:
(i) administering a probe compound comprising:
(a) a small molecule lysyl oxidase like-2 (LOXL2) inhibitor (LOXL2i);
(b) a tag moiety (Q) that is suitable for the detection of the small molecule LOXL2i bound to LOXL2 using positron emission tomography (PET) analysis or magnetic resonance imaging (MRI); and
(c) an optional linker (L) that separates the LOXL2i of (a) from the tag moiety of (b);
(ii) performing positron emission tomography (PET) analysis or magnetic resonance imaging (MRI) on the mammal.

115. A method for identifying tissues or organs in a mammal that overexpress LOXL2 comprising:
(i) administering to the mammal a probe compound comprising:
(a) a small molecule lysyl oxidase like-2 (LOXL2) inhibitor (LOXL2i);
(b) a tag moiety (Q) that is suitable for the detection of the small molecule LOXL2i bound to LOXL2 using positron emission tomography (PET) analysis or magnetic resonance imaging (MRI); and
(c) an optional linker (L) that separates the LOXL2i of (a) from the tag moiety of (b);
(ii) performing positron emission tomography (PET) analysis or magnetic resonance imaging (MRI) on the mammal.

116. A method for ex vivo imaging a tissue or organ that expresses LOXL2 comprising:
(i) incubating a tissue or organ with a probe compound comprising:
(a) a small molecule lysyl oxidase like-2 (LOXL2) inhibitor (LOXL2i); and
(b) a tag moiety (Q) that is suitable for the detection of the small molecule LOXL2i bound to LOXL2 using immunofluorence (IF), immunohistochemistry (IHC) or autoradiography
(ii) performing immunofluorescence (IF), immunohistochemistry (IHC) or autoradiography

117. The method of claim 114 or claim 115, wherein the mammal was diagnosed with fibrosis or cancer.

118. A kit comprising:
a probe compound as described in any one of claims 1-105; and
a container.

119. The kit of claim 118, further comprising instructions or information on the use of the probe compound.
